(19) United States
(12) Patent Application Publication
(10) Pub. No.: US 2010/0158992 A1
(43) Pub. Date: Jun. 24, 2010

(54) PIPERAZINE-SUBSTITUTED PYRIDAZINONE DERIVATIVES USEFUL AS GLUCAN SYNTHASE INHIBITORS

(75) Inventors: Todd A. Black, New Providence, NJ (US); Paul M. McNicholas, Westfield, NJ (US); Scott S. Walker, Basking Ridge, NJ (US); Yiming Xu, Princeton, NJ (US); Pauline C. Ting, New Providence, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation, Kenilworth, NJ (US)

(21) Appl. No.: 12/528,944

(22) PCT Filed: Mar. 12, 2008
(86) PCT No.: PCT/US08/03292

§ 371 Date: Dec. 8, 2009

Related U.S. Application Data
(60) Provisional application No. 60/918,182, filed on Mar. 15, 2007.

(51) Int. Cl. A61K 31/501; C07D 403/04; C07D 413/14; A61K 31/5377; A61K 9/127; A61K 31/7048; A61K 31/69; A61P 31/10

(52) U.S. Cl. 424/450; 544/238; 544/121; 514/252.02; 514/236.5; 514/31; 514/64

(57) ABSTRACT

There is disclosed a method for treating or preventing fungal infections comprising the administration of at least one glucan synthase inhibitor of a formula as described above in the specification or a pharmaceutically acceptable salt thereof; also claimed are methods of preparing pharmaceutical compositions comprising a compound as described above in the specification and a carrier, method of treating or preventing fungal infections comprising administration of combinations of glucan synthase inhibitor of a formula as described above in the specification and other antifungal agents, and method of treating or preventing fungal infections comprising administration of pharmaceutical compositions prepared according to a method described above in the specification, and a method of preparing a kit in a single package of the above described pharmaceutical composition and other antifungal agents.

### FIELD OF THE INVENTION

This invention relates to a method of treating or preventing fungal infections with a piperazine-substituted pyridazinone derivative glucan synthase inhibitor.

### BACKGROUND OF THE INVENTION

The enzymes involved in fungal cell wall biogenesis are attractive targets for antifungal intervention. These enzymes are unique to fungi and therefore provide highly selective antifungal targets. Furthermore, disruption of cell wall synthesis generally leads to a fungicidal response due to cell lysis induced by the osmotic instability of cells lacking an intact wall. Major structural components of fungal cell walls are β(1,3)-linked D-glucan polymers. These polymers are generated by β(1,3)-D-glucan synthase, an integral membrane protein complex that is required for fungal cell viability. Compounds described as inhibitors of glucan synthase have been described previously. Reference is made to Károlyházy, László et al. Arzneim.-Forsch./Drug Res. 2003, Vol. 53, No. 10, 738-743, which discloses 3-(2H)-pyridazinones of the formula:

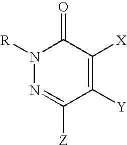

where the various elements are defined therein. An illustrative compound of that series is:

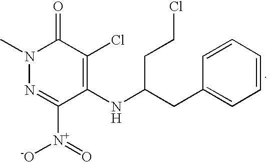

Reference is made to Kondoh, Osamu et al., Biol. Pharm. Bull. 2005, 28, 2138-2141, which discloses piperazine propanol derivatives. An illustrative compound of that series is:

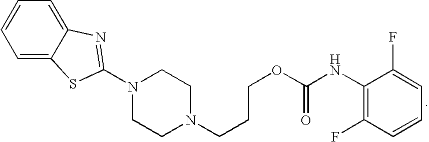

Reference is made to Brayman, Timothy et al., Antimicrobial Agents and Chemotherapy 2003 Vol. 47, No. 10, 3305-3310, which discloses the use of several compounds identified as glucan synthase inhibitors to test an assay for antifungal activity of glucan synthase inhibitors that uses germ tube formation in Candida albicans as an end point. An illustrative compound used to test the assay is:

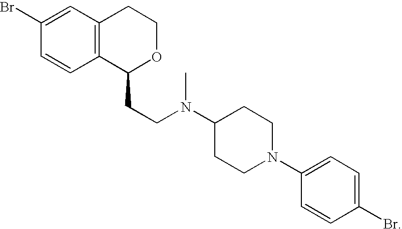

Reference is made to Gomez, Gil et al., ES 540813 1985, which discloses 1,2-diazin-3(2H)-ones as compounds of pharmaceutical interest useful as antihypertensives, β-adrenergic blockers, antiulcer agents or as intermediates thereof. An illustrative compound of that series is:

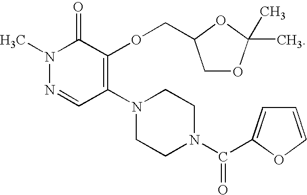

Reference is made to Pauline C. Ting and Scott S. Walker, “New Agents to Treat Life-Threatening Fungal Infections” in Current Topics in Medicinal Chemistry, 2007, which discloses Antifungals that are inhibitors of glucan synthase. These Antifungals include cyclic hexapeptides that are either approved for antifungal chemotherapy (caspofungin, micafungin anidulafungin) or under clinical development (aminocandin).

### SUMMARY OF THE INVENTION

This invention relates to a method of treating or preventing fungal infections in animals comprising administering to an animal, plant, or inanimate surface in need of such treatment an effective amount of one or more compounds of the formula:

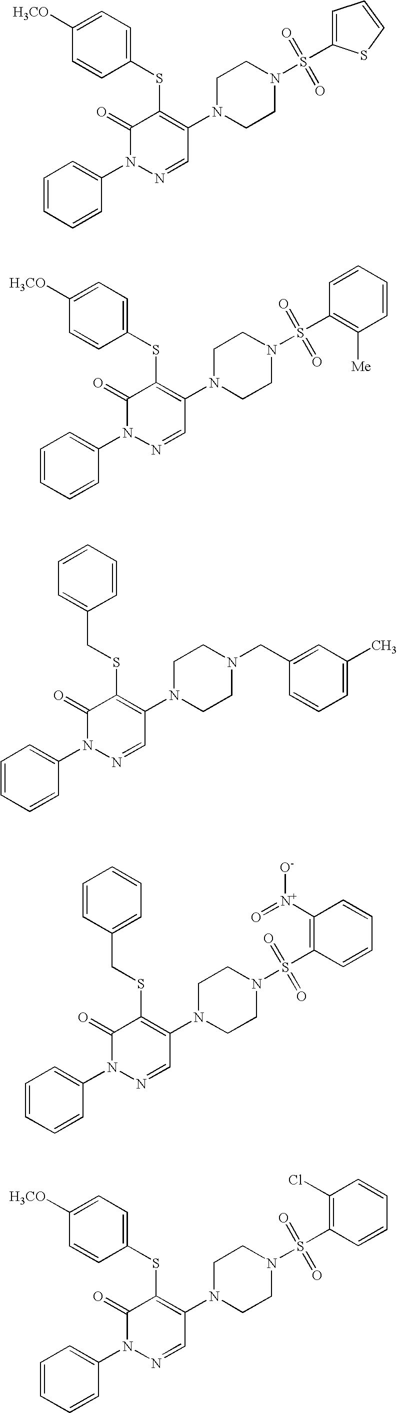

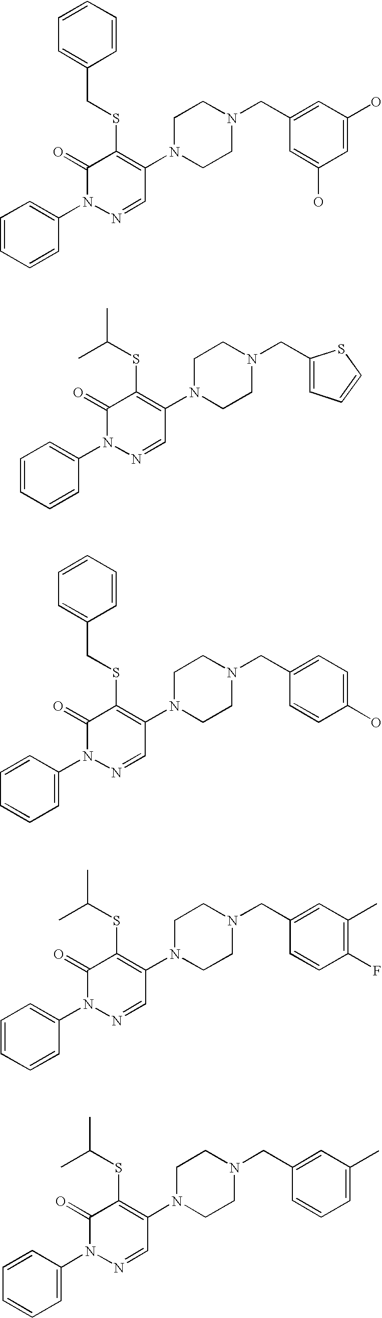

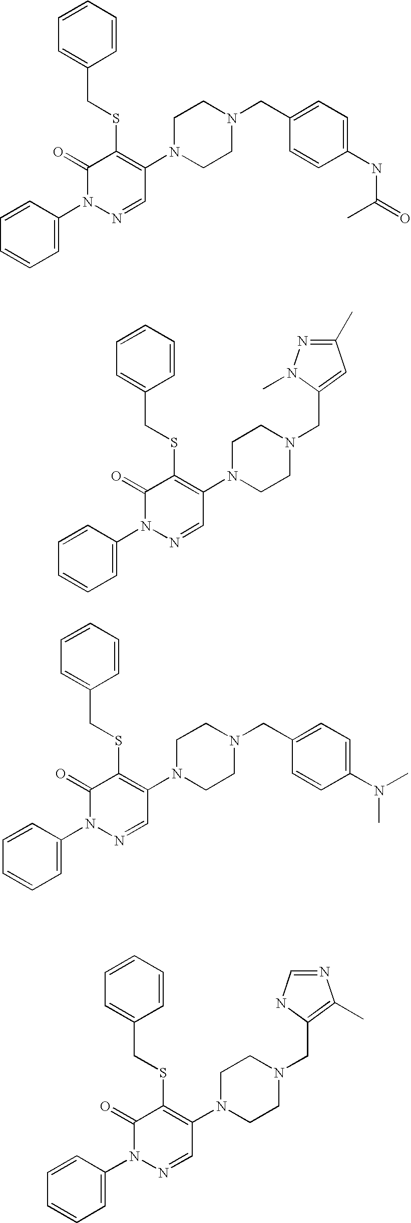

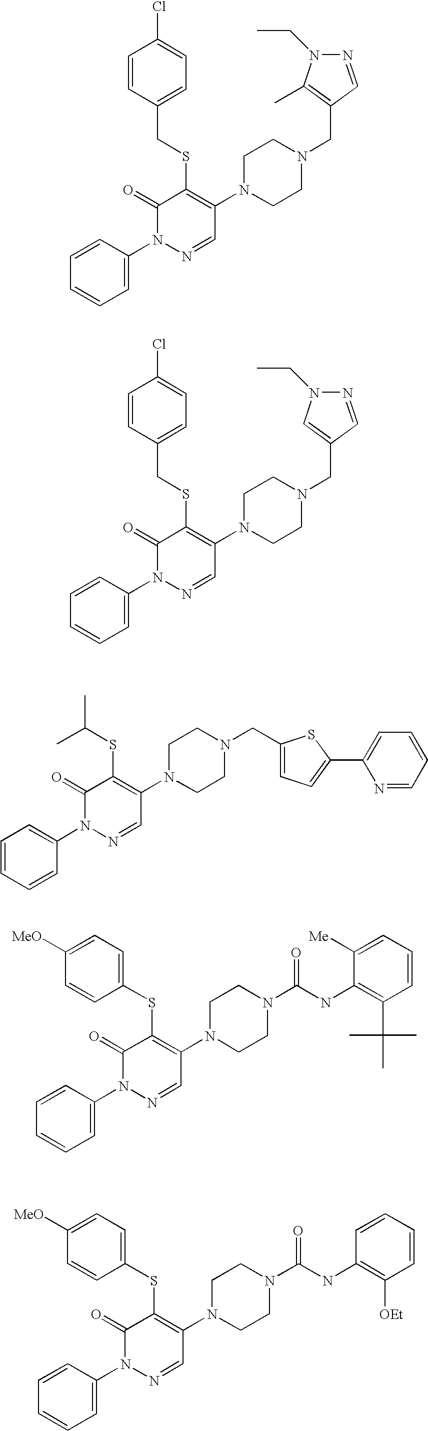

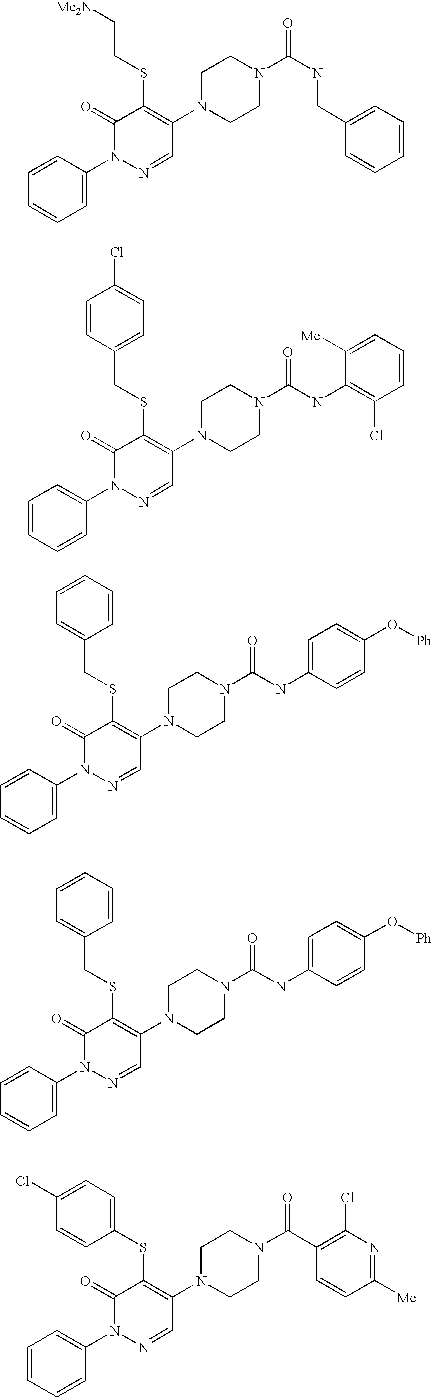

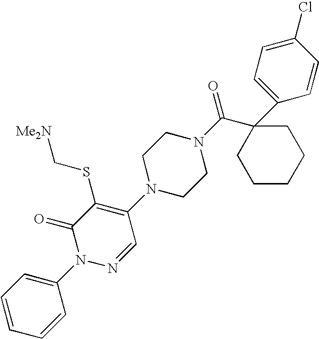

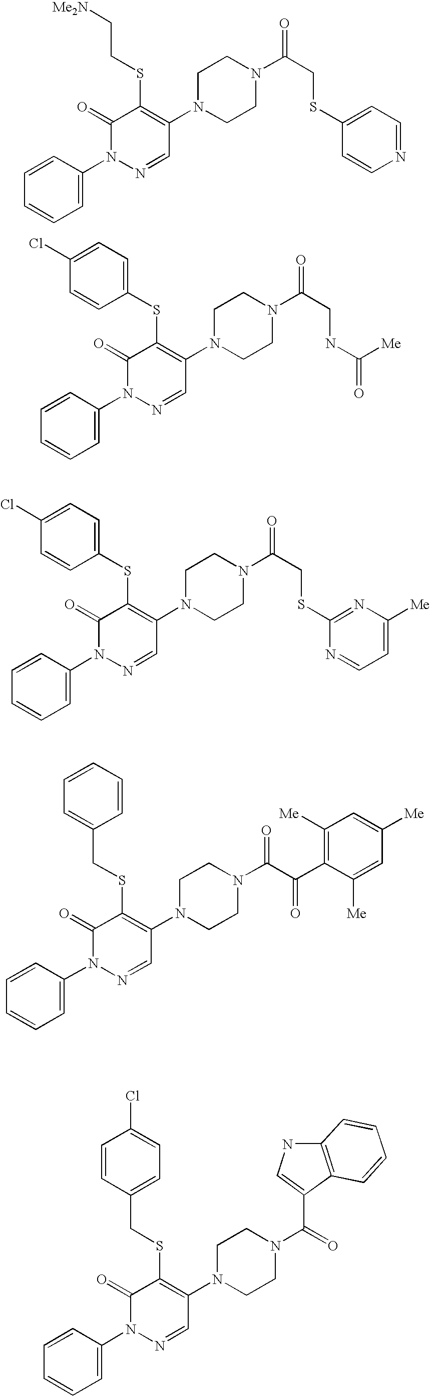

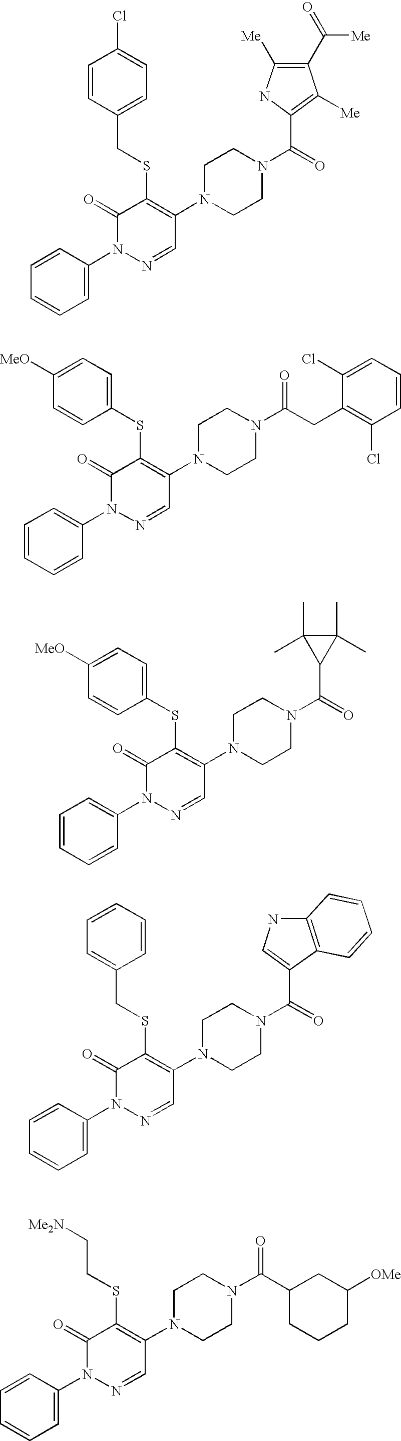

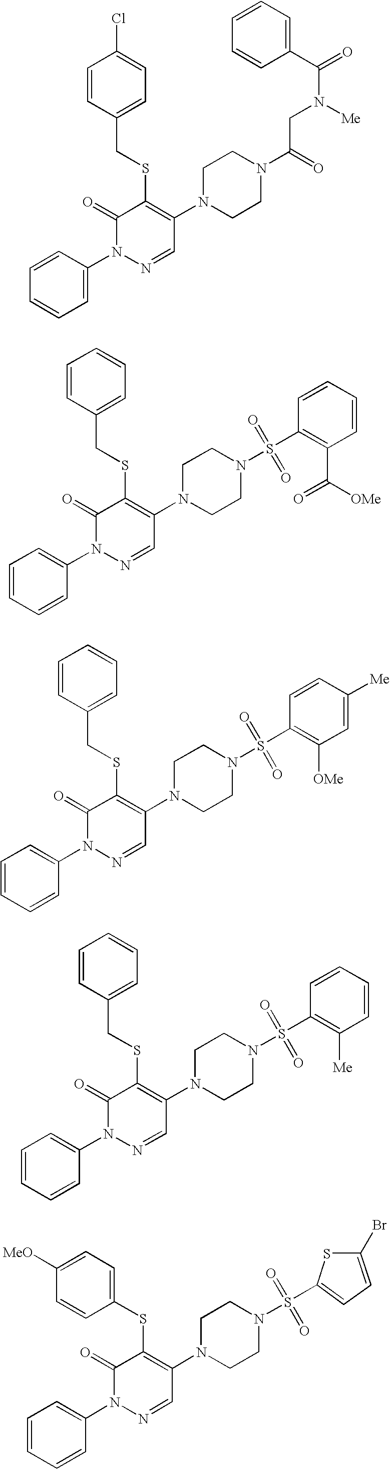

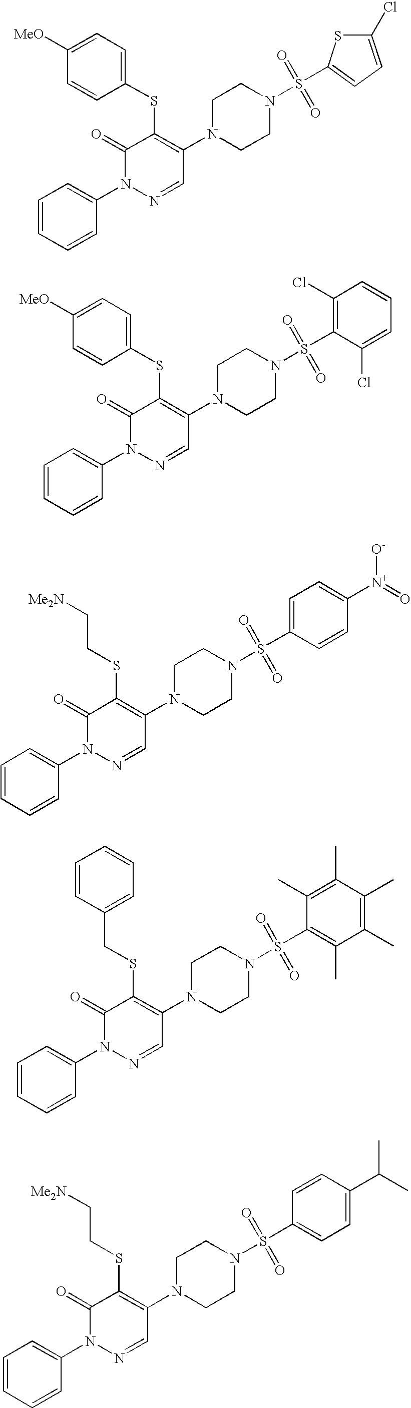

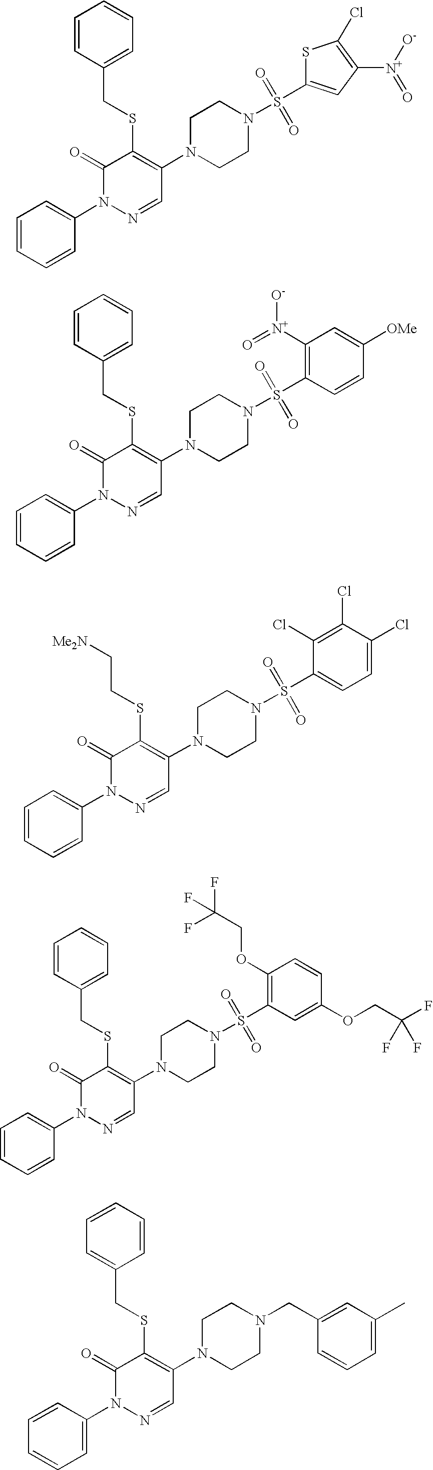

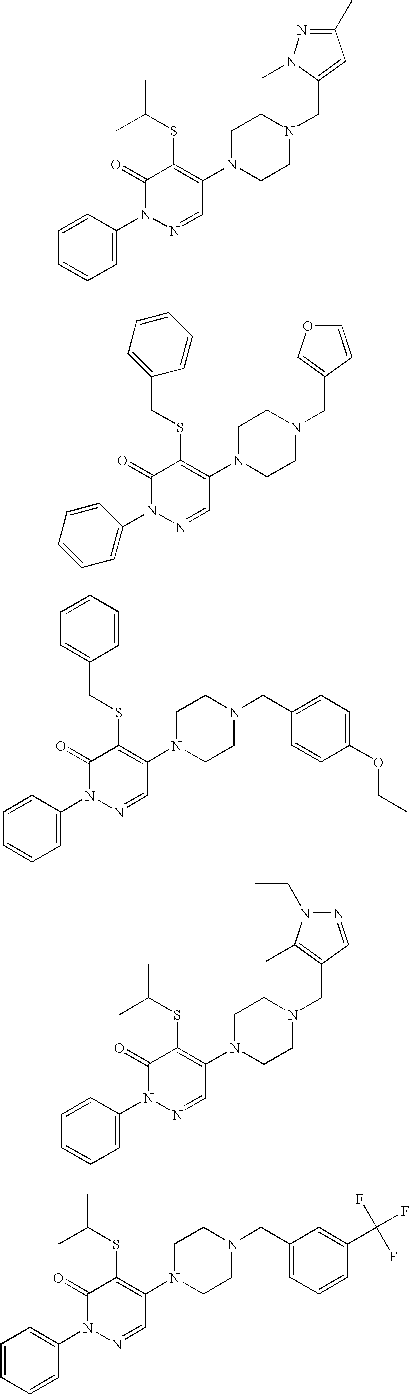

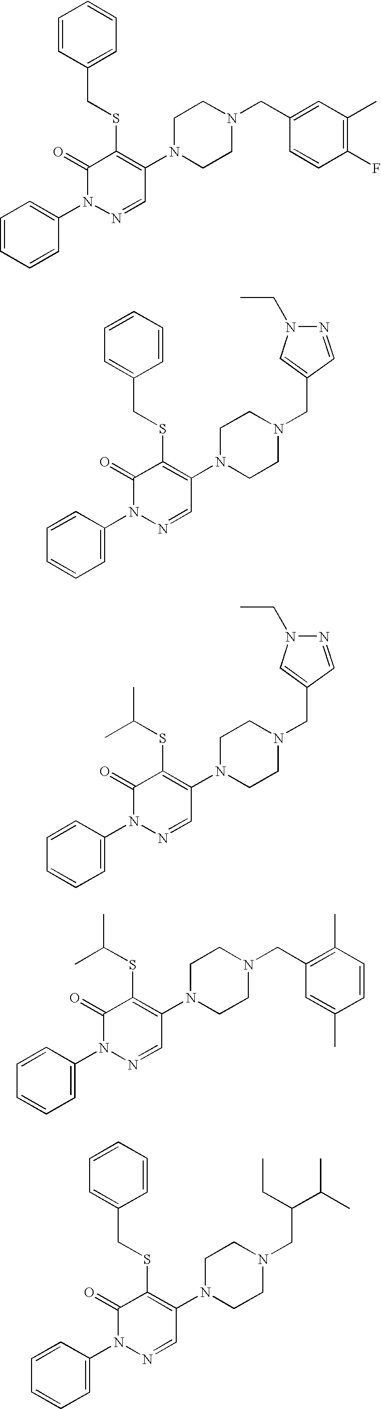

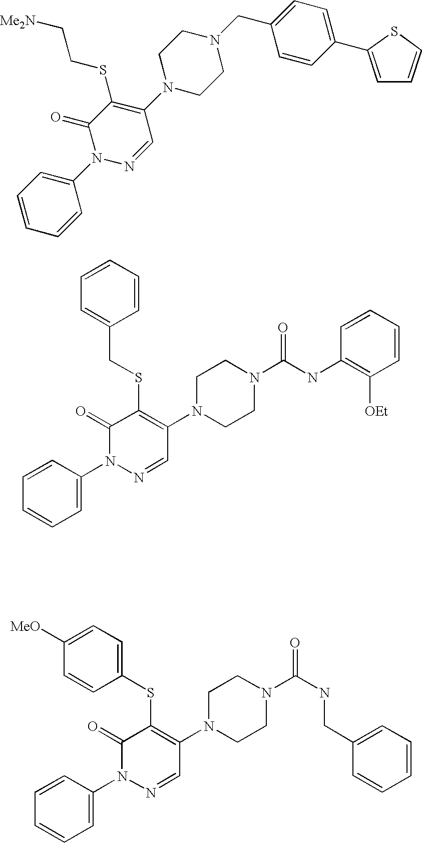

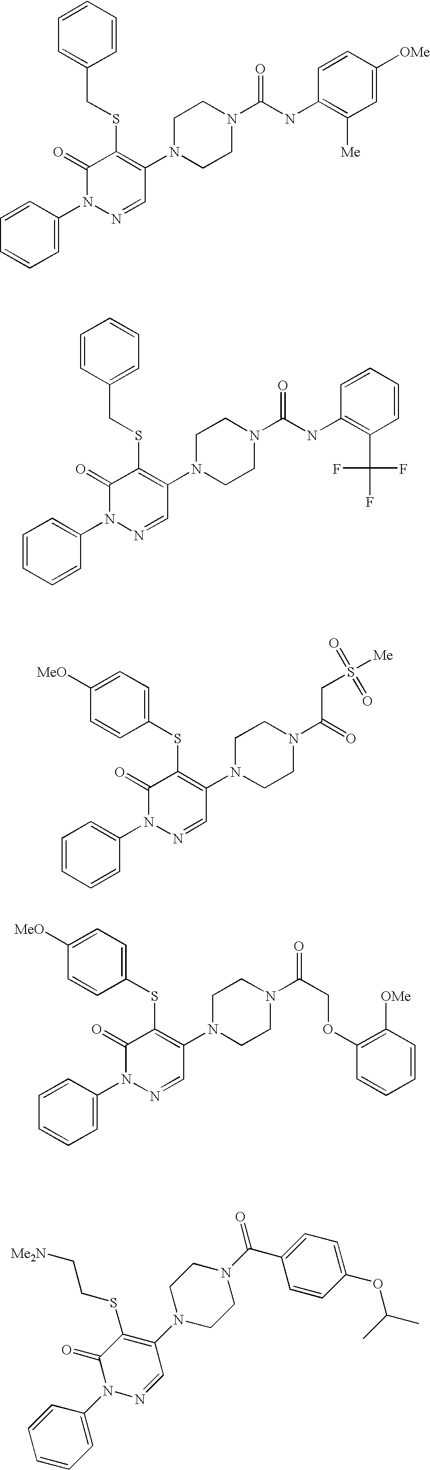

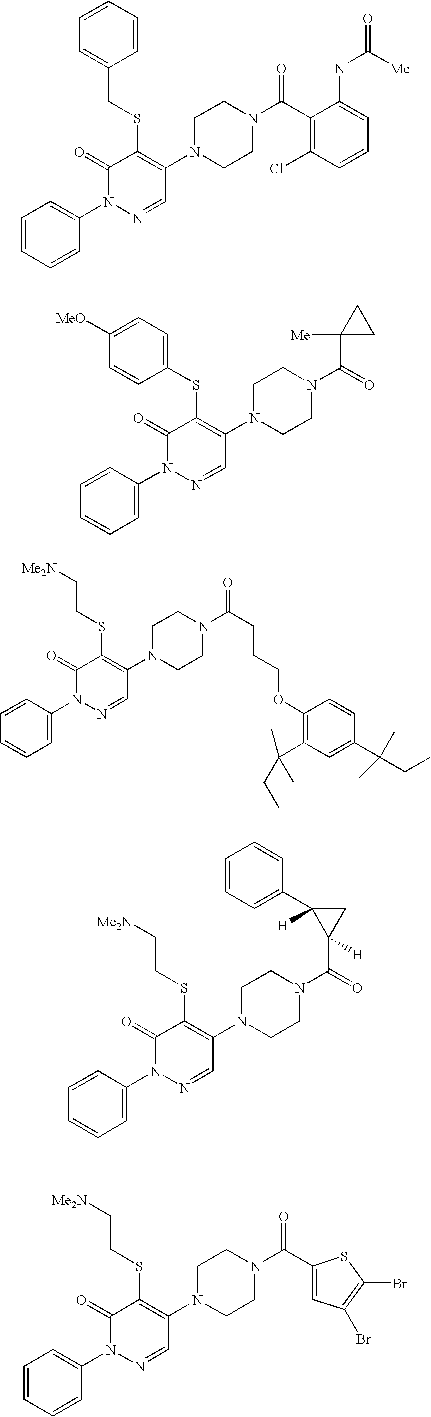

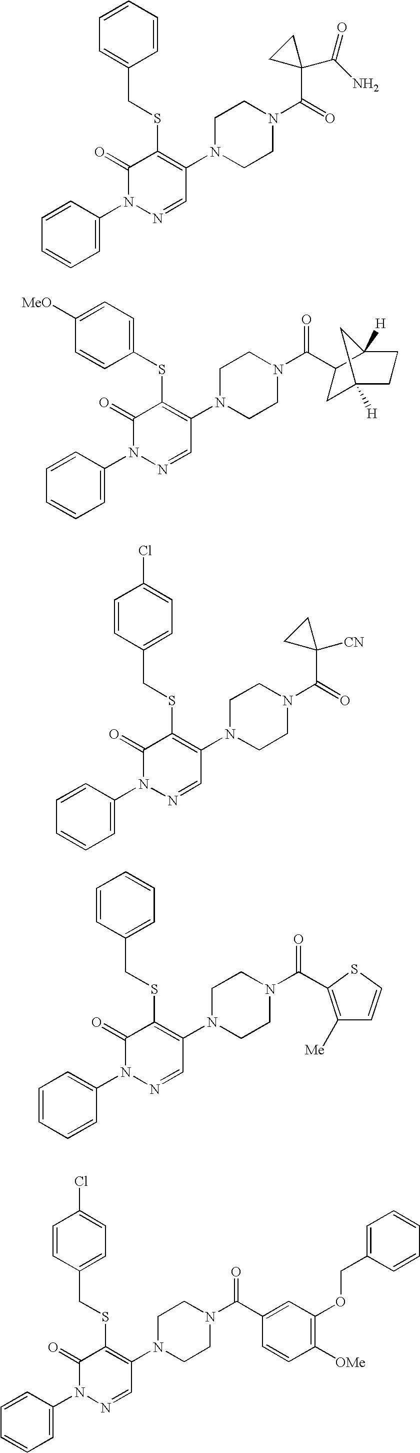

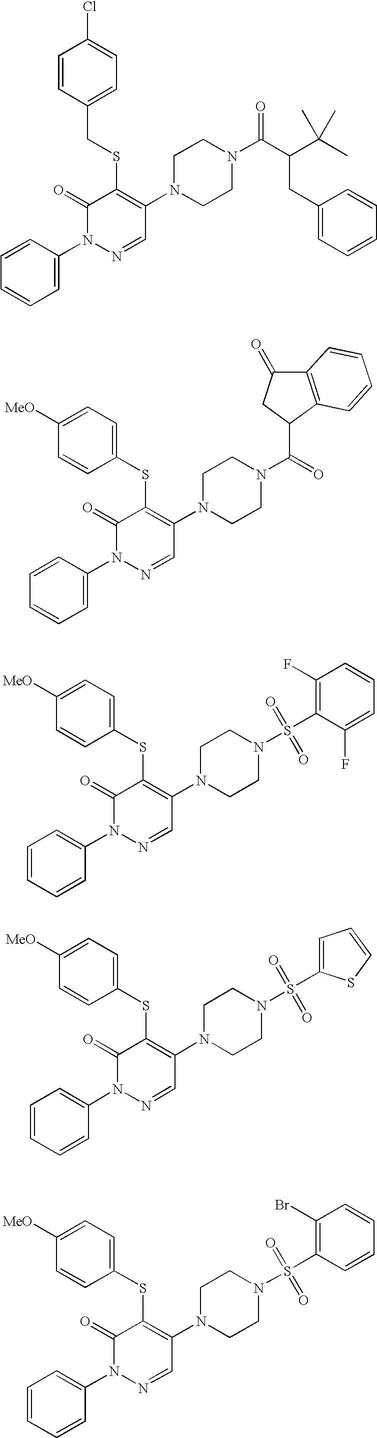

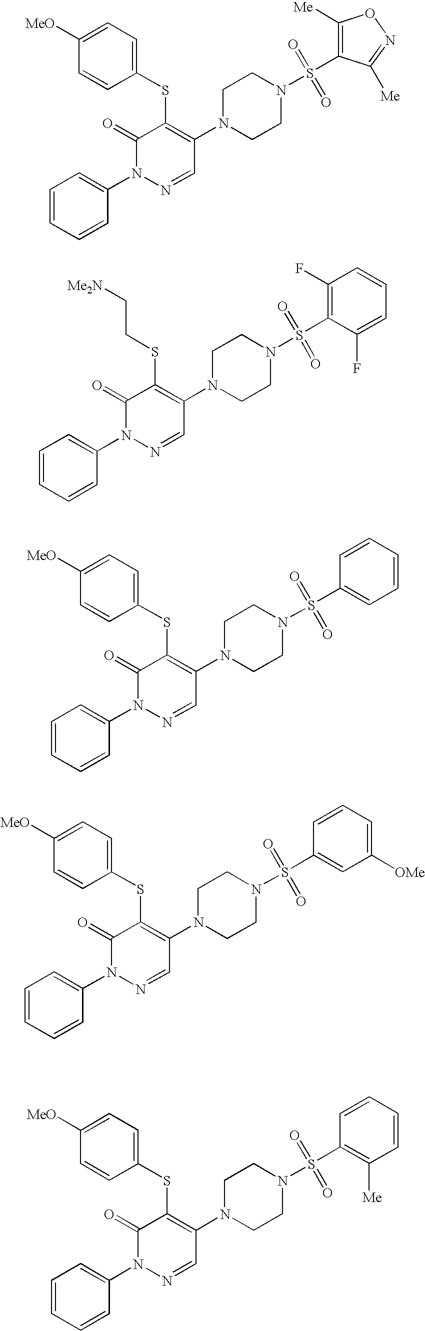

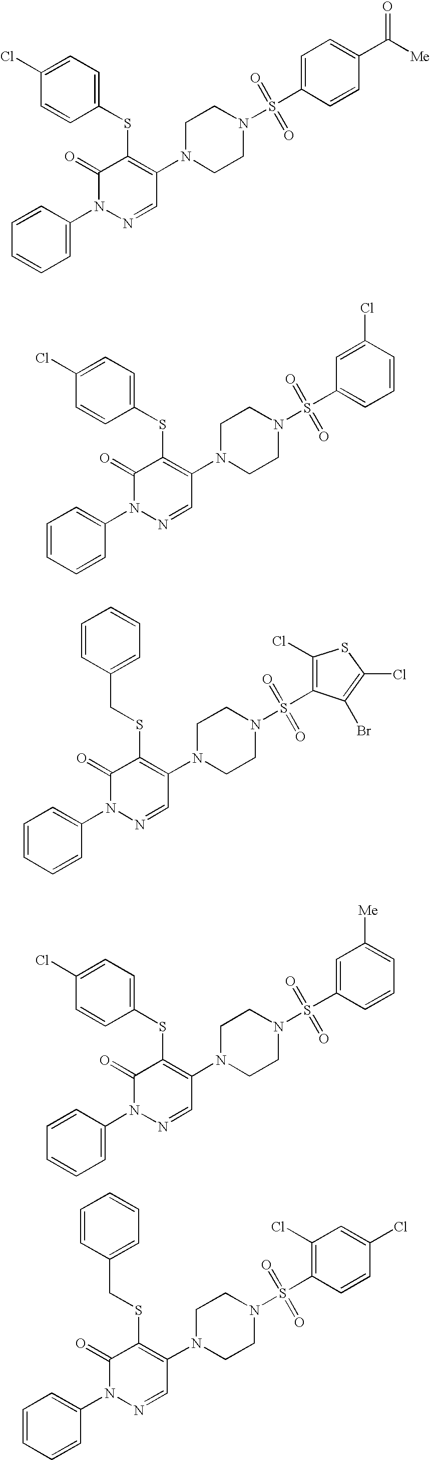

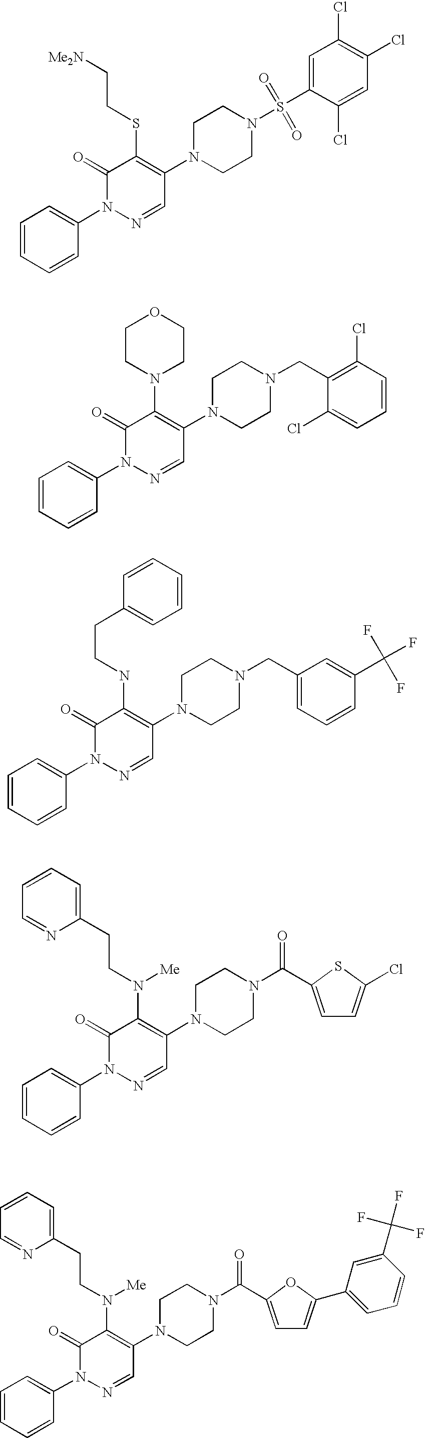

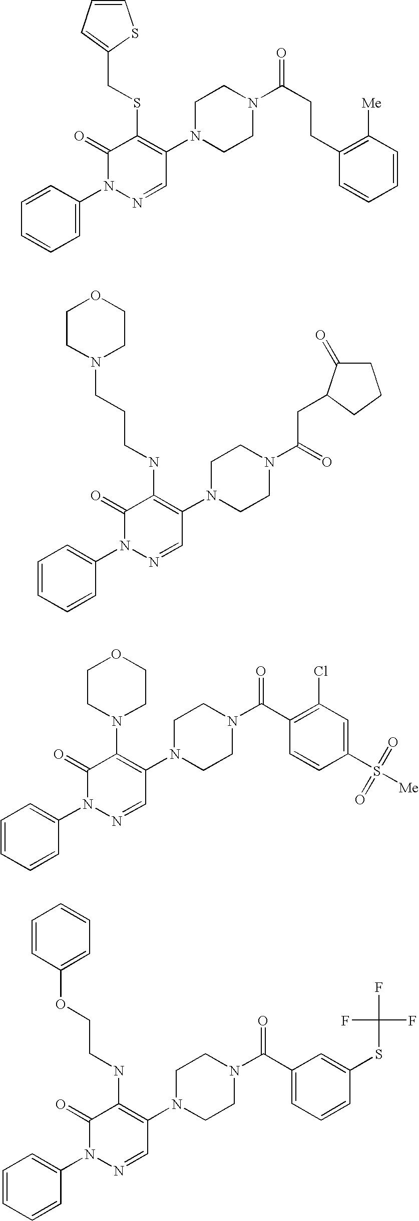

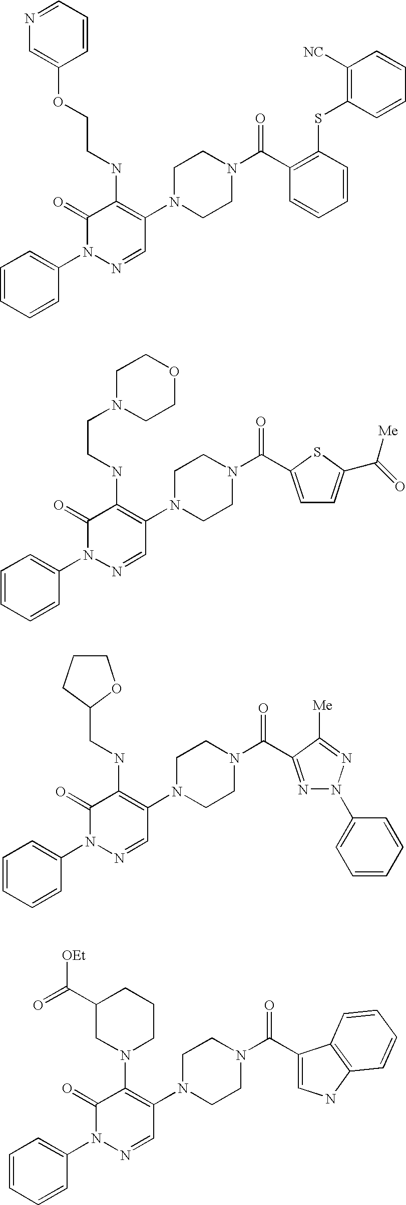

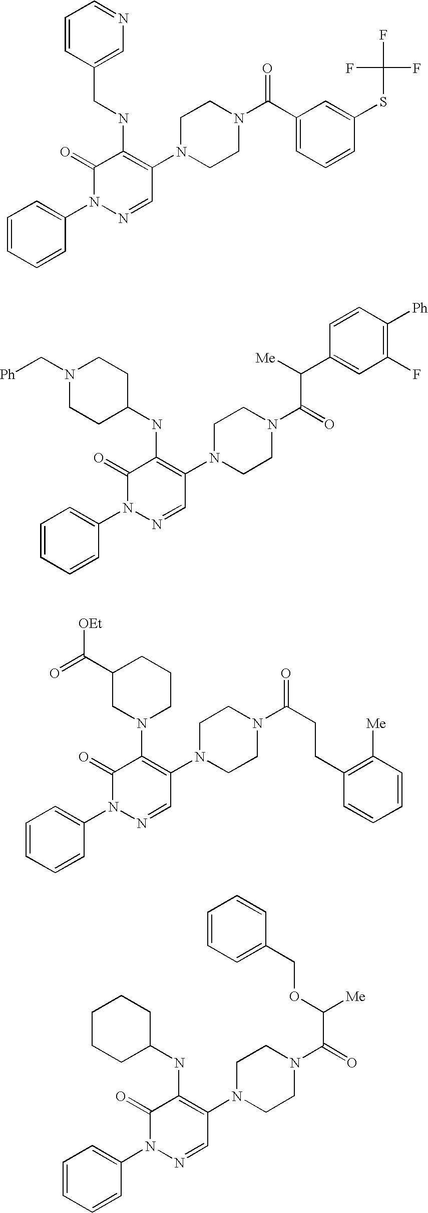

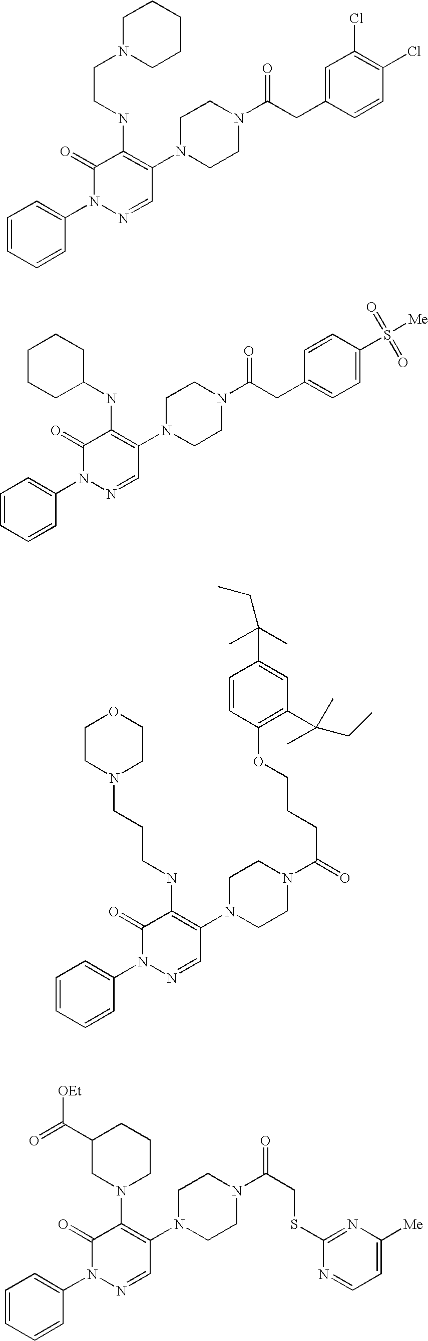

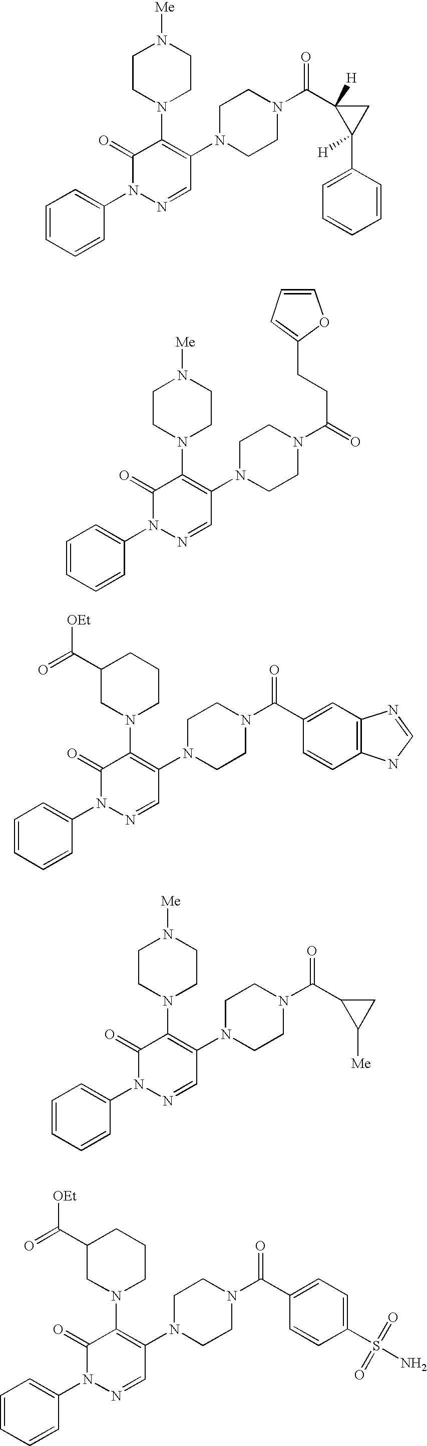

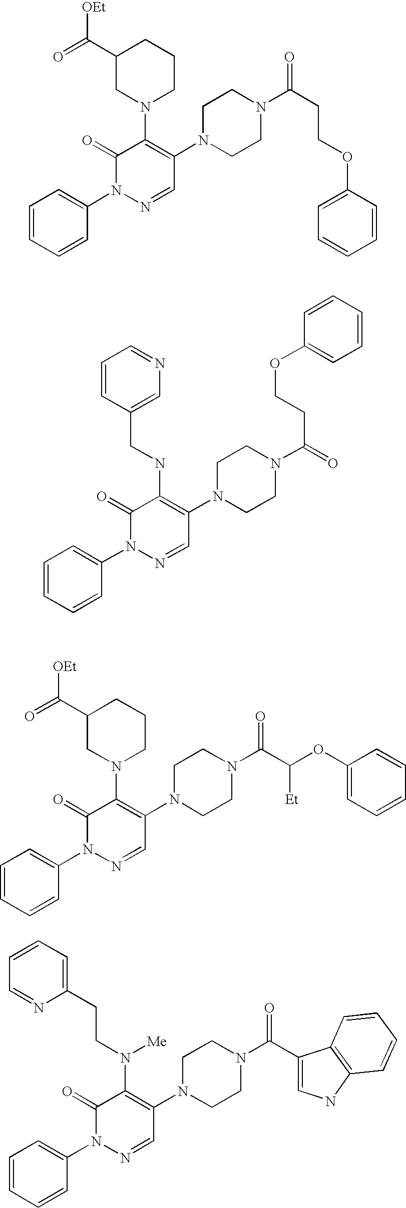

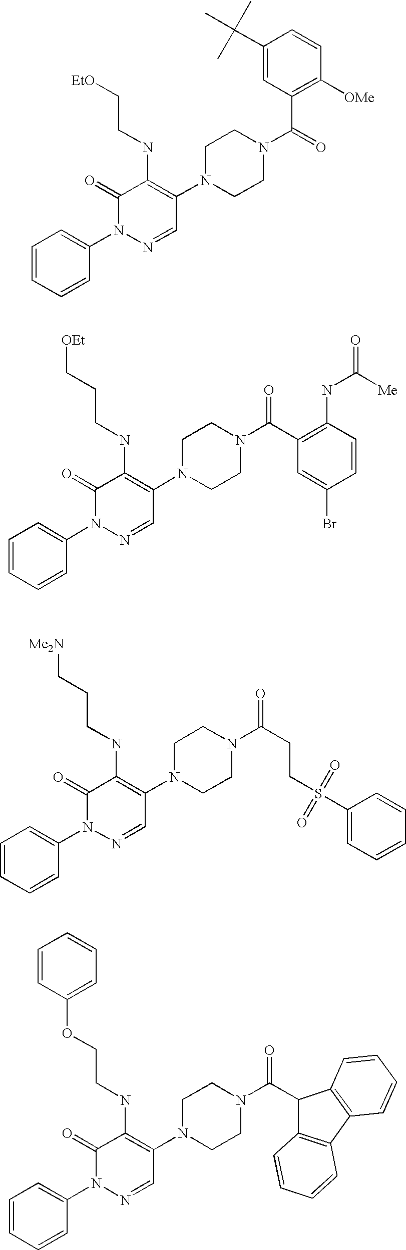

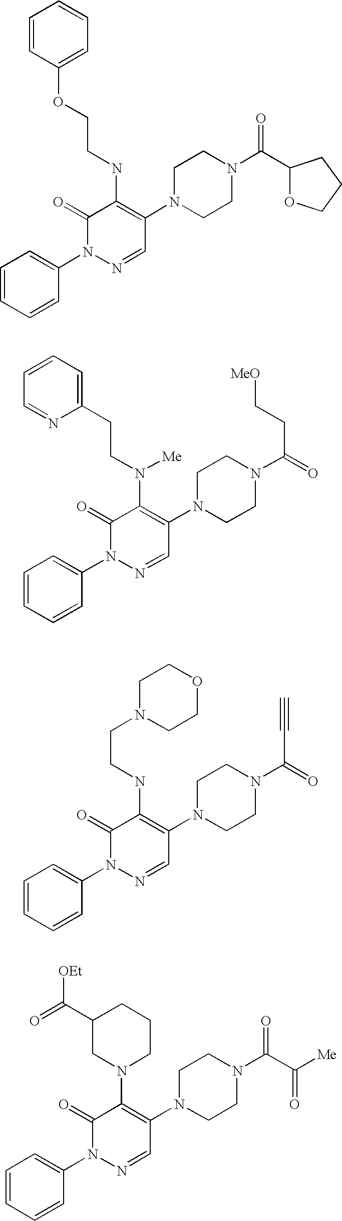

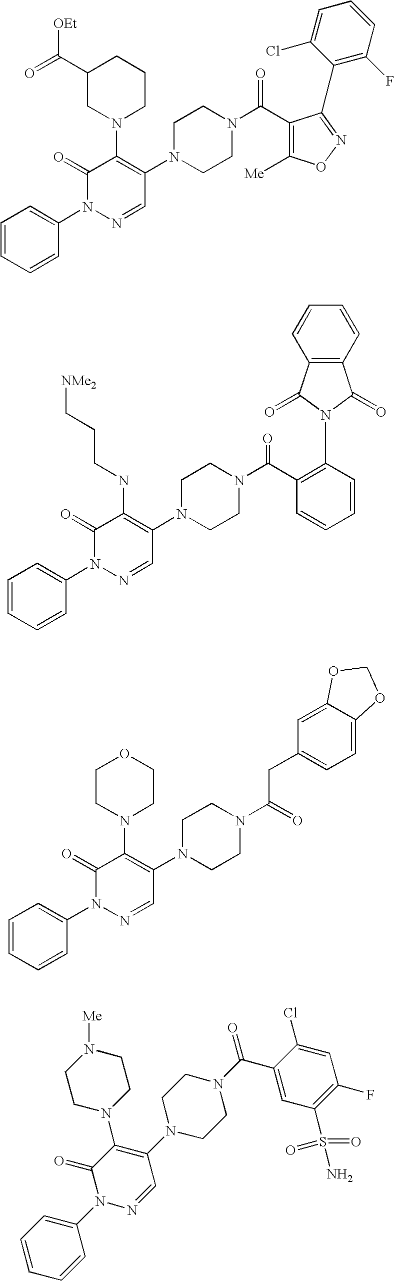

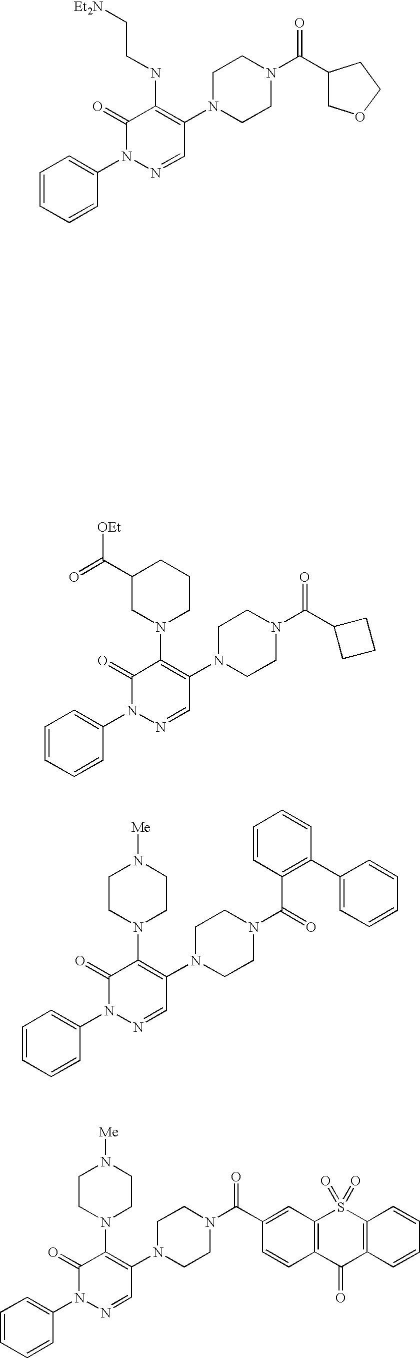

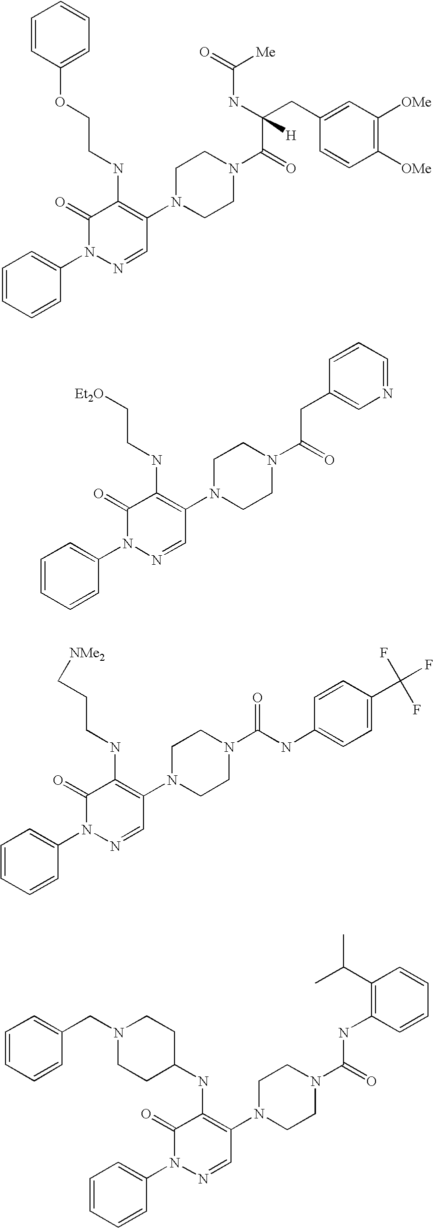

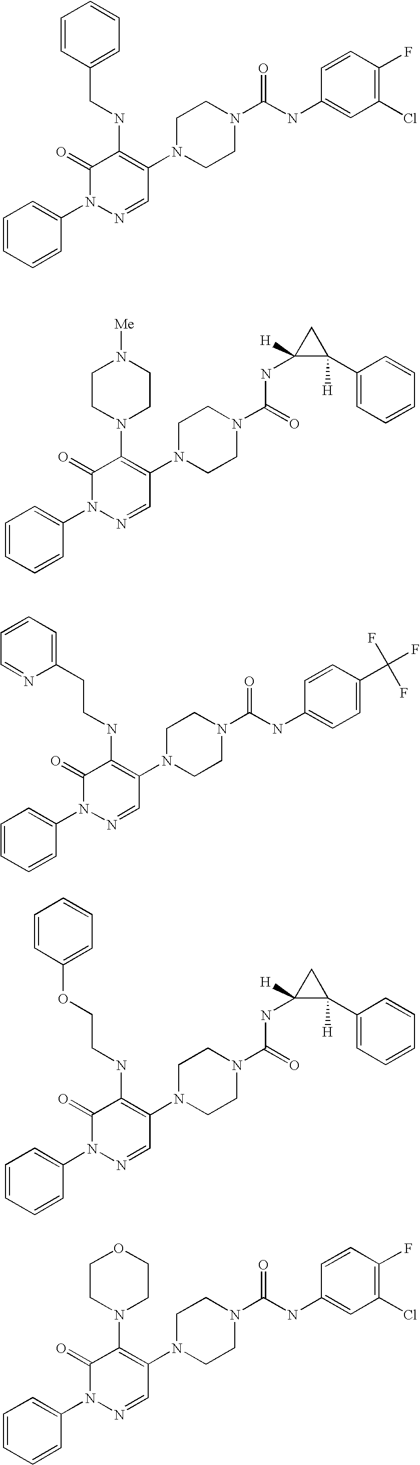

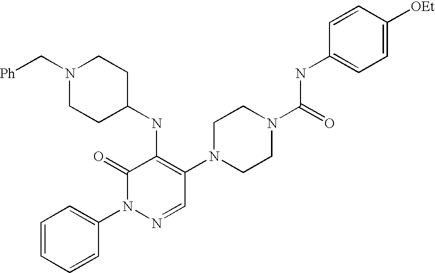

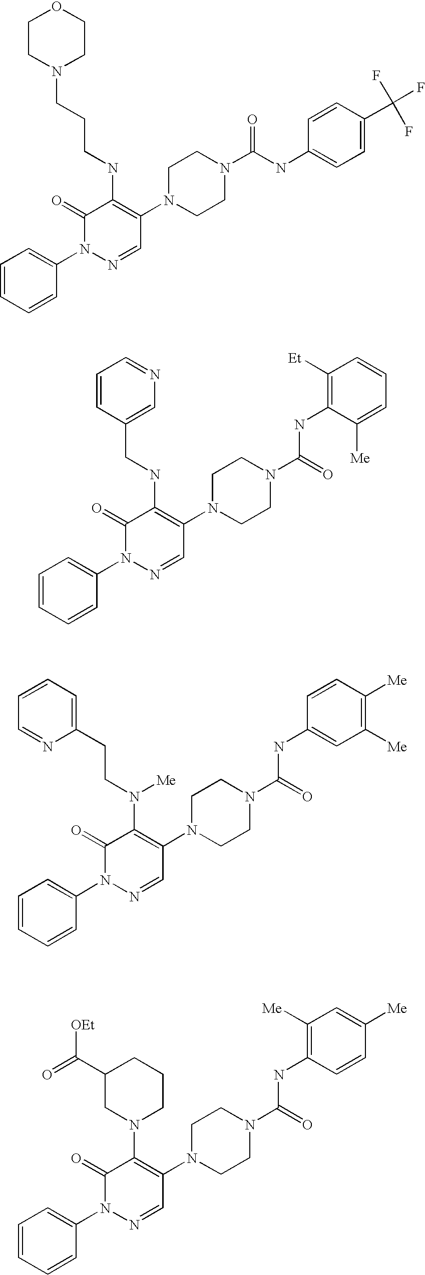

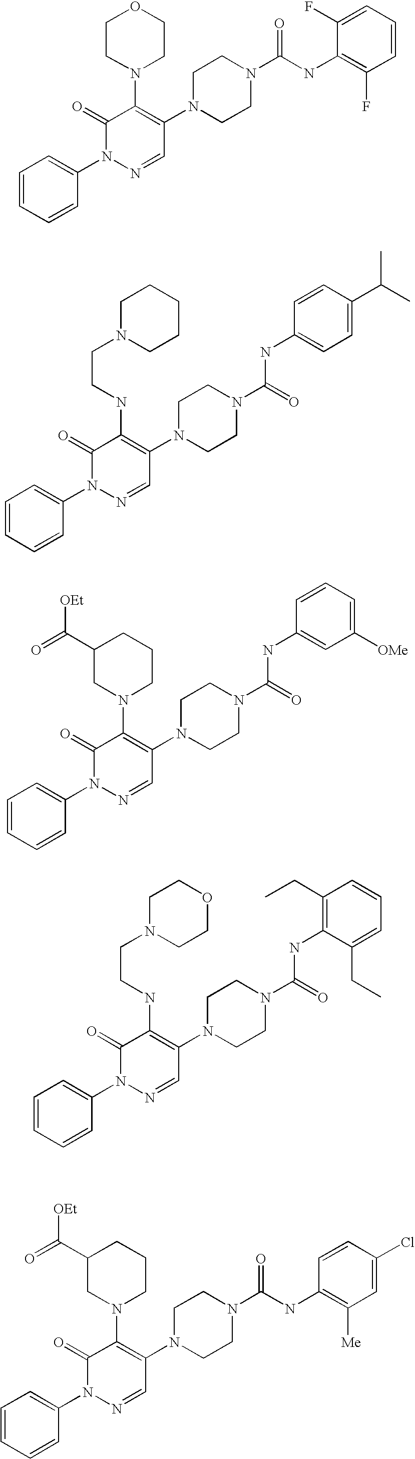

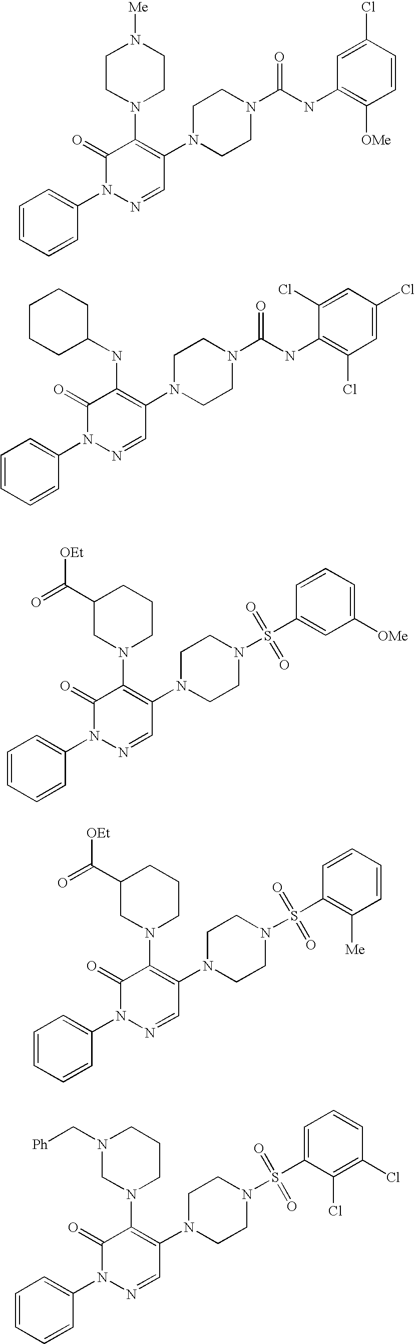

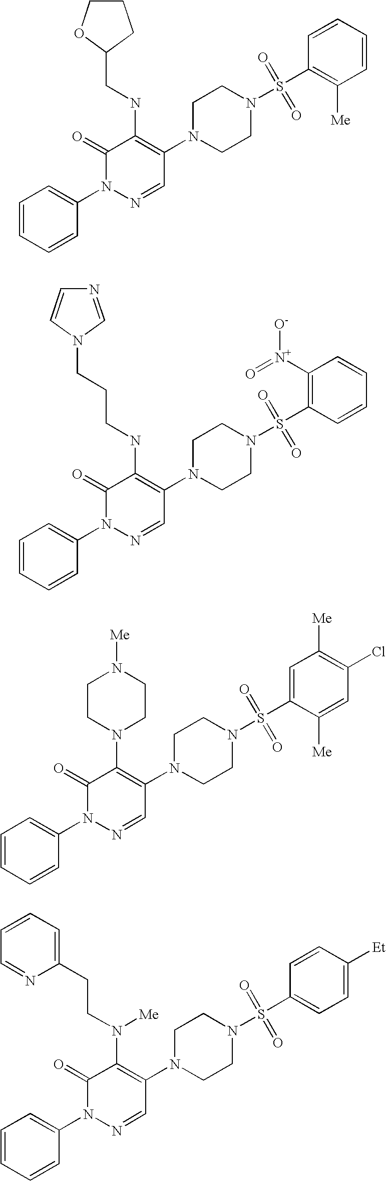

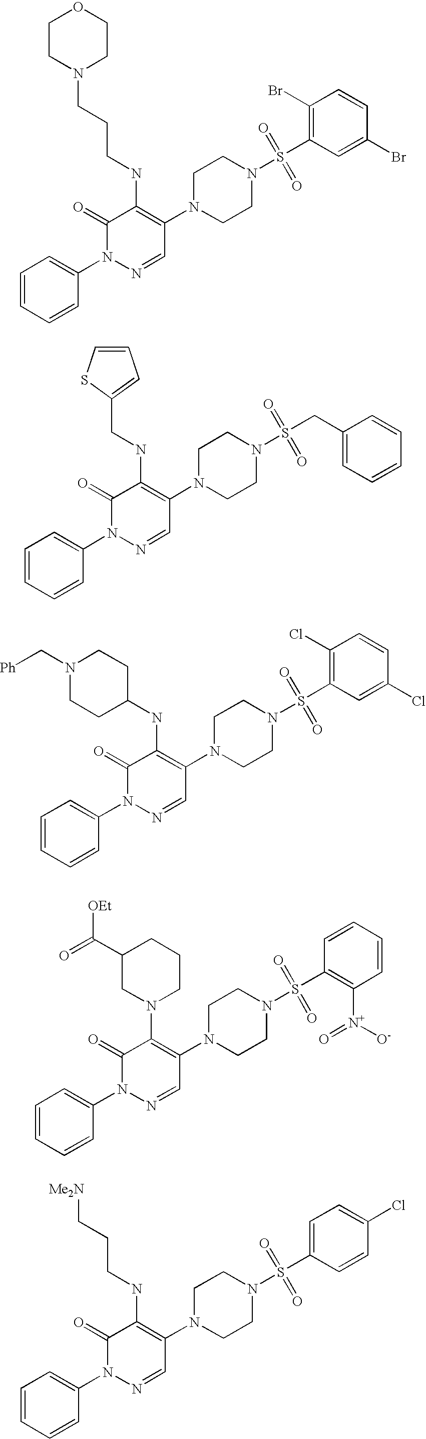

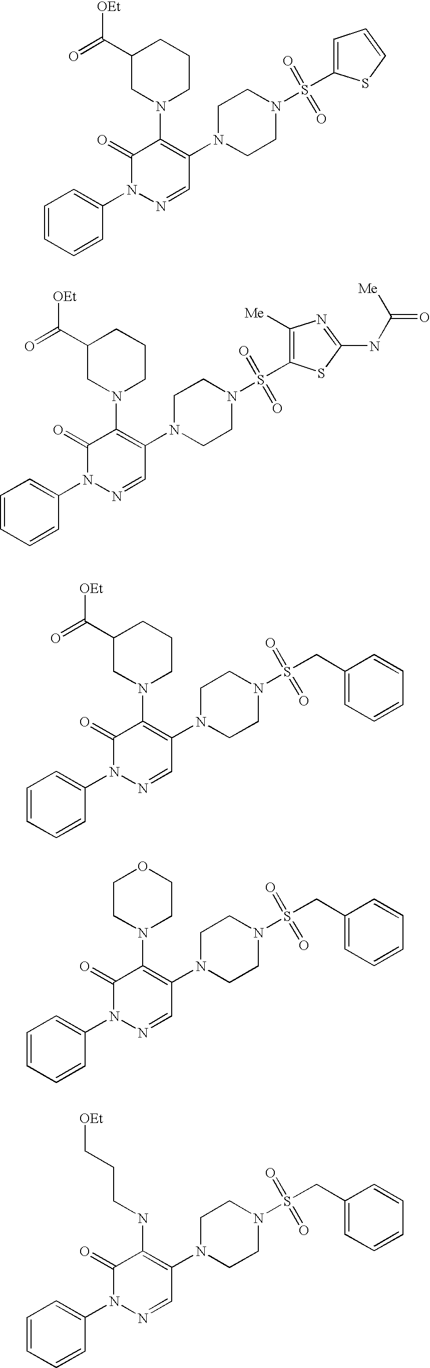

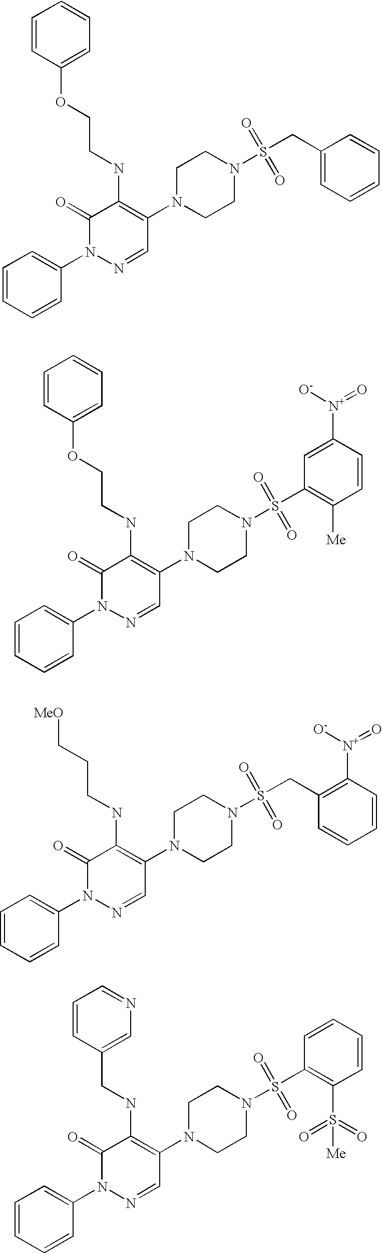

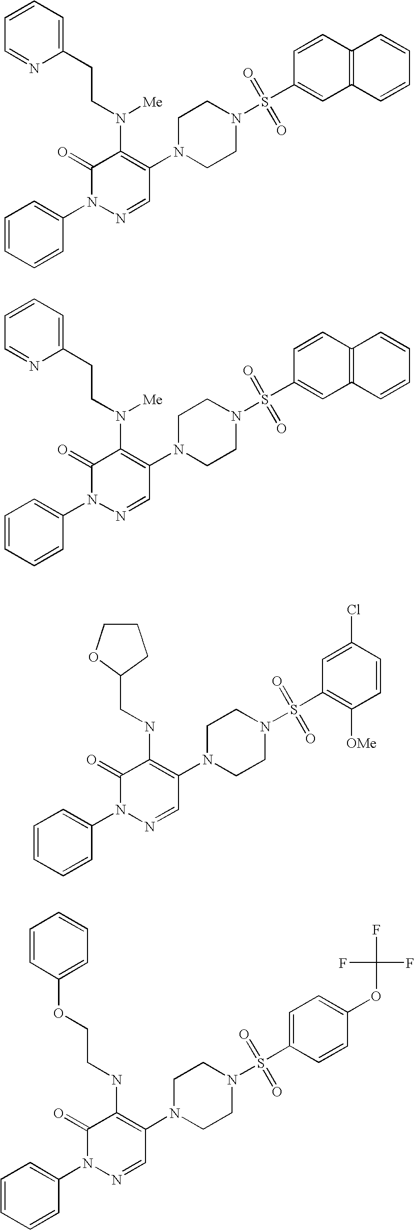

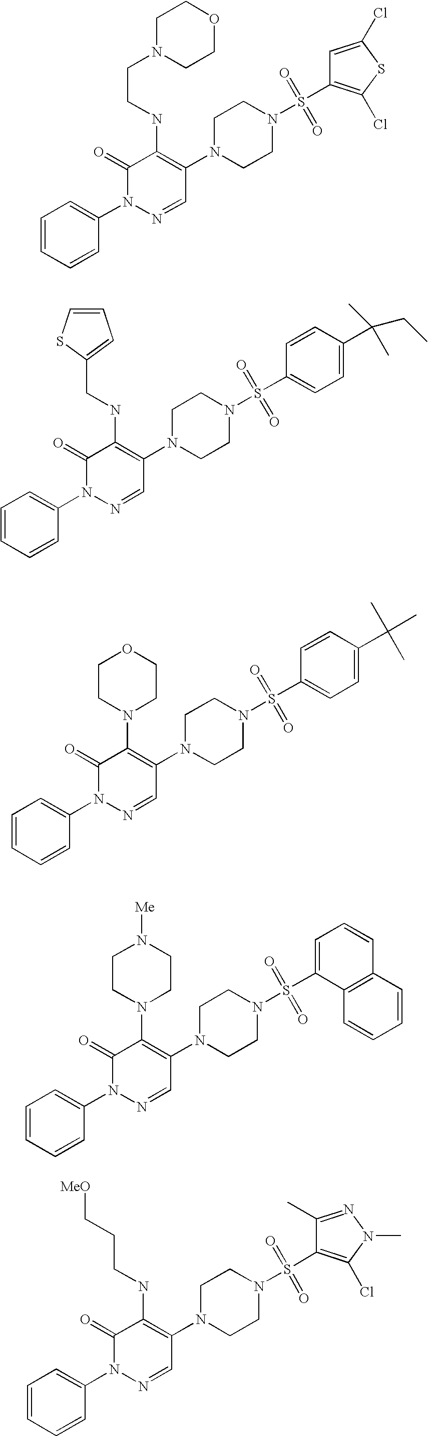

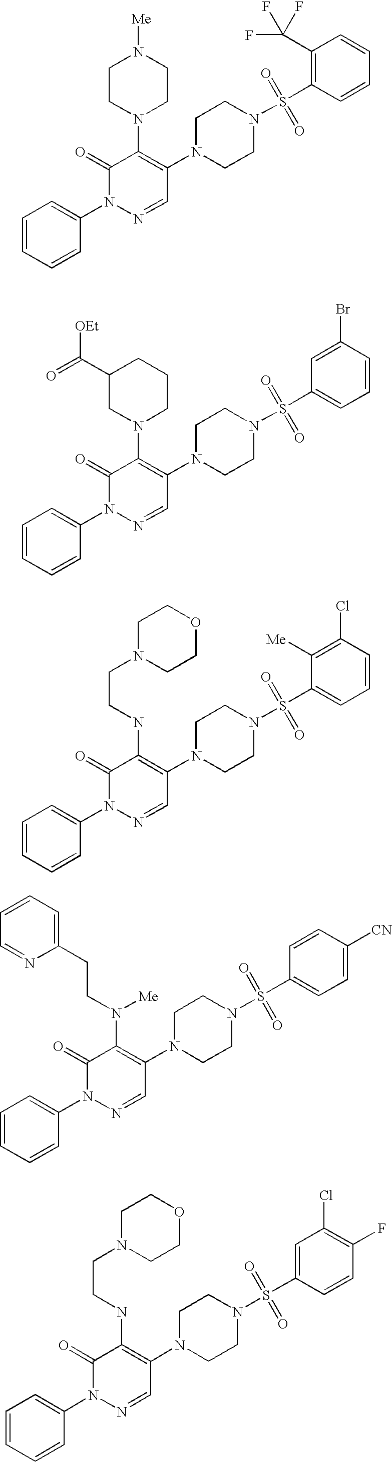

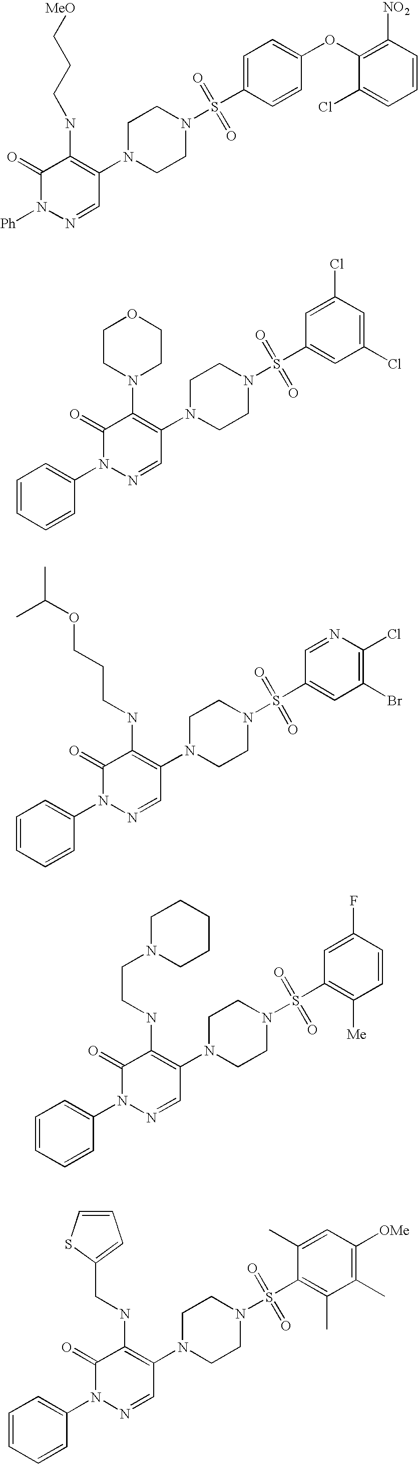

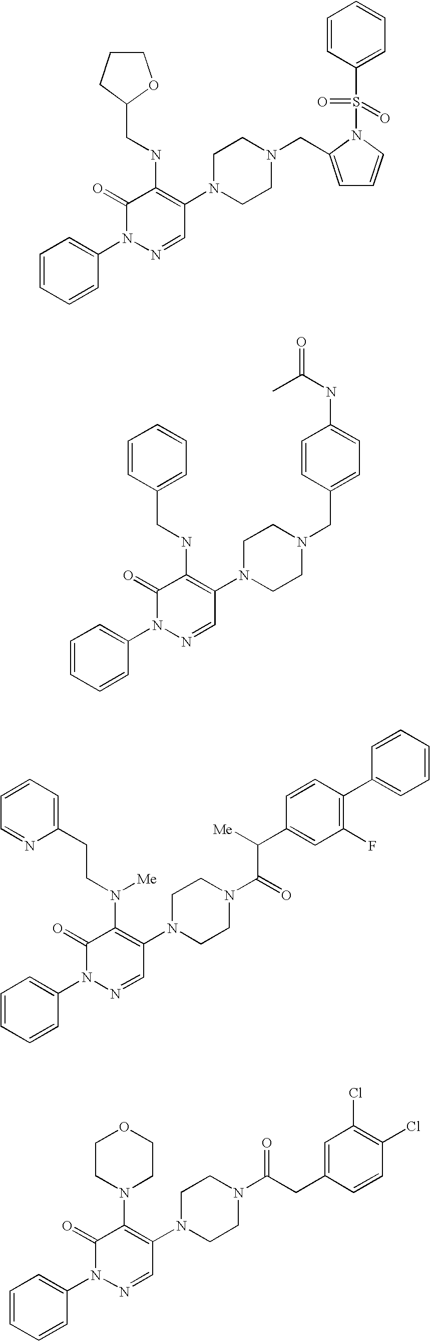

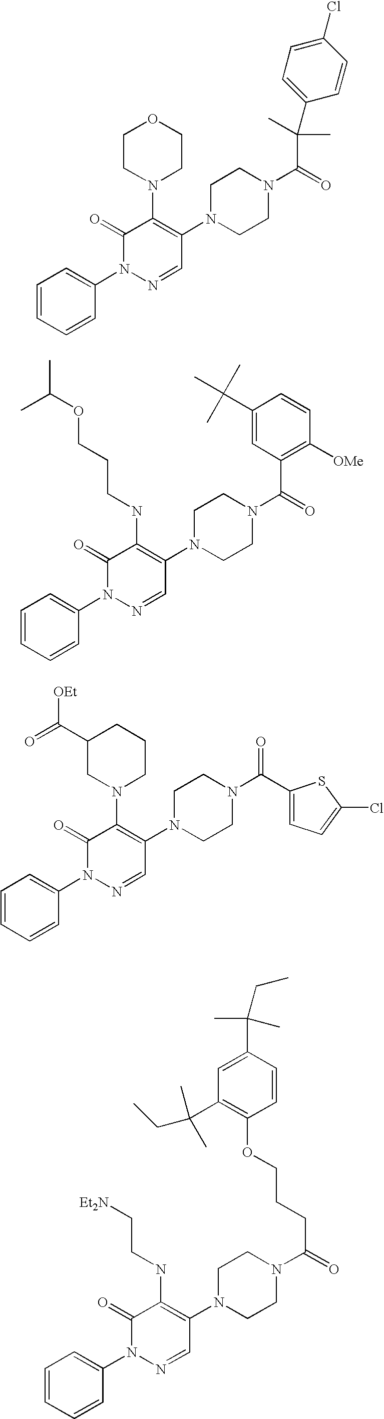

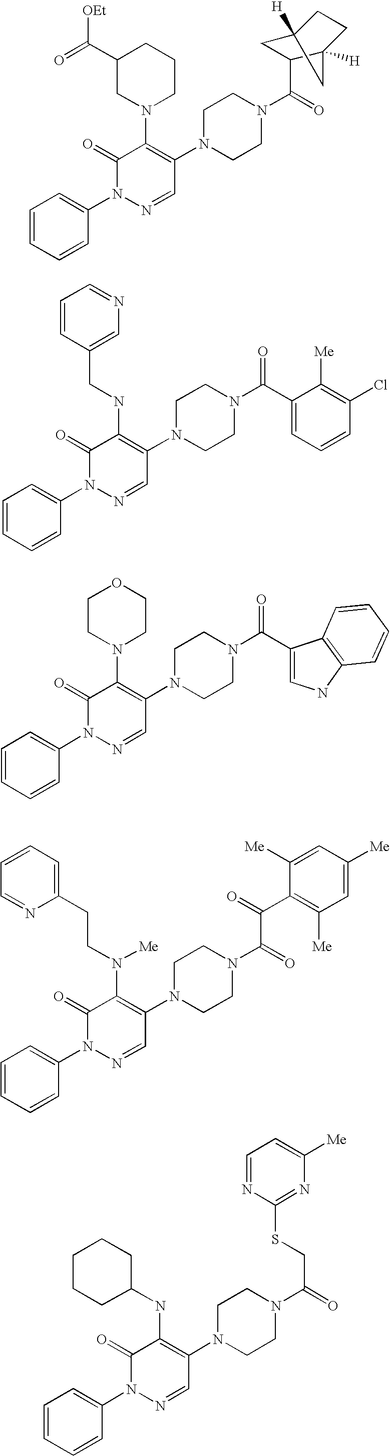

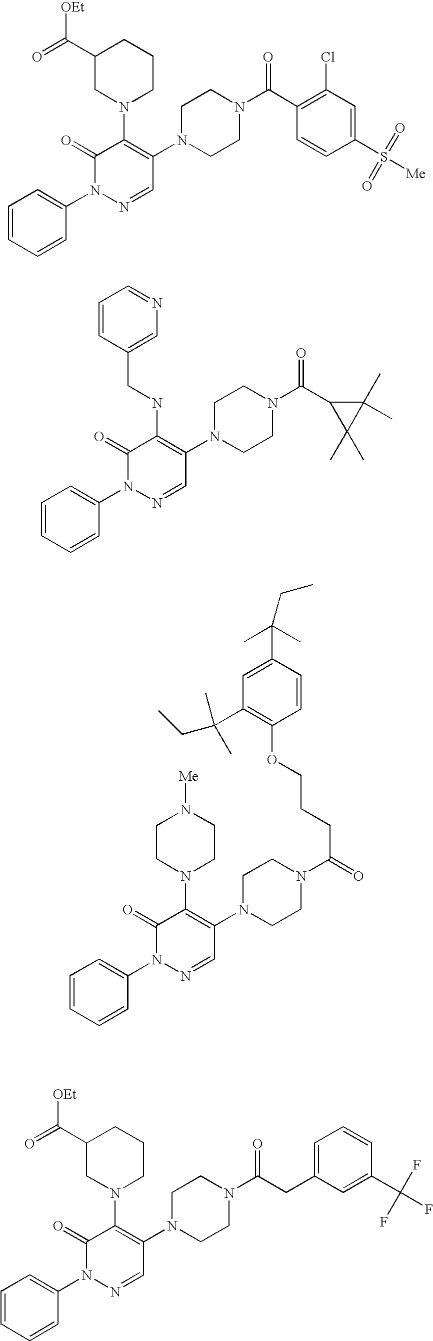

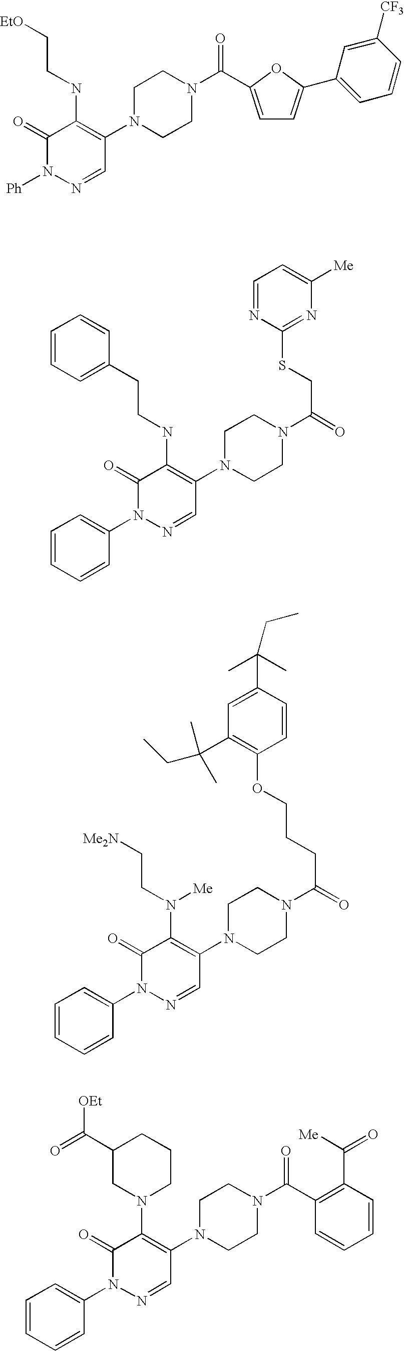

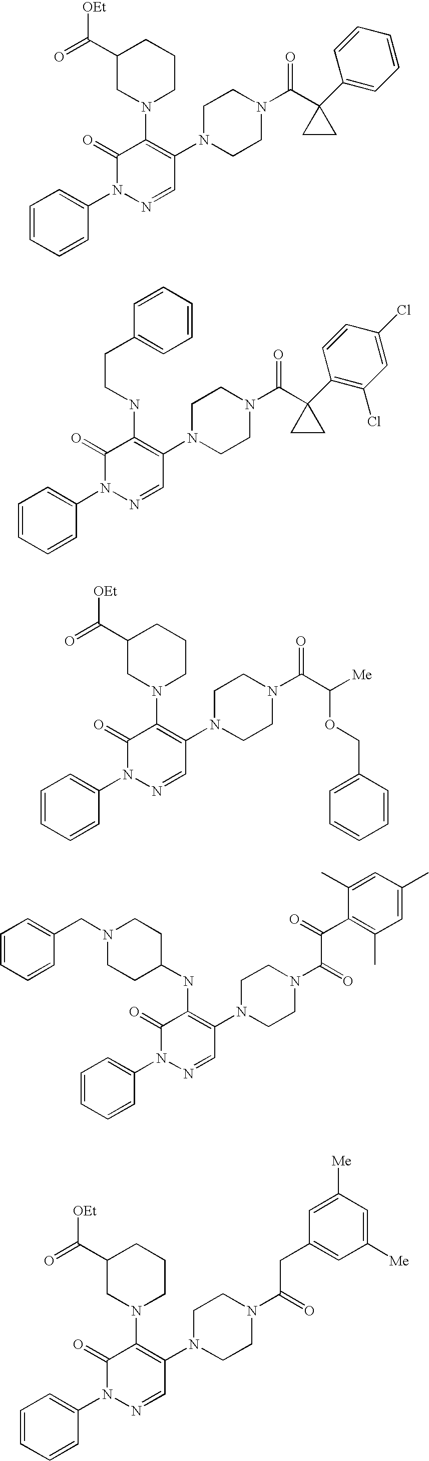

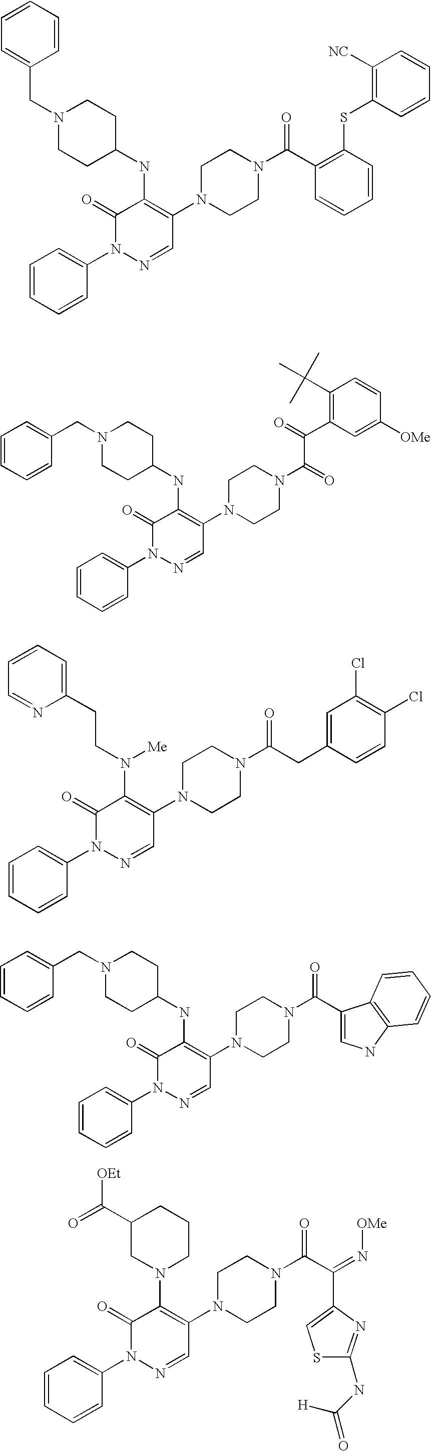

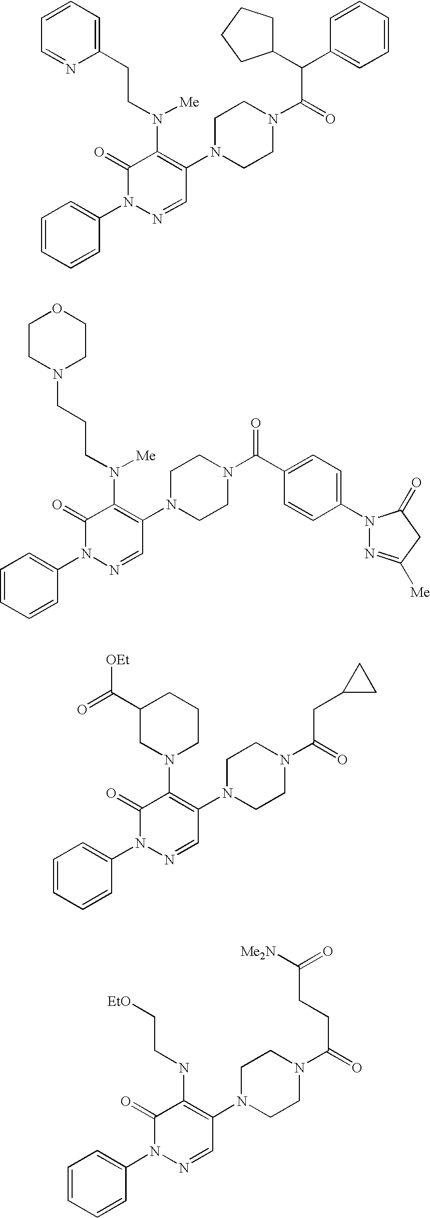

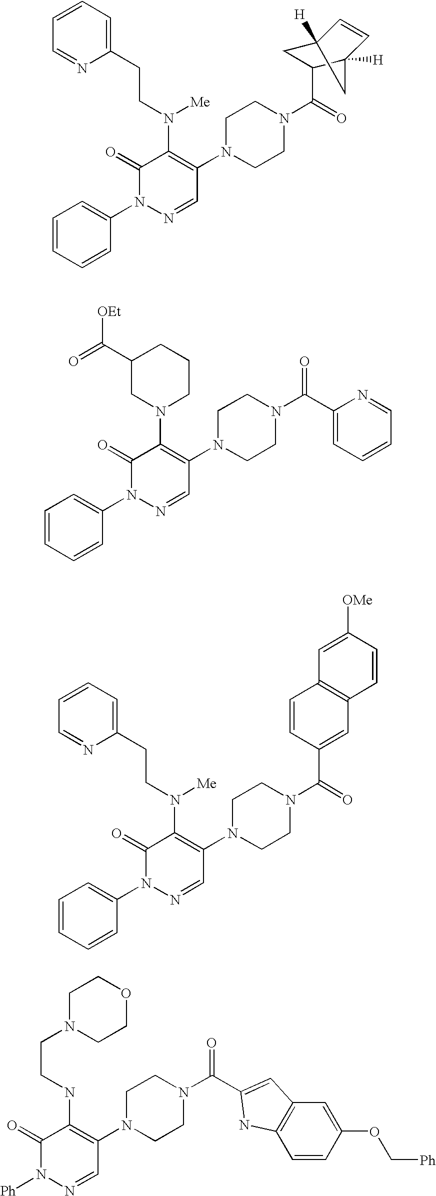

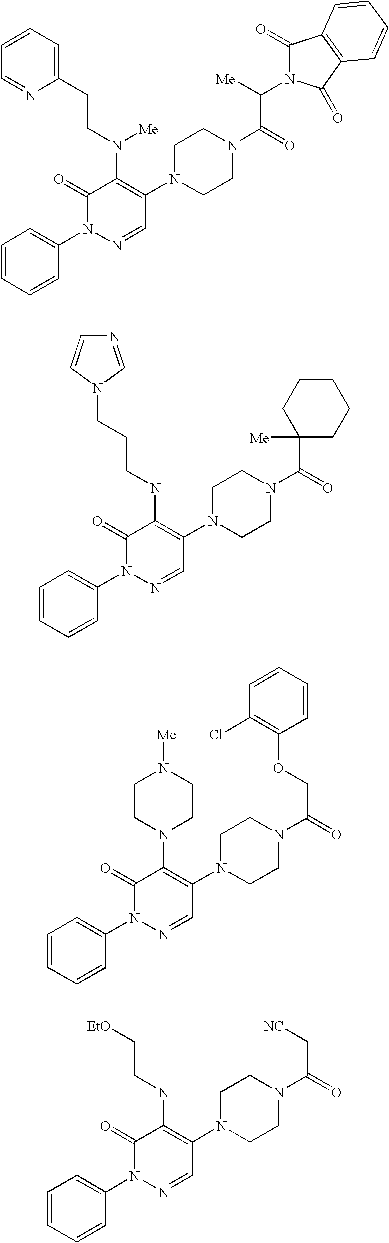

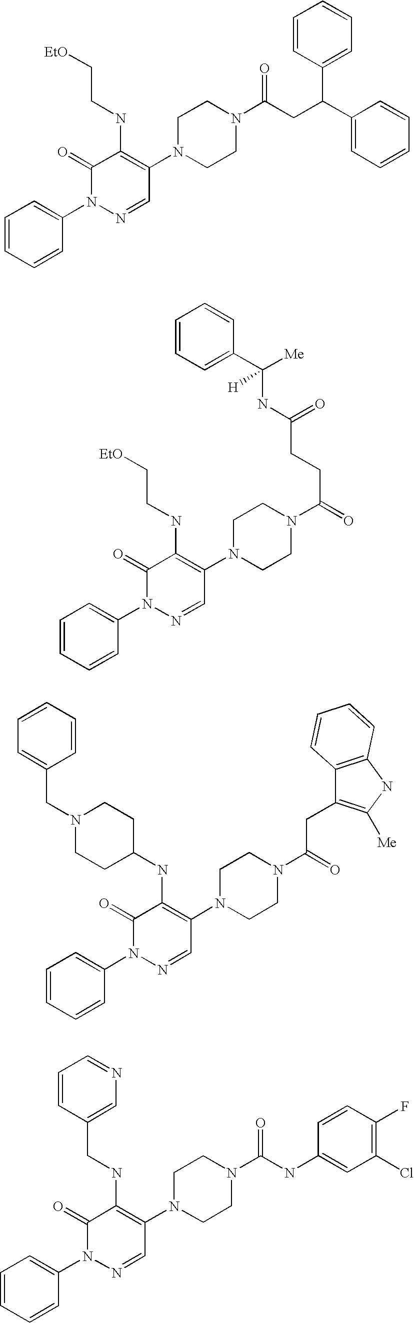

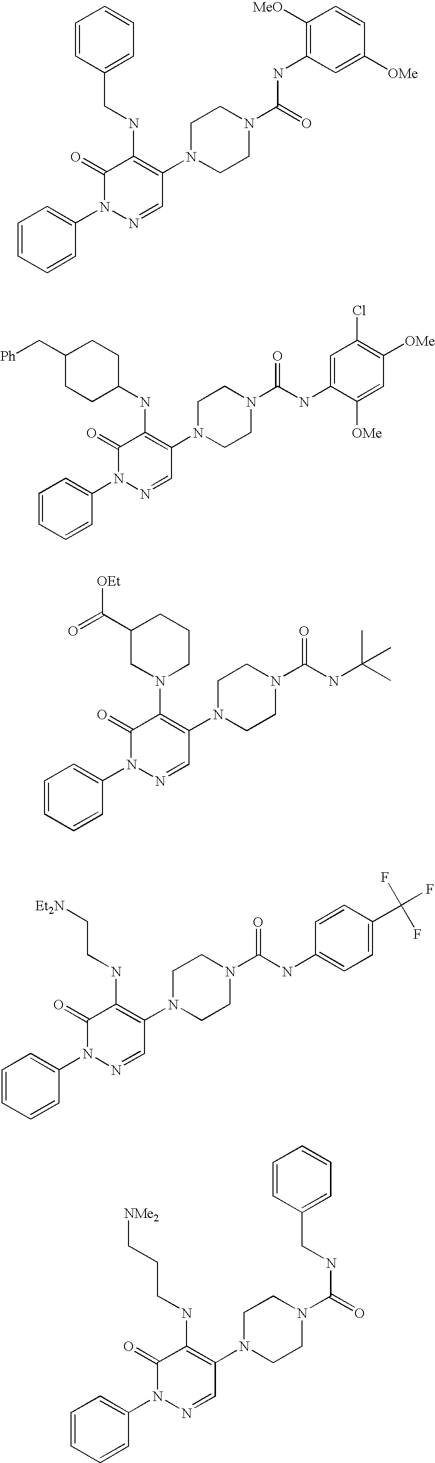

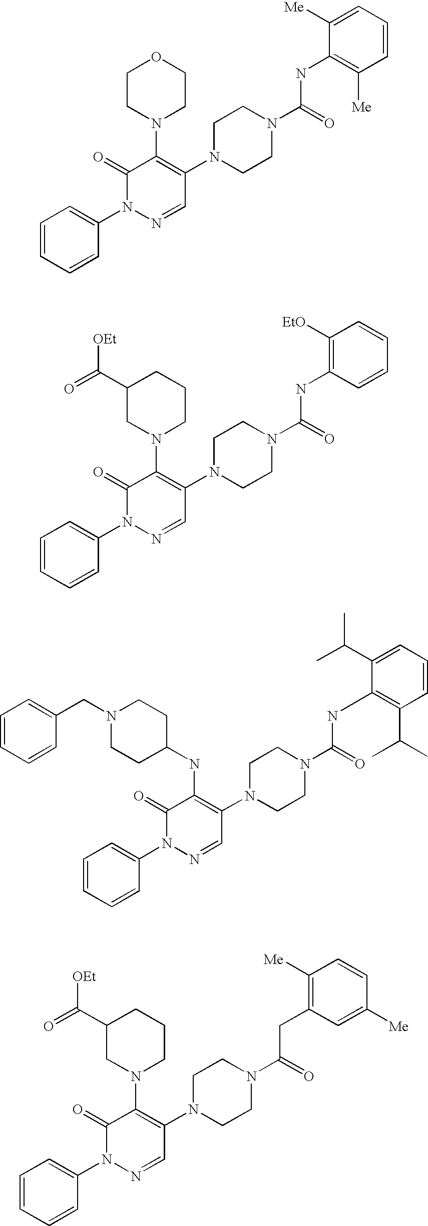

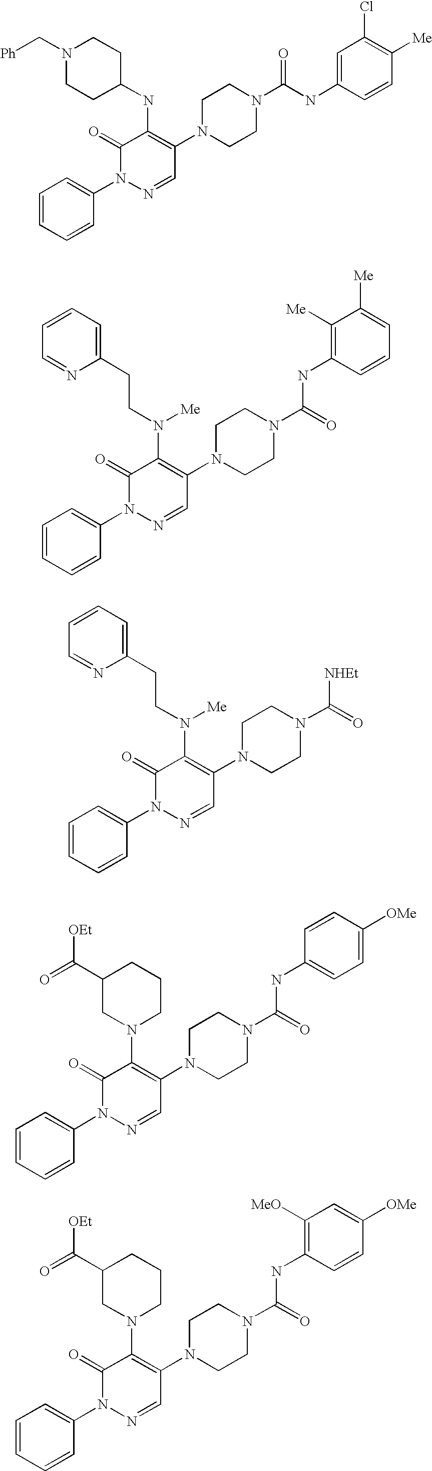

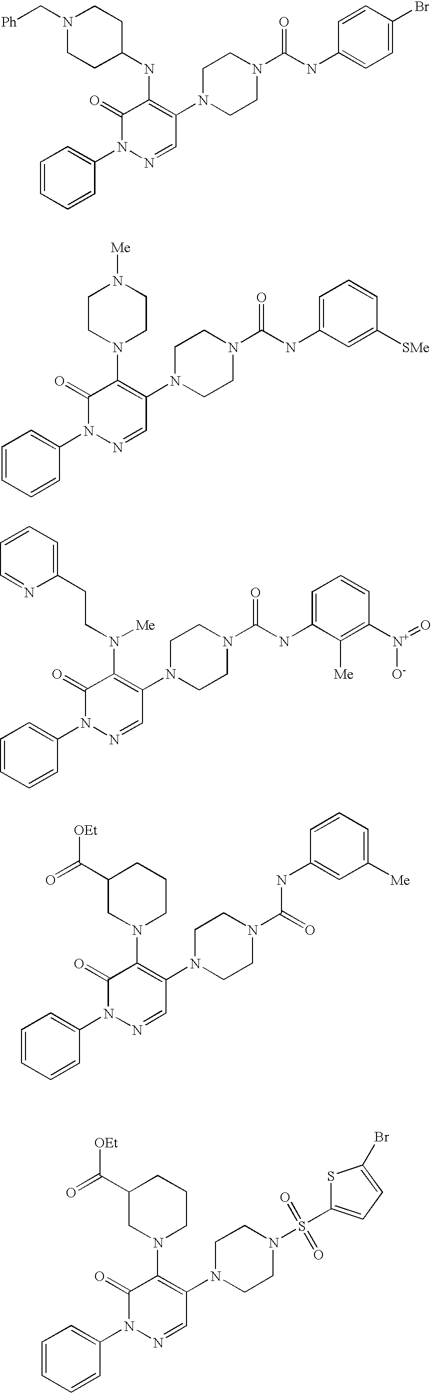

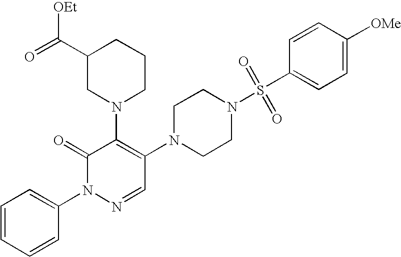

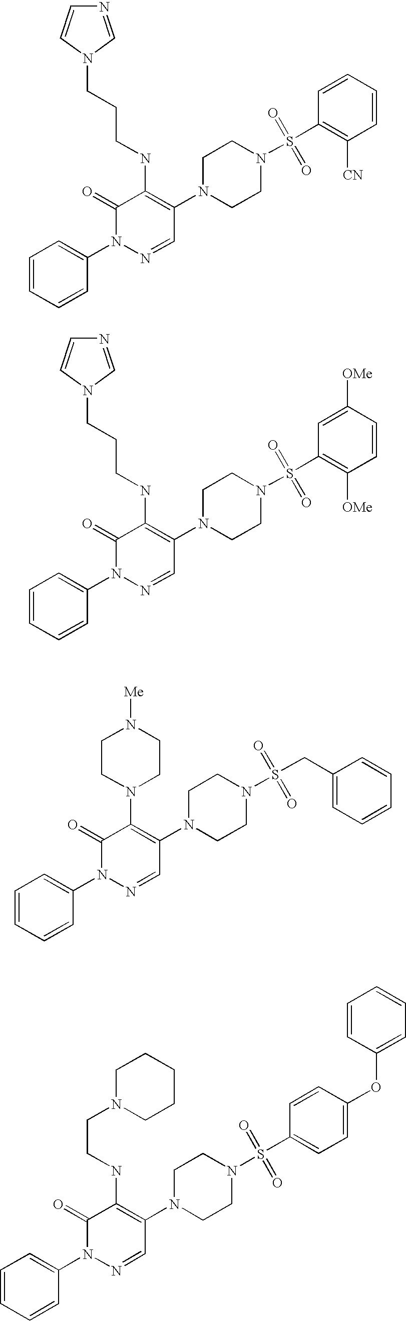

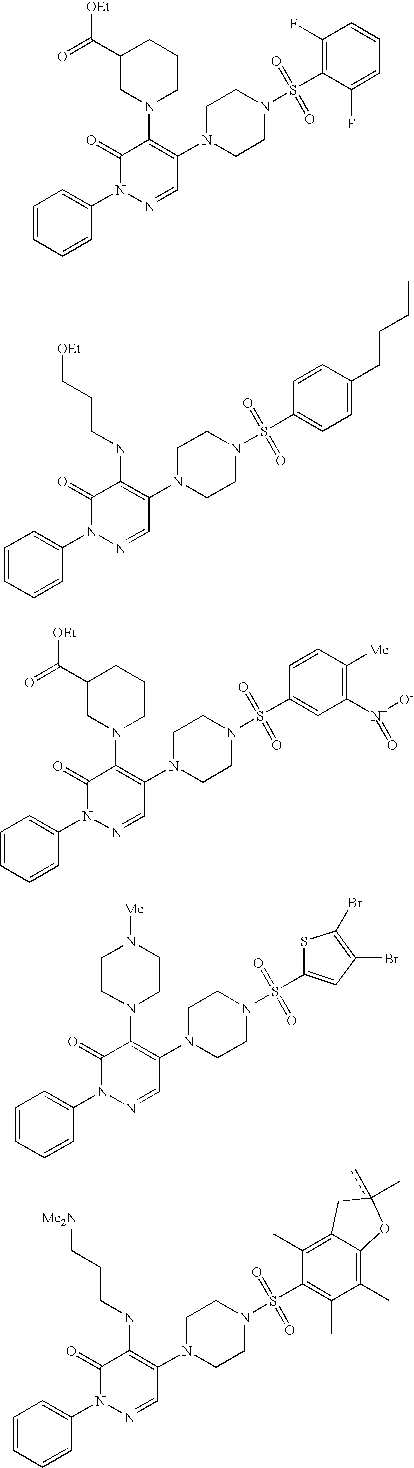

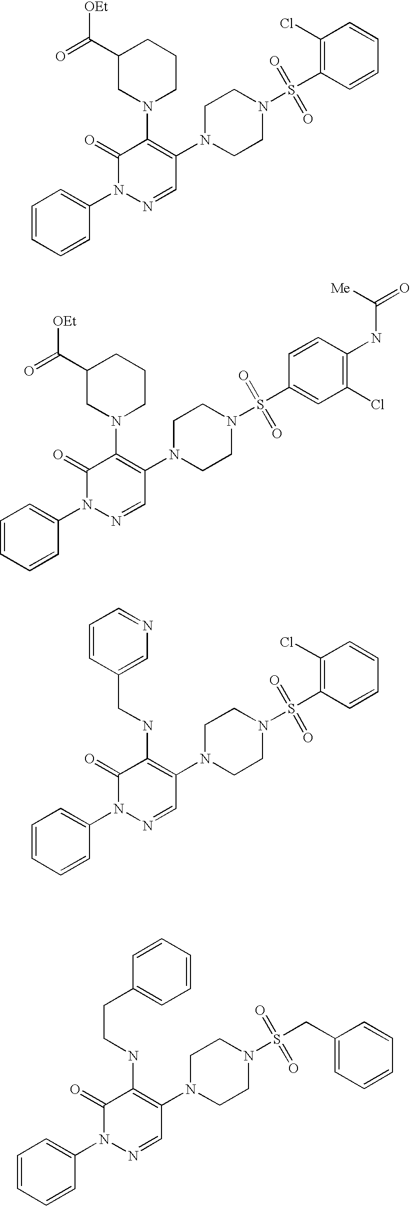

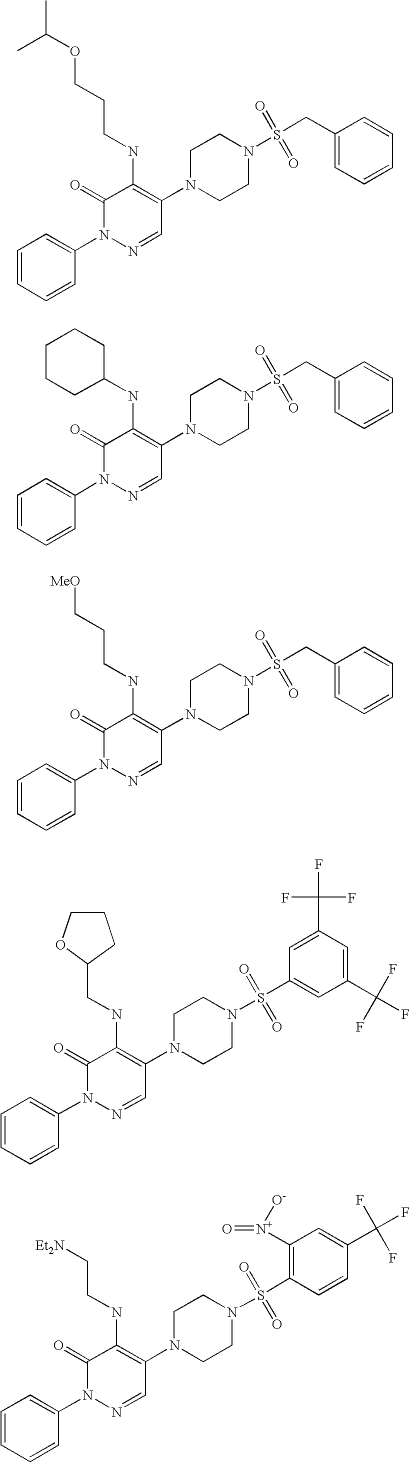

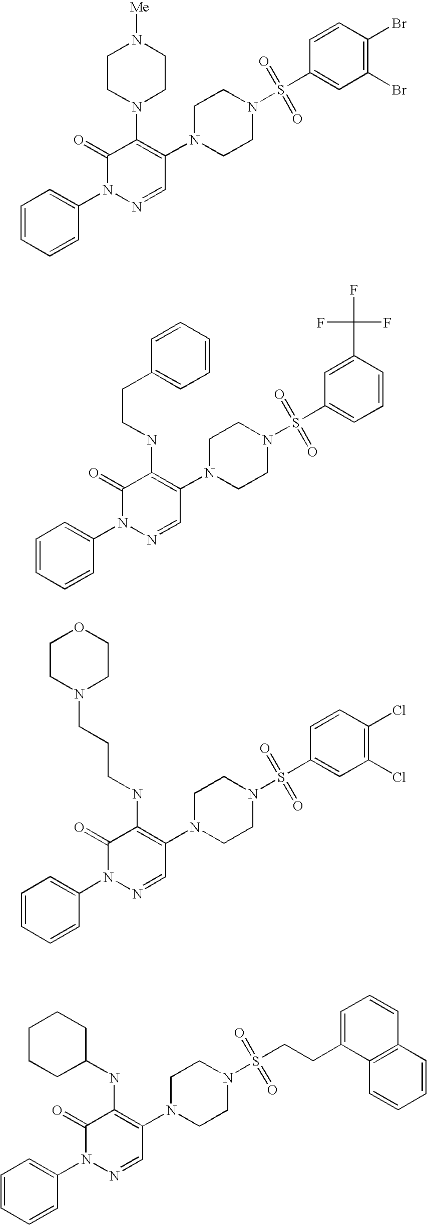

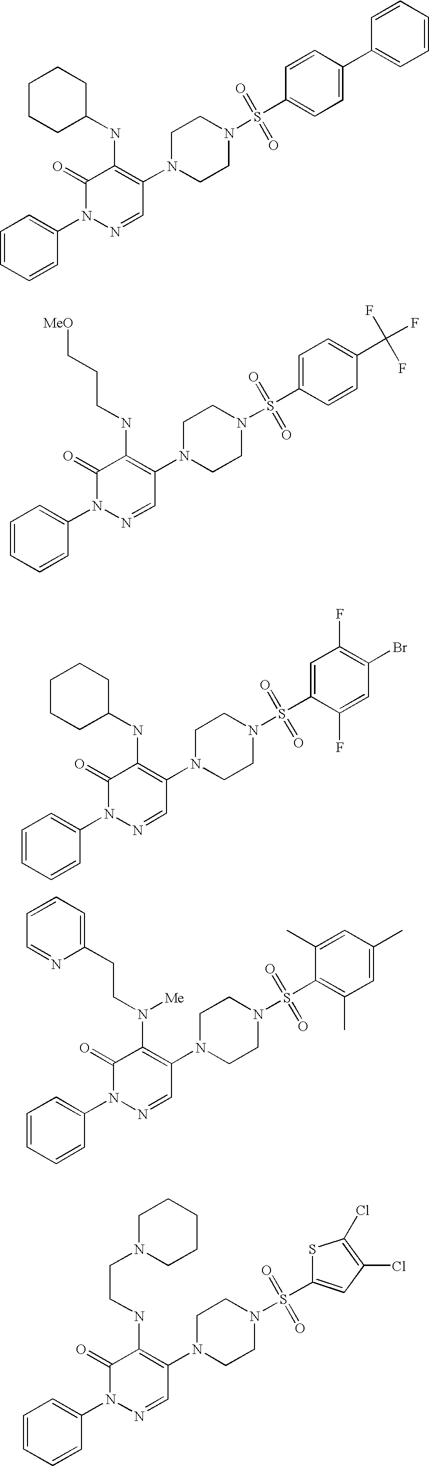

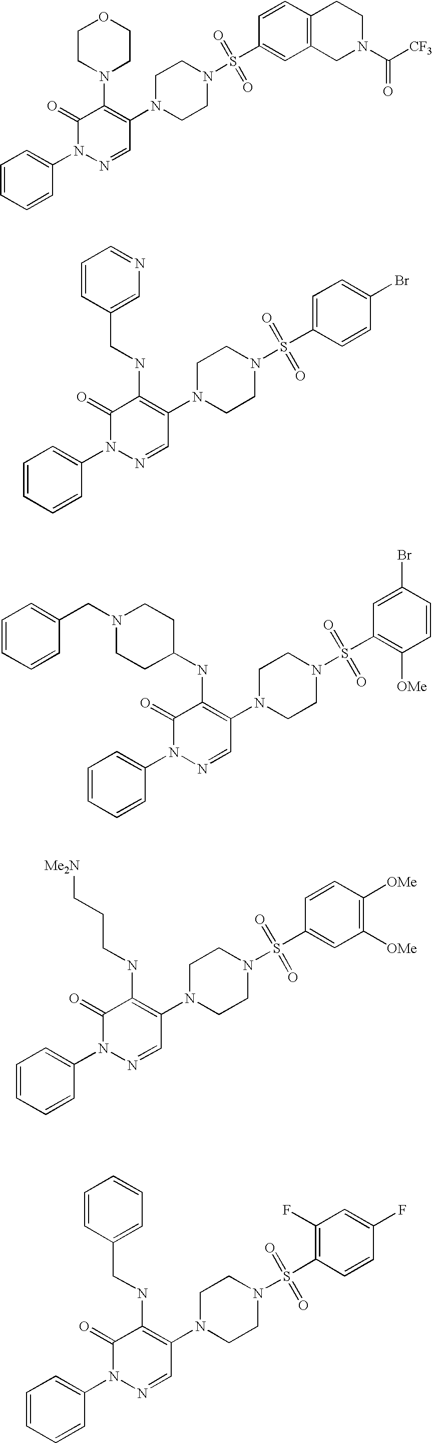

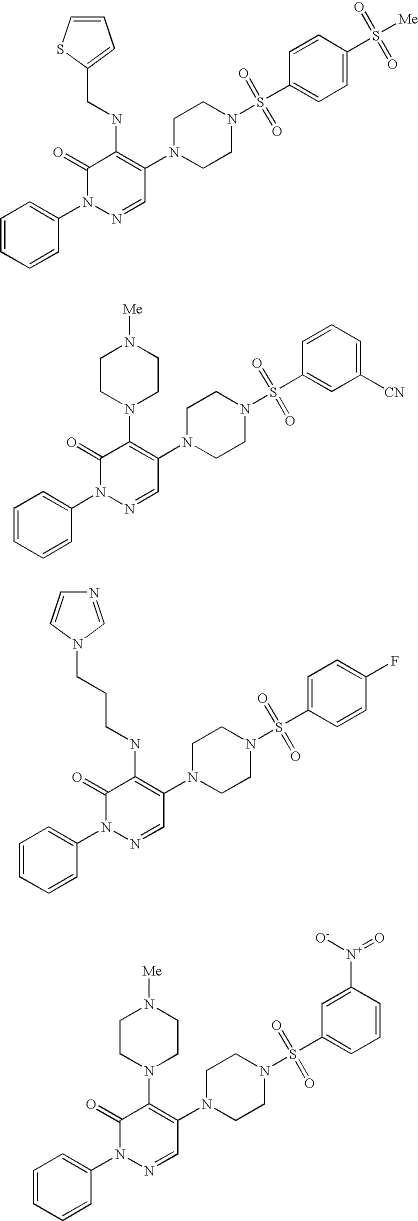

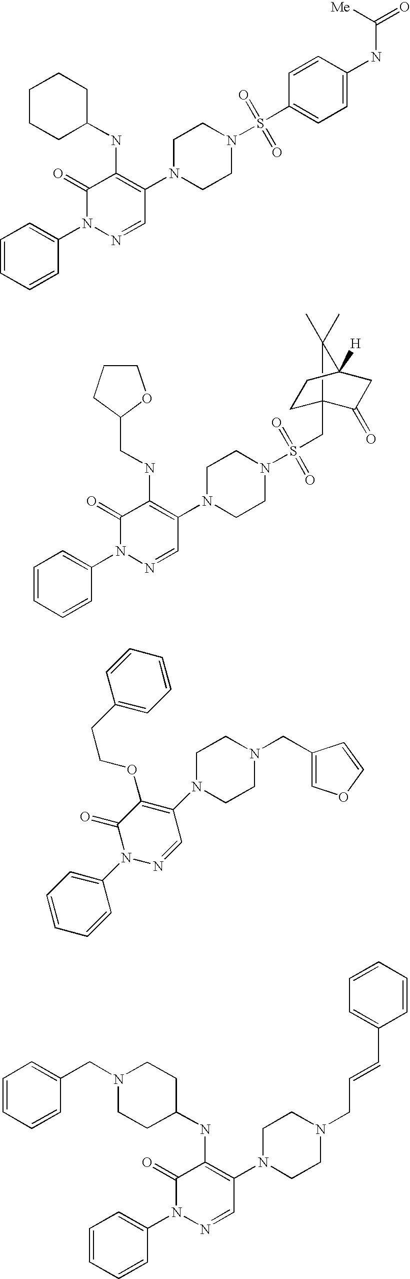

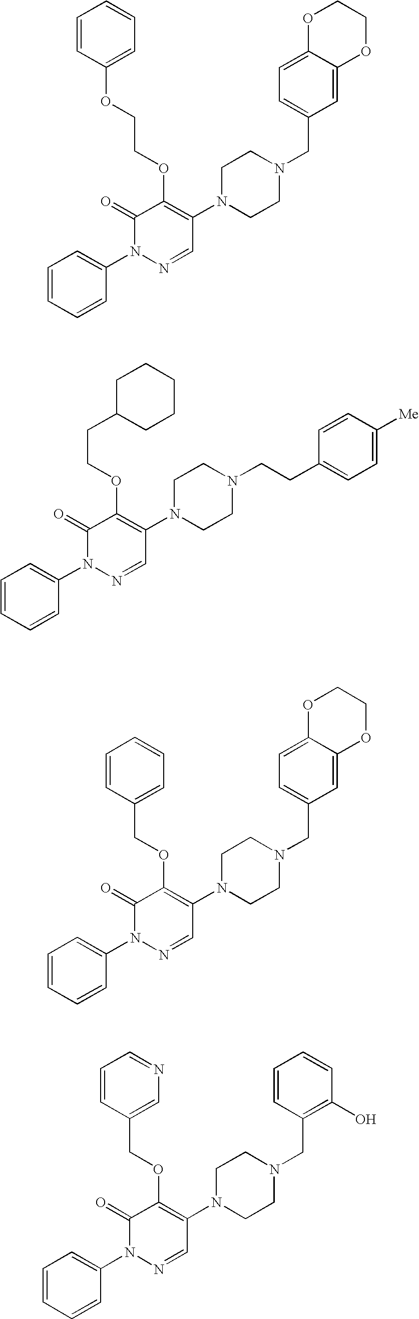

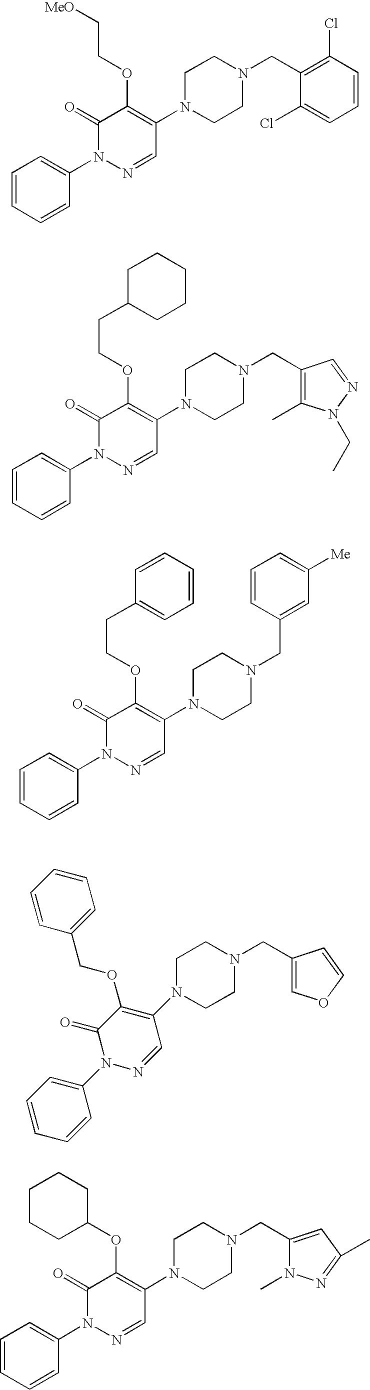

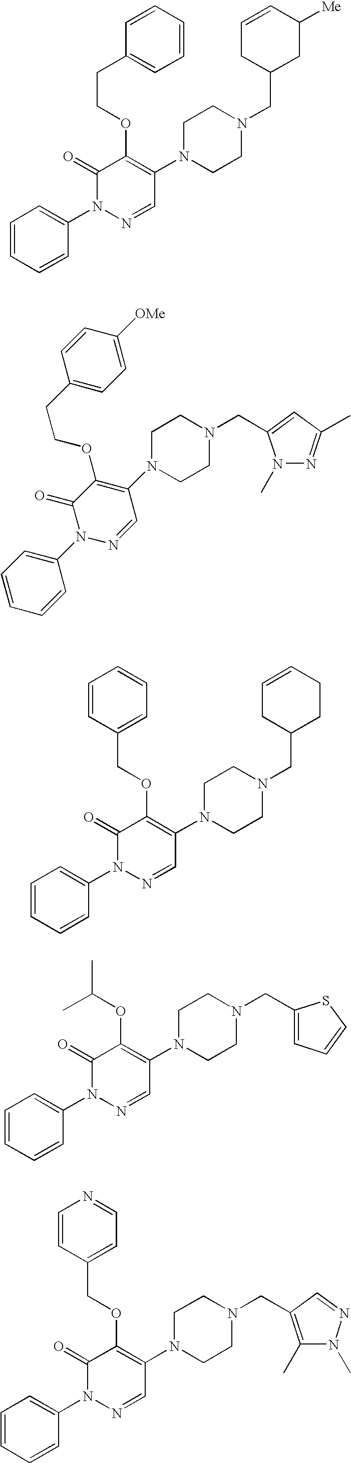

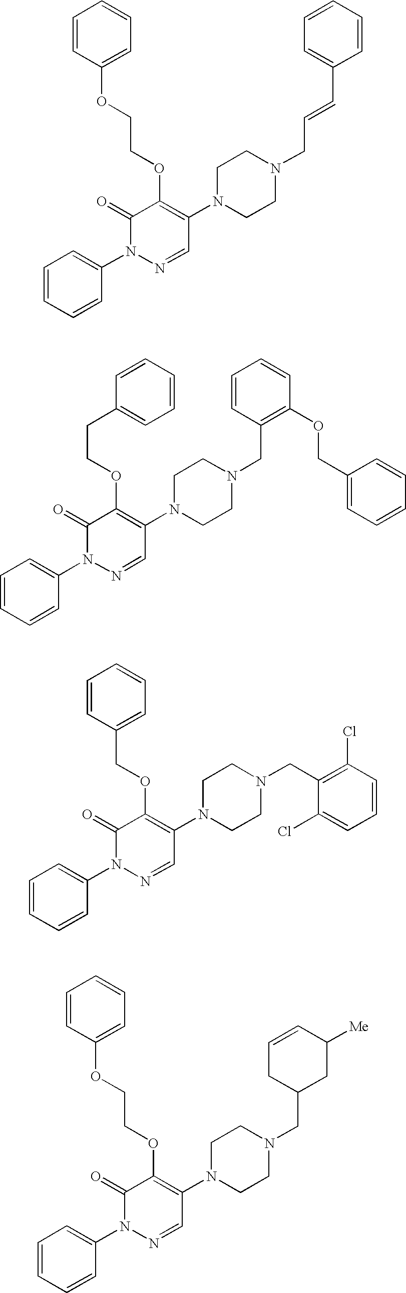

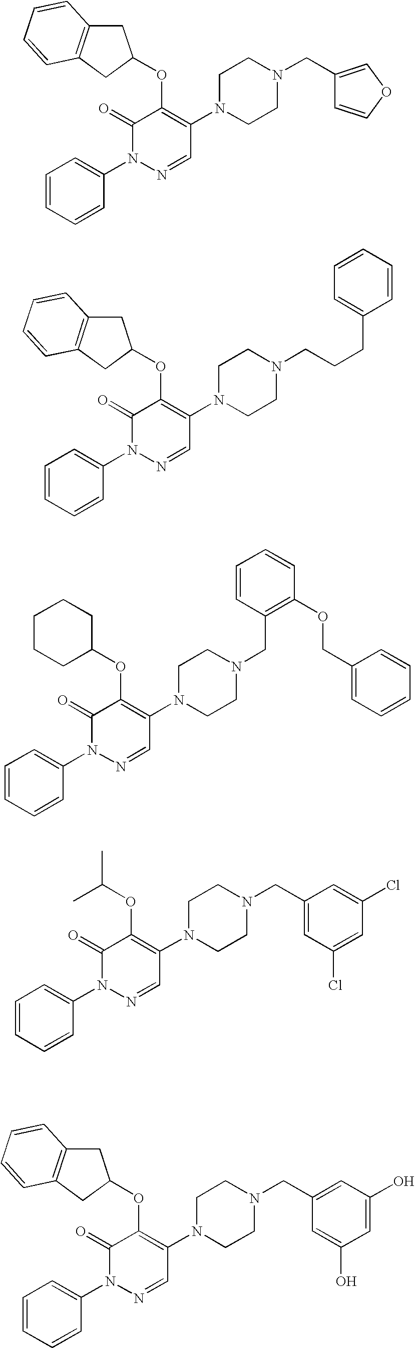

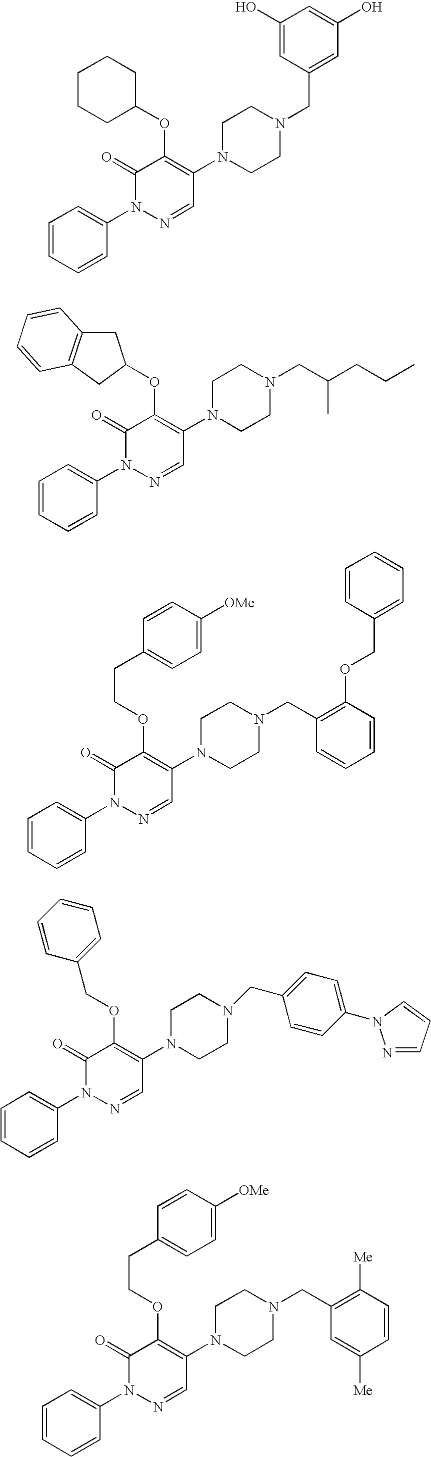

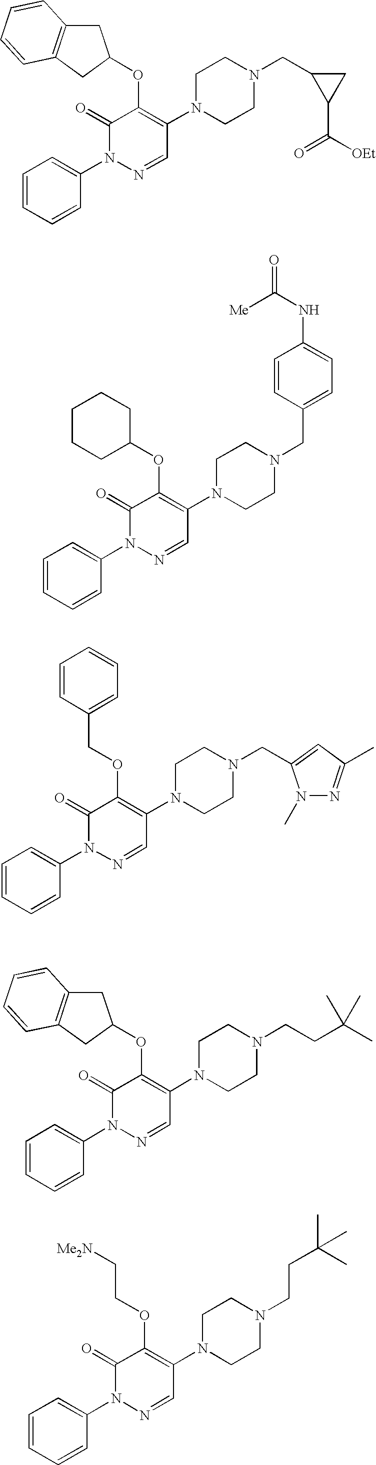

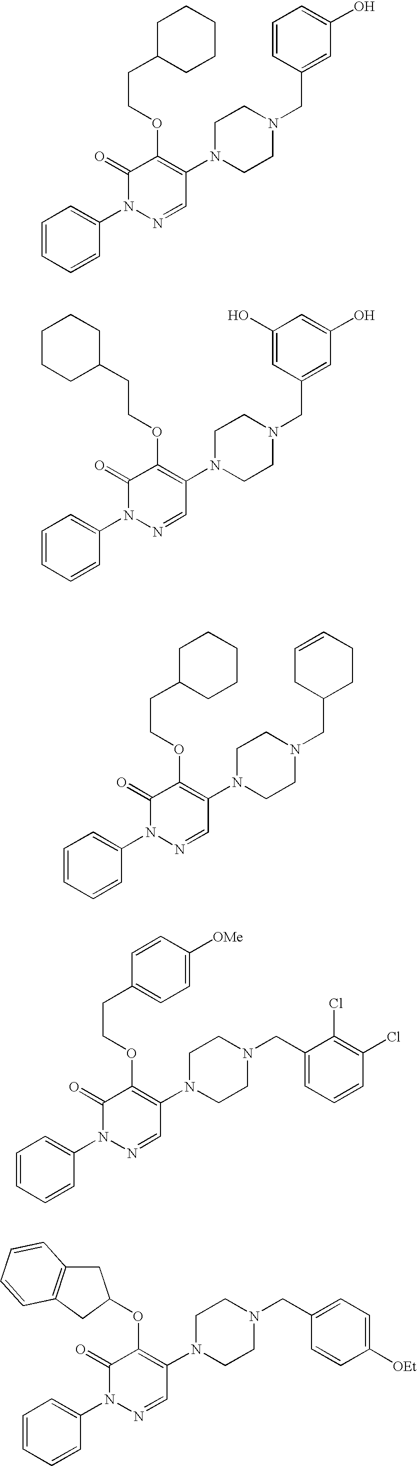

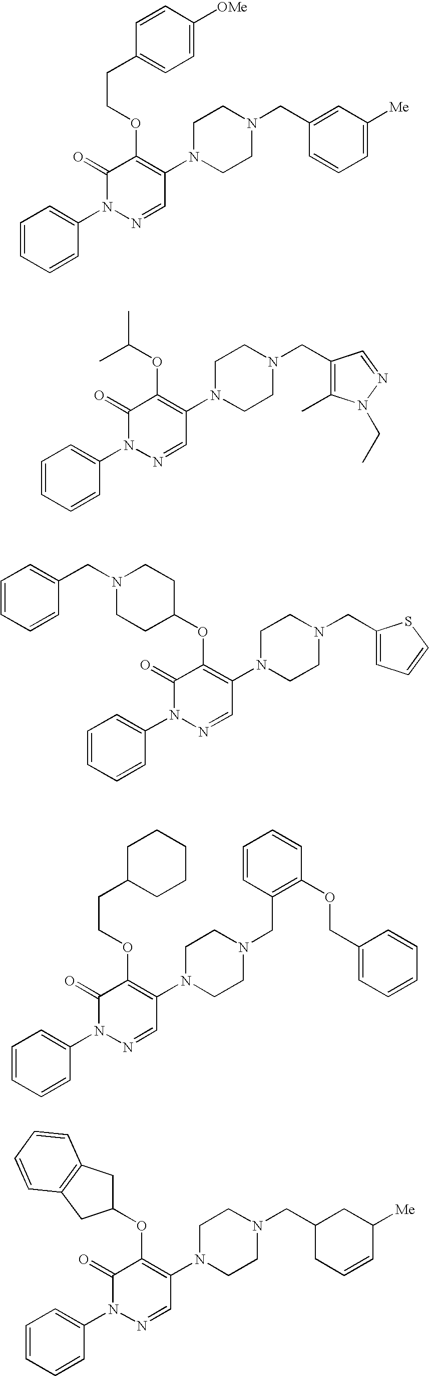

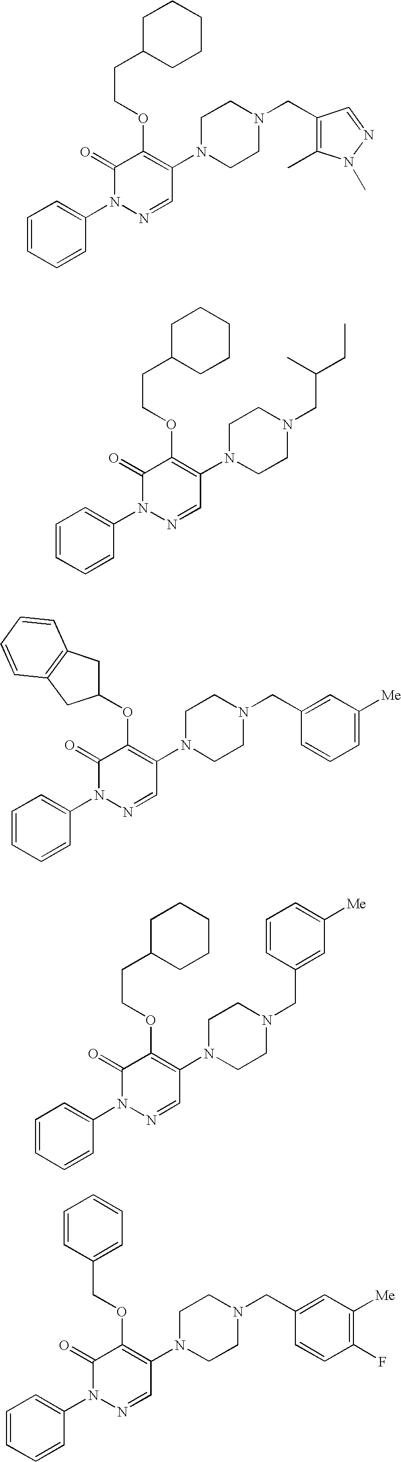

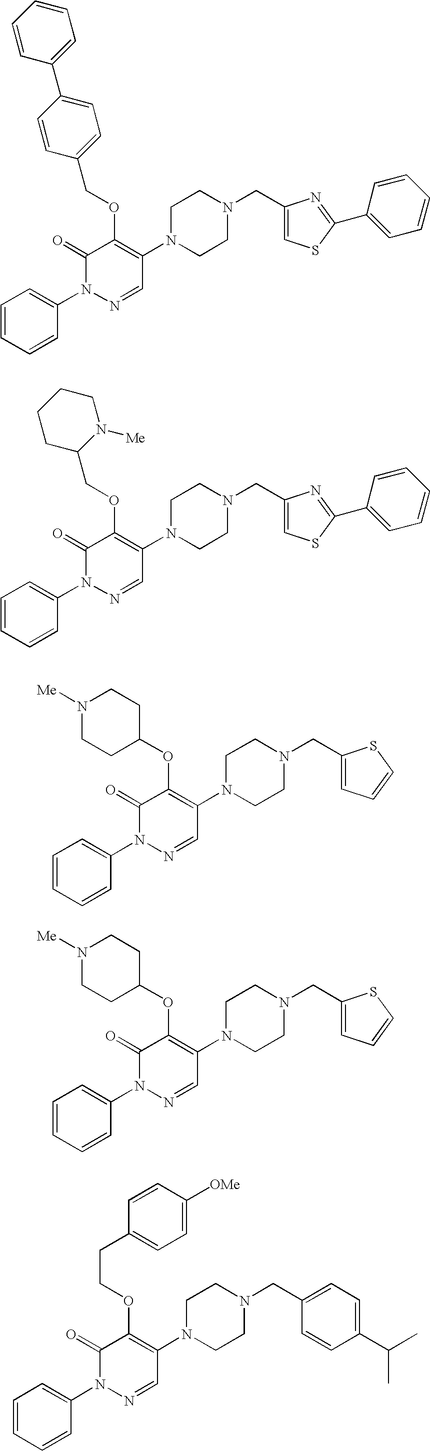

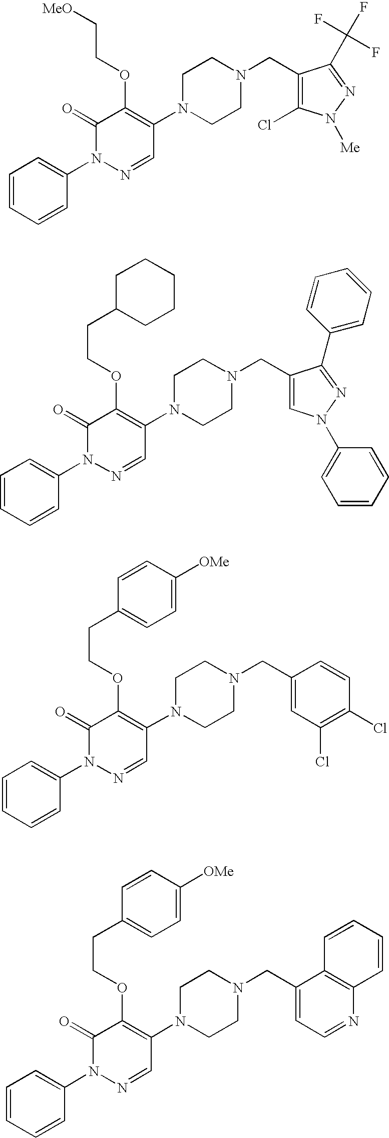

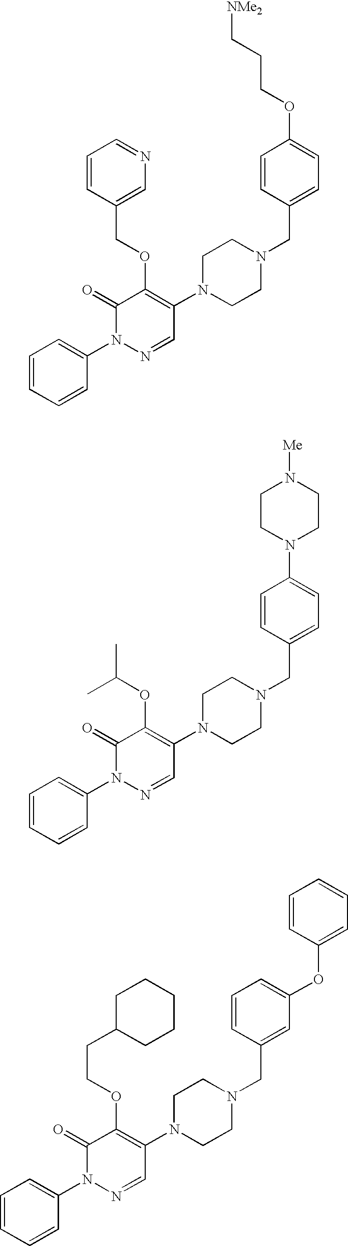

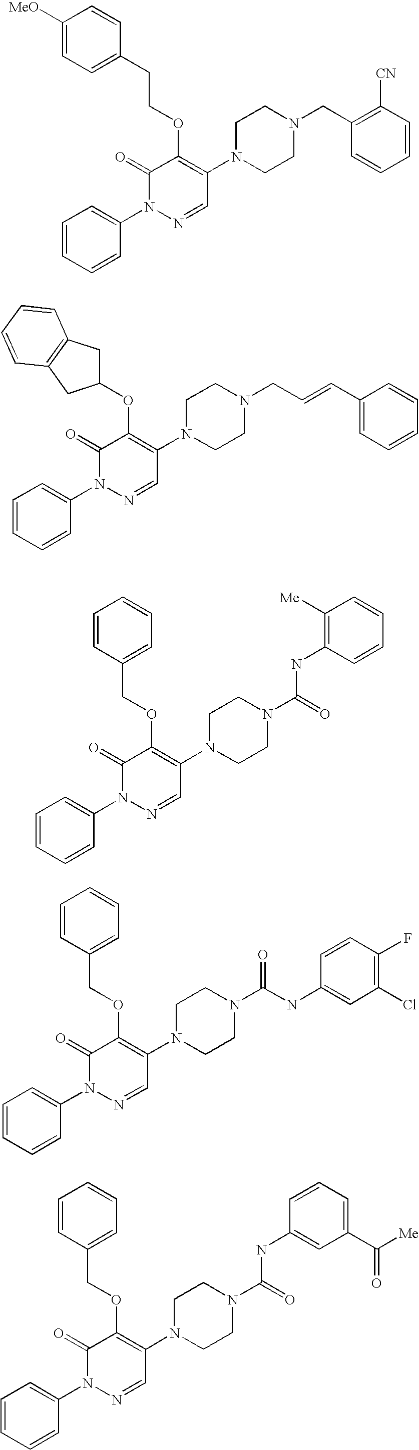

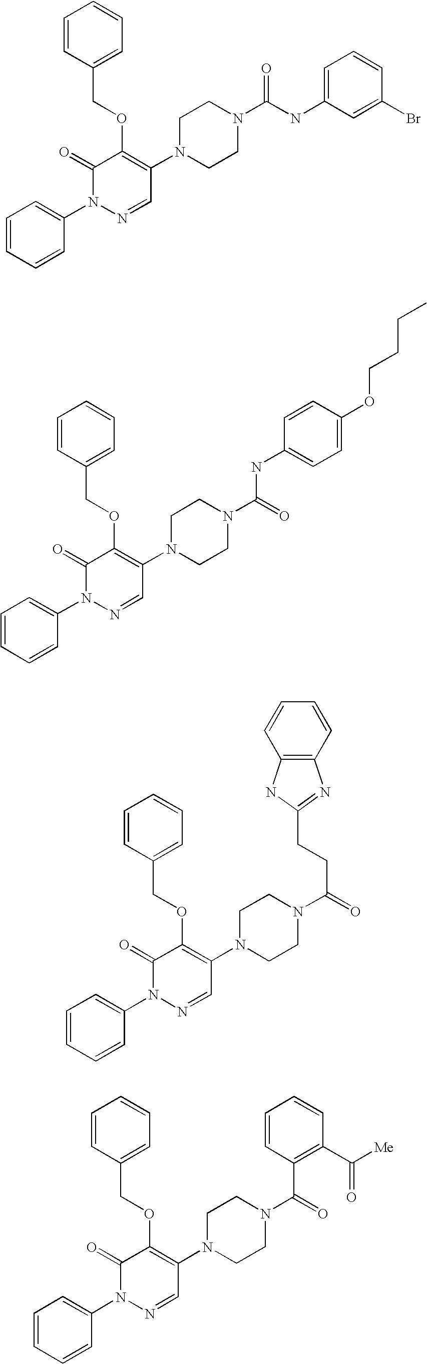

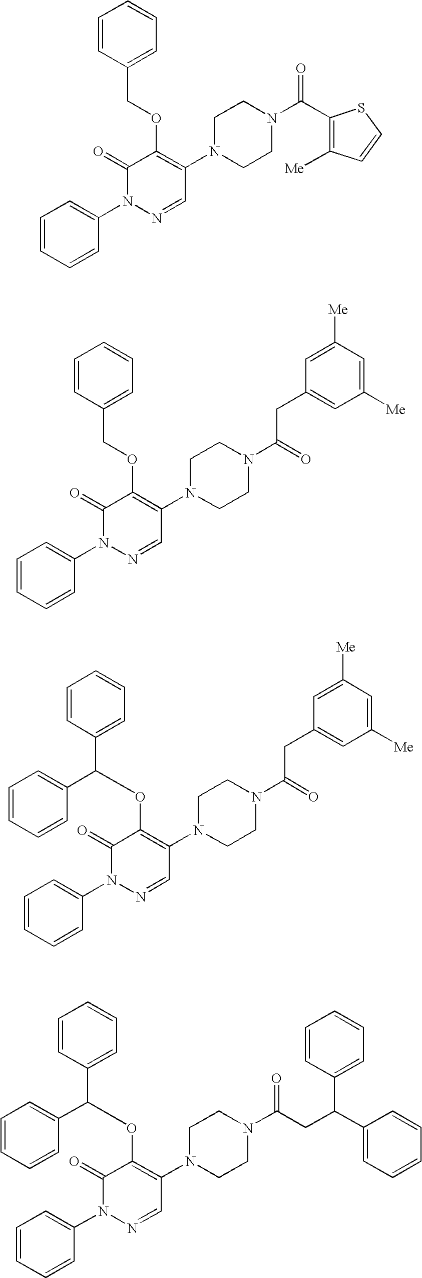

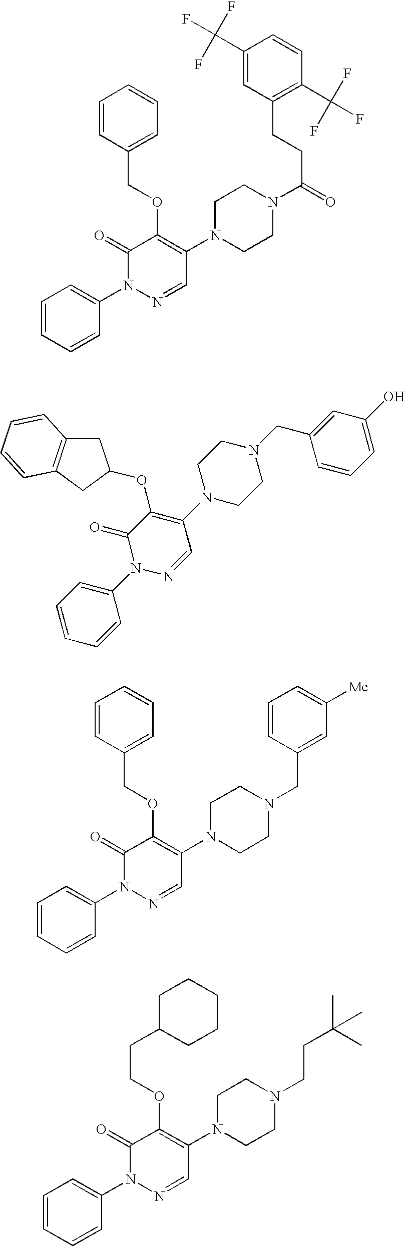

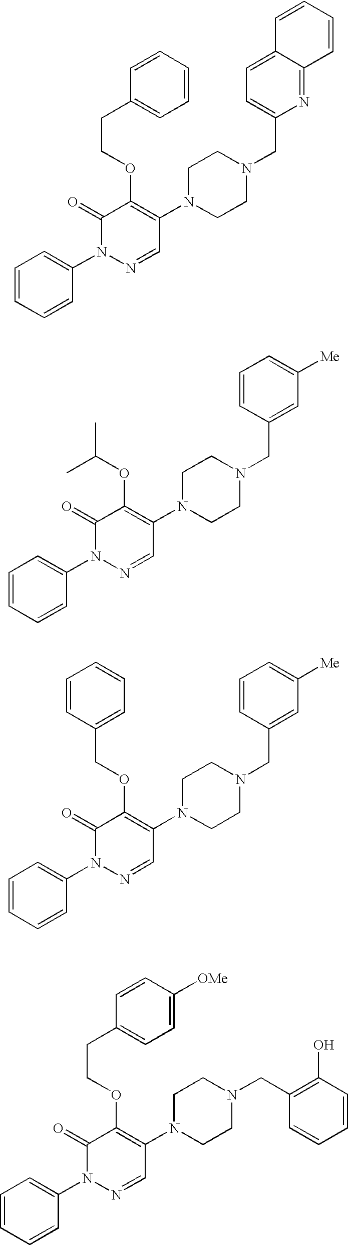

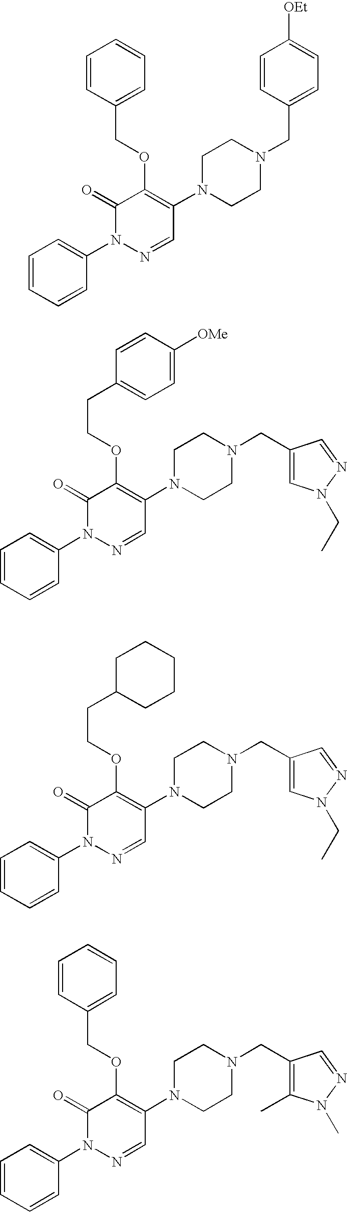

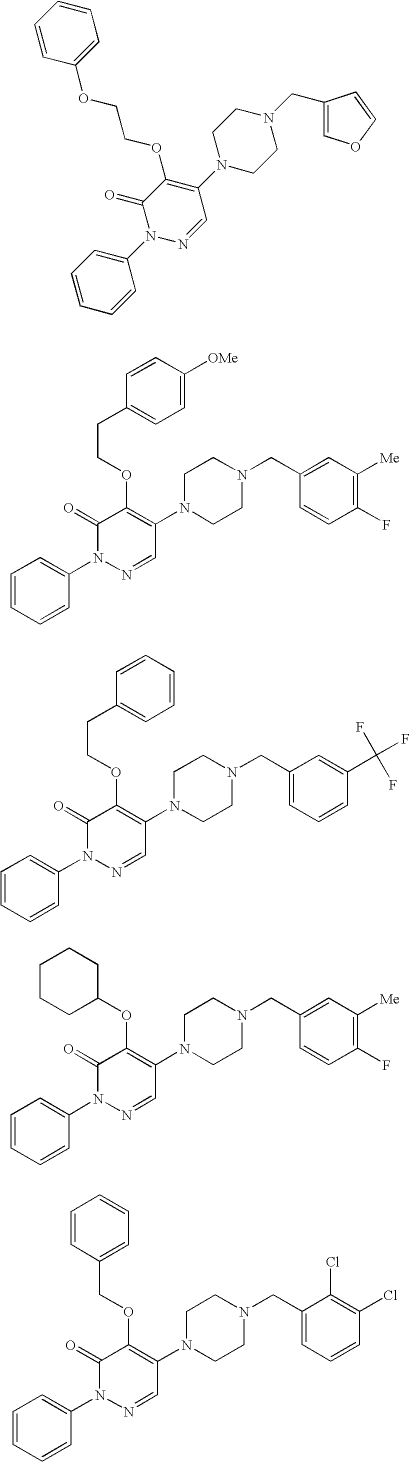

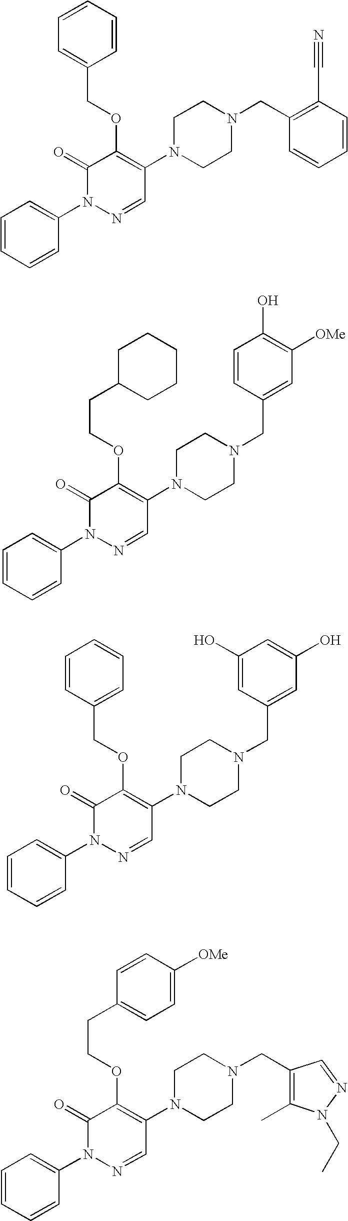

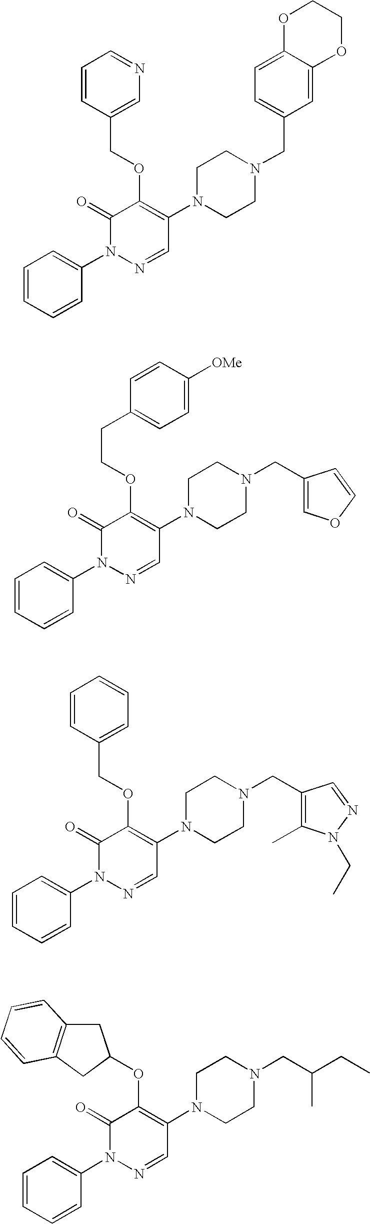

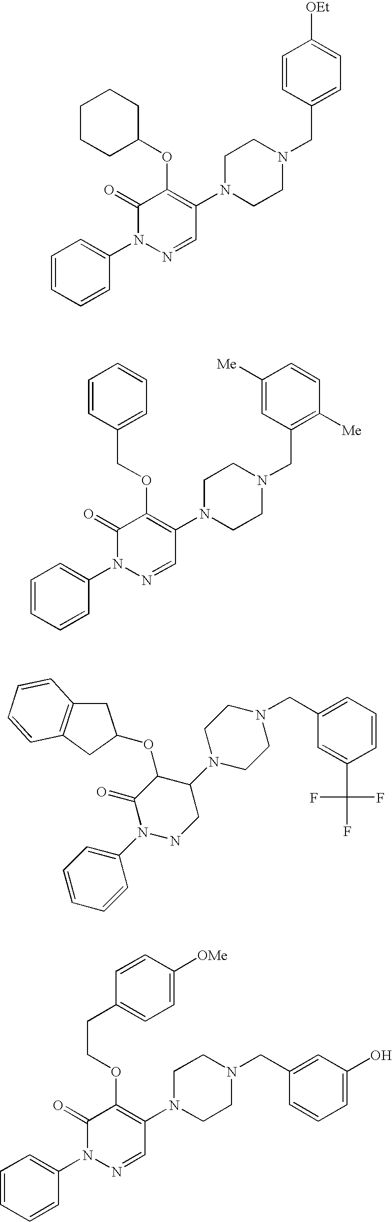

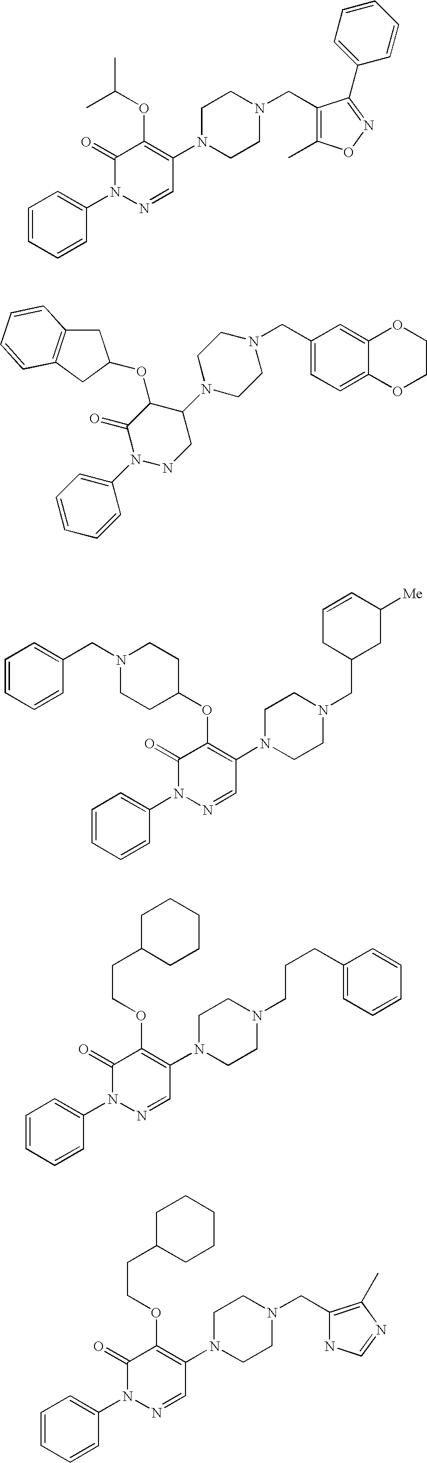

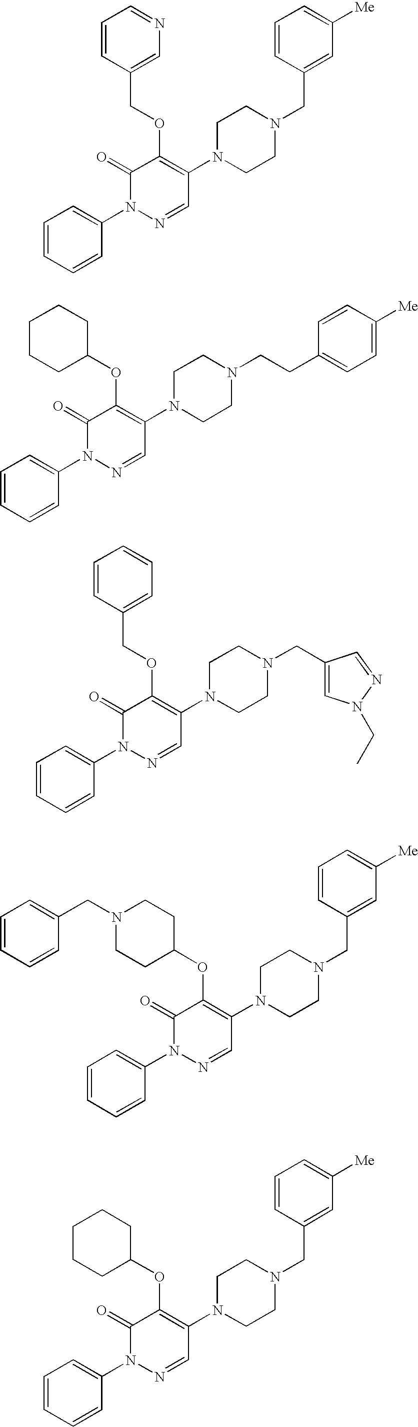

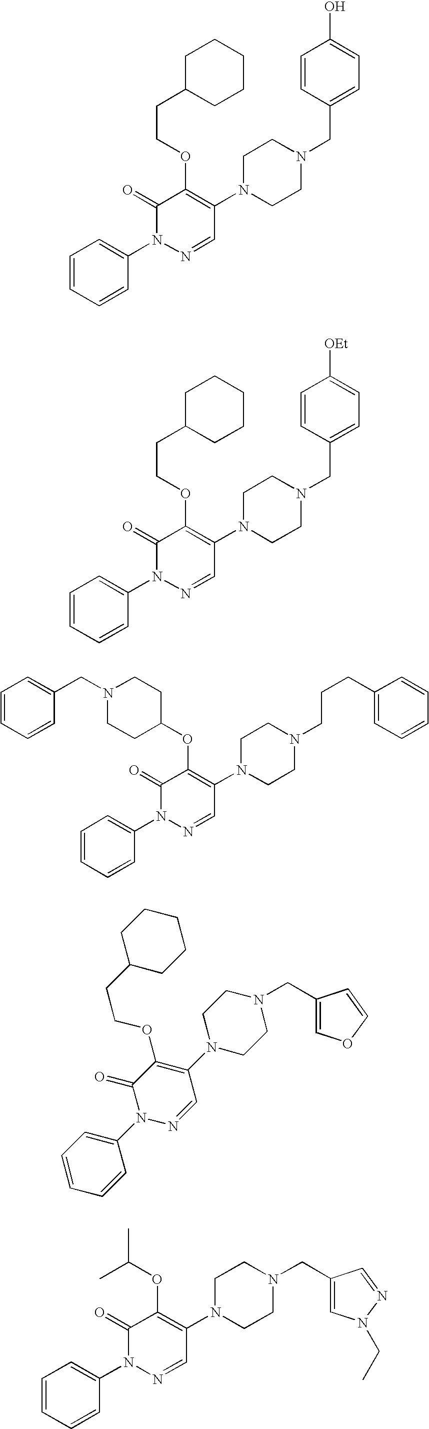

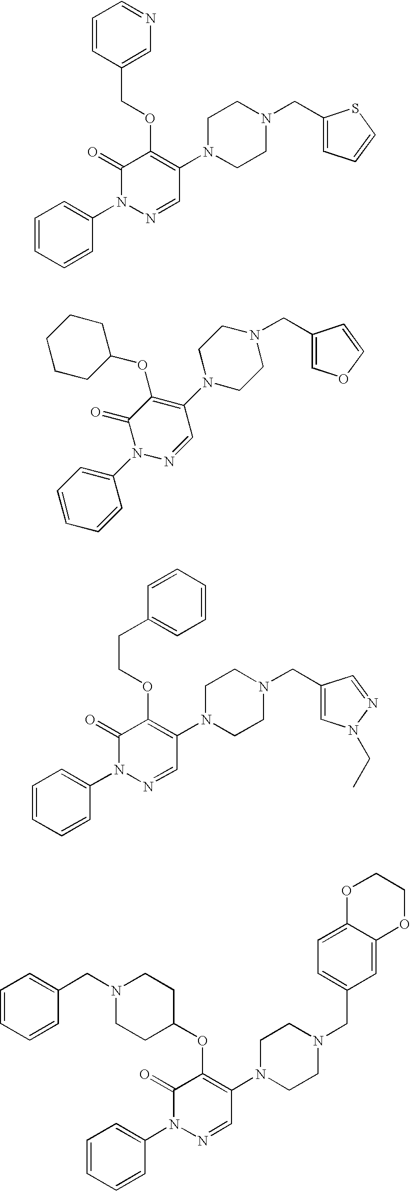

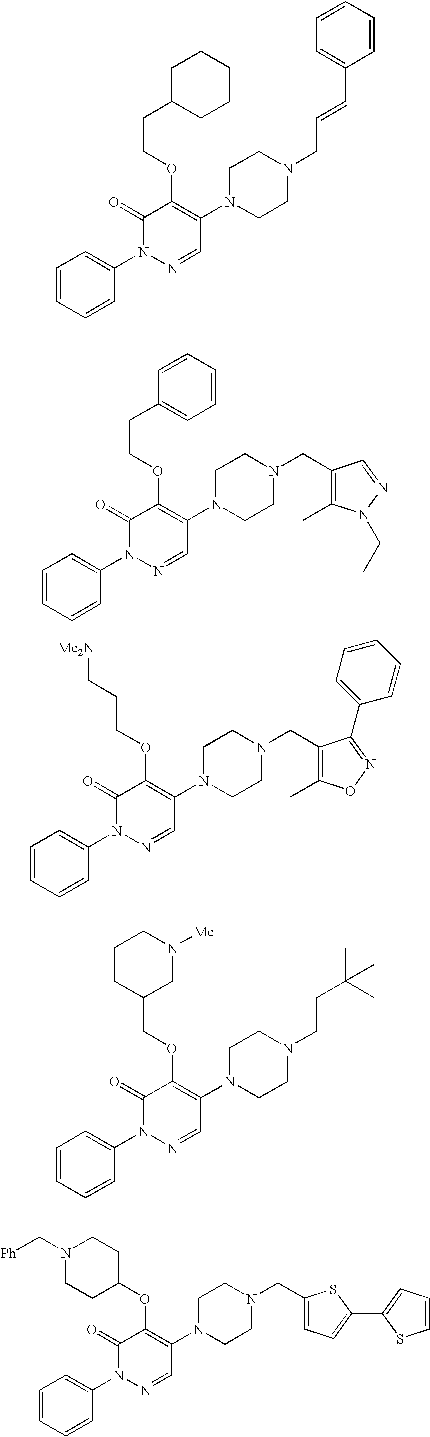

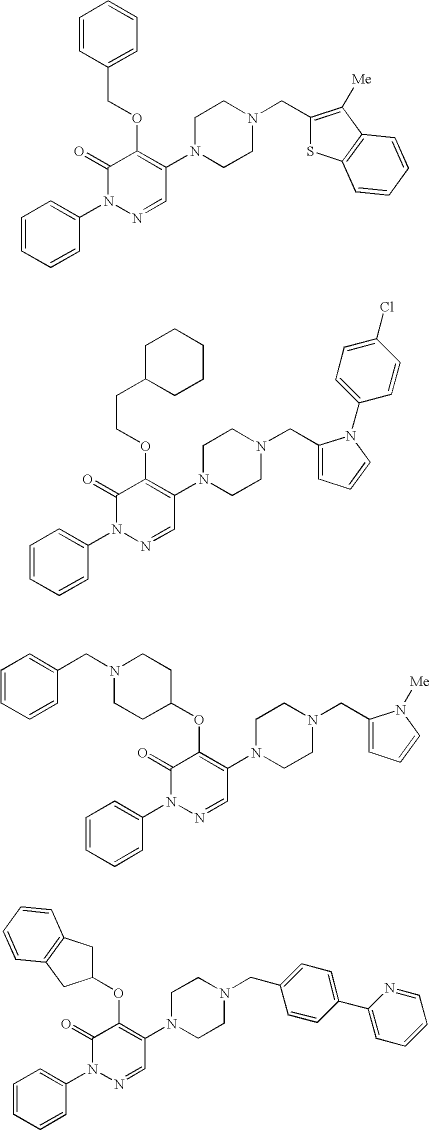

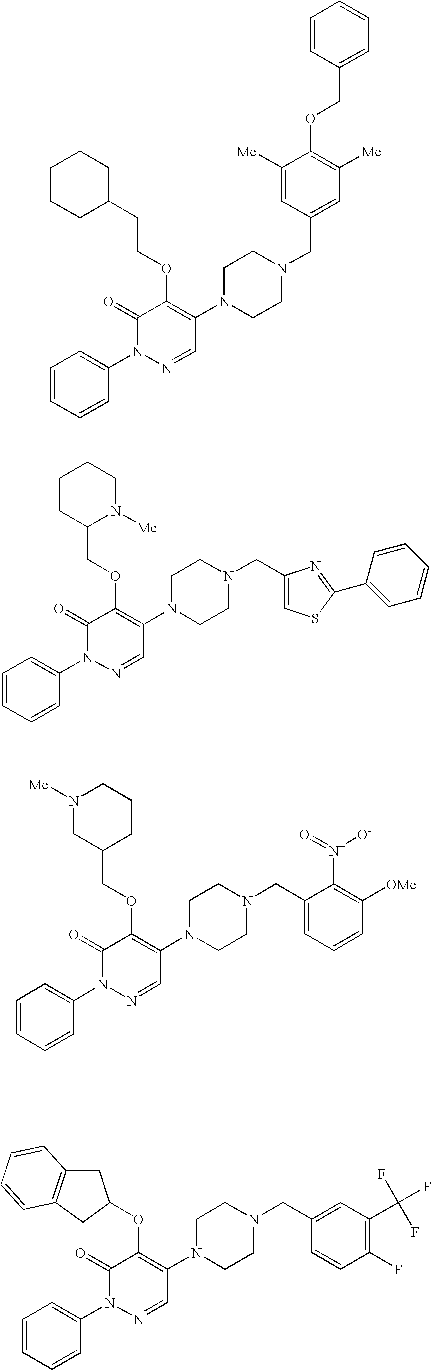

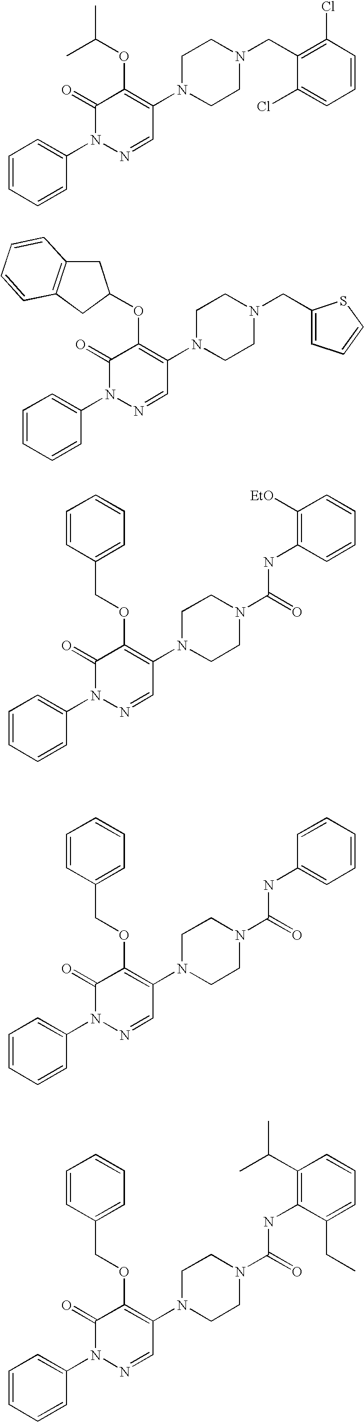

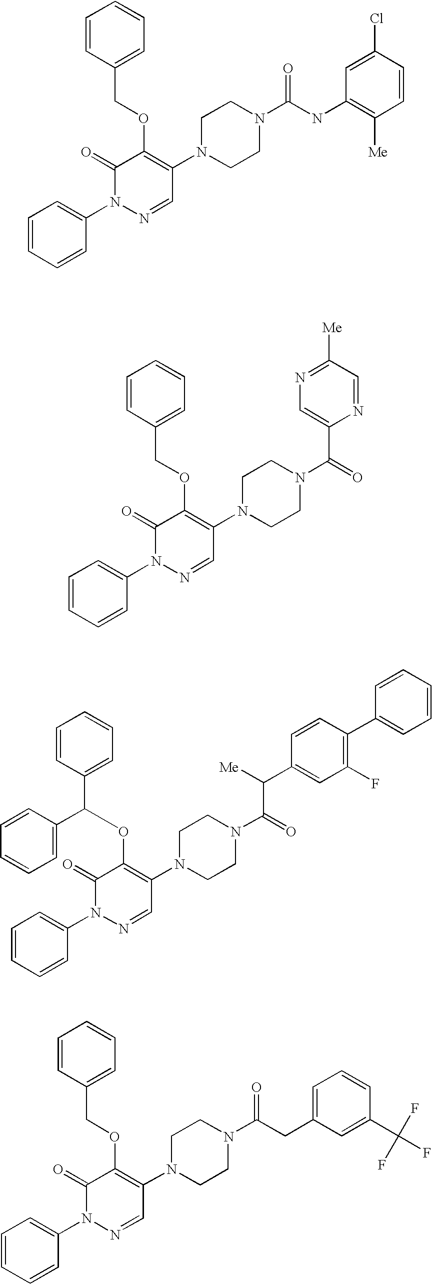

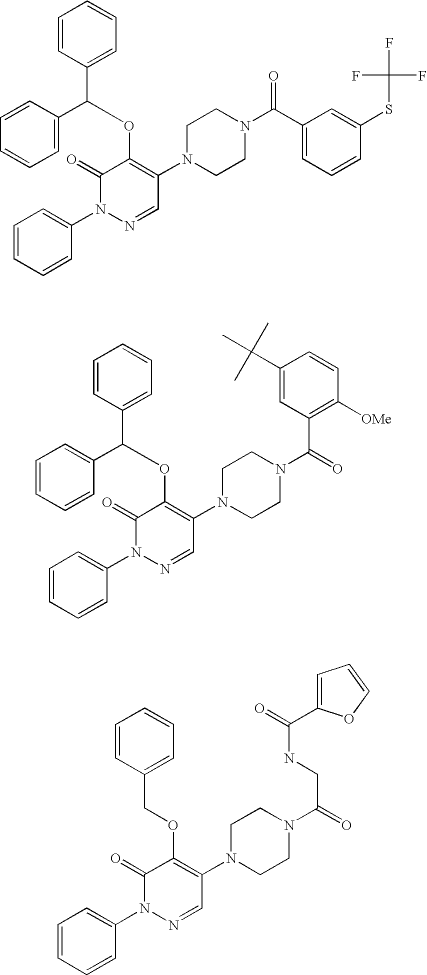

The invention also relates to a method of treating or preventing growth of fungal pathogens in plants, and to a method of reducing or eliminating fungal growth on inanimate surfaces, comprising applying one or more compounds listed above to said plant or surface.

The invention also relates to a method of treating or preventing growth of fungal pathogens on inanimate surface, comprising applying one or more compounds listed above to said surface.

The invention also relates to a method of treating or preventing growth of fungal pathogens on inanimate surface by applying one or more compounds listed above and one or more other antifungal agents to said surface.

The invention also relates to a method of treating fungal pathogens by administering pharmaceutical compositions for human or veterinary use comprising one or more compounds listed above and a pharmaceutically acceptable carrier.

The invention also relates to the use of a glucan synthase inhibitor listed above for the preparation of a medicament for treating or preventing fungal infections.

The invention also relates to a method of treating or preventing fungal infections by administering a combination of one or more compounds listed above and one or more other antifungal agents.

Further, the invention relates to a method of treating or preventing fungal infections by administering a human or veterinary pharmaceutical composition comprising one or more compounds listed above and one or more other antifungal agents in a pharmaceutically acceptable carrier. Also contemplated the method of preparing a kit comprising in a single package, one container comprising one or more compounds listed above in a pharmaceutically acceptable carrier, and a separate container comprising one or more other antifungal agents in a pharmaceutically acceptable carrier, with the compounds listed above and the other antifungal agents being present in amounts such that the combination is therapeutically effective.

### DETAILED DESCRIPTION OF THE INVENTION

The preferred method of treating or preventing fungal infections in animals comprise administering to an animal in need of such treatment an effective amount of one or more compounds of the formula:

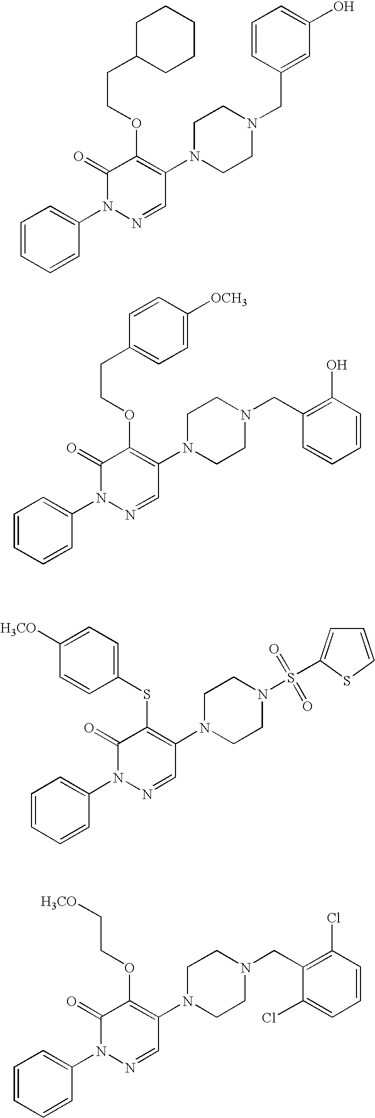

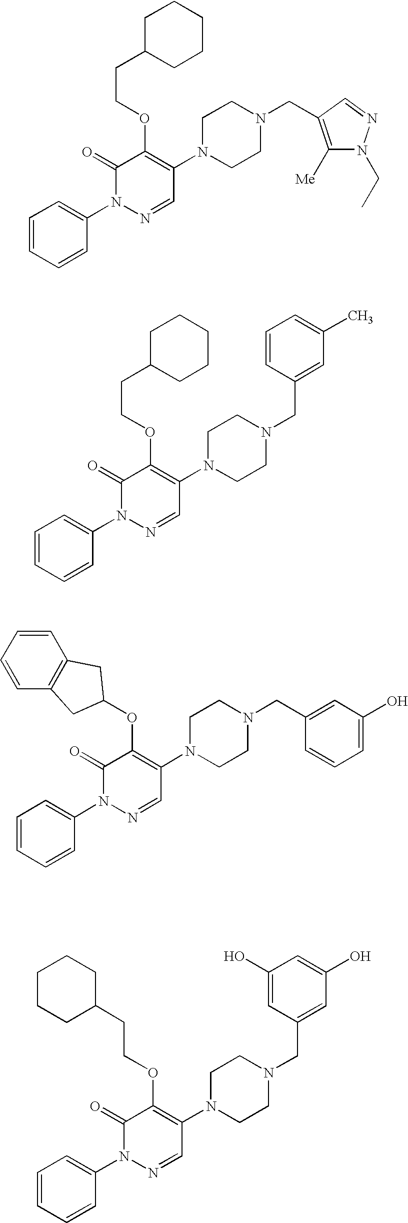

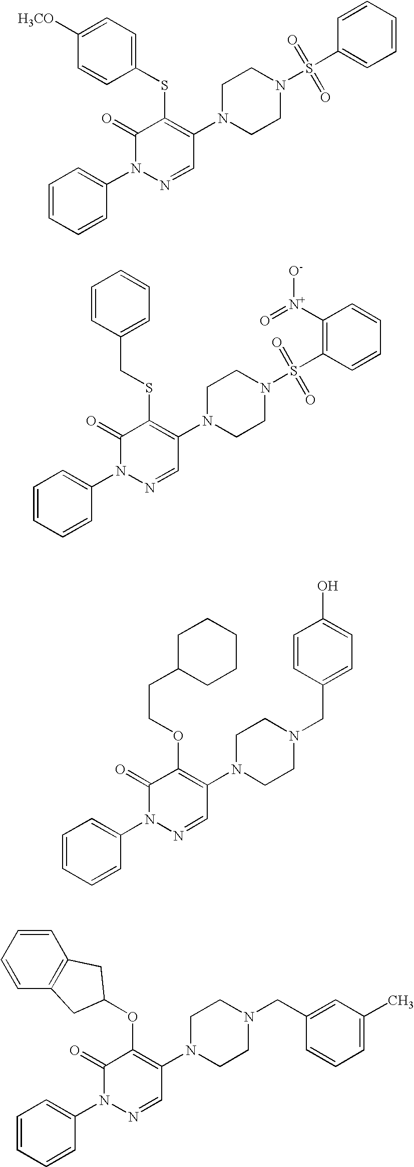

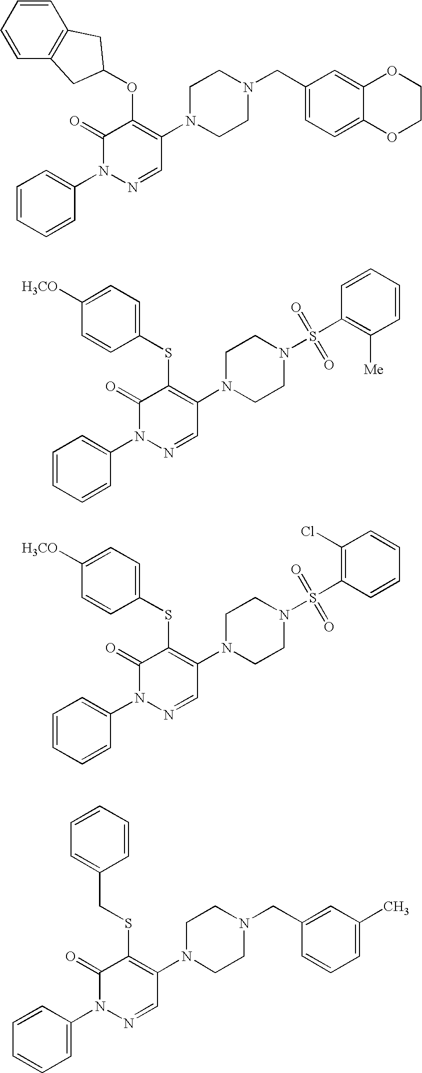

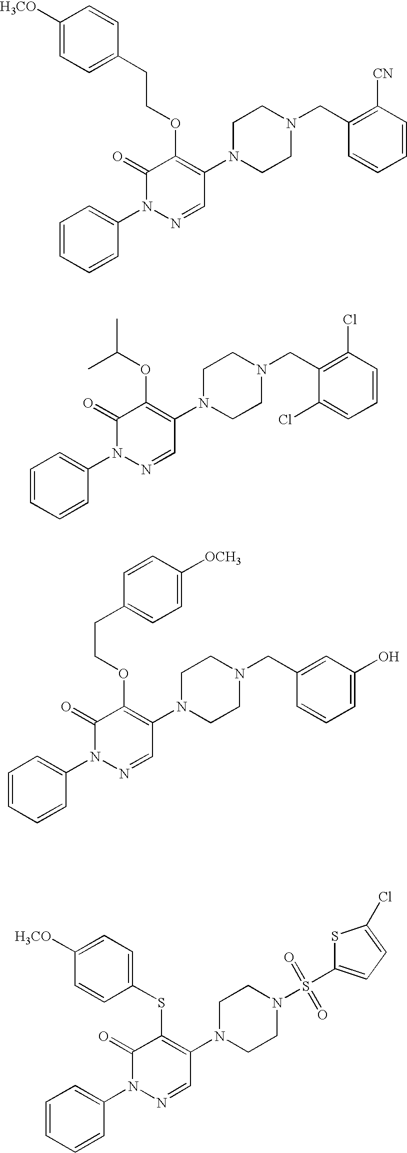

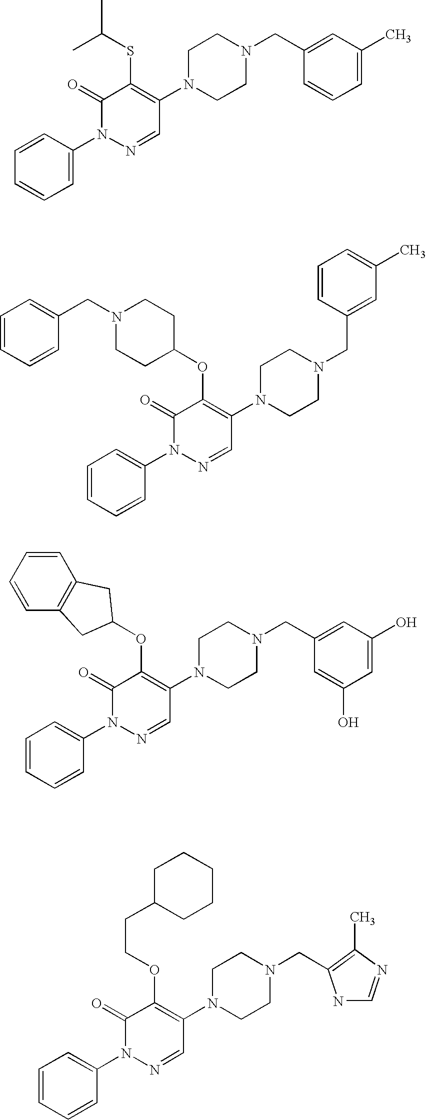

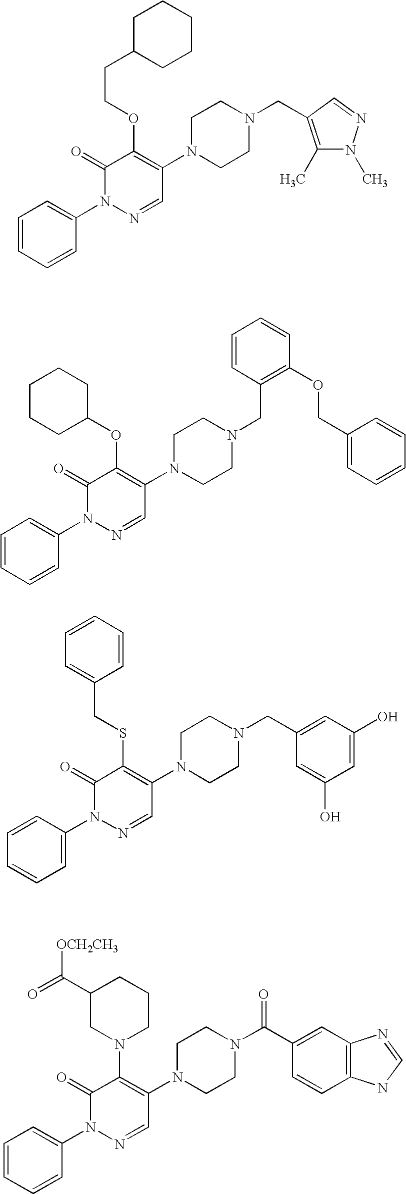

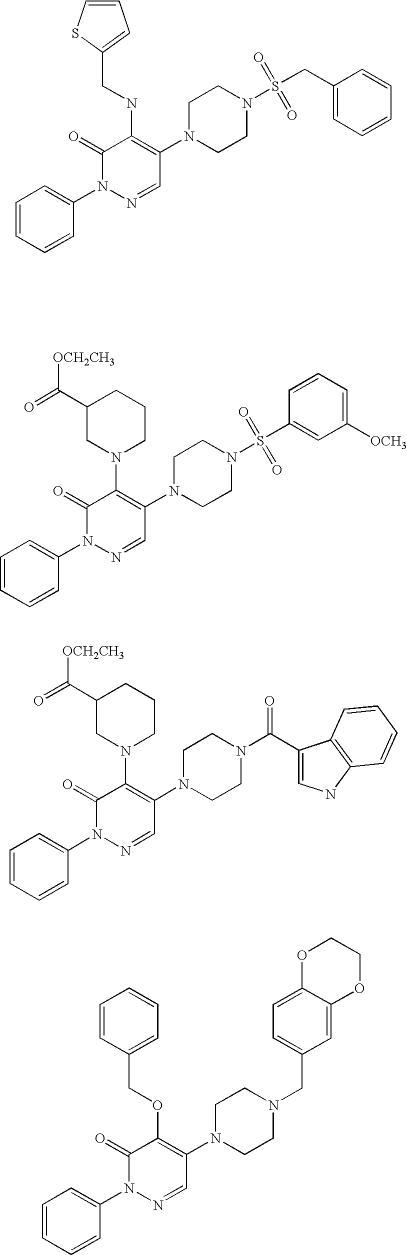

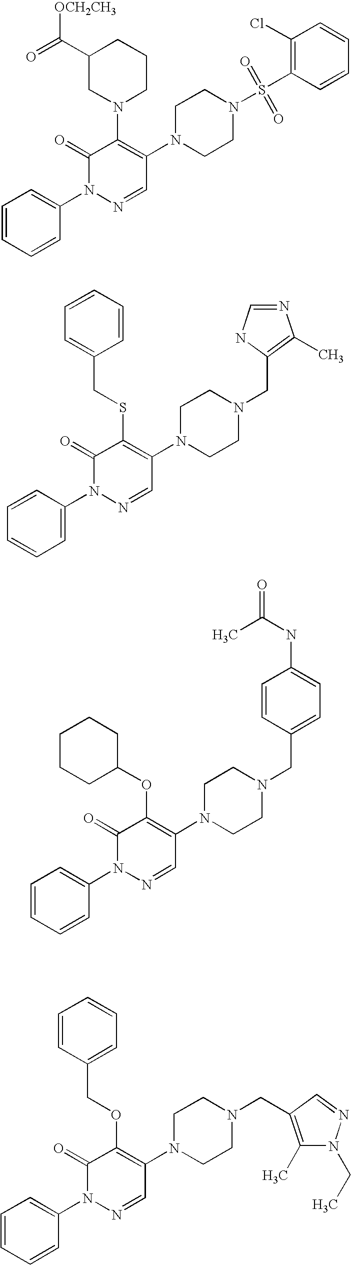

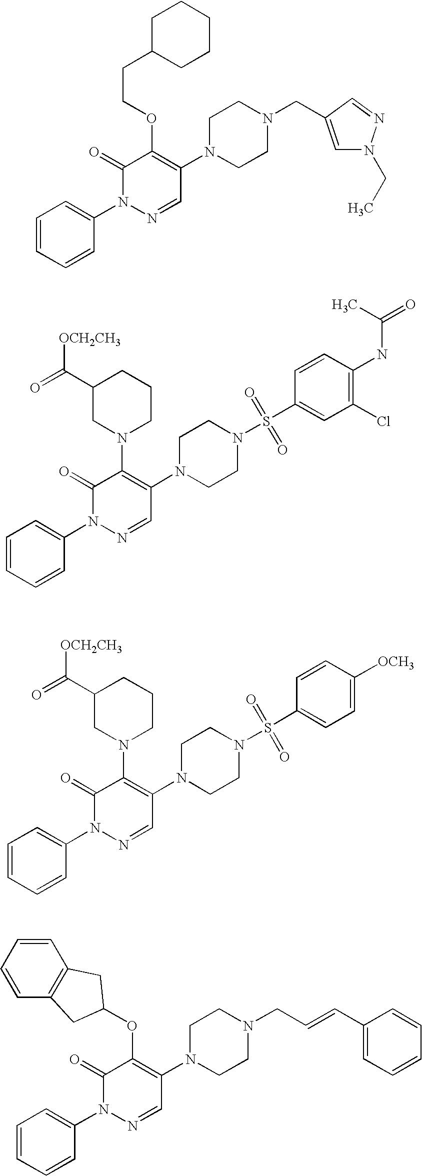

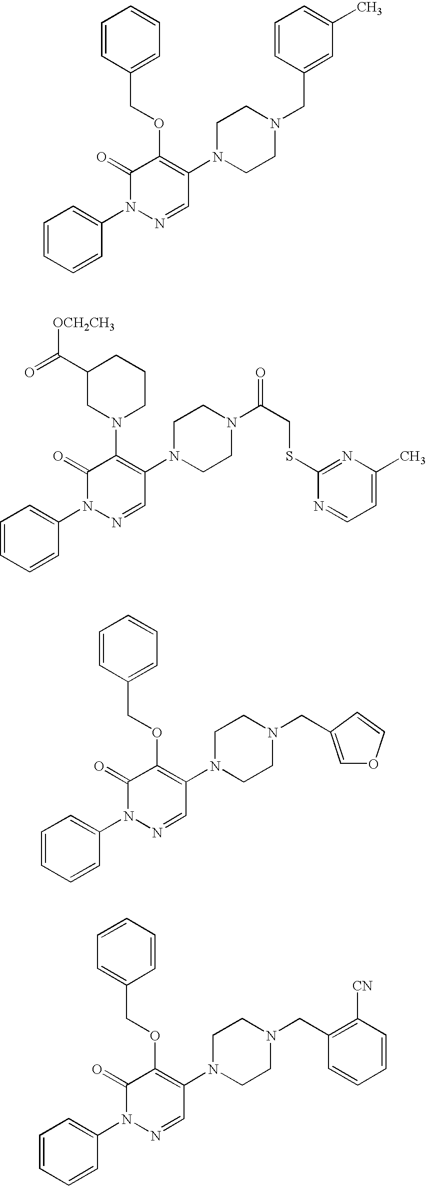

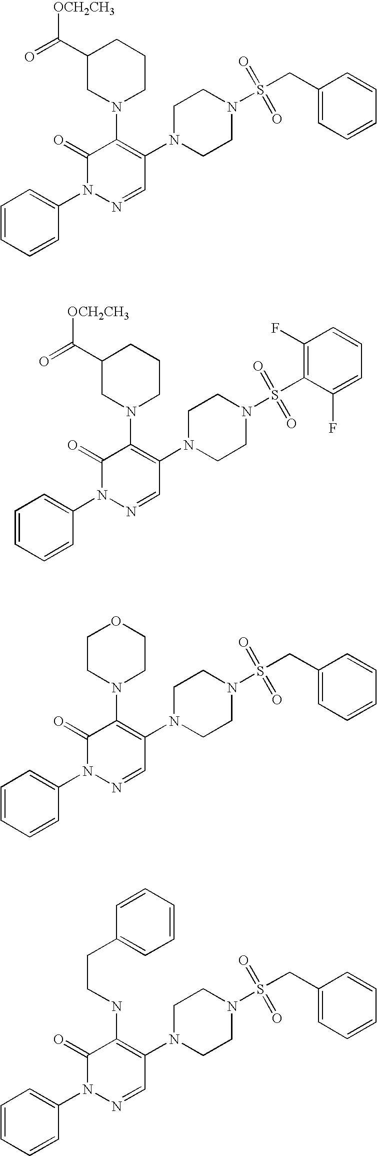

The compounds listed above are inhibitors of glucan synthase and therefore are useful in the treatment or prevention of fungal infections caused by pathogens such as, for example, Absidia corymbifera; Absidia spp; Acremonium spp; Ajellomyces capsulatus; Ajellomyces dermatitidis; Alternaria spp; Aphanoascus fulvescens; Apophysomyces spp; Arthroderma benhamiae; Arthroderma fulvum; Arthroderma gypseum; Arthroderma incurvatum; Arthroderma otae; Arthroderma vanbreuseghemii; Aspergillus flavus; Aspergillus fumigatus; Aspergillus glaucus; Aspergillus nidulans; Aspergillus niger; Aspergillus oryzae; Aspergillus spp; Aspergillus sydowi; Aspergillus terreus; Aspergillus ustus; Aspergillus versicolor; Aureobasidium pullulans; Basidiomycetes; Beauveria spp; Bipolaris hawaiiensis; Bipolaris spicifera; Bipolaris spp; Bjerkandera adusta; Blastomyces dermatitidis; Blastoschizomyces capitatus; Candida albicans; Candida beigelii; Candida colluculosa; Candida dubliniensis; Candida dubliniensis; Candida famata; Candida famata; Candida glabrata; Candida guilliermondii; Candida haemulonii; Candida holmii; Candida inconspicua; Candida intermedia; Candida keyfyr; Candida krusei; Candida krusei; Candida lambica; Candida lipolytica; Candida lusitaniae; Candida marls; Candida melibiosica; Candida norvegensis; Candida parapsilosis; Candida parapsilosis; Candida pelliculosa; Candida pelliculosa; Candida pseudotropicalis; Candida pulcherrima; Candida rugosa; Candida sake; Candida sphaerica; Candida spp; Candida stellatoidea; Candida tropicalis; Candida tropicalis; Candida viswanathii; Candida zeylanoides; Chrysosporium spp; Cladophialophora bantiana; Cladophialophora carrionii; Cladosporium spp; Coccidioides immitis; Cokeromyces recurvatus; Coprinus spp; Cryptococcus albidus; Cryptococcus gattii; Cryptococcus laurentii; Cryptococcus neoformans; Cunninghamella bertholletiae; Cunninghamella spp; Curvularia lunata; Curvularia spp; Dekkera bruxellensis; Epidermophyton floccosum; Epidermophyton floccosum; Exophiala dermatitidis; Exophiala jeanselmei; Exophiala moniliae; Exserohilum rostratum; Filobasidiella neoformans; Fonsecaea pedrosoi; Fusarium dimerum; Fusarium moniliforme; Fusarium oxysporum; Fusarium proliferatum; Fusarium solani; Fusarium spp; Geotrichum candidum; Geotrichum spp; Histoplasma capsulatum; Hortaea werneckii; Issatschenkia orientalis; Kluveromyces lactis; Kluyveromyces marxianus; Madurella grisae; Malassezia furfur; Malassezia globosa; Malassezia obtusa; Malassezia pachydermatis; Malassezia restricta; Malassezia slooffiae; Malassezia sympodialis; Metarrhizium anisopliae; Microsporum audouinii; Microsporum canis; Microsporum fulvum; Microsporum gypseum; Microsporum persicolor; Mucor circinelloides; Mucor hiemalis; Mucor racemosus; Mucor rouxii; Mucor spp; Nattrassia mangiferae; Nectria haematococca; Onychocola canadensis; Paecilomyces lilacinus; Paecilomyces spp; Paecilomyces variotii; Paracoccidioides brasiliensis; Penicillium marneffei; Penicillium spp; Phialophora spp; Phialophora verrucosa; Phoma spp; Pichia anomala; Pichia etchellsii; Pichia guilliermondii; Pichia ohmeri; Pithomyces spp; Pneumocystis carinii; Pseudallescheria boydii; Ramichloridium obovoideum; Rhizomucor miehei; Rhizomucor pusillus; Rhizomucor spp; Rhizopus arrhizus; Rhizopus microsporus; Rhizopus oryzae; Rhizopus schipperae; Rhizopus spp; Rhodotorula mucilaginosa; Rhodotorula rubra; Rhodotorula spp; Saccharomyces cerevisiae; Saccharomyces spp; Sagrahamala spp; Saksenaea vasiformis; Scedosporium apiospermum; Scedosporium prolificans; Schizophyllum commune; Schizosaccharomyces pombe; Scopulariopsis brevicaulis; Scytalidium dimidiatum Ulocladium spp; Sporobolomyces spp; Sporothrix schenckii; Trichoderma spp; Trichophyton krajdenii; Trichophyton mentagrophytes; Trichophyton raubitschekii; Trichophyton rubrum; Trichophyton soudanense; Trichophyton spp; Trichophyton terrestre; Trichophyton tonsurans; Trichophyton verrucosum; Trichophyton violaceum; Trichosporon asahii; Trichosporon beigelii; Trichosporon capitatum; Trichosporon cutaneum; Trichosporon inkin; Trichosporon mucoides; Trichosporon spp; Tritirachium spp; Wangiella dermatitidis or Yarrowia lipolytica.

Another embodiment discloses a method of treating or preventing fungal infections in animals comprise administering to an animal in need of such treatment an effective amount of one or more compounds of the formula:

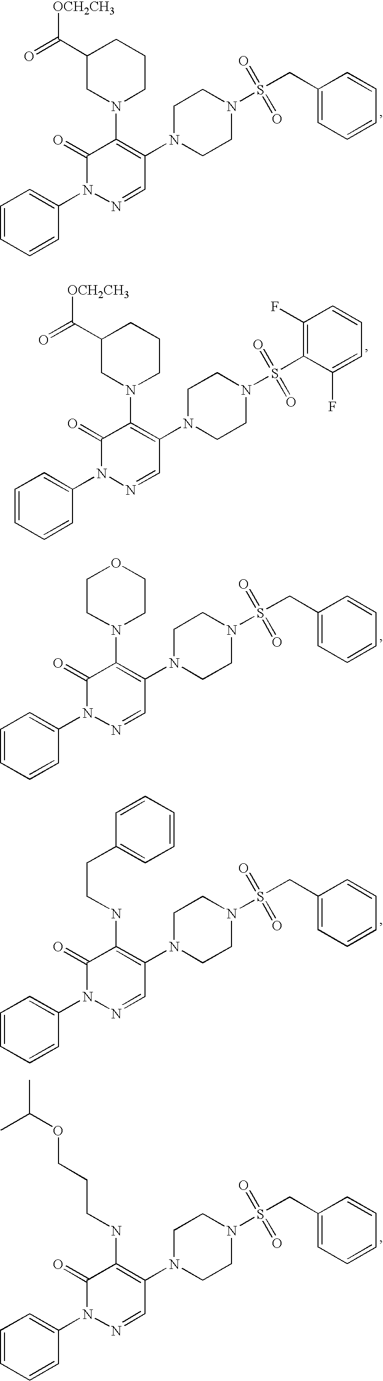

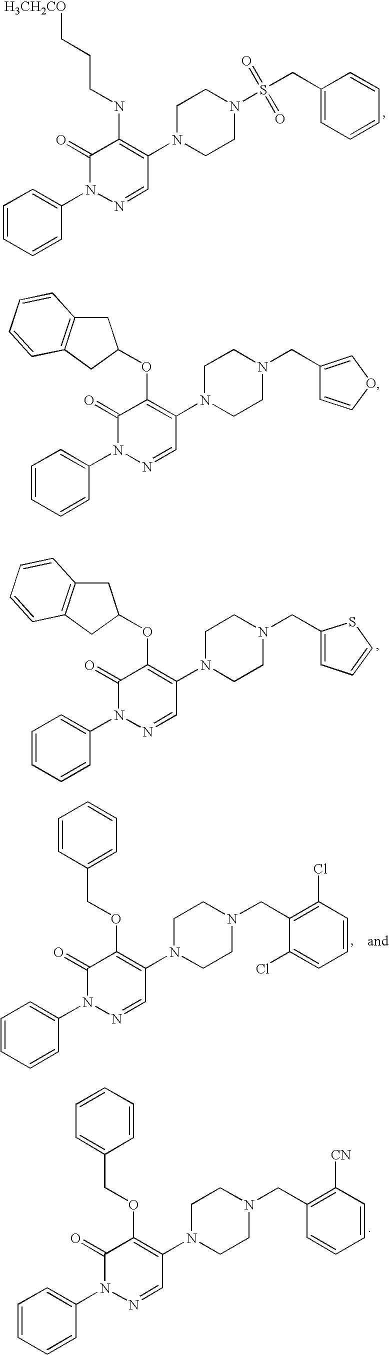

For pharmaceutical use, treatment of yeasts (e.g., Candida, Cryptococcus, Pichia, Rhodotorula, Saccharomyces, and Trichosporon) and moulds (e.g., Absidia, Alternaria, Apophysomyces, Arthroderma, Aspergillus, Bjerkandera, Blastomyces, Coccidioides, Cunninghamella, Epidermophyton, Exophiala, Fusarium, Histoplasma, Malassezia, Microsporum, Mucor, Paecilomyces, Penicillium, Pseudallescheria, Ramichloridium, Rhizomucor, Rhizopus, Saksenaea, Scedosporium, Sporothrix, Trichophyton and Wangiella) are preferred.

As used herein, the terms “treat” or “treating” mean eliminating the fungal infection, reducing the fungal burden, or stopping the progression of fungal growth.

The terms “prevent” or “preventing”, as used herein, mean administering at least one compound listed above before exposure to a potential fungal pathogen. For: example at least one compound listed above can be administered to an animal before organ transplant surgery, a procedure known to frequently result in fungal infections, or an animal known to be susceptible to fungal infections can be treated in advance of likely exposure. In the case of fungal plant pathogens, at least one compound listed above can be applied to a plant regularly throughout the growing season, before a potential pathogen can cause any harm to the plant.

When used to treat plant pathogens, at least one compound listed above can be applied to the leaves and stems of the plant using a method well known in the art, for example as a topical spray (e.g., an aqueous solution) or powder, or as a solution or powder added to the soil to allow systemic absorption. Topical application to plants is preferred. Similarly, when applied to the surfaces of inanimate objects to reduce or eliminate fungal growth, at least one compound listed above can be applied as a solution, a spray or a powder.

As indicated above, it is contemplated that more than one compound as listed above can be administered to treat fungal infections. When used herein, the terms “at least one” or “one or more” preferably mean one to three compounds, but more preferably one compound listed above is administered. When administered in combination with another antifungal agent, preferably one compound listed above and one other antifungal agent are administered.

Other antifungal agents for use in combination are for example: azoles (e.g. fluconazole, miconazole, itraconazole, voriconazole, posaconazole), echinocandins (e.g. caspofungin, micafungin, anidulafungin), polyenes (e.g. amphotericin B, including liposomal formulations of amphotericin B, and nystatin), allylamines (e.g. terbinafine), thiocarbamates (e.g. tolnaftate), nikkomycins, pradimicins, 5-fluorocytosines, oxaboroles, ciclopiroxolamine, griseofulvin, and morpholines (e.g., fenpropimorph).

As used herein, “animal” means a mammalian or non-mammalian (e.g., birds, fish, crustaceans, reptiles) species, preferably a mammal and more preferably a human. As used herein, “patient” refers to an animal, more preferably a human.

As well known in the art, a bond drawn from a particular atom wherein no moiety is depicted at the terminal end of the bond indicates a methyl group bound through that bond to the atom, unless stated otherwise.

For example:

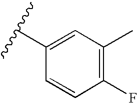

represents

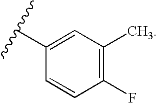

It should also be noted that any carbon or heteroatom with unsatisfied valences in the text, schemes, examples, structural formulae, and any Tables herein is assumed to have the hydrogen atom or atoms to satisfy the valences.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein.

A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound listed above or a pharmaceutically acceptable salt, hydrate or solvate of the compound. The transformation may occur by various mechanisms (e.g., by metabolic or chemical processes), such as, for example, through hydrolysis in blood.

For example, if a compound listed above or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a compound listed above contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound listed above incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy (C₁-C₆)alkyl, amino(C₁-C₄)alkyl or mono-N— or di-N,N-(C₁-C₆)alkylaminoalkyl, —C(Y⁴)Y⁵ wherein Y⁴ is H or methyl and Y⁵ is mono-N— or di-N,N-(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds listed above can form salts which are also within the scope of this invention. Reference to a compound listed above herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound listed above contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds listed above may be formed, for example, by reacting a compound listed above with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalky (for example, methoxymethyl), aralkyl (for example, benzyl), aryloxyalkyl (for example, phenoxymethyl), aryl (for example, phenyl optionally substituted with, for example, halogen; C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds listed above, and salts, solvates, esters and prodrugs thereof, may exist in their tautomeric form (for example, as an amide or imino ether). All such tautomeric forms are contemplated herein as part of the present invention.

The compounds listed above may contain asymmetric or chiral centers, and, therefore, exist in different stereoisomeric forms. It is intended that all stereoisomeric forms of the compounds listed above as well as mixtures thereof, including racemic mixtures, form part of the present invention. In addition, the present invention embraces all geometric and positional isomers. For example, if a compound listed above incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds listed above may be atropisomers (e.g., substituted biaryls) and are considered as part of this invention. Enantiomers can also be separated by use of chiral HPLC column.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. (For example, if a compound listed above incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labeled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled compounds listed above (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of a formula as described above can generally be prepared by following procedures analogous to those disclosed in the Schemes and/or in the Examples herein below, by substituting an appropriate isotopically labeled reagent for a non-isotopically labeled reagent.

Polymorphic forms of the compounds listed above, and of the salts, solvates, esters and prodrugs of the compounds listed above, are intended to be included in the present invention.

The term “pharmaceutical composition” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

Compounds listed above are prepared by methods known in the art. For example, a non-limiting method is according to the general reaction sequence shown in Scheme 1 and the preparative example following it:

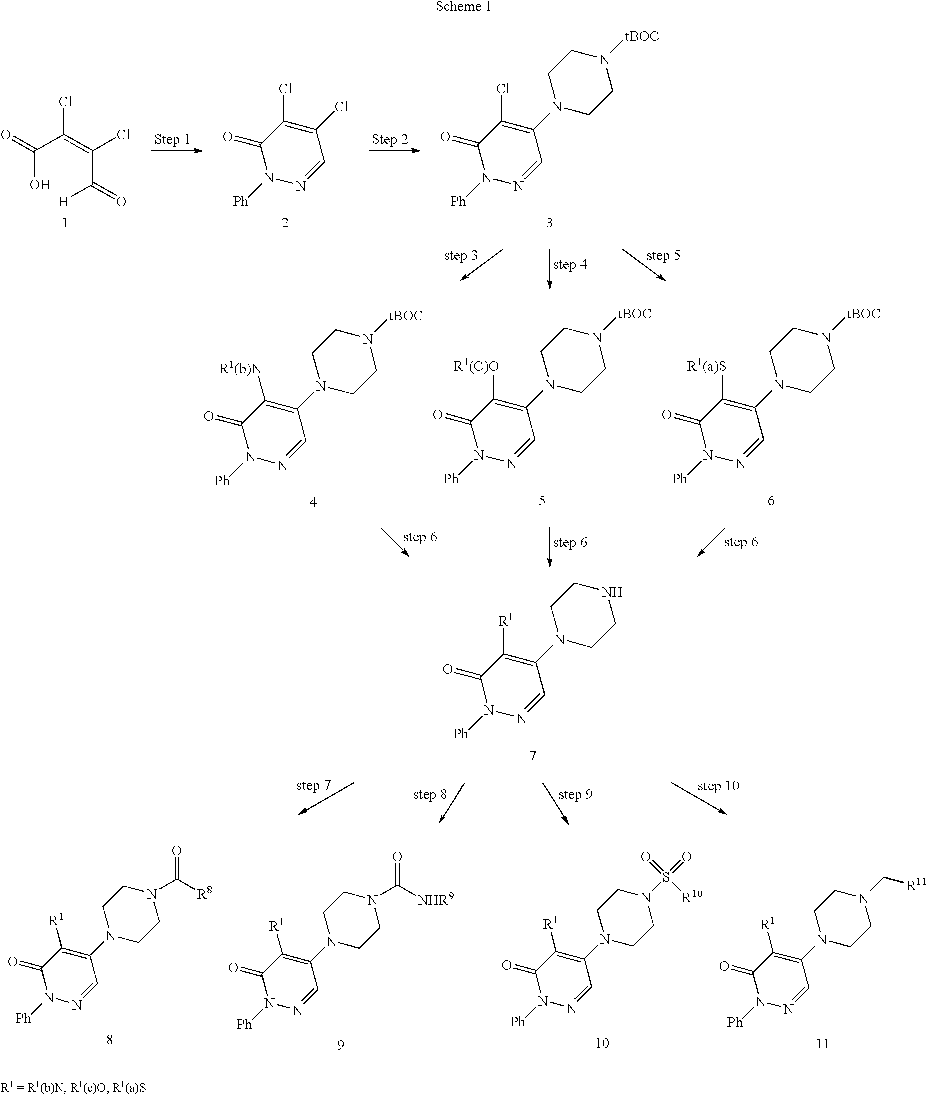

In Scheme 1, addition of R¹, R⁸, R⁹, R¹⁰ and R¹¹ refer to the corresponding positions of the compounds of the present invention listed above and are exemplified in Steps 6 to 10 below.

In this specification, the following abbreviations are used: RT=room temperature; DMF=dimethylformamide; Et=ethyl; EtOAc=ethyl acetate; Me=methyl; Ph=phenyl; tBOC=tert-butylcarbonyl; BINAP=2,2′-bis(diphenyl-phosphino)-1,1′ binaphthyl; THF=tetrahydrofuran; HATU=N-[(dimethylamino)-1H-1,2,3-triazolo-[4,5-b]pyridine-1-ylmethylene]N-methylmethanaminium hexafluorophosphate N-oxide.

### General Procedure:

### Step 1:

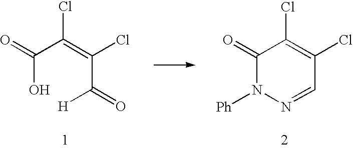

To a solution of NaOH (11.8 g, 0.296 mol) dissolved in water (25 ml) was added EtOH (150 ml) and phenylhydrazine hydrochloride (42.8 g, 0.296 mol). The reaction mixture was stirred at RT for 10 min, and then mucochloric acid 1 (50 g, 0.296 mol) was added. The resulting slurry was heated at 100° C. for 16 h then cooled to RT. Water (1500 ml) was added. The slurry was stirred and then filtered. The brown solid was air-dried for 10 min, then triturated with CH₃OH (100 ml) and filtered. The solid was washed with CH₃OH and air dried for 60 min to give 64.2 g (90%) of the product 2 as a beige solid. MS (M+1): m/e 241.

### Step 2:

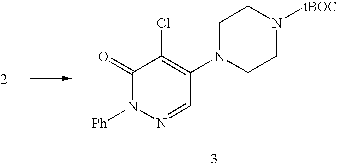

To a solution of compound 2 (15.00 g, 0.0622 mol) dissolved in EtOH (200 ml) was added N—BOC-piperazine (13.91 g, 0.0747 mol) and Hunigs base (11.26 g, 14.4 ml, 0.0871 mol). The reaction mixture was heated at reflux for 24 h and then cooled to RT. The solvent was evaporated, water (300 ml) was added, and the aqueous solution was extracted with CH₂Cl₂. The combined organic extract was dried (MgSO₄), filtered, and concentrated. Purification by silica gel chromatography (eluant: 15-20% EtOAc—CH₂Cl₂) gave 23.96 g (99%) of the product 3 as a light yellow solid. MS (M+1): m/e 391.

### Step 3 (Method 1):

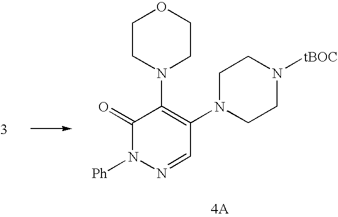

To a solution of compound 3 (461 mg, 1.18 mmol) dissolved in toluene (5 ml) was added morpholine (1 ml). The reaction mixture was heated in a sealed tube at 140° C. for 48 h and then cooled to RT. The solvent was evaporated. Purification by silica gel chromatography (eluant: EtOAc-hexane gradient) gave 115 mg (22%) of product 4A as an oil. MS (M+1): m/e 442.

Similar intermediates can be synthesized using the above procedure.

### Step 3 (Method 2):

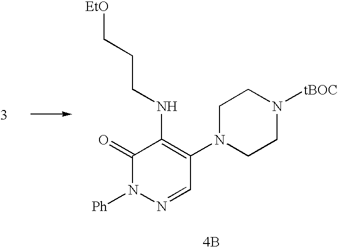

To a solution of compound 3 (5.0 g, 12.8 mmol) dissolved in toluene (250 ml) was added 3-ethoxypropylamine (1.59 g, 15.4 mmol), K₂CO₃ (8.64 g, 62.5 mmol), palladium acetate (86 mg, 0.38 mmol), and racemic BINAP (237 mg, 0.38 mmol) under N₂. The reaction mixture was heated at 120° C. for 30 h then cooled to RT. EtOAc was added, and the organic solution was washed with saturated aqueous NaCl, dried (MgSO₄), filtered and concentrated. Purification by silica gel chromatography (eluant: 2-10% EtOAc—CH₂Cl₂) gave 1.71 g (45%) of the product 4B as a white solid. MS (M+1): m/e 458.

Similar intermediates can be synthesized using the above procedure.

### Step 4:

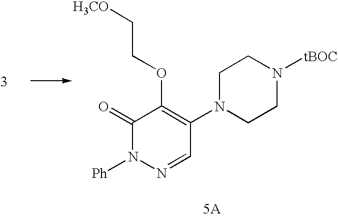

To a solution of 2-methoxyethanol (0.20 g, 2.69 mmol) dissolved in dry THF (10 ml) under N₂ was added sodium bis(trimethylsilyl)amide (1 M in THF, 2.3 ml, 2.30 mmol) via syringe. The reaction mixture was stirred at RT for 15 min, then compound 3 (0.75 g, 1.92 mmol) was added in dry THF (5 ml). The resulting solution was stirred at RT for 16 h. The solvent was evaporated, water (30 ml) was added, and the aqueous solution was extracted with EtOAc. The combined organic extract was dried (MgSO₄), filtered and concentrated. Purification by silica gel chromatography (eluant: 15-40% EtOAc—CH₂Cl₂) gave 0.65 g (78%) of the product 5A as a light yellow solid. MS (M+1): m/e 431.

Similar intermediates can be synthesized using the above procedure.

### Step 5:

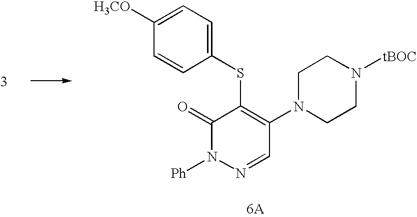

To a solution of compound 3 (195 mg, 0.50 mmol) dissolved in CH₃CN (2 ml) was added K₂CO₃ (70 mg, 0.50 mmol) and 4-methoxythiophenol (114 mg, 0.10 ml, 0.81 mmol). The reaction mixture was heated at reflux for 6 h and then cooled to RT. The solvent was evaporated, EtOAc was added, and the organic solution was washed with water, 1 N NaOH, and then saturated aqueous NaCl. The organic solution was dried (MgSO₄), filtered, and concentrated. Purification by silica gel chromatography (eluant: EtOAc-hexane gradient) gave 230 mg (93%) of the product 6A as a light yellow solid. MS (M+1): m/e 495.

Similar intermediates can be synthesized using the above procedure.

### Step 6:

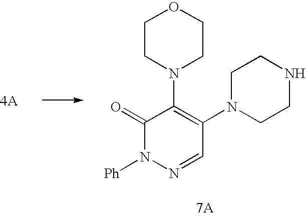

To a solution of compound 4A (644 mg, 1.46 mmol) dissolved in CH₂Cl₂ (10 ml) was added HCl-dioxane (4 N, 3.7 ml, 14.6 mmol). The reaction mixture was stirred at RT for 3 h. The solvent was evaporated, and the product was dried under high vacuum to give 498 mg (100%) of the HCl salt of the product 7A as a white solid. MS (M+1): m/e 342.

Similar intermediates can be synthesized using the above procedure.

### Step 7:

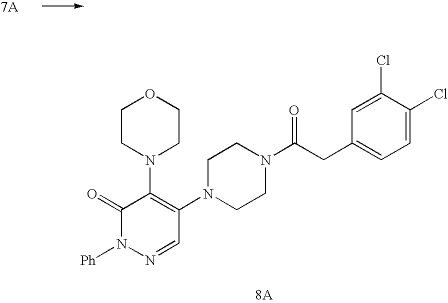

To a solution of the HCl salt of compound 7A (100 mg, 0.209 mmol) dissolved in DMF (3 ml) was added Et₃N (64 mg, 0.09 mL, 0.628 mmol), HATU (159 mg, 0.419 mmol), and 3,4-dichlorophenylacetic acid (64 mg, 0.314 mmol). The reaction mixture was stirred at RT for 16 h. The solvent was evaporated. Water was added, and the aqueous solution was extracted with CH₂Cl₂. The combined organic extract was dried (MgSO₄), filtered, and concentrated. Purification by silica gel chromatography (eluant: EtOAc—CH₂Cl₂ gradient) gave the product 8A. MS (M+1): m/e 528.

Similar compounds can be synthesized using the above procedure.

### Step 8:

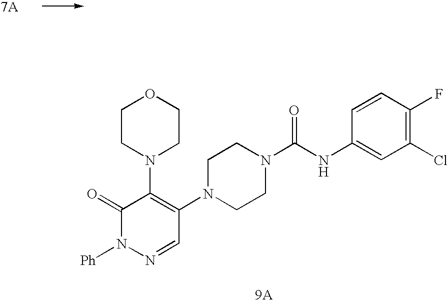

To a solution of the HCl salt of compound 7A (100 mg, 0.209 mmol) dissolved in dry THF (3 ml) was added Et₃N (42 mg, 0.06 ml, 0.418 mmol) and 3-chloro-4-fluorophenyl-isocyanate (72 mg, 0.418 mmol). The reaction mixture was heated at reflux for 24 h and then cooled to room temperature. The solvent was evaporated, water was added, and the aqueous solution was extracted with EtOAc. The combined organic extract was dried (MgSO₄), filtered, and concentrated. Purification by silica gel chromatography (eluant: EtOAc—CH₂Cl₂ gradient) gave the product 9A. (MS (M+1): m/e 513.

Similar compounds can be synthesized using the above procedure.

### Step 9:

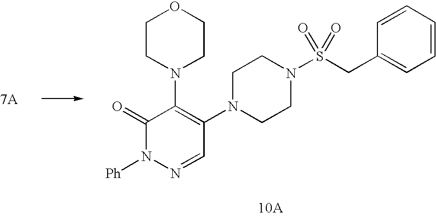

To a solution of the HCl salt of compound 7A (100 mg, 0.209 mmol) dissolved in DMF (3 ml) was added Hunigs base (81 mg, 0.10 ml, 0.628 mmol) and α-toluenesulfonyl chloride (60 mg, 0.314 mmol). The reaction mixture was stirred at RT for 3 h. Water was added, and the aqueous solution was extracted with CH₂Cl₂. The combined organic extract was dried (MgSO₄), filtered, and concentrated. Purification by silica gel chromatography (eluant: EtOAc—CH₂Cl₂ gradient) gave the product 10A. MS (M+1): m/e 496.

Similar compounds can be synthesized using the above procedure.

### Step 10:

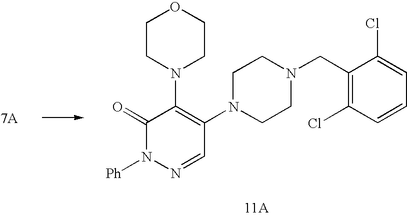

The HCl salt of compound 7A was converted to the free base by neutralization with diethylaminomethylpolystyrene resin in CH₃OH. To a solution of compound 7A (100 mg, 0.227 mmol) dissolved in dichloroethane (3 ml) was added 2,6-dichloro-benzaldehyde (59 mg, 0.340 mmol), sodium triacetoxyborohydride (72 mg, 0.340 mmol), and glacial acetic acid (10 mg, 0.170 mmol). The reaction mixture was stirred at RT for 24 h. 0.5 N NaOH was added, and the aqueous solution was extracted with CH₂Cl₂. The combined organic extract was dried (MgSO₄), filtered, and concentrated. Purification by silica gel chromatography (eluant: EtOAc—CH₂Cl₂ gradient) gave the product 11A. MS (M+1): m/e 500.

Similar compounds can be synthesized using the above procedure.

Using procedures analogous to those described above, the following compounds were made:

| | | MS |
|---|---|---|
| Ex. | Structure | M + 1 |
| 1 | 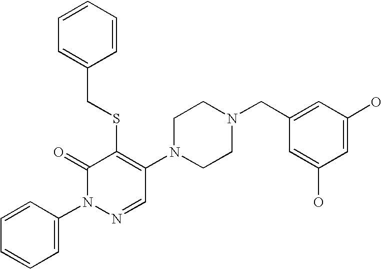 | 501 |
| 2 | 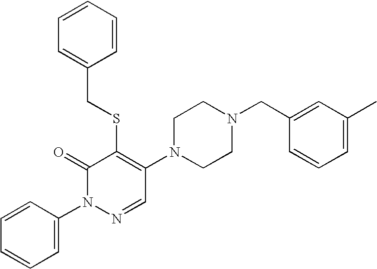 | 483 |
| 3 | 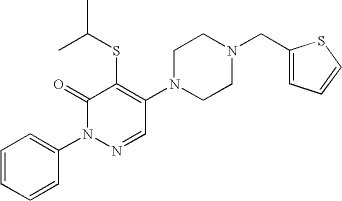 | 427 |
| 4 | 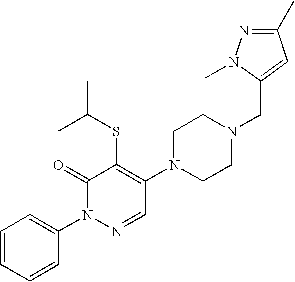 | 439 |
| 5 | 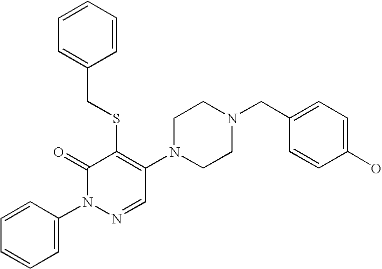 | 485 |
| 6 | 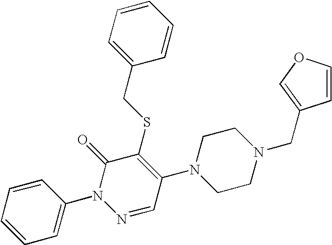 | 459 |
| 7 | 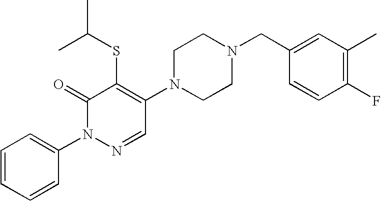 | 453 |
| 8 | 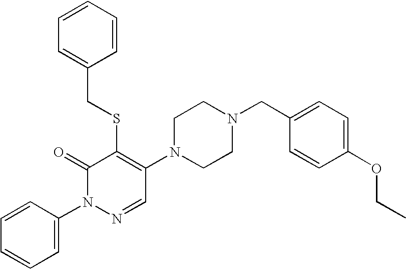 | 513 |
| 9 | 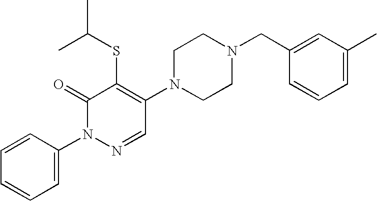 | 435 |
| 10 | 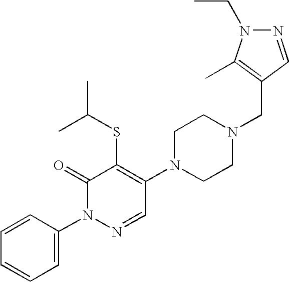 | 453 |
| 11 | 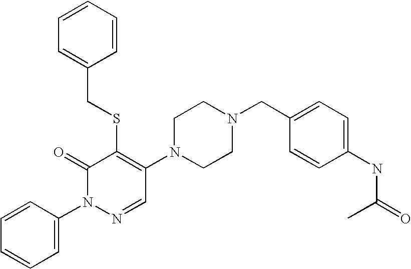 | 526 |
| 12 | 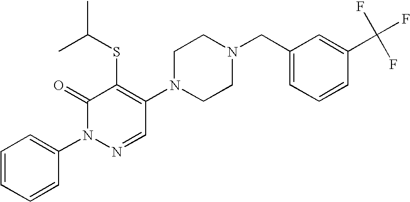 | 489 |
| 13 | 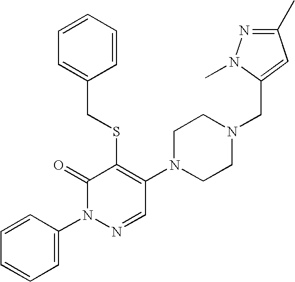 | 487 |
| 14 | 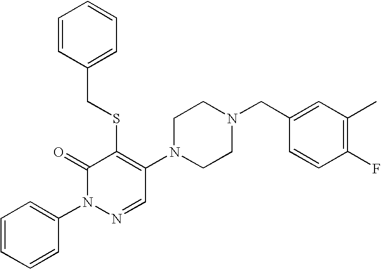 | 501 |
| 15 | 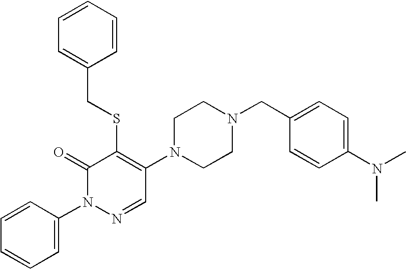 | 512 |
| 16 | 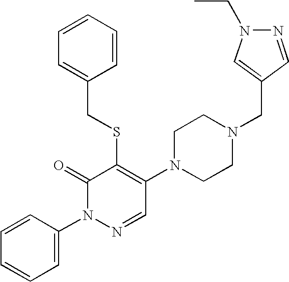 | 487 |
| 17 | 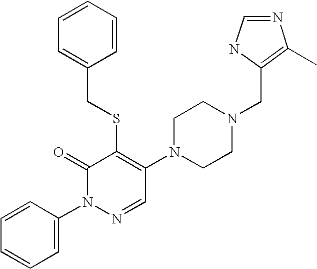 | 473 |
| 18 | 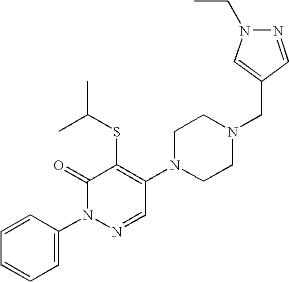 | 439 |
| 19 | 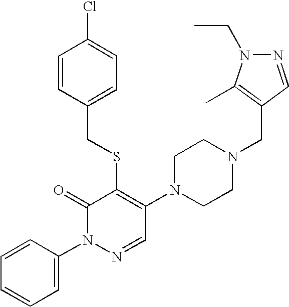 | 535 |
| 20 | 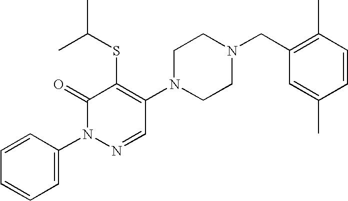 | 449 |
| 21 | 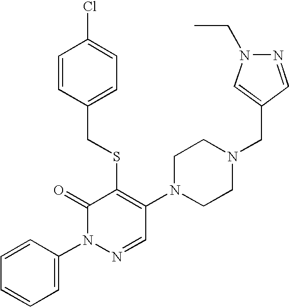 | 521 |
| 22 | 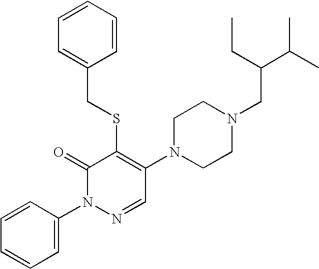 | 477 |
| 23 | 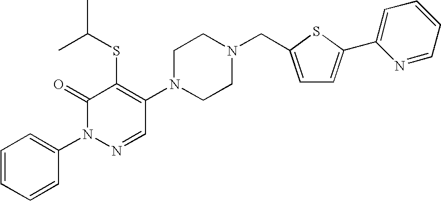 | 504 |
| 24 | 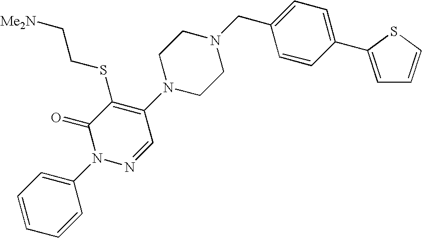 | 532 |
| 25 | 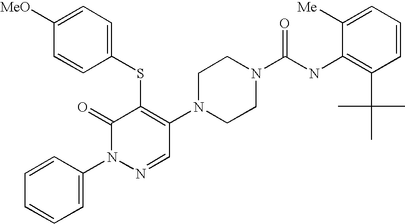 | 584 |
| 26 | 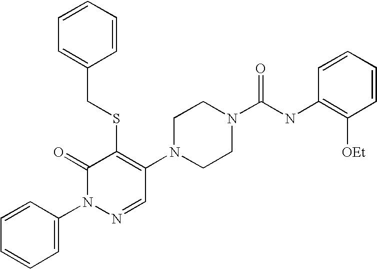 | 542 |
| 27 | 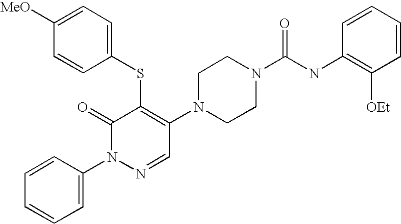 | 558 |
| 28 | 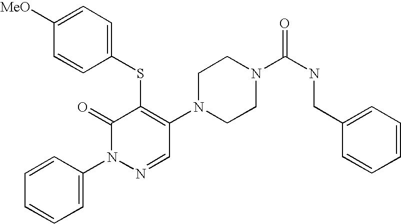 | 528 |
| 29 | 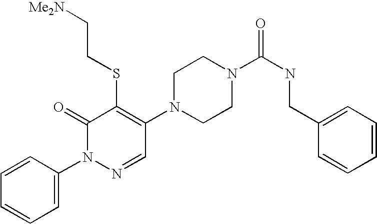 | 493 |
| 30 | 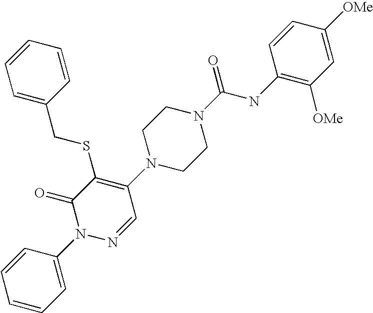 | 542 |
| 31 | 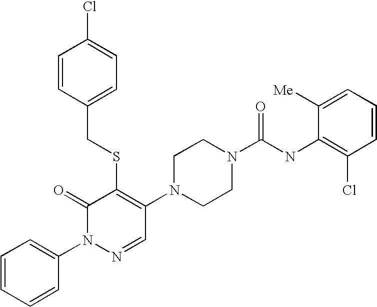 | 581 |
| 32 | 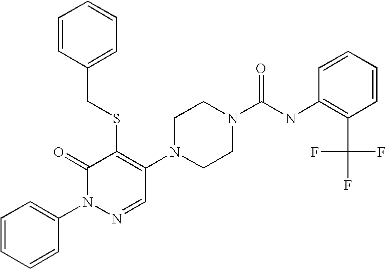 | 566 |
| 33 | 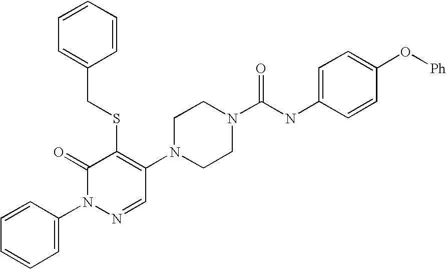 | 590 |
| 34 | 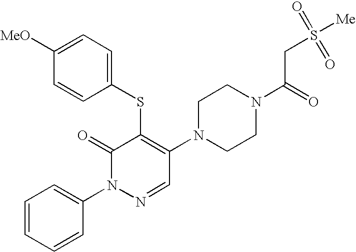 | 515 |
| 35 | 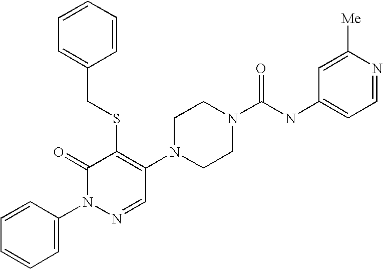 | 498 |
| 36 | 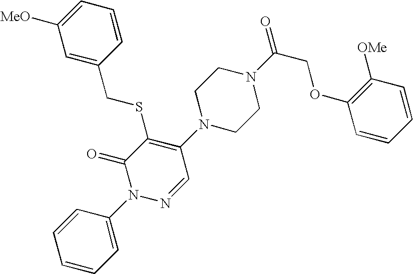 | 559 |
| 37 | 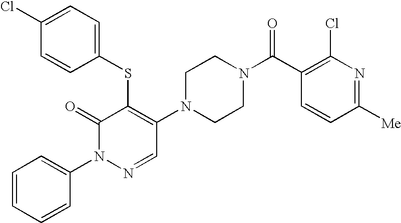 | 552 |
| 38 | 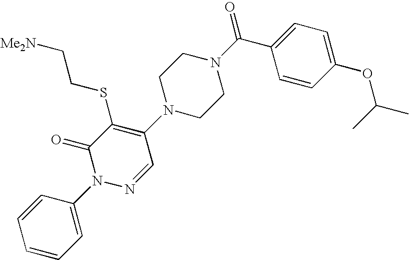 | 522 |
| 39 | 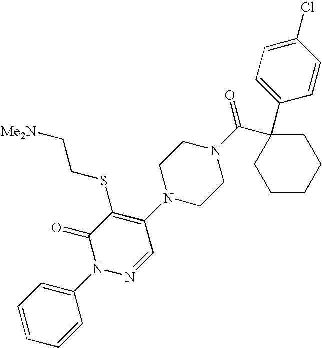 | 580 |
| 40 | 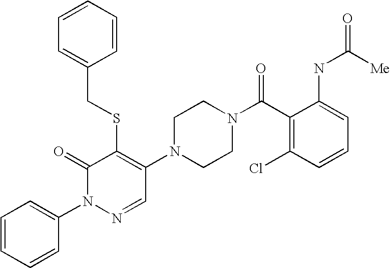 | 574 |
| 41 | 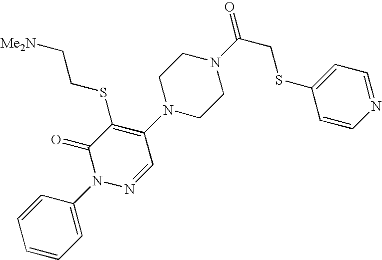 | 511 |
| 42 | 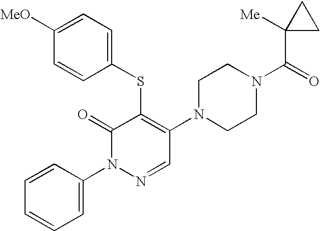 | 477 |
| 43 | 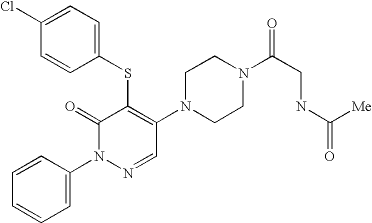 | 498 |
| 44 | 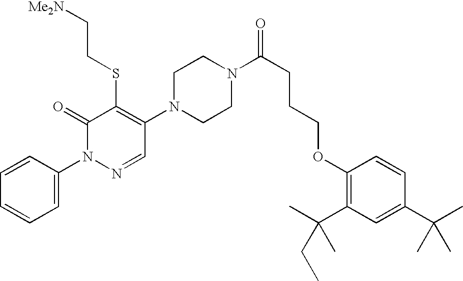 | 662 |
| 45 | 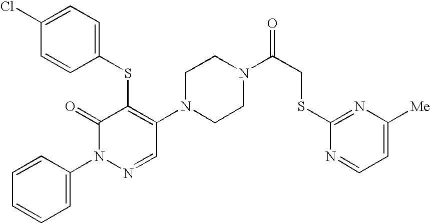 | 565 |
| 46 | 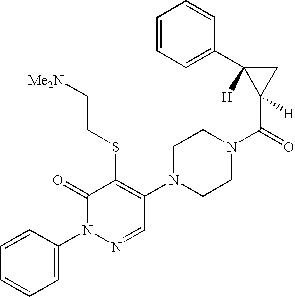 | 504 |
| 47 | 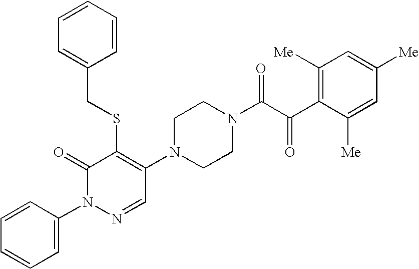 | 553 |
| 48 | 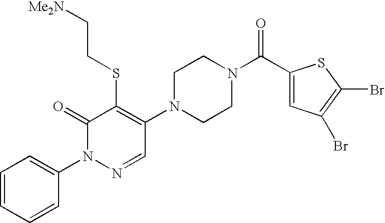 | 627 |
| 49 | 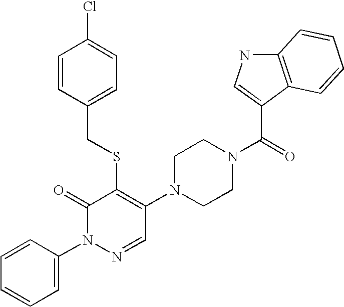 | 556 |
| 50 | 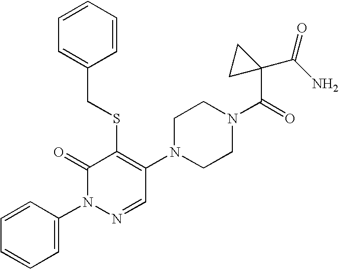 | 490 |
| 51 | 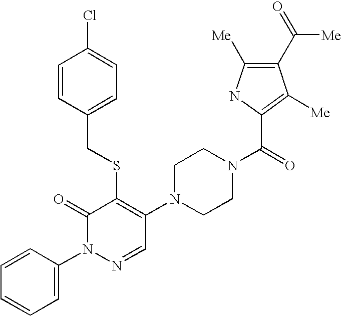 | 576 |
| 52 | 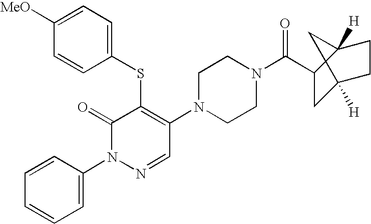 | 517 |
| 53 | 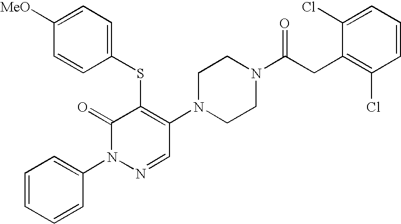 | 582 |
| 54 | 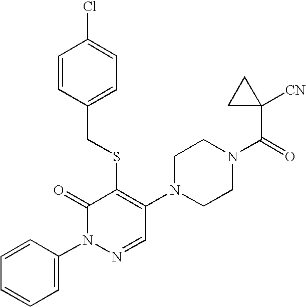 | 506 |
| 55 | 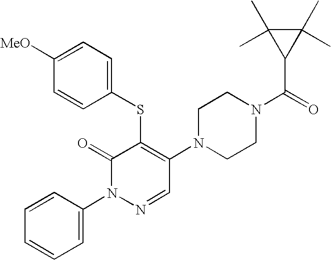 | 519 |
| 56 | 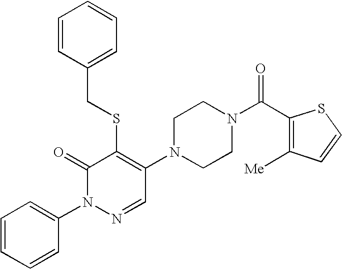 | 503 |
| 57 | 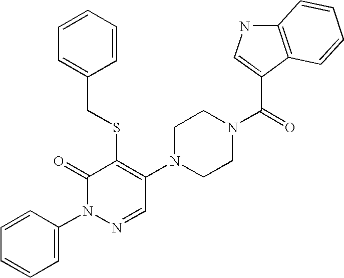 | 522 |
| 58 | 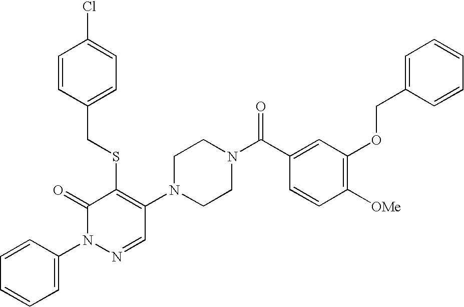 | 653 |
| 59 | 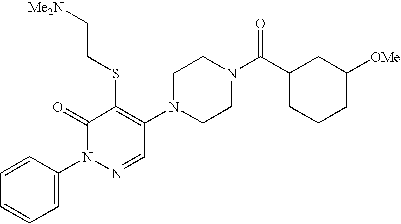 | 500 |
| 60 | 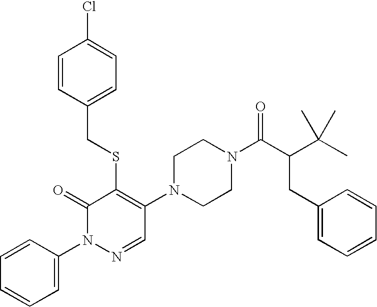 | 601 |
| 61 | 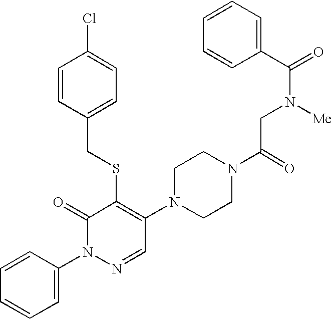 | 588 |
| 62 | 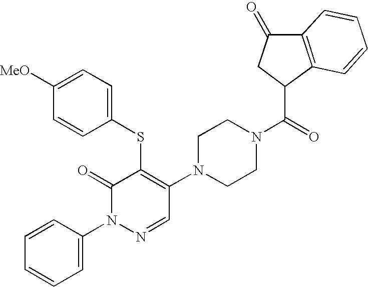 | 553 |
| 63 | 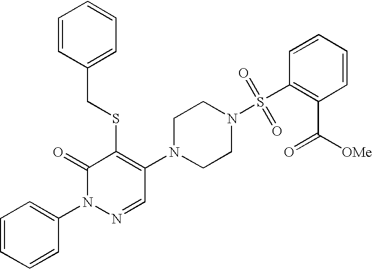 | 577 |
| 64 | 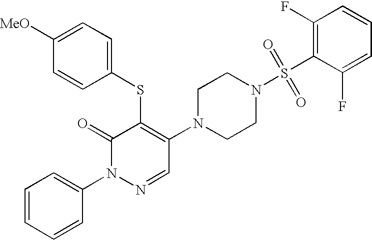 | 571 |
| 65 | 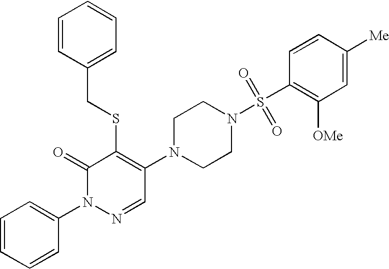 | 563 |
| 66 | 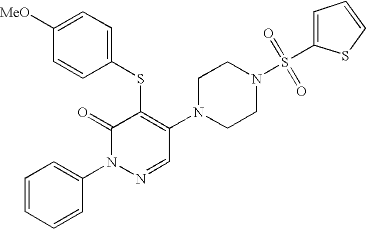 | 541 |
| 67 | 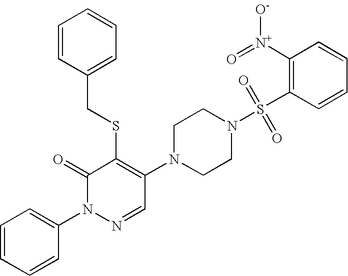 | 564 |
| 68 | 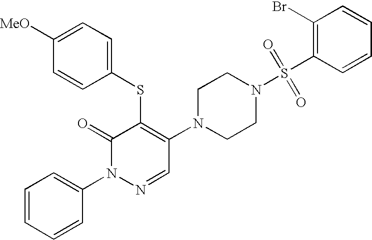 | 614 |
| 69 | 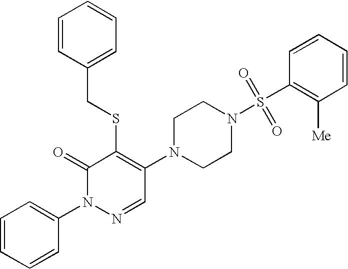 | 533 |
| 70 | 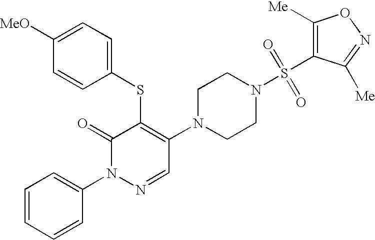 | 554 |
| 71 | 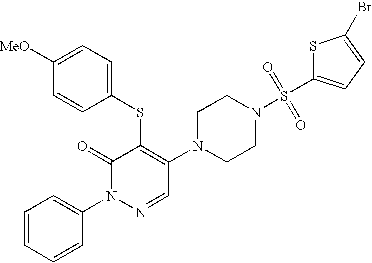 | 620 |
| 72 | 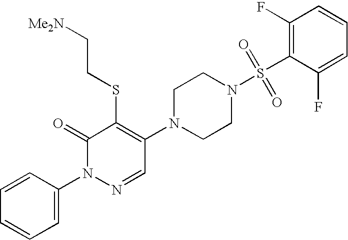 | 536 |
| 73 | 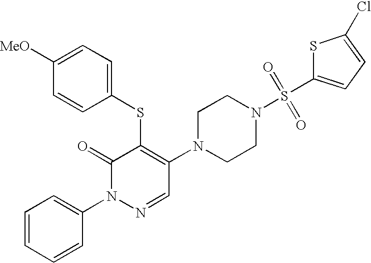 | 575 |
| 74 | 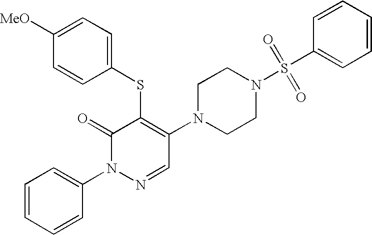 | 535 |
| 75 | 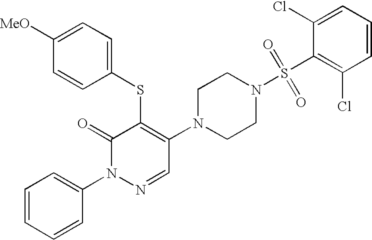 | 604 |
| 76 | 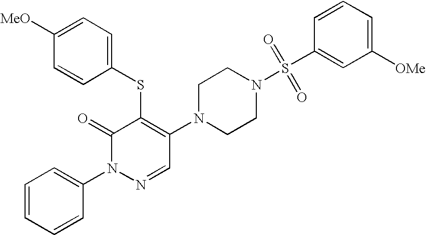 | 565 |
| 77 | 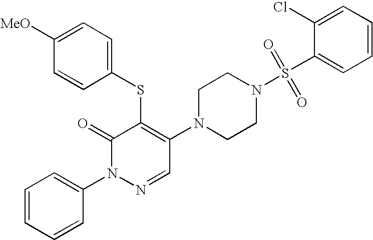 | 569 |
| 78 | 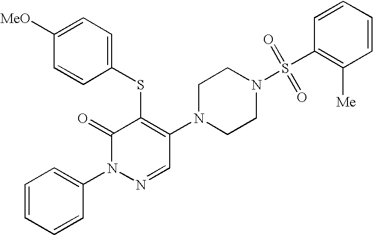 | 549 |
| 79 | 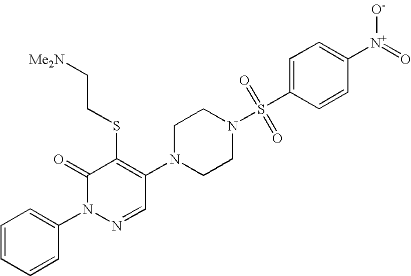 | 545 |
| 80 | 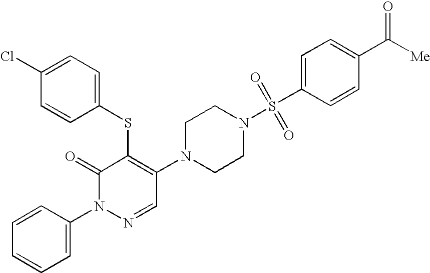 | 581 |
| 81 | 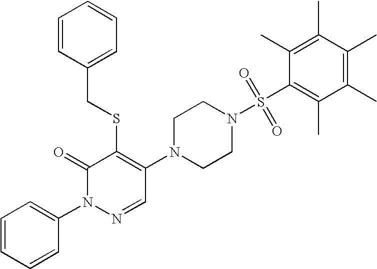 | 589 |
| 82 | 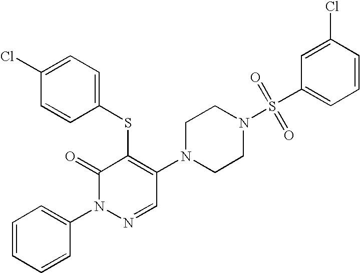 | 574 |
| 83 | 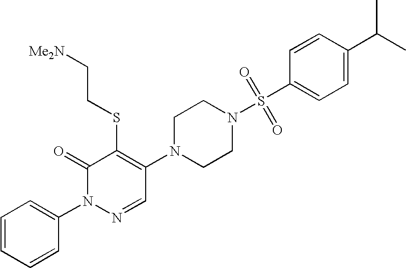 | 542 |
| 84 | 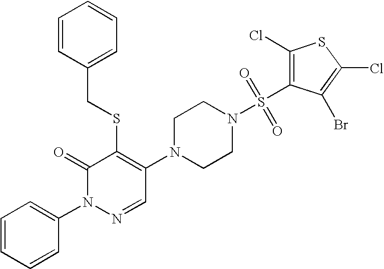 | 672 |
| 85 | 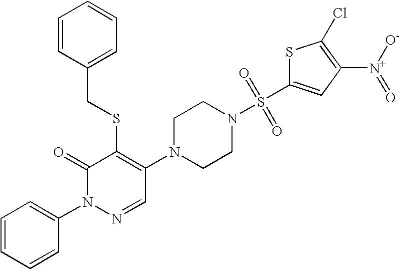 | 604 |
| 86 | 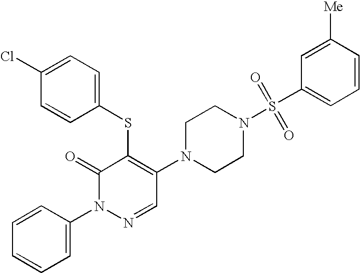 | 553 |
| 87 | 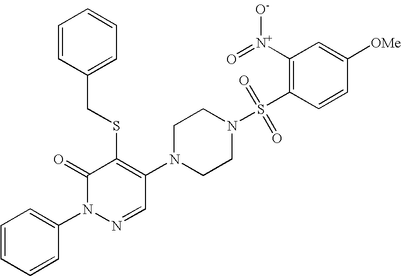 | 594 |
| 88 | 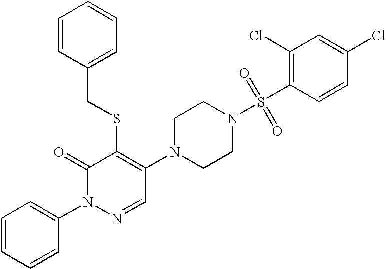 | 588 |
| 89 | 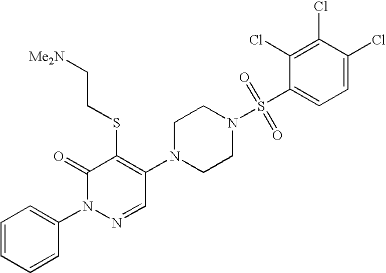 | 603 |
| 90 | 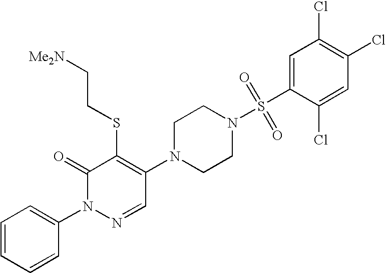 | 603 |
| 91 | 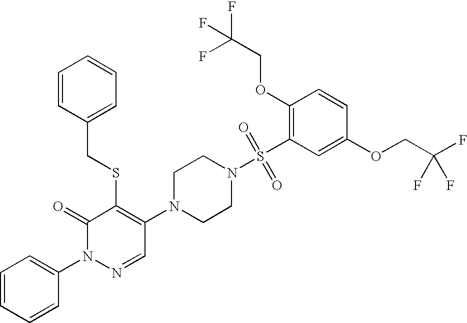 | 715 |
| 92 | 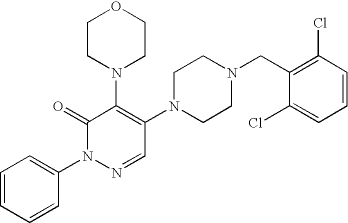 | 500 |
| 93 | 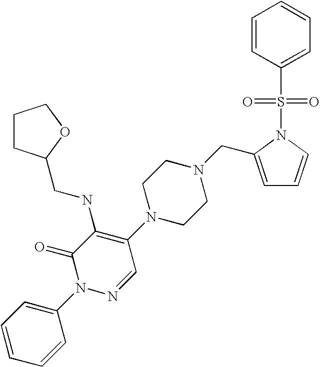 | 575 |
| 94 | 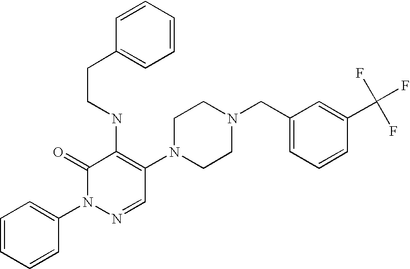 | 534 |
| 95 | 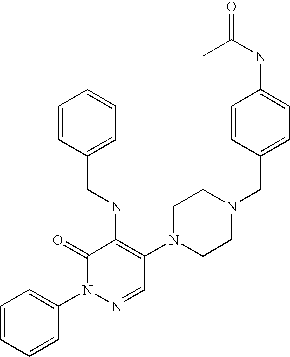 | 509 |
| 96 | 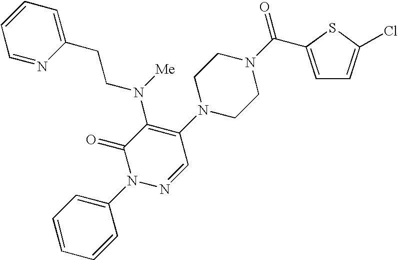 | 535 |
| 97 | 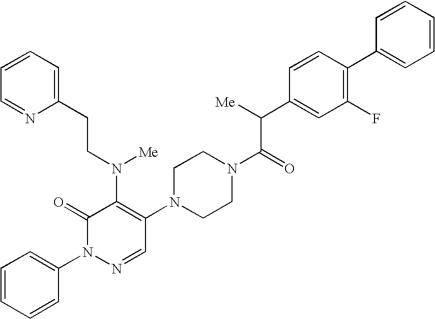 | 617 |
| 98 | 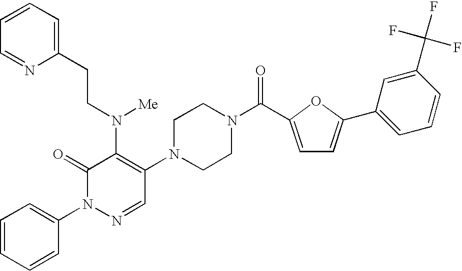 | 629 |
| 99 | 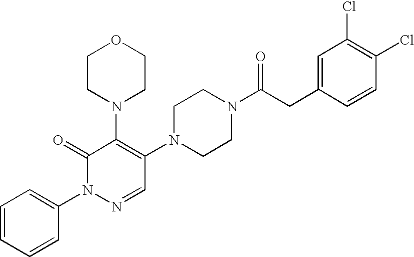 | 528 |
| 100 | 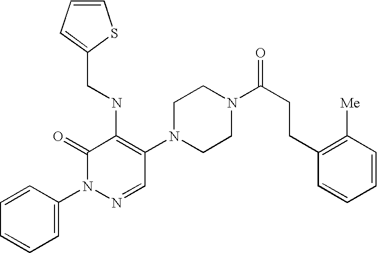 | 514 |
| 101 | 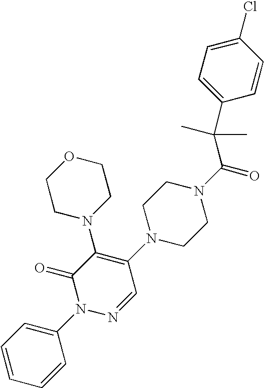 | 522 |
| 102 | 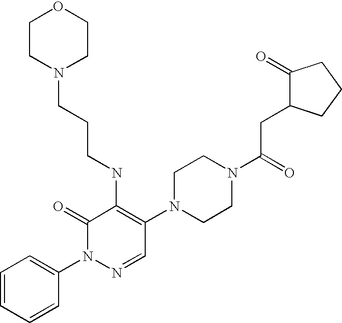 | 523 |
| 103 | 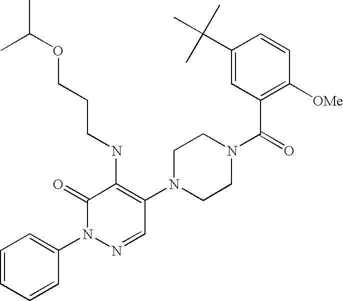 | 562 |
| 104 | 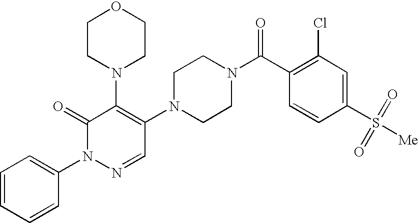 | 558 |
| 105 | 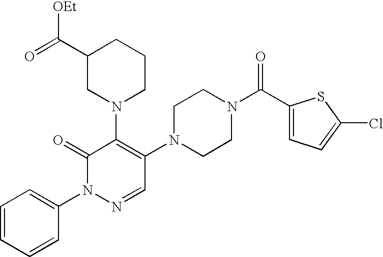 | 556 |
| 106 | 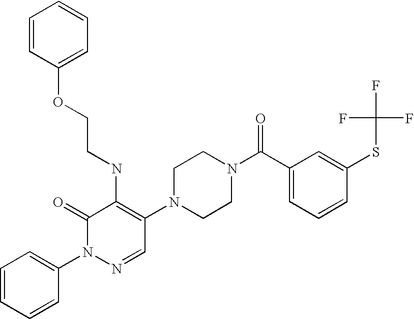 | 596 |
| 107 | 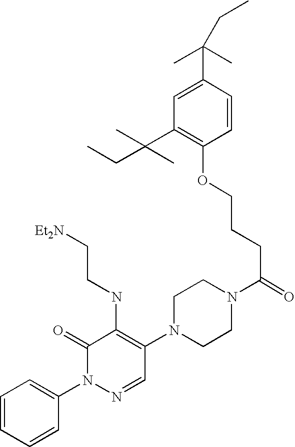 | 673 |
| 108 | 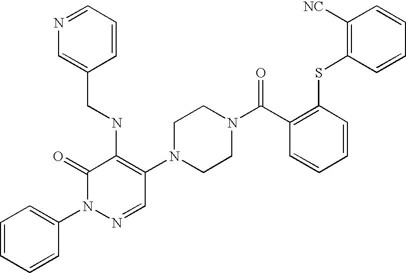 | 600 |
| 109 | 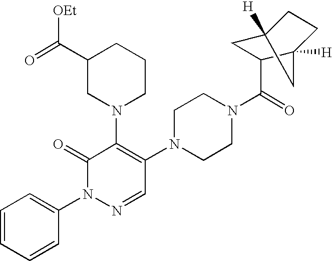 | 534 |
| 110 | 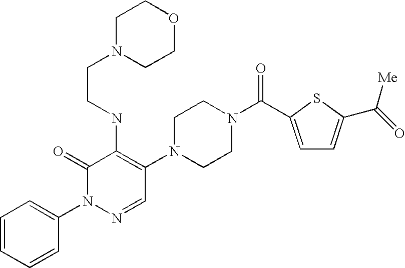 | 537 |
| 111 | 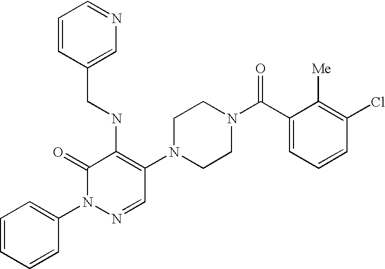 | 515 |
| 112 | 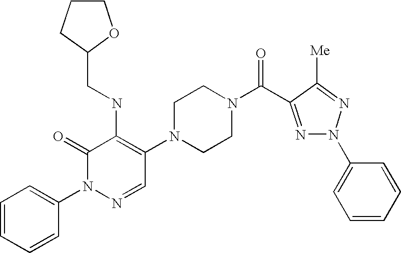 | 541 |
| 113 | 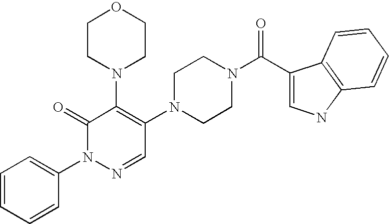 | 485 |
| 114 | 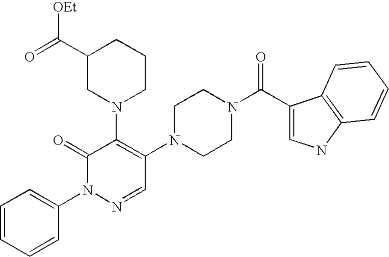 | 555 |
| 115 | 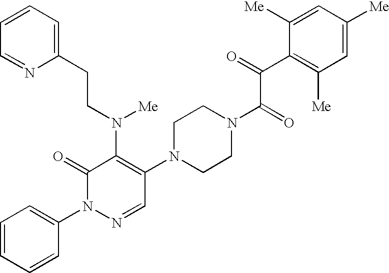 | 565 |
| 116 | 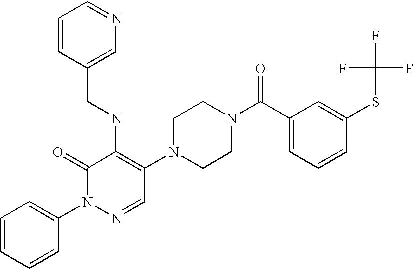 | 567 |
| 117 | 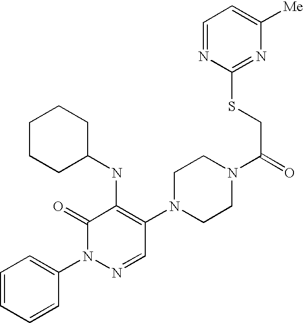 | 520 |
| 118 | 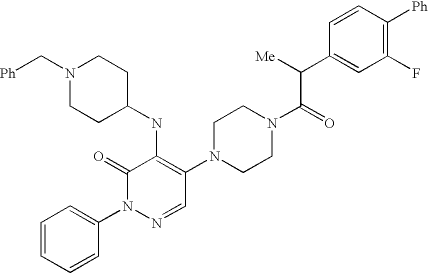 | 671 |
| 119 | 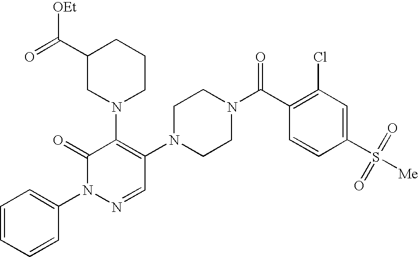 | 628 |
| 120 | 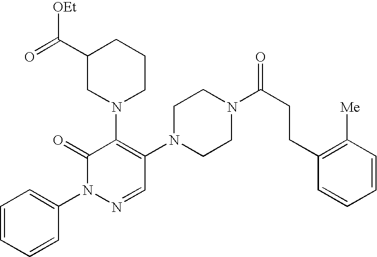 | 558 |
| 121 | 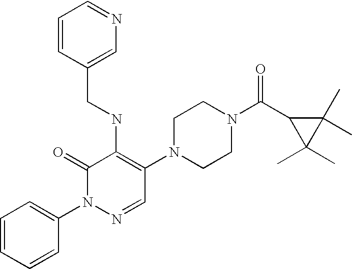 | 487 |
| 122 | 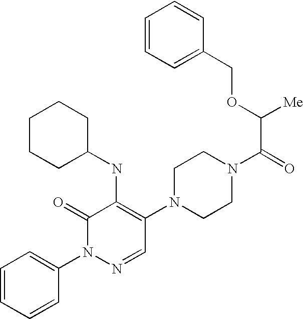 | 516 |
| 123 | 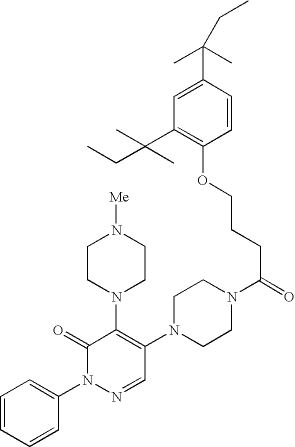 | 657 |
| 124 | 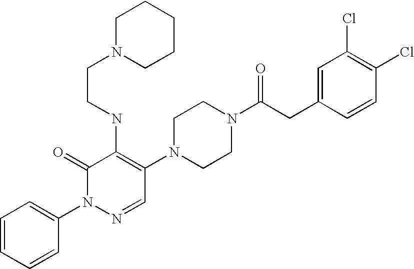 | 570 |
| 125 | 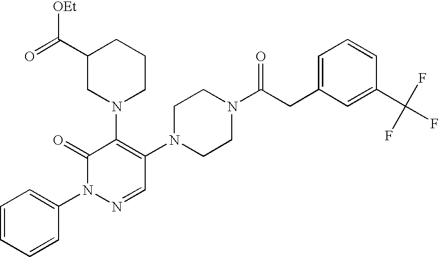 | 598 |
| 126 | 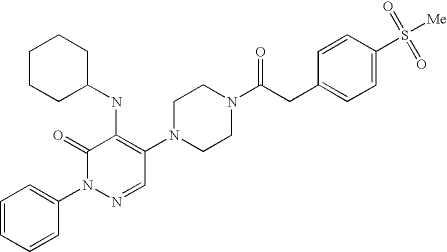 | 550 |
| 127 | 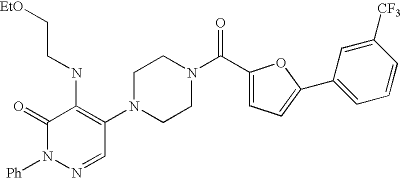 | 582 |
| 128 | 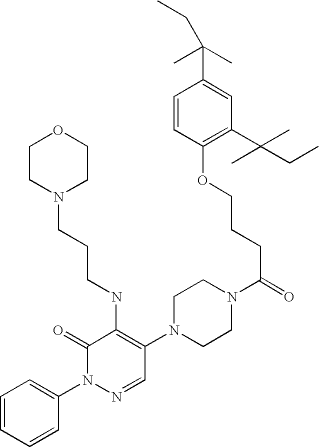 | 701 |
| 129 | 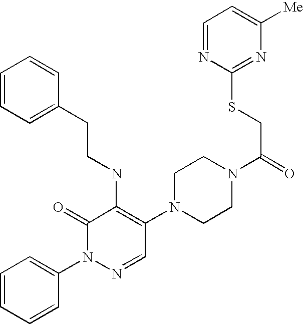 | 542 |
| 130 | 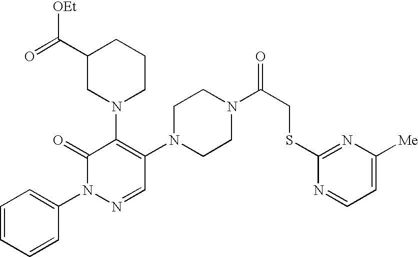 | 578 |
| 131 | 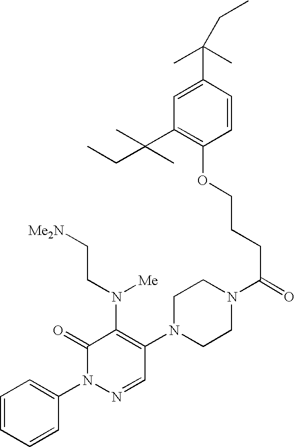 | 659 |
| 132 | 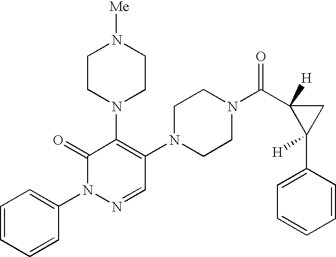 | 499 |
| 133 | 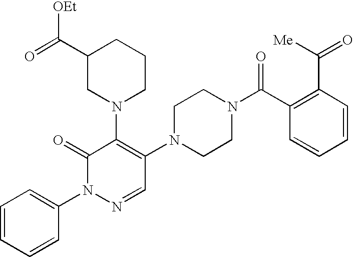 | 558 |
| 134 | 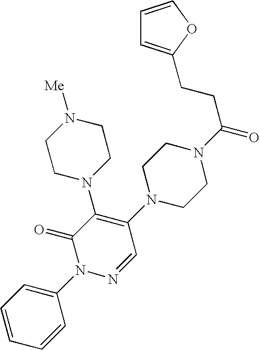 | 477 |
| 135 | 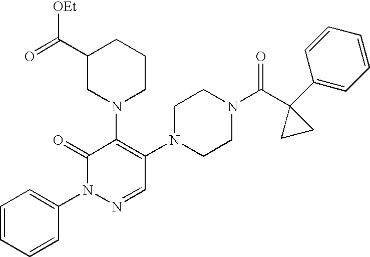 | 556 |
| 136 | 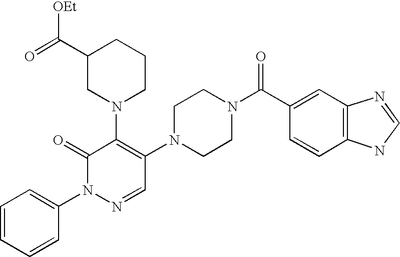 | 556 |
| 137 | 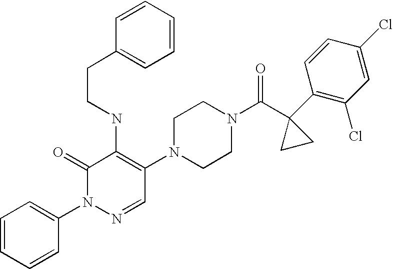 | 589 |
| 138 | 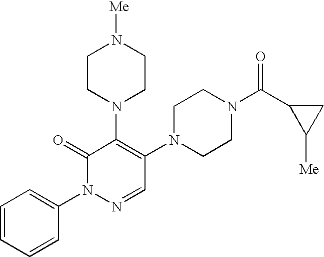 | 437 |
| 139 | 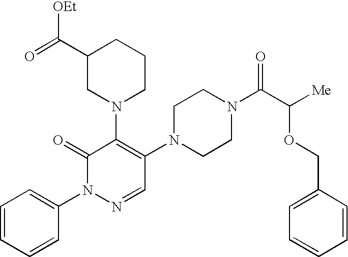 | 574 |
| 140 | 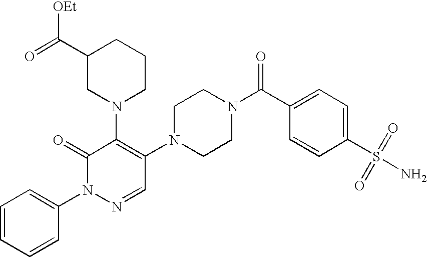 | 595 |
| 141 | 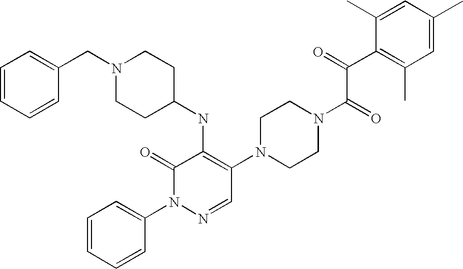 | 619 |
| 142 | 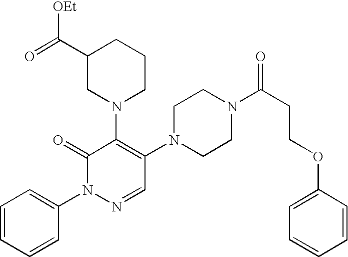 | 560 |
| 143 | 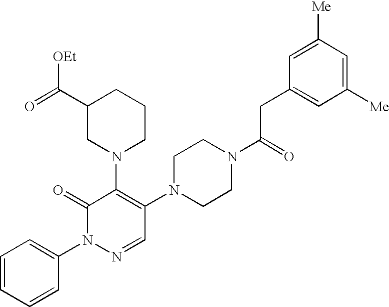 | 558 |
| 144 | 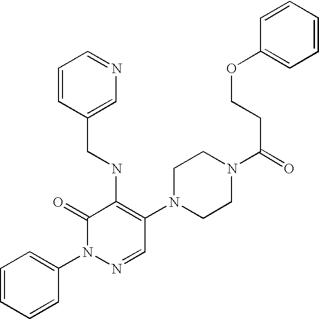 | 511 |
| 145 | 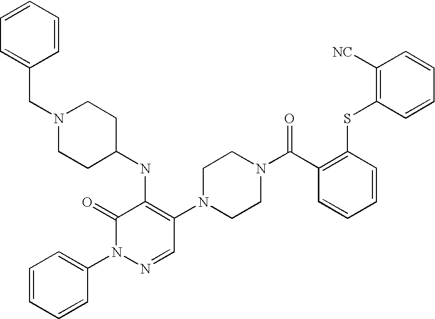 | 682 |
| 146 | 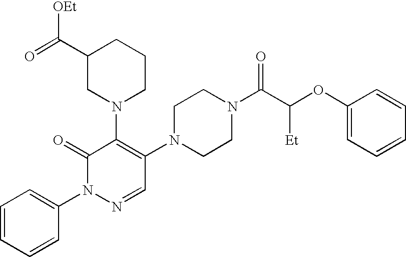 | 574 |
| 147 | 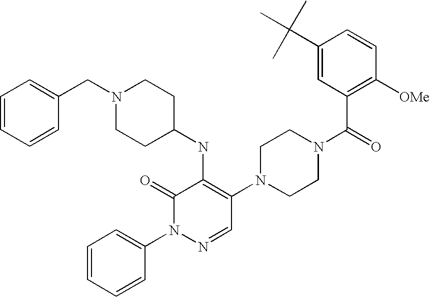 | 635 |
| 148 | 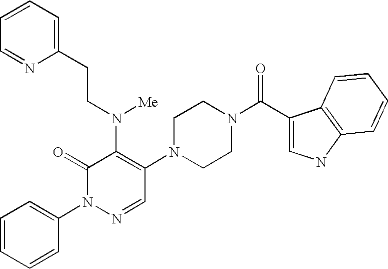 | 534 |
| 149 | 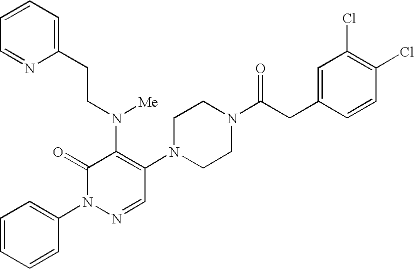 | 578 |
| 150 | 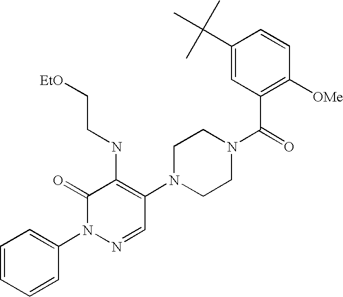 | 534 |
| 151 | 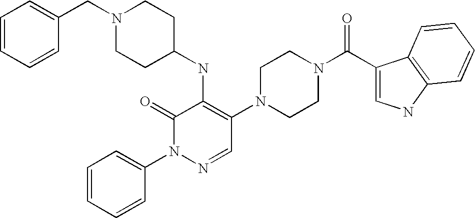 | 588 |
| 152 | 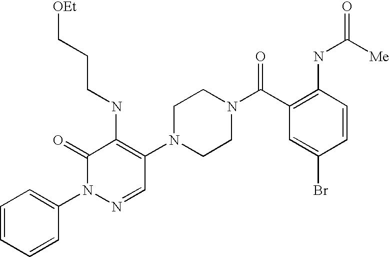 | 598 |
| 153 | 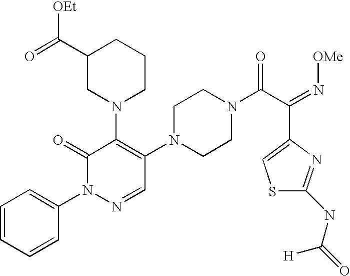 | 623 |
| 154 | 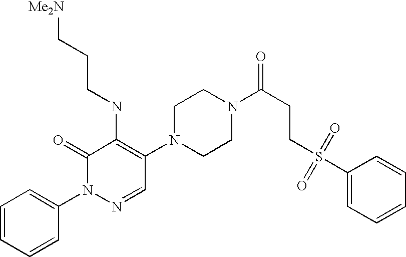 | 553 |
| 155 | 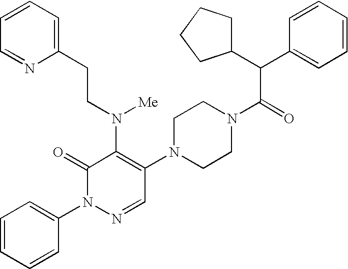 | 577 |
| 156 | 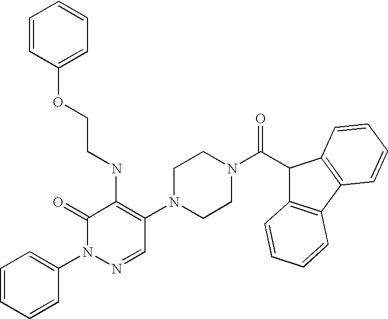 | 584 |
| 157 | 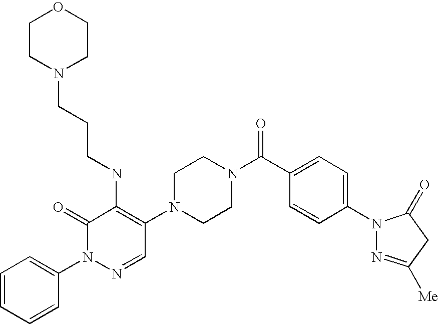 | 599 |
| 158 | 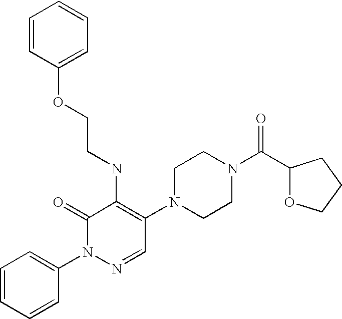 | 490 |
| 159 | 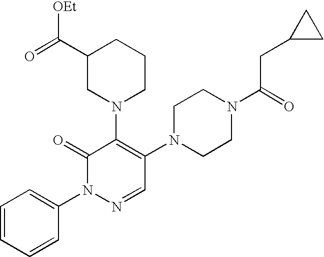 | 494 |
| 160 | 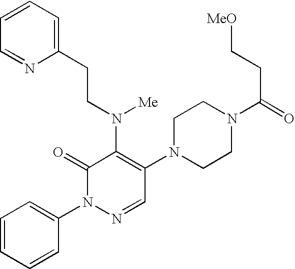 | 477 |
| 161 | 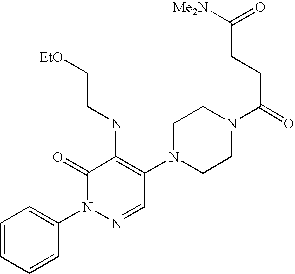 | 471 |
| 162 | 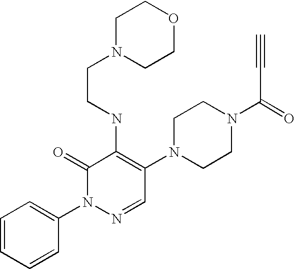 | 437 |
| 163 | 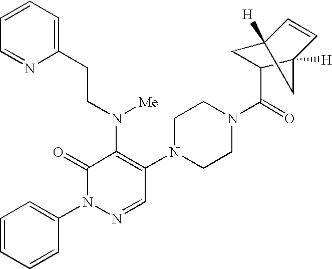 | 511 |
| 164 | 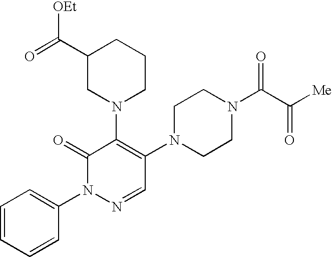 | 482 |
| 165 | 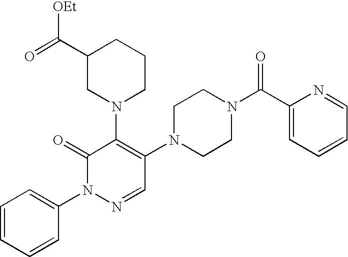 | 517 |
| 166 | 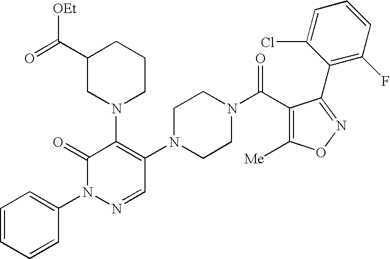 | 649 |
| 167 | 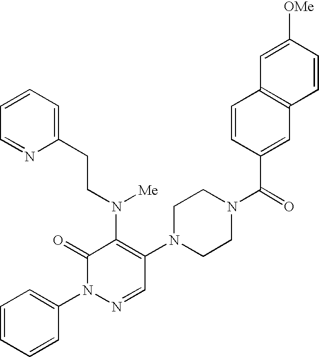 | 575 |
| 168 | 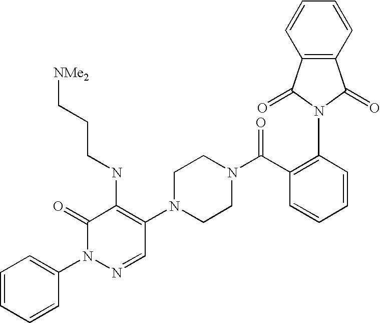 | 606 |
| 169 | 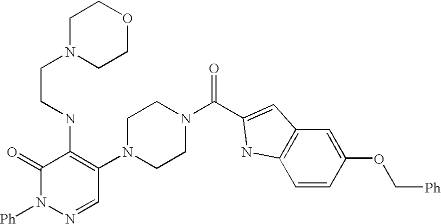 | 634 |
| 170 | 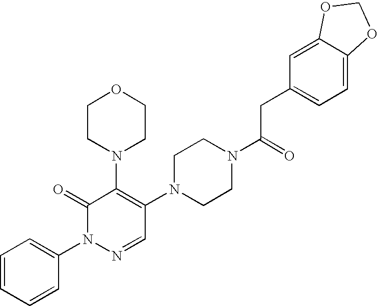 | 504 |
| 171 | 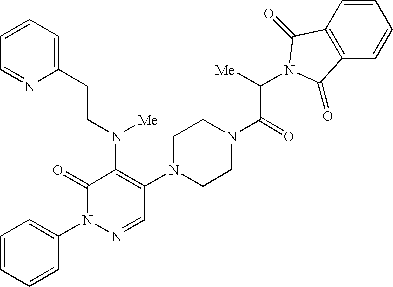 | 592 |
| 172 | 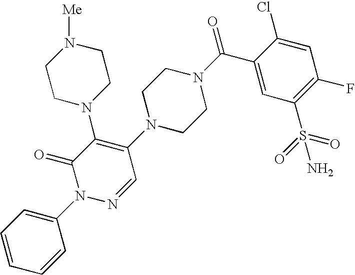 | 590 |
| 173 | 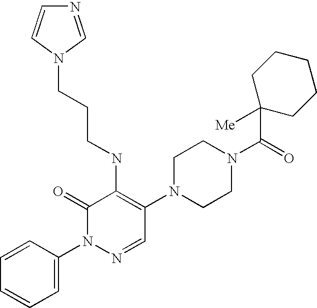 | 504 |
| 174 | 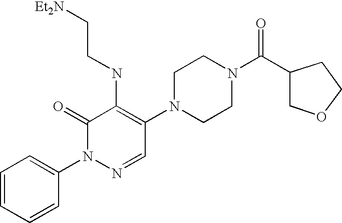 | 469 |
| 175 | 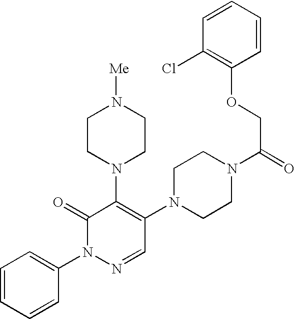 | 523 |
| 176 | 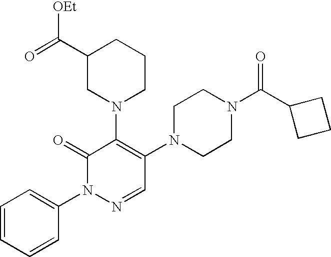 | 494 |
| 177 | 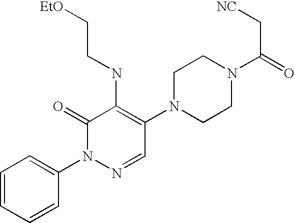 | 410 |
| 178 | 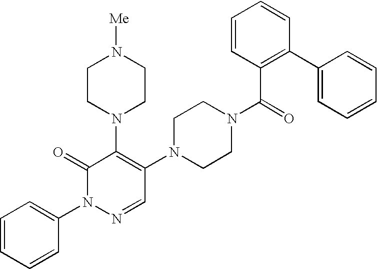 | 535 |
| 179 | 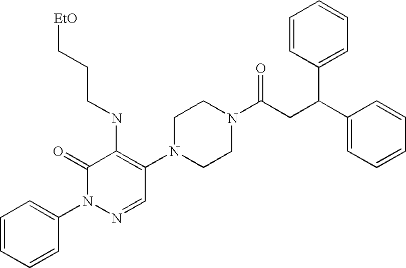 | 566 |
| 180 | 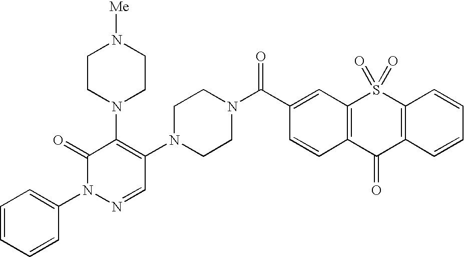 | 625 |
| 181 | 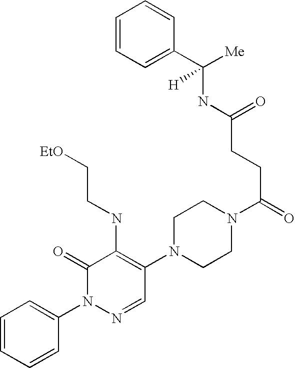 | 547 |
| 182 | 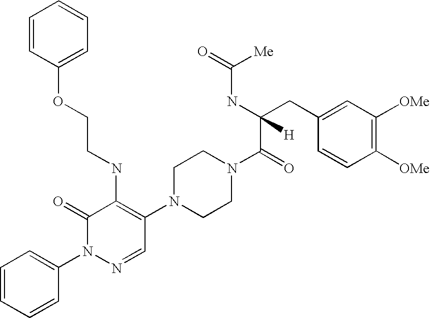 | 641 |
| 183 | 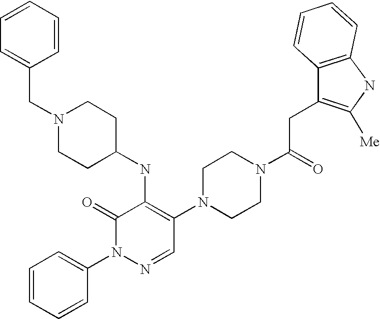 | 616 |
| 184 | 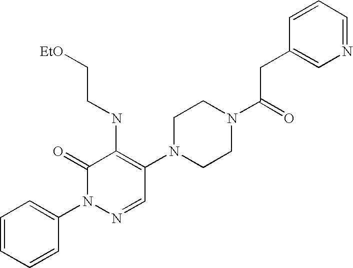 | 463 |
| 185 | 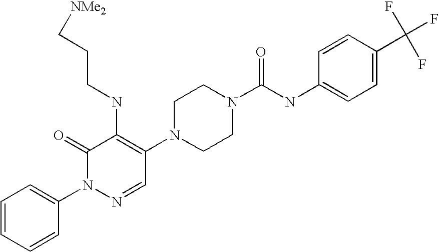 | 544 |
| 186 | 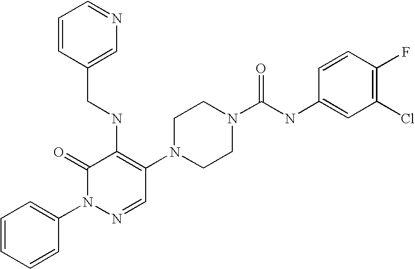 | 534 |
| 187 | 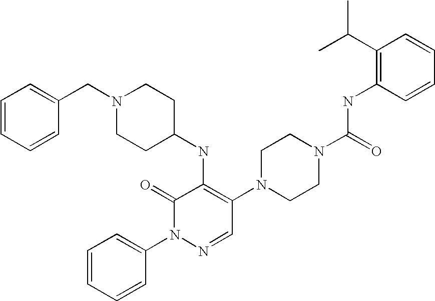 | 606 |
| 188 | 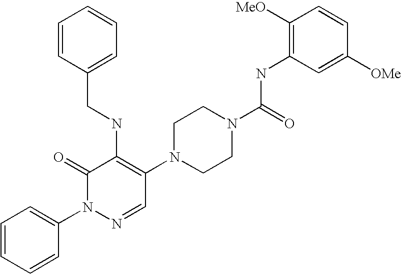 | 541 |
| 189 | 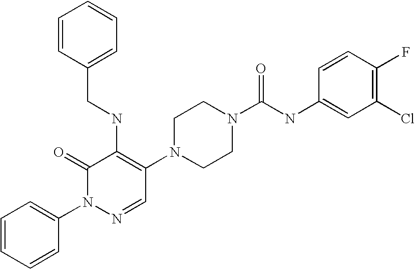 | 533 |
| 190 | 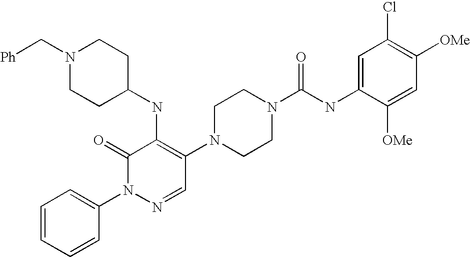 | 658 |
| 191 | 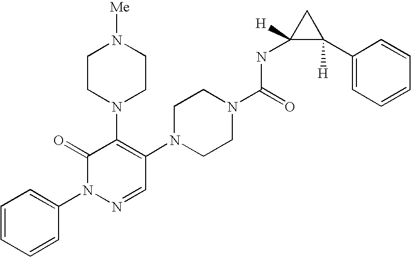 | 514 |
| 192 | 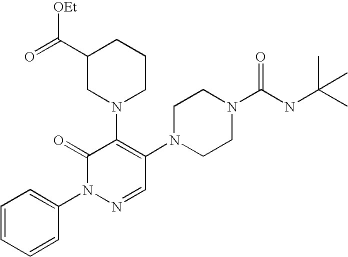 | 511 |
| 193 | 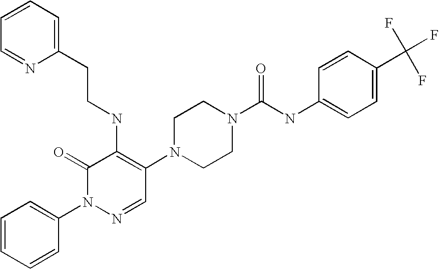 | 578 |
| 194 | 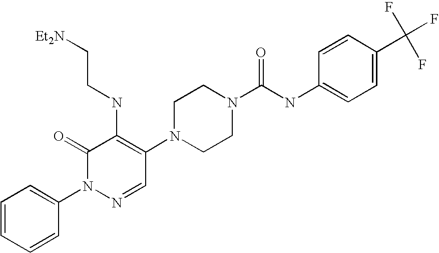 | 558 |
| 195 | 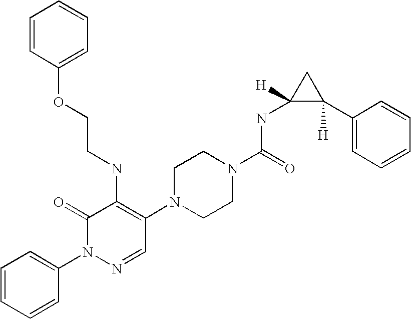 | 551 |
| 196 | 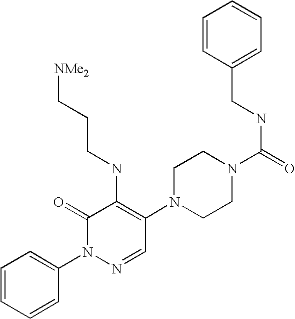 | 490 |
| 197 | 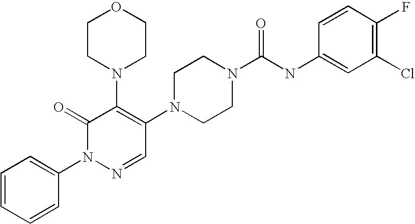 | 513 |
| 198 | 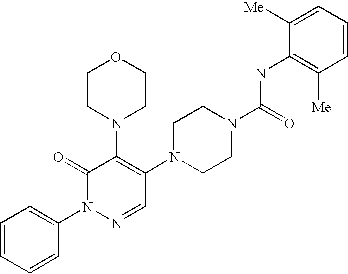 | 489 |
| 199 | 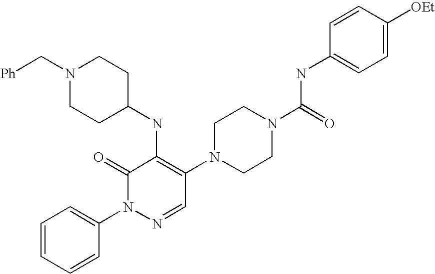 | 608 |
| 200 | 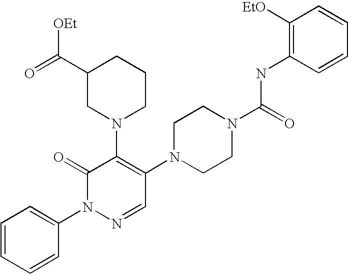 | 575 |
| 201 | 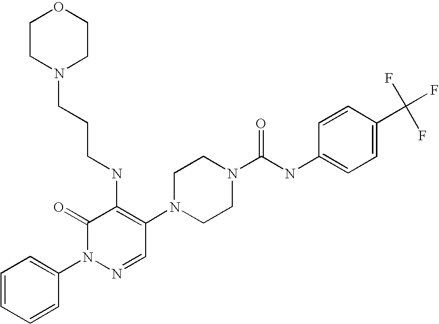 | 586 |
| 202 | 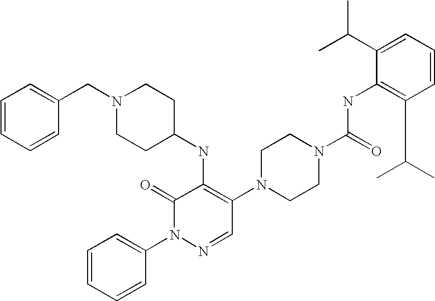 | 648 |
| 203 | 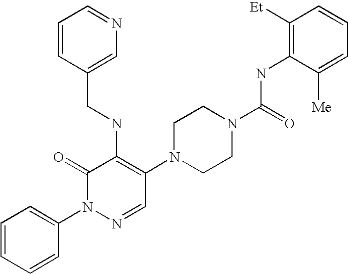 | 524 |
| 204 | 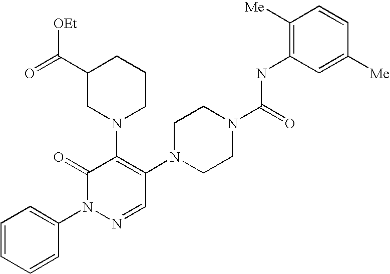 | 559 |
| 205 | 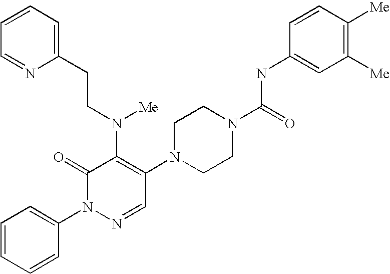 | 538 |
| 206 | 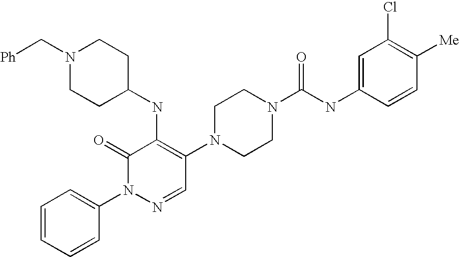 | 612 |
| 207 | 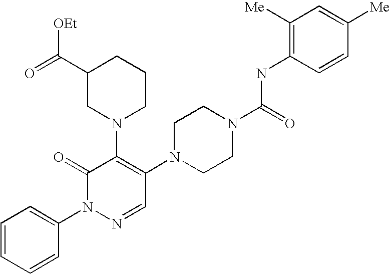 | 559 |
| 208 | 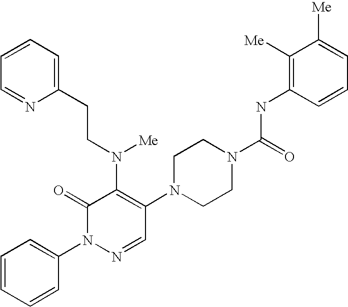 | 539 |
| 209 | 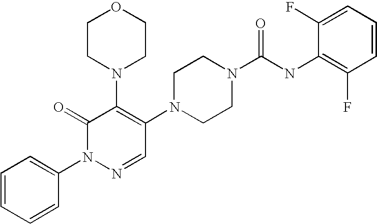 | 497 |
| 210 | 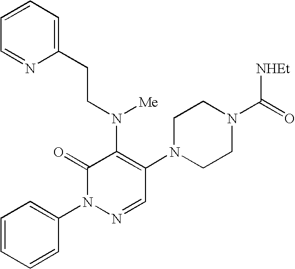 | 462 |
| 211 | 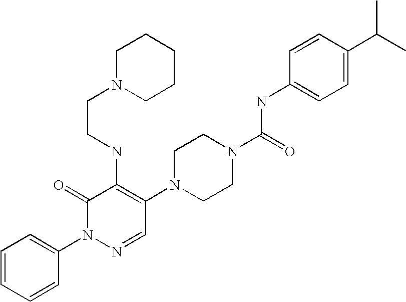 | 544 |
| 212 | 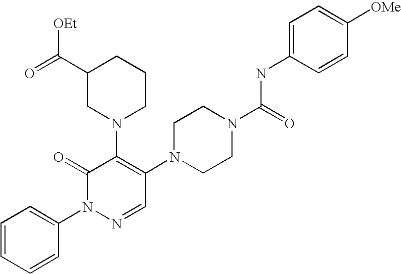 | 561 |
| 213 | 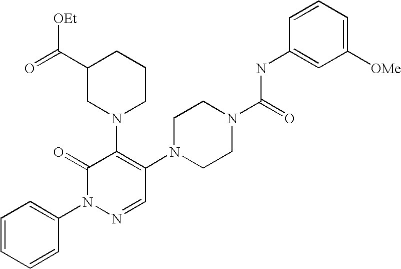 | 561 |
| 214 | 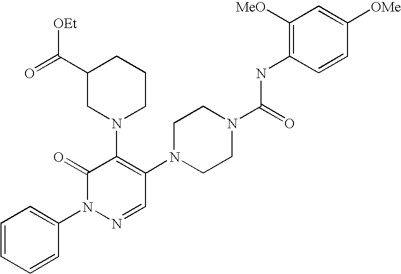 | 591 |
| 215 | 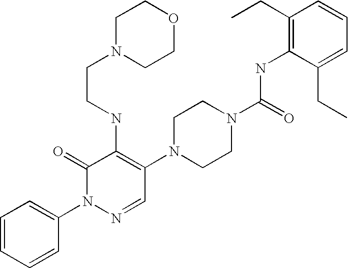 | 560 |
| 216 | 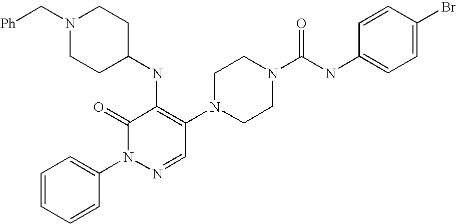 | 643 |
| 217 | 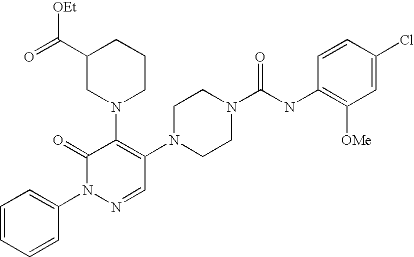 | 579 |
| 218 | 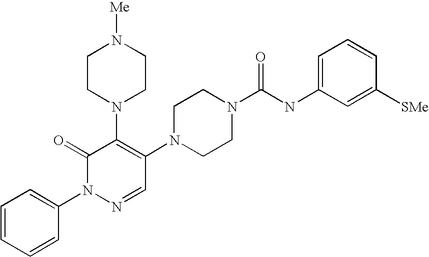 | 520 |
| 219 | 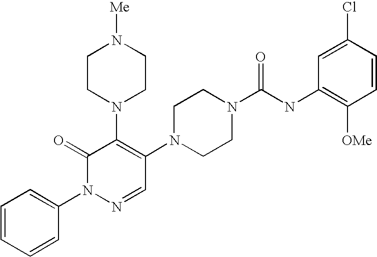 | 538 |
| 220 | 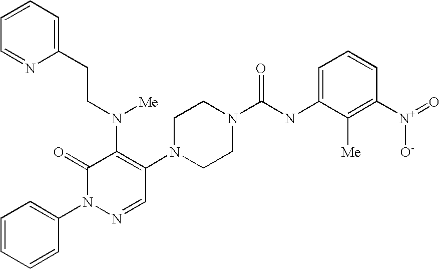 | 569 |
| 221 | 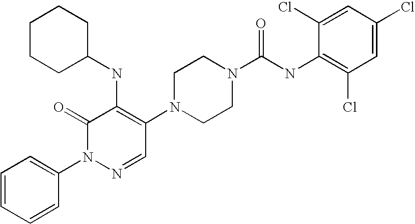 | 576 |
| 222 | 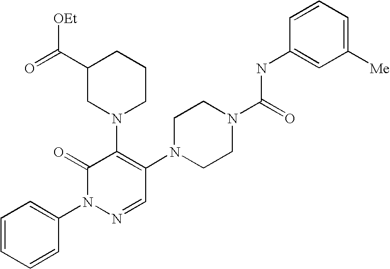 | 545 |
| 223 | 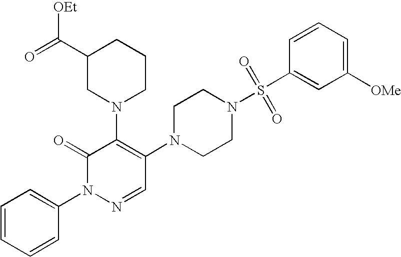 | 582 |
| 224 | 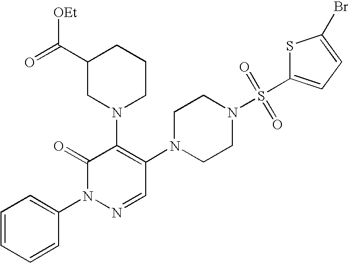 | 637 |
| 225 | 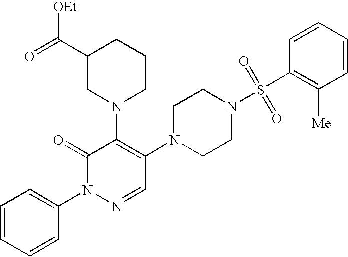 | 566 |
| 226 | 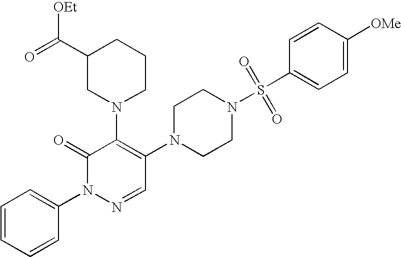 | 582 |
| 227 | 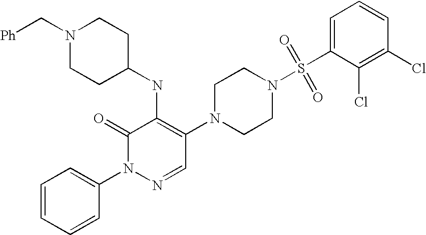 | 654 |
| 228 | 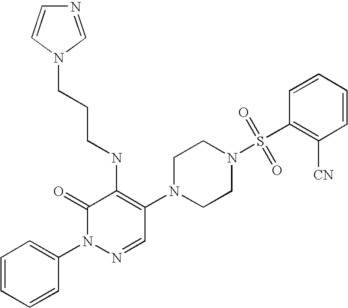 | 545 |
| 229 | 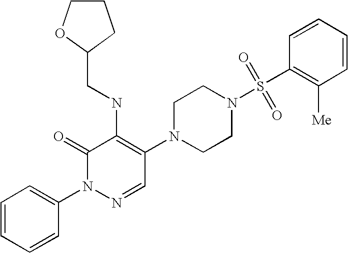 | 510 |
| 230 | 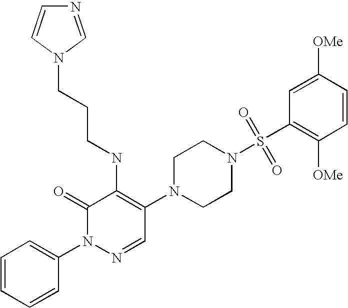 | 580 |
| 231 | 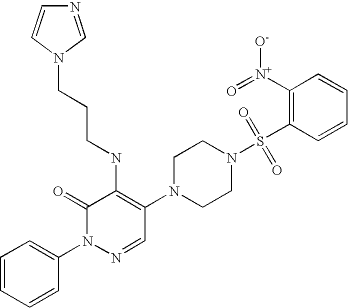 | 565 |
| 232 | 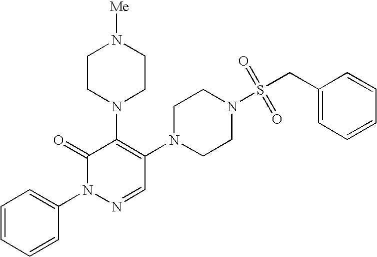 | 509 |
| 233 | 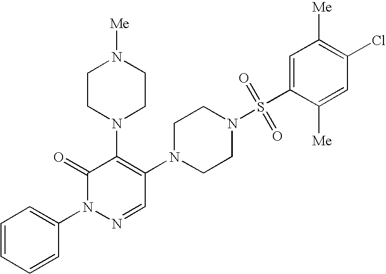 | 557 |
| 234 | 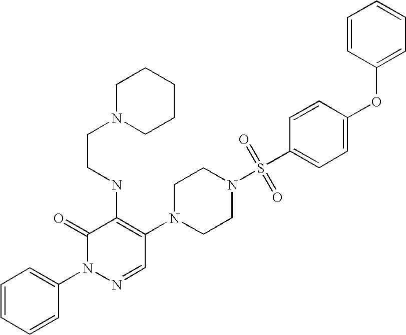 | 615 |
| 235 | 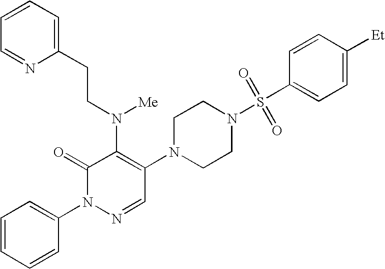 | 559 |
| 236 | 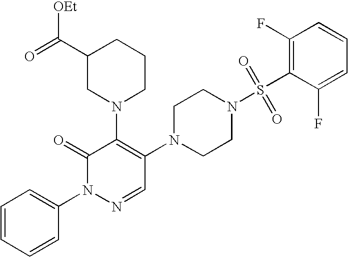 | 588 |
| 237 | 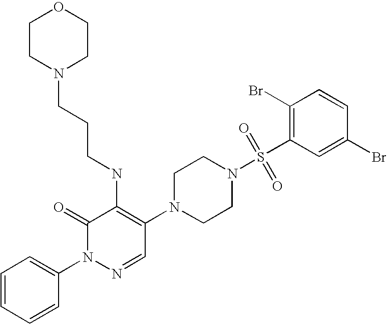 | 696 |
| 238 | 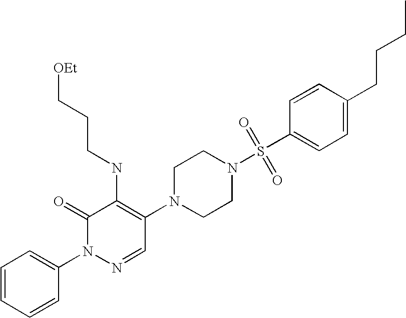 | 554 |
| 239 | 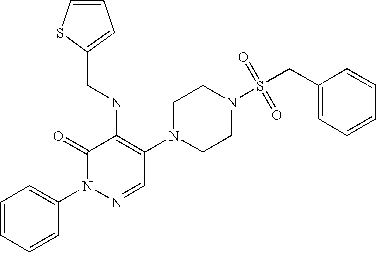 | 522 |
| 240 | 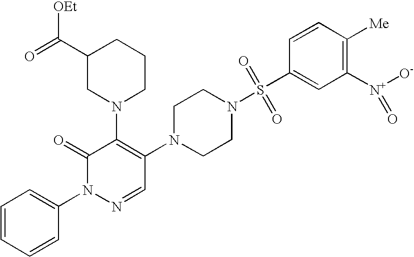 | 611 |
| 241 | 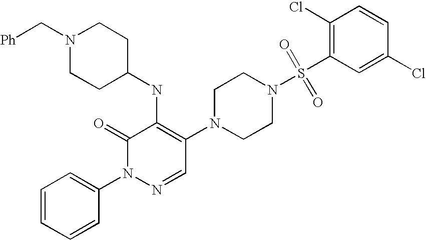 | 654 |
| 242 | 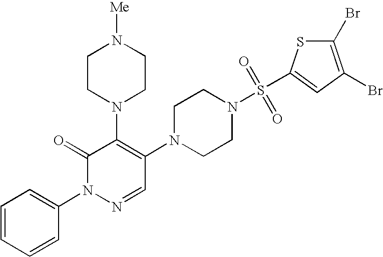 | 658 |
| 243 | 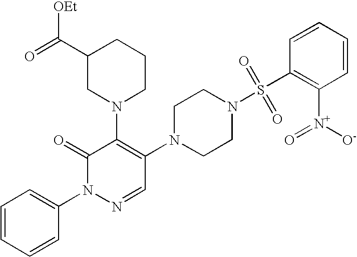 | 597 |
| 244 | 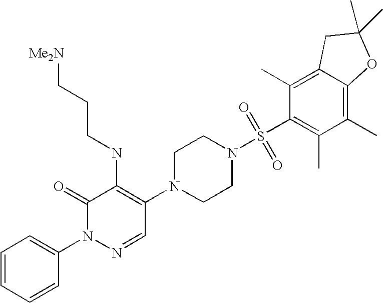 | 609 |
| 245 | 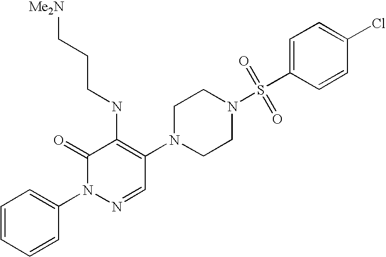 | 531 |
| 246 | 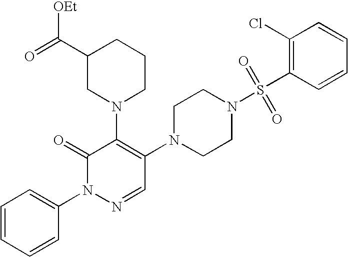 | 586 |
| 247 | 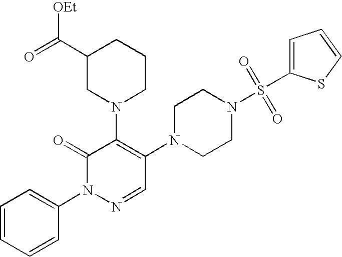 | 558 |
| | 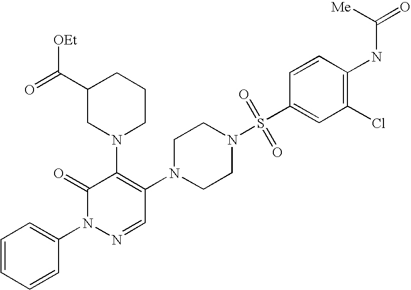 | 643 |
| 249 | 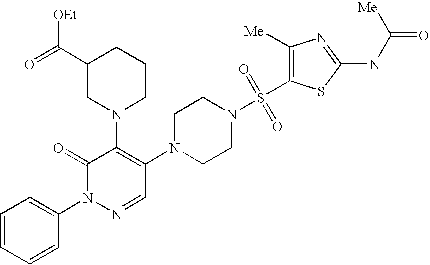 | 630 |
| 250 | 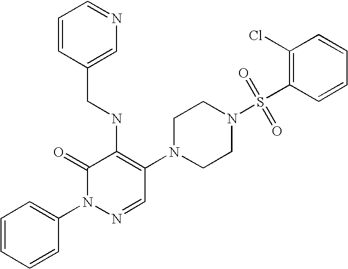 | 537 |
| 251 | 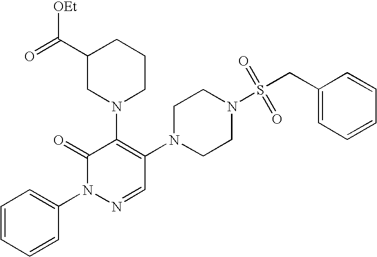 | 566 |
| 252 | 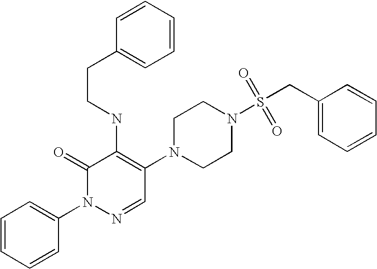 | 530 |
| 253 | 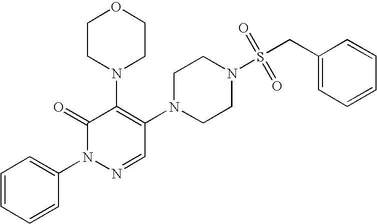 | 496 |
| 254 | 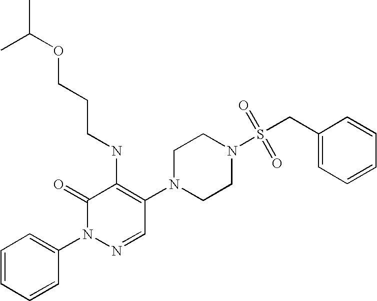 | 526 |
| 255 | 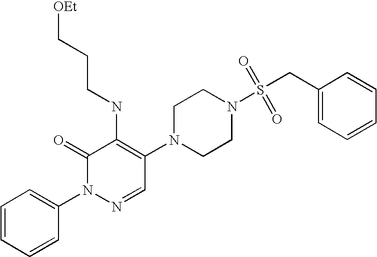 | 512 |
| 256 | 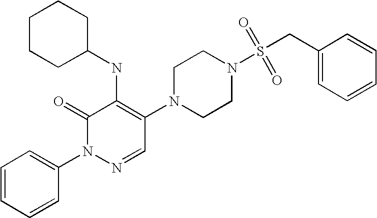 | 508 |
| 257 | 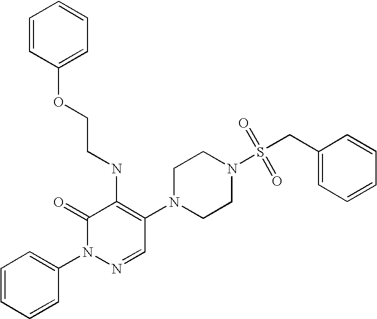 | 546 |
| 258 | 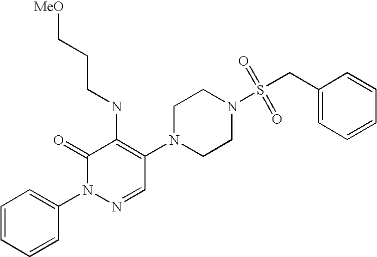 | 498 |
| 259 | 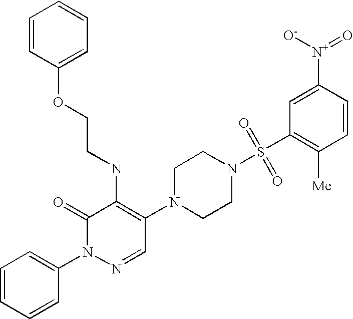 | 591 |
| 260 | 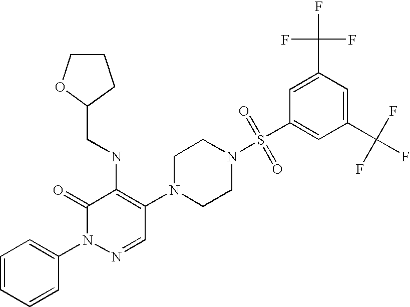 | 632 |
| 261 | 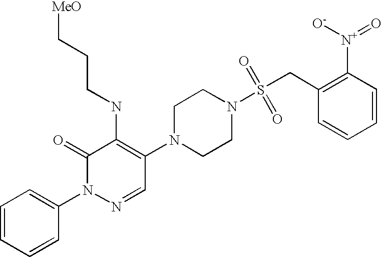 | 543 |
| 262 | 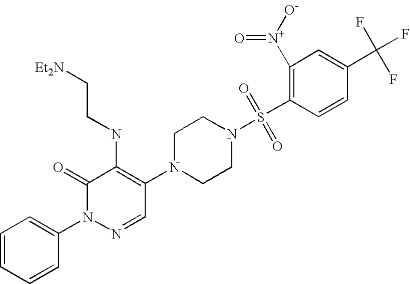 | 624 |
| 263 | 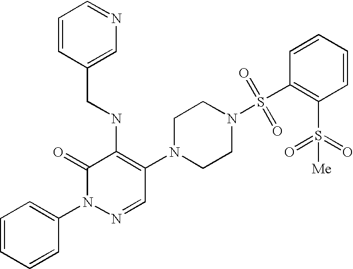 | 581 |
| 264 | 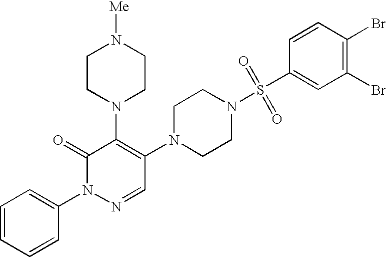 | 652 |
| 265 | 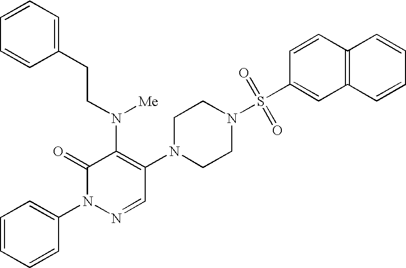 | 581 |
| 266 | 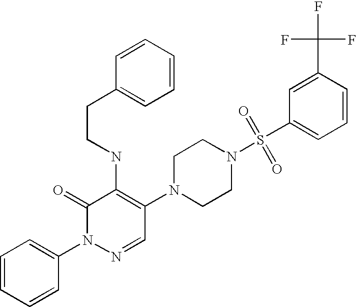 | 584 |
| 267 | 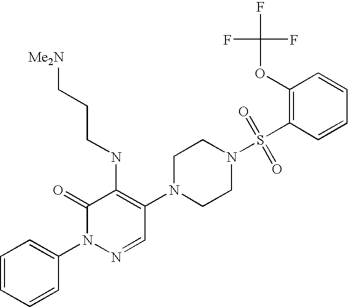 | 581 |
| 268 | 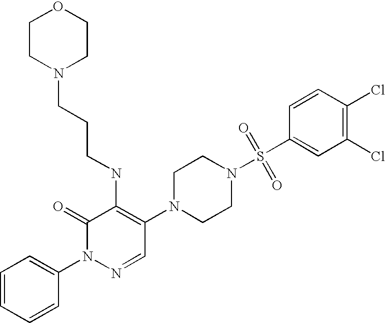 | 608 |
| 269 | 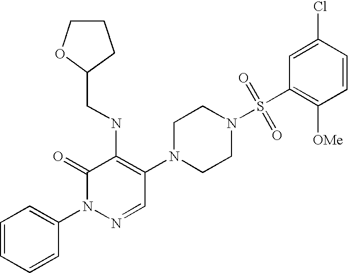 | 560 |
| 270 | 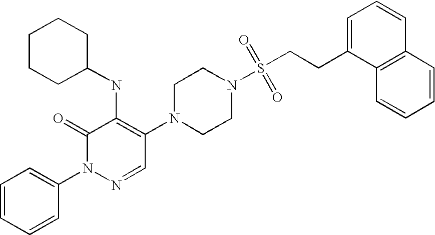 | 572 |
| 271 | 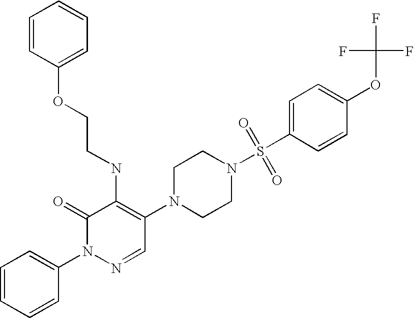 | 616 |
| 272 | 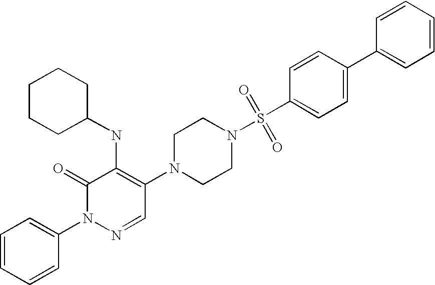 | 570 |
| 273 | 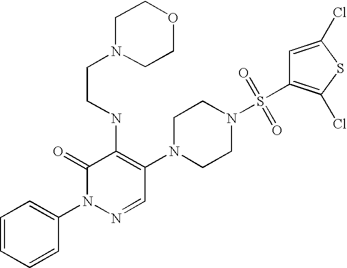 | 600 |
| 274 | 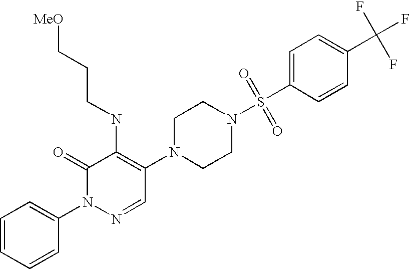 | 552 |
| 275 | 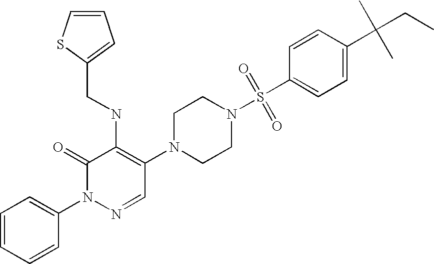 | 578 |
| 276 | 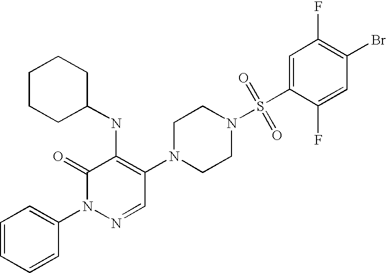 | 609 |
| 277 | 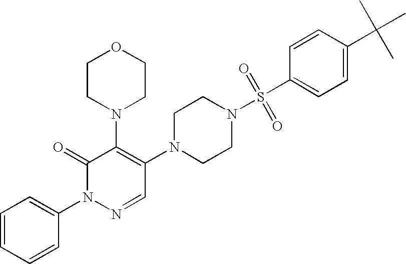 | 538 |
| 278 | 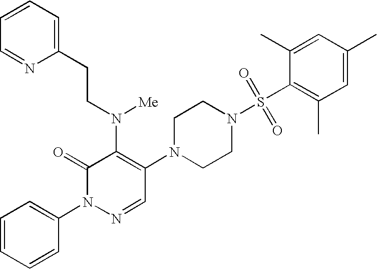 | 573 |
| 279 | 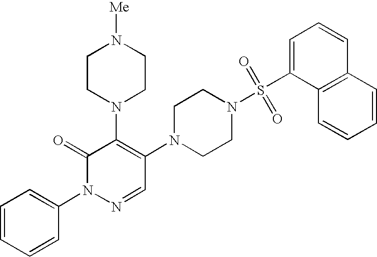 | 545 |
| 280 | 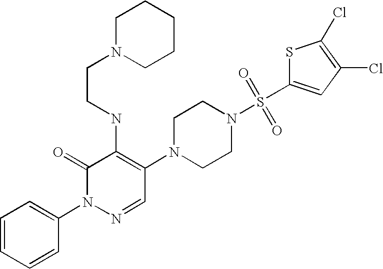 | 598 |
| 281 | 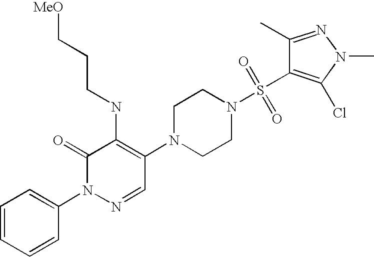 | 536 |
| 282 | 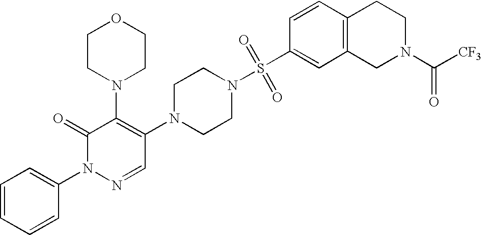 | 633 |
| 283 | 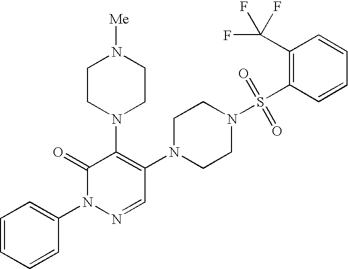 | 536 |
| 284 | 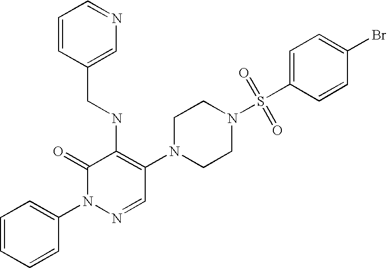 | 581 |
| 285 | 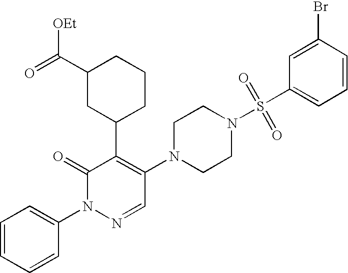 | 631 |
| 286 | 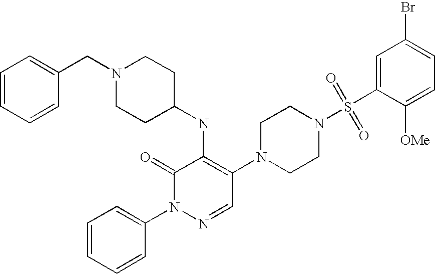 | 694 |
| 287 | 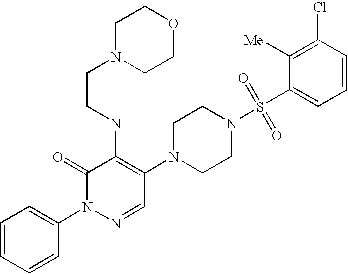 | 573 |
| 288 | 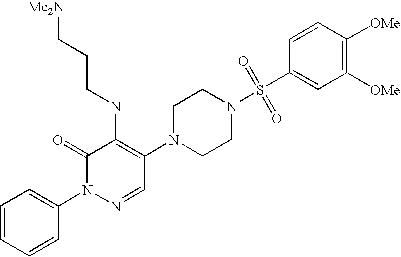 | 557 |
| 289 | 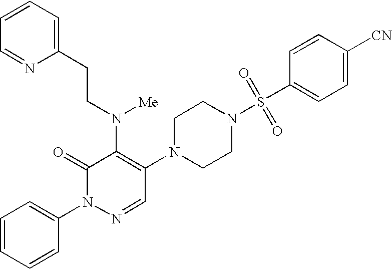 | 556 |
| 290 | 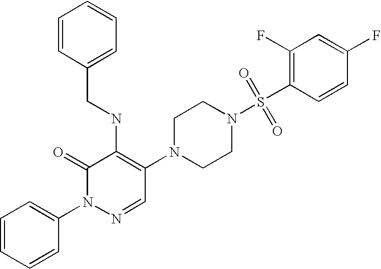 | 538 |
| 291 | 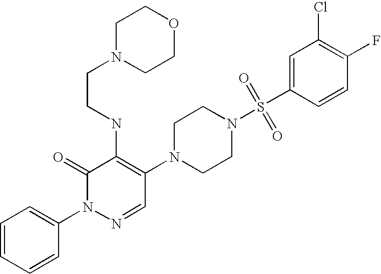 | 577 |
| 292 | 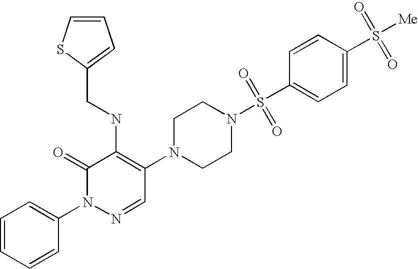 | 586 |
| 293 | 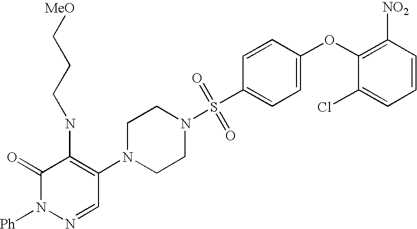 | 655 |
| 294 | 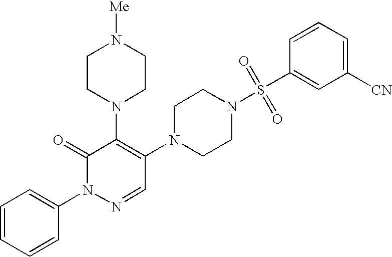 | 520 |
| 295 | 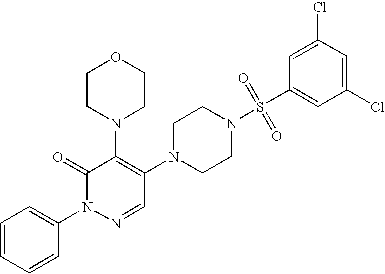 | 550 |
| 296 | 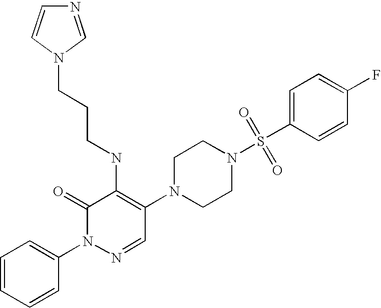 | 538 |
| 297 | 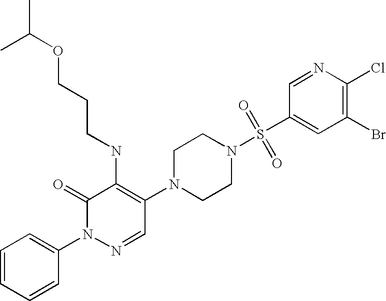 | 626 |
| 298 | 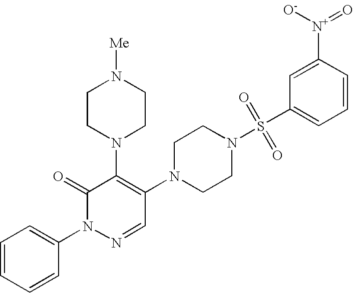 | 540 |
| 299 | 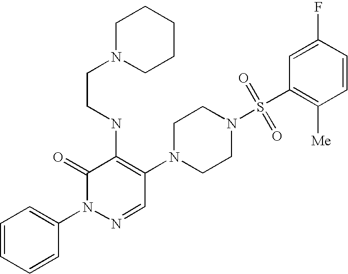 | 555 |
| 300 | 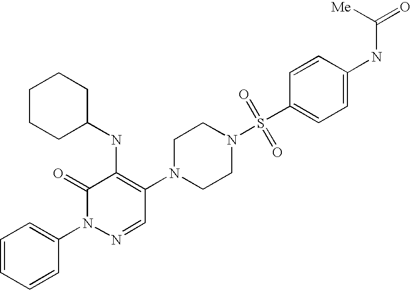 | 551 |
| 301 | 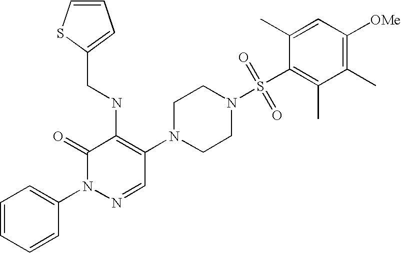 | 580 |
| 302 | 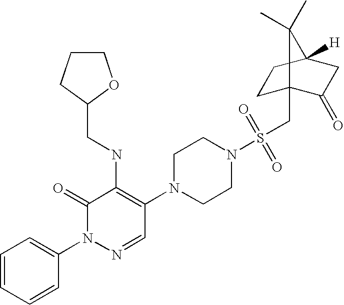 | 570 |
| 303 | 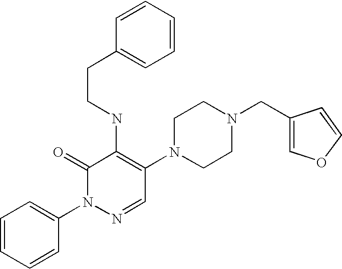 | 457 |
| 304 | 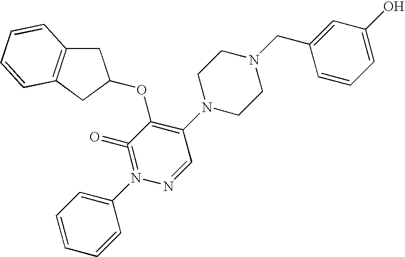 | 495 |
| 305 | 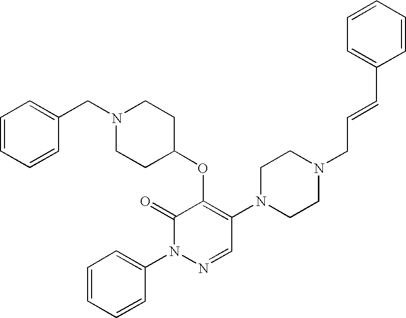 | 562 |
| 306 | 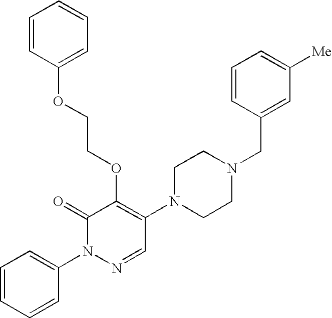 | 497 |
| 307 | 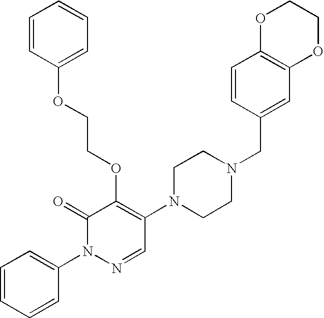 | 541 |
| 308 | 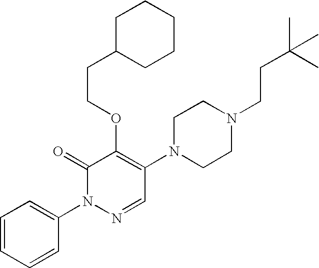 | 467 |
| 309 | 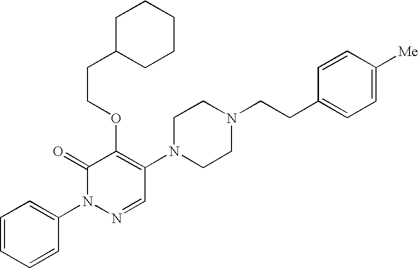 | 501 |
| 310 | 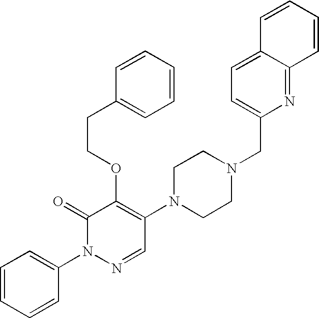 | 518 |
| 311 | 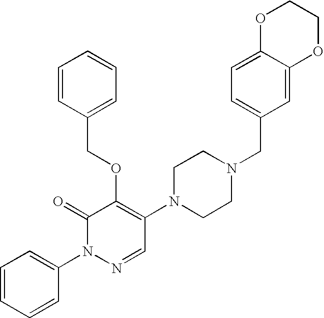 | 511 |
| 312 | 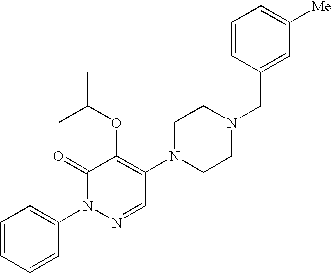 | 419 |
| 313 | 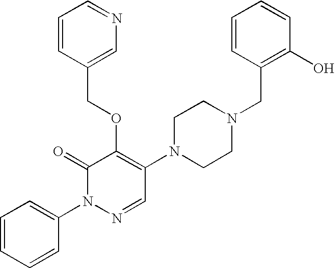 | 470 |
| 314 | 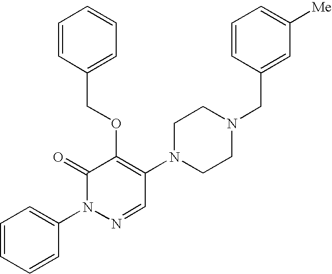 | 467 |
| 315 | 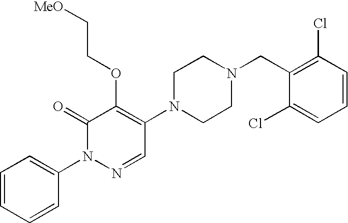 | 489 |
| 316 | 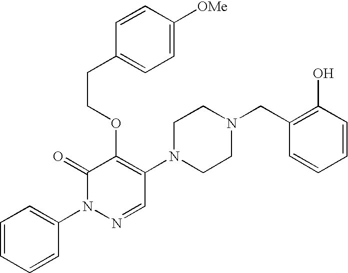 | 513 |
| 317 | 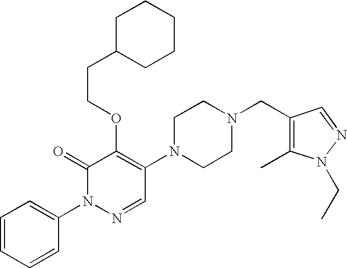 | 505 |
| 318 | 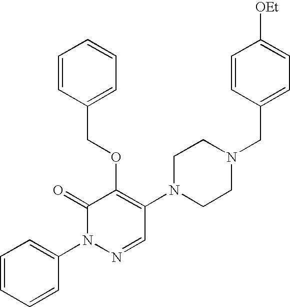 | 497 |
| 319 | 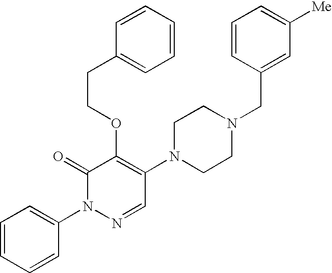 | 481 |
| 320 | 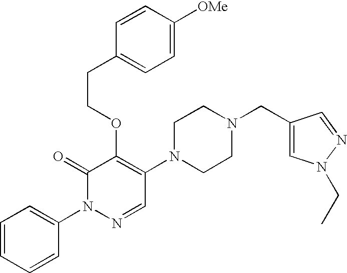 | 515 |
| 321 | 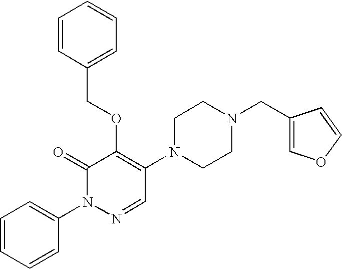 | 443 |
| 322 | 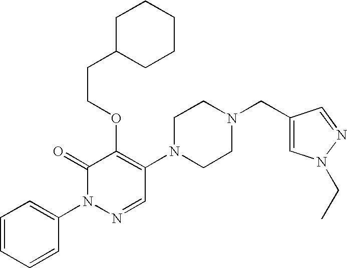 | 491 |
| 323 | 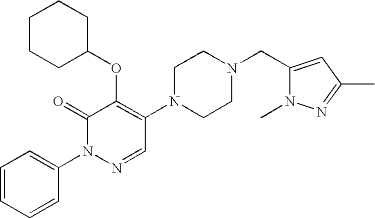 | 463 |
| 324 | 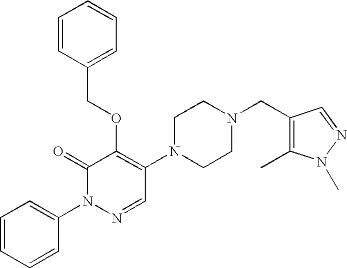 | 471 |
| 325 | 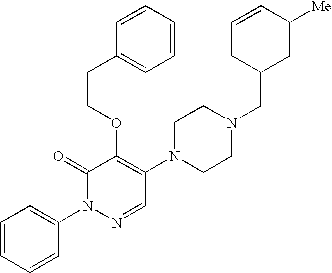 | 485 |
| 326 | 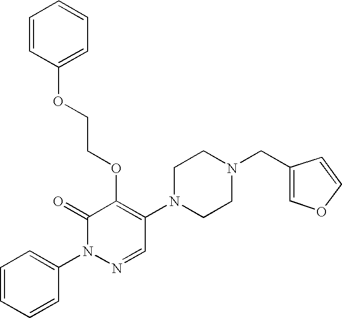 | 473 |
| 327 | 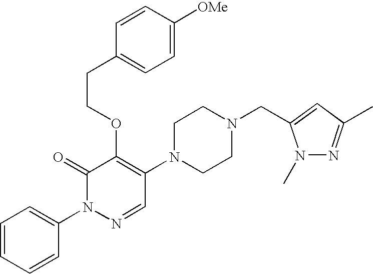 | 515 |
| 328 | 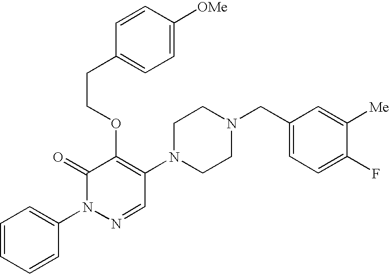 | 529 |
| 329 | 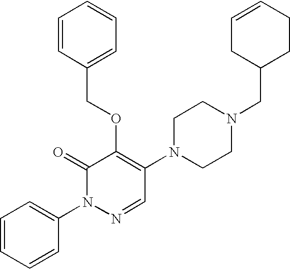 | 457 |
| 330 | 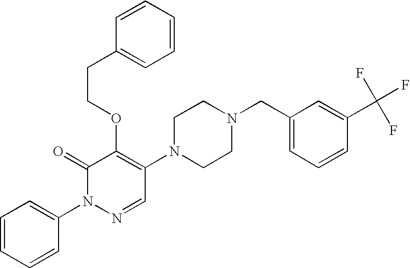 | 535 |
| 331 | 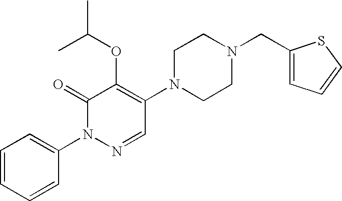 | 411 |
| 332 | 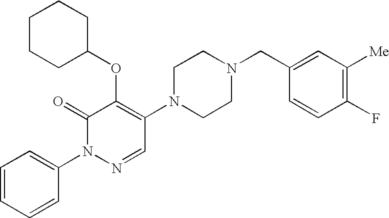 | 477 |
| 333 | 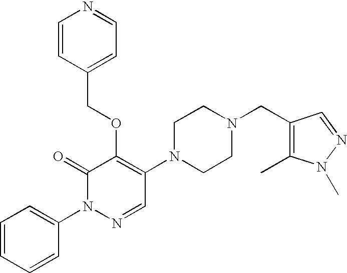 | 472 |
| 334 | 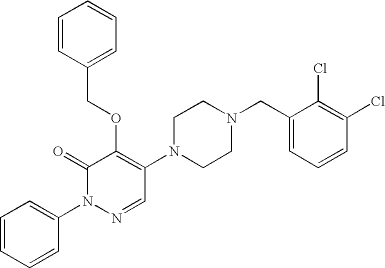 | 521 |
| 335 | 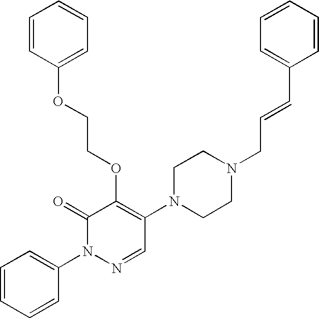 | 509 |
| 336 | 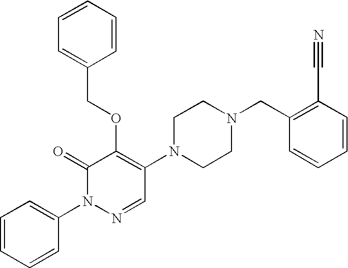 | 478 |
| 337 | 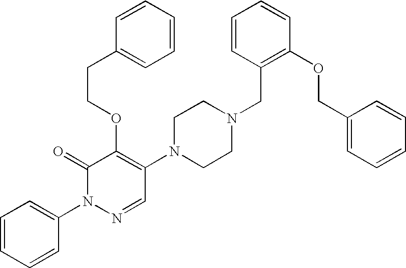 | 573 |
| 338 | 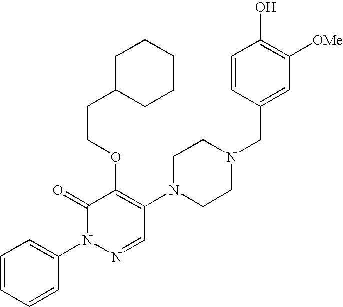 | 519 |
| 339 | 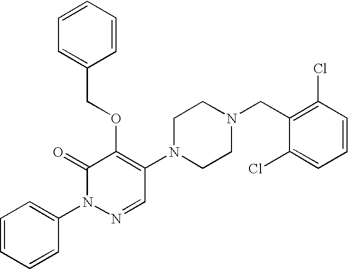 | 521 |
| 340 | 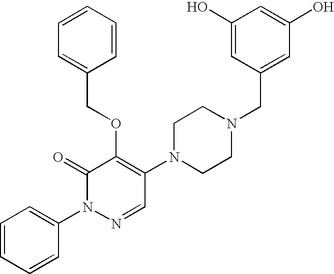 | 485 |
| 341 | 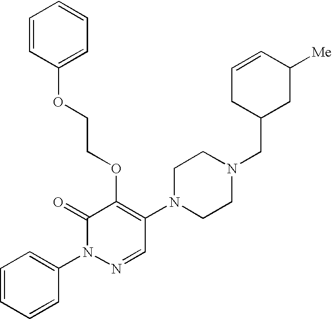 | 501 |
| 342 | 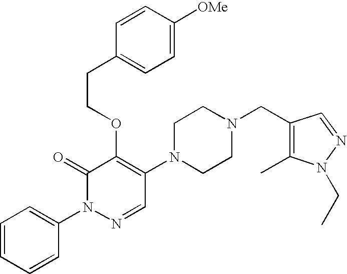 | 529 |
| 343 | 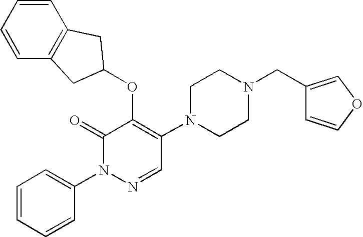 | 469 |
| 344 | 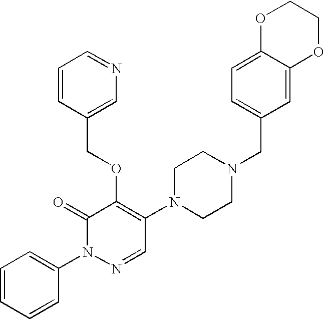 | 512 |
| 345 | 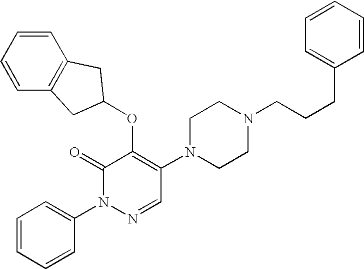 | 507 |
| 346 | 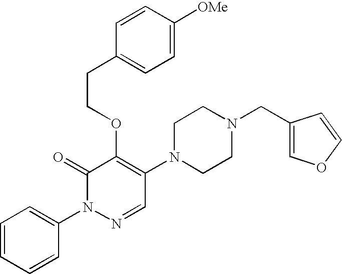 | 487 |
| 347 | 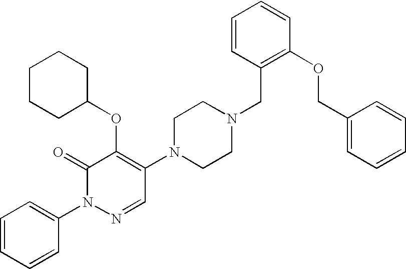 | 551 |
| 348 | 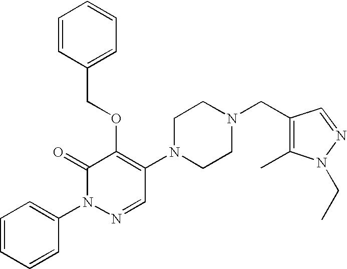 | 485 |
| 349 | 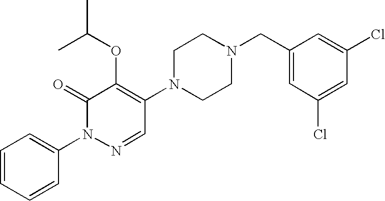 | 473 |
| 350 | 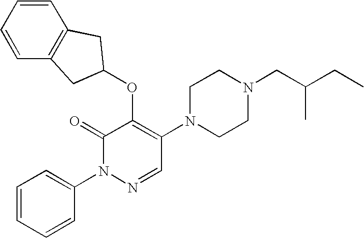 | 459 |
| 351 | 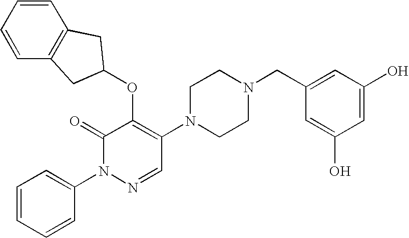 | 511 |
| 352 | 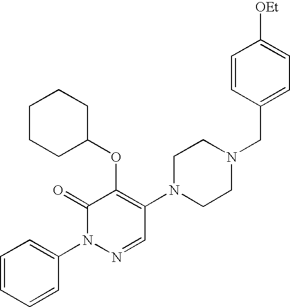 | 489 |
| 353 | 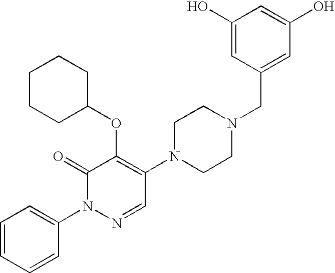 | 477 |
| 354 | 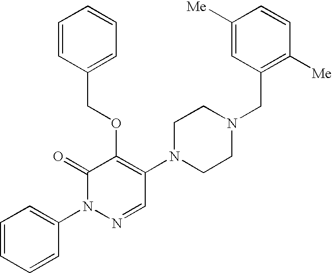 | 481 |
| 355 | 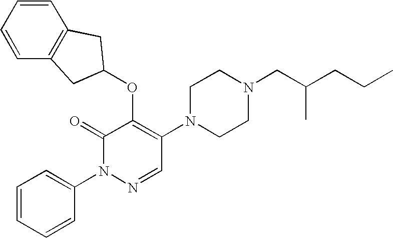 | 473 |
| 356 | 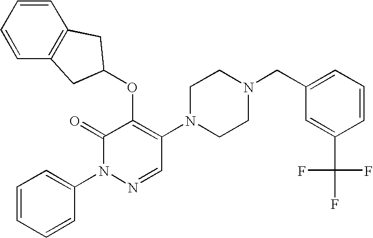 | 547 |
| 357 | 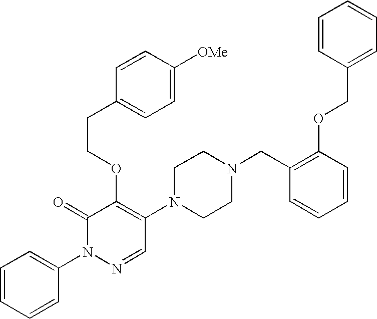 | 603 |
| 358 | 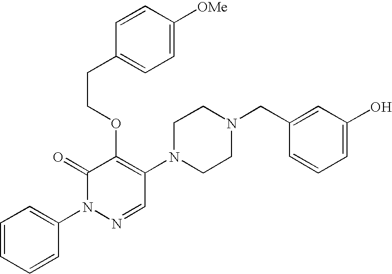 | 513 |
| 359 | 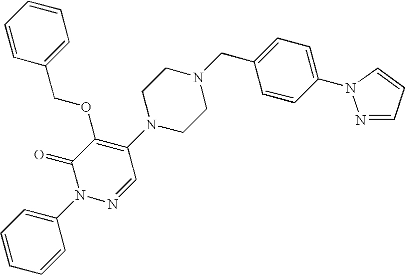 | 519 |
| 360 | 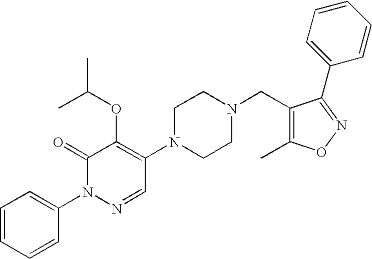 | 486 |
| 361 | 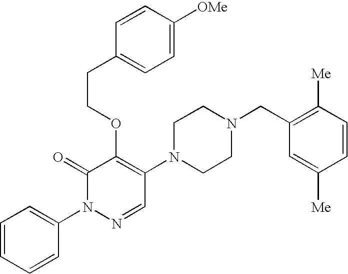 | 525 |
| 362 | 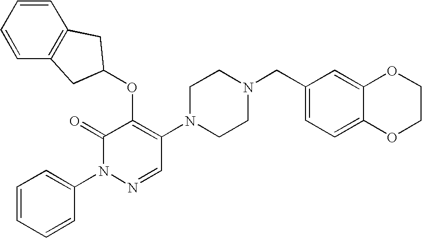 | 537 |
| 363 | 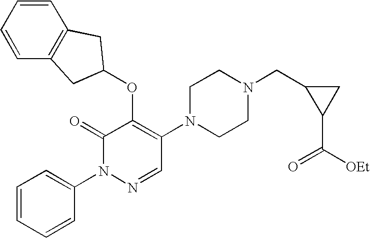 | 515 |
| 364 | 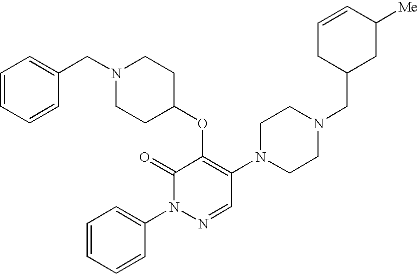 | 554 |
| 365 | 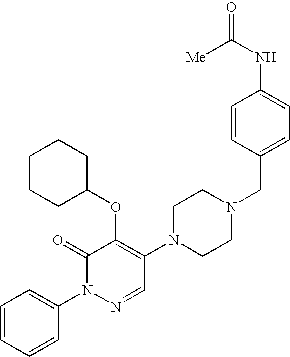 | 502 |
| 366 | 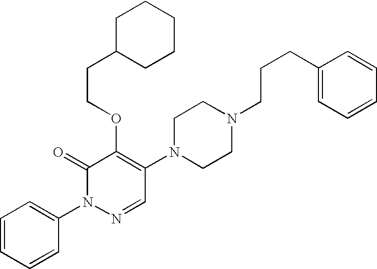 | 501 |
| 367 | 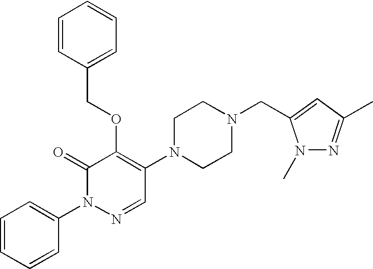 | 471 |
| 368 | 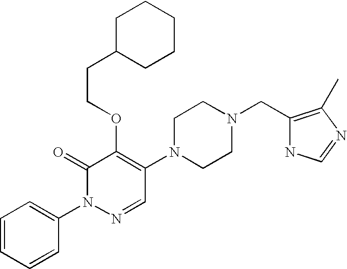 | 477 |
| 369 | 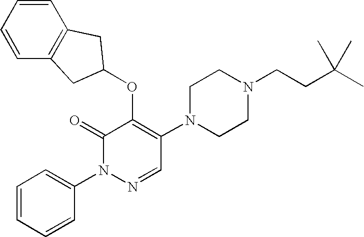 | 473 |
| 370 | 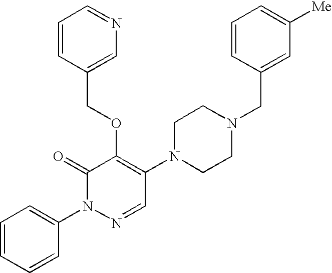 | 468 |
| 371 | 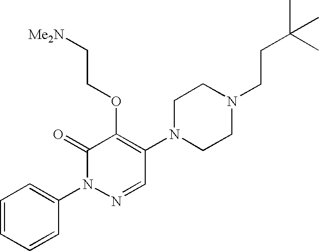 | 428 |
| 372 | 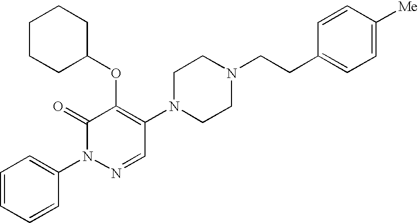 | 473 |
| 373 | 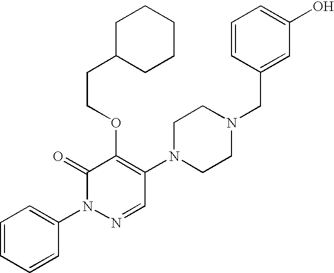 | 489 |
| 374 | 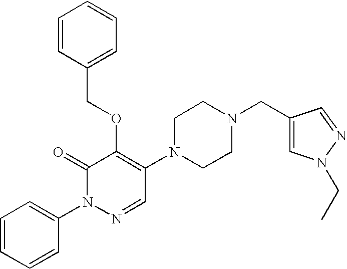 | 471 |
| 375 | 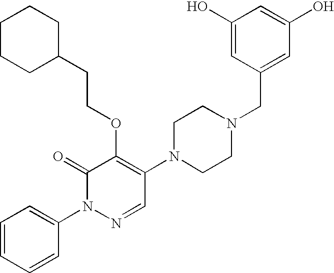 | 505 |
| 376 | 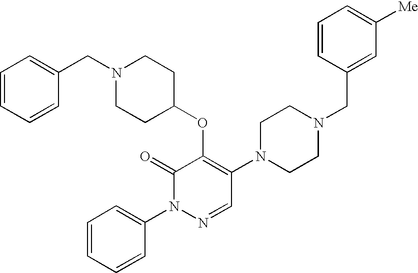 | 550 |
| 377 | 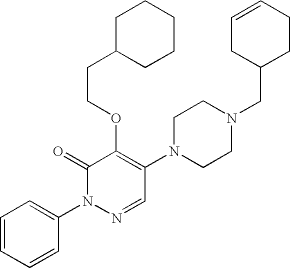 | 477 |
| 378 | 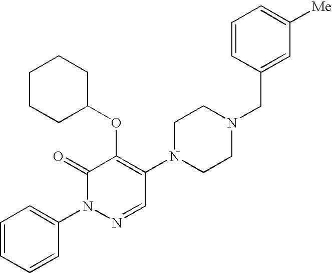 | 459 |
| 379 | 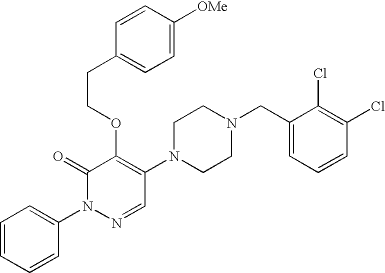 | 565 |
| 380 | 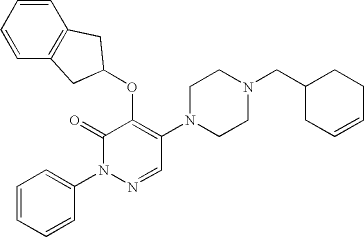 | 483 |
| 381 | 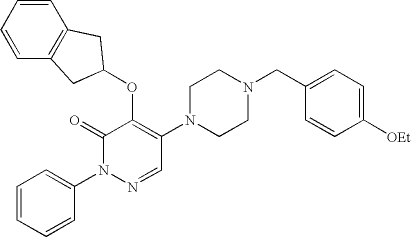 | 460 |
| 382 | 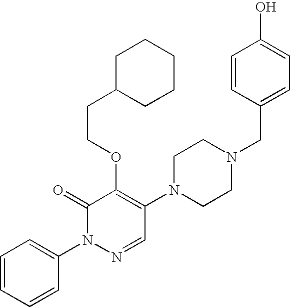 | 489 |
| 383 | 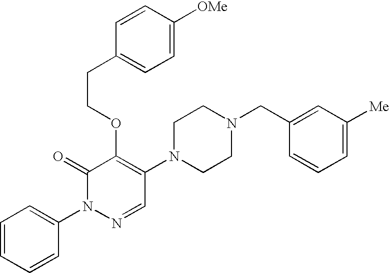 | 511 |
| 384 | 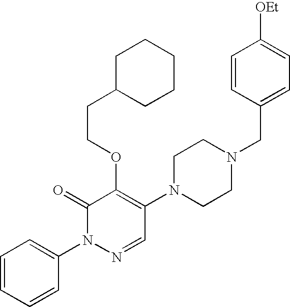 | 517 |
| 385 | 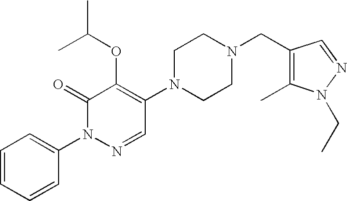 | 437 |
| 386 | 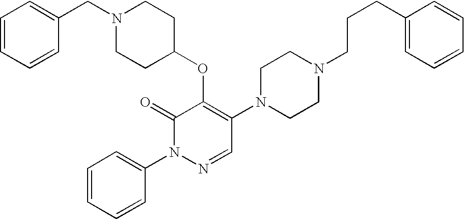 | 564 |
| 387 | 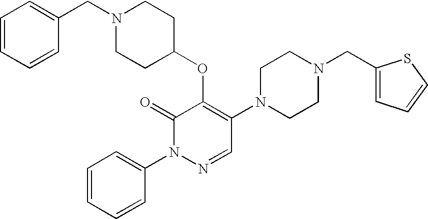 | 542 |
| 388 | 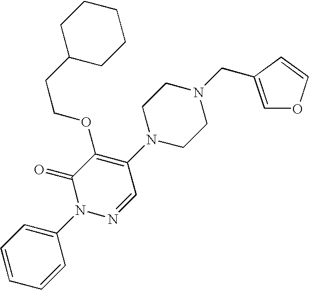 | 463 |
| 389 | 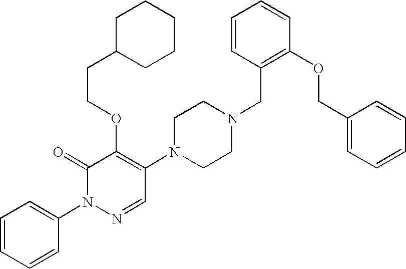 | 579 |
| 390 | 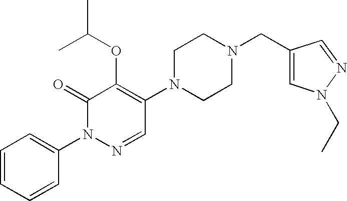 | 423 |
| 391 | 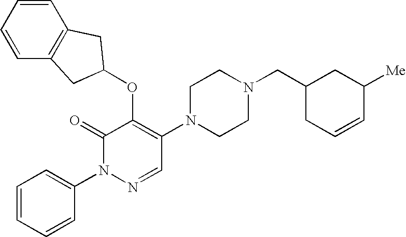 | 497 |
| 392 | 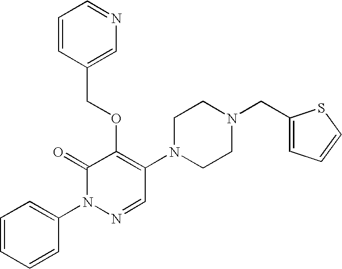 | 460 |
| 393 | 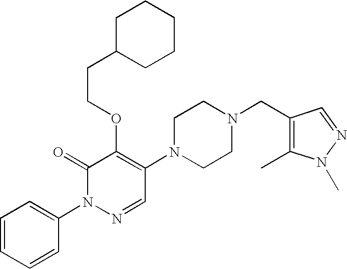 | 491 |
| 394 | 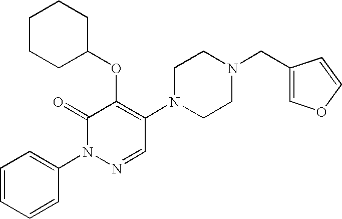 | 435 |
| 395 | 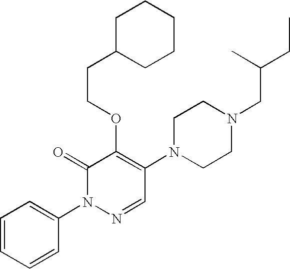 | 453 |
| 396 | 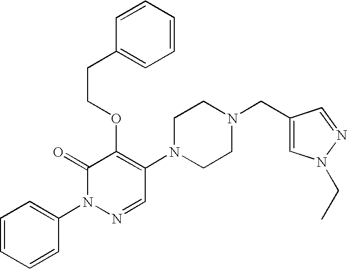 | 485 |
| 397 | 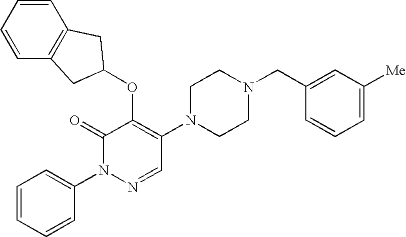 | 493 |
| 398 | 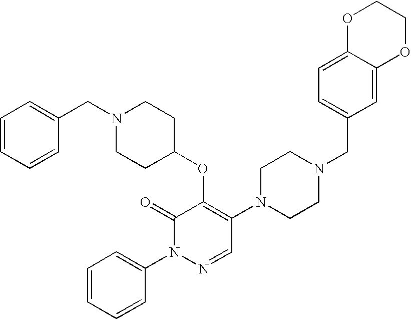 | 594 |
| 399 | 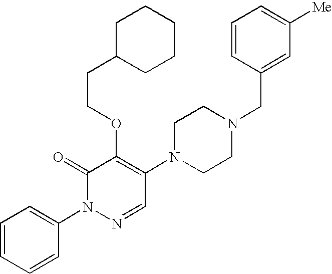 | 487 |
| 400 | 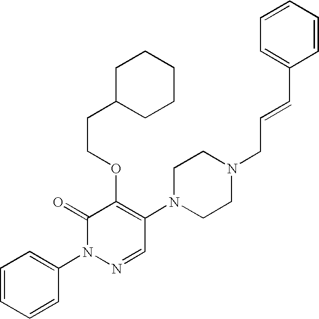 | 499 |
| 401 | 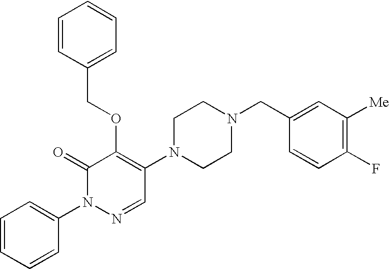 | 485 |
| 402 | 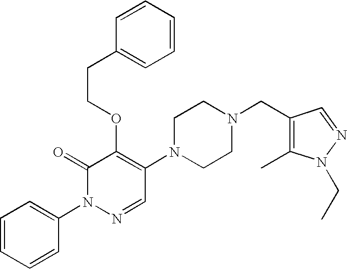 | 499 |
| 403 | 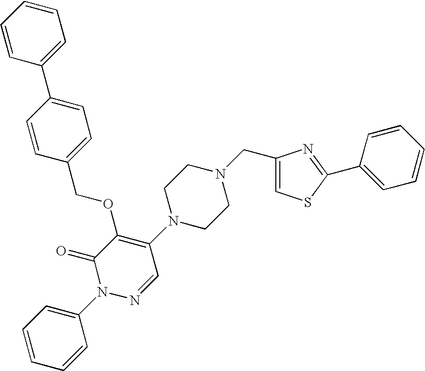 | 612 |
| 404 | 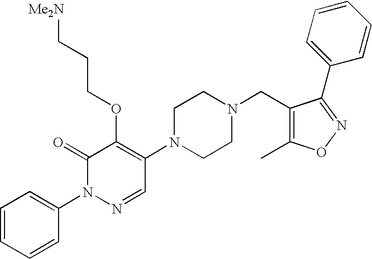 | 529 |
| 405 | 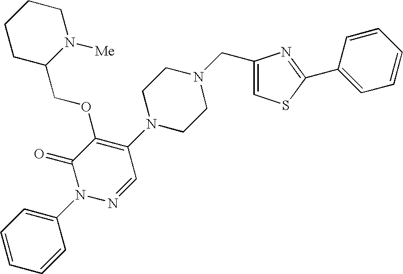 | 557 |
| 406 | 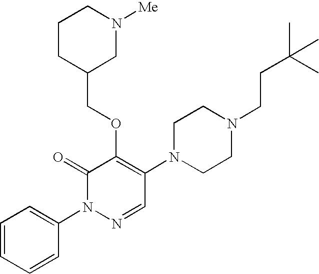 | 468 |
| 407 | 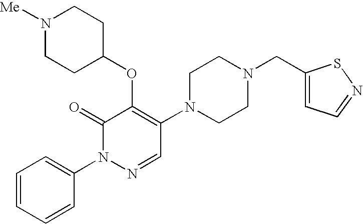 | 466 |
| 408 | 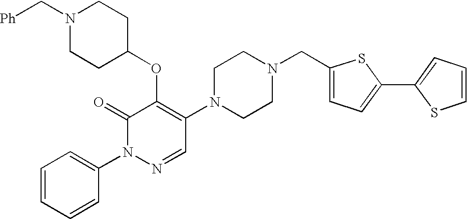 | 624 |
| 409 | 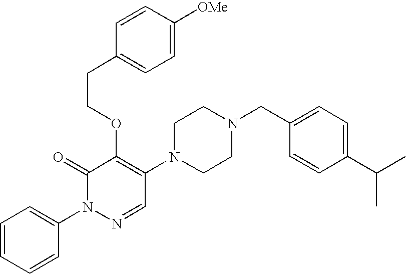 | 539 |
| 410 | 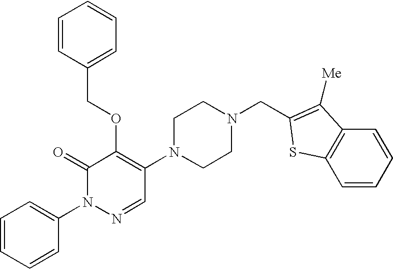 | 523 |
| 411 | 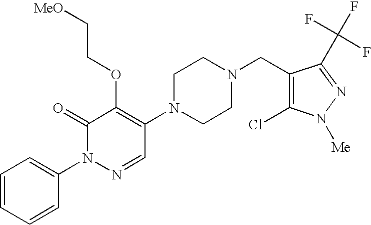 | 527 |
| 412 | 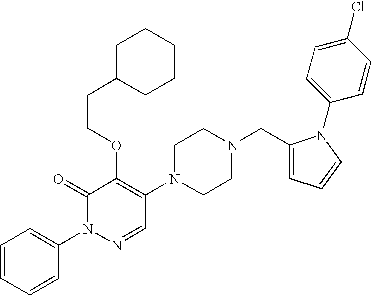 | 572 |
| 413 | 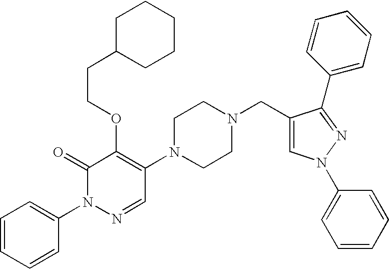 | 615 |
| 414 | 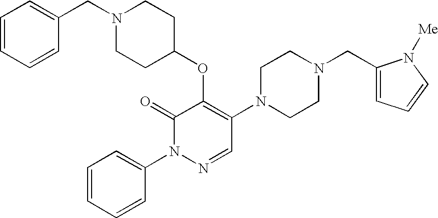 | 539 |
| 415 | 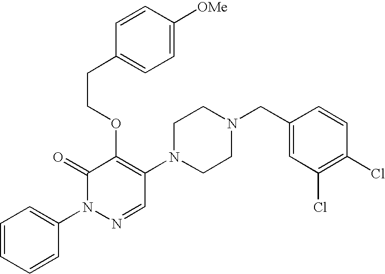 | 565 |
| 416 | 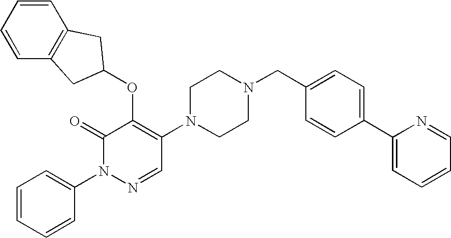 | 556 |
| 417 | 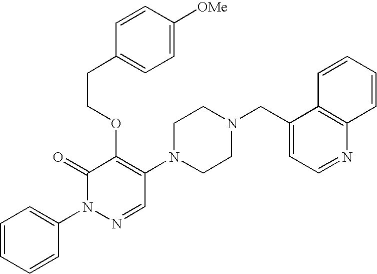 | 548 |
| 418 | 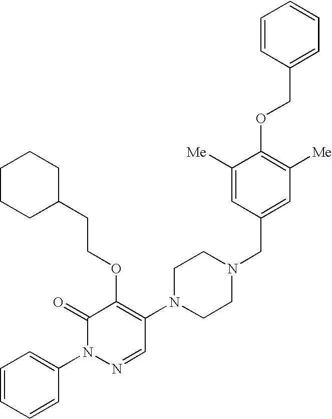 | 607 |
| 419 | 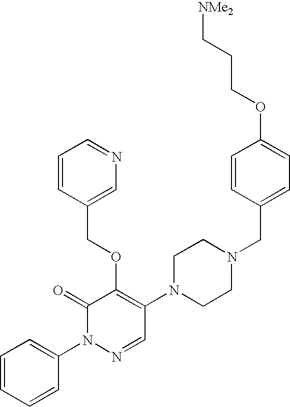 | 555 |
| 420 | 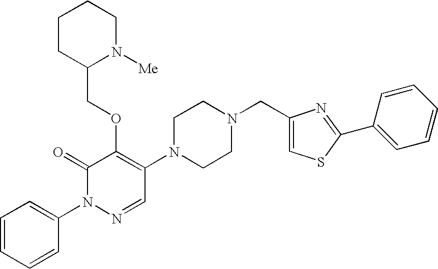 | 557 |
| 421 | 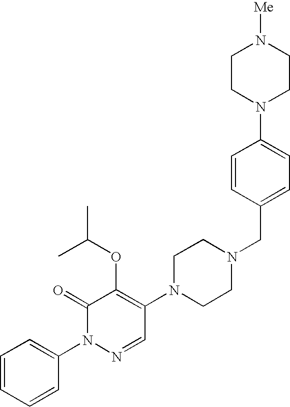 | 503 |
| 422 | 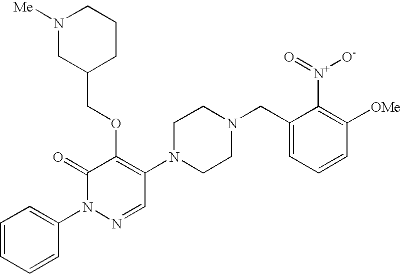 | 549 |
| 423 | 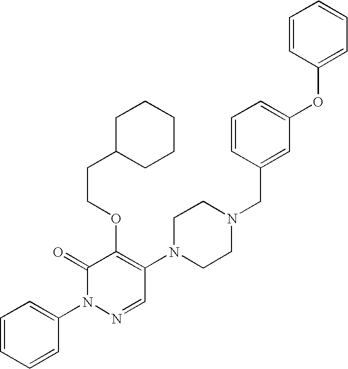 | 565 |
| 424 | 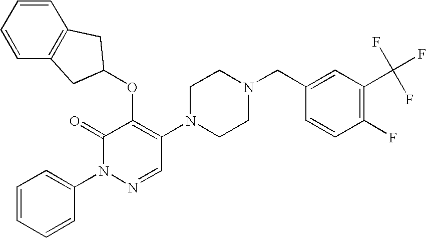 | 565 |
| 425 | 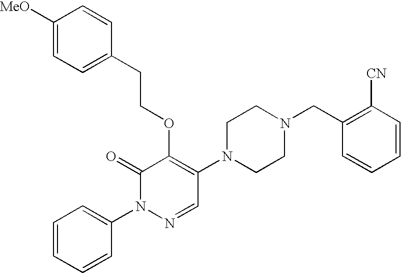 | 522 |
| 426 | 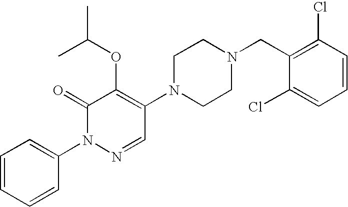 | 473 |
| 427 | 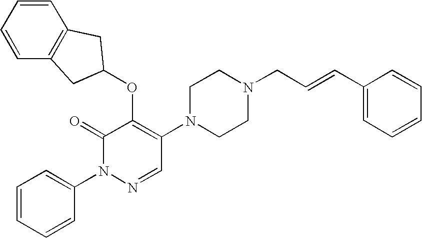 | 505 |
| 428 | 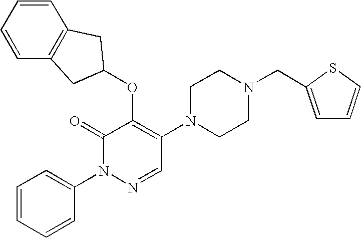 | 485 |
| 429 | 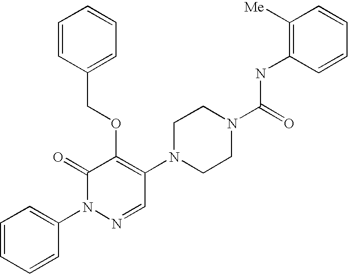 | 496 |
| 430 | 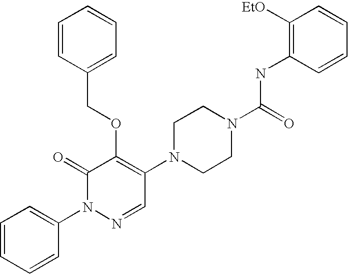 | 526 |
| 431 | 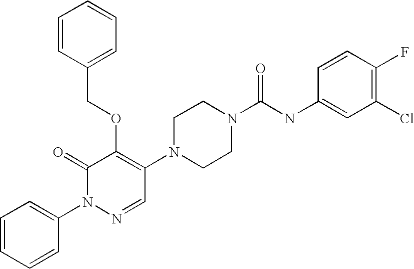 | 534 |
| 432 | 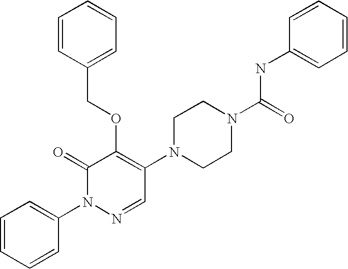 | 482 |
| 433 | 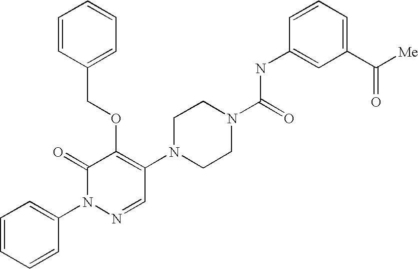 | 524 |
| 434 | 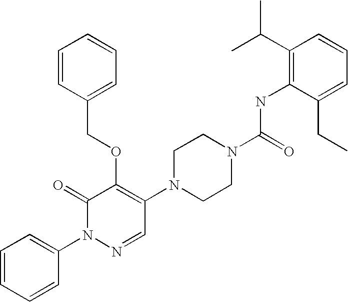 | 552 |
| 435 | 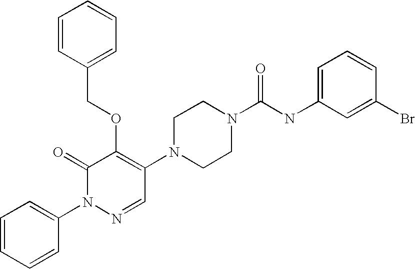 | 560 |
| 436 | 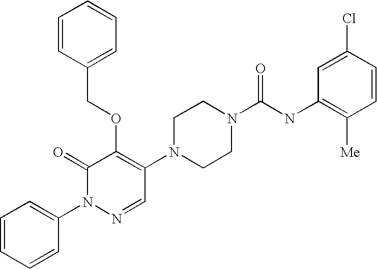 | 530 |
| 437 | 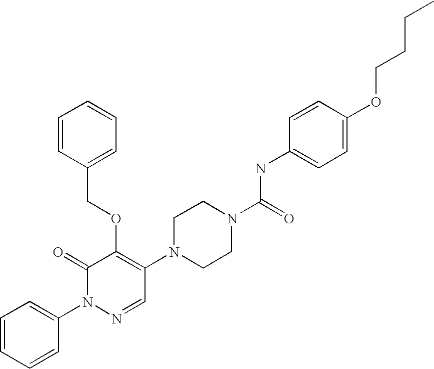 | 554 |
| 438 | 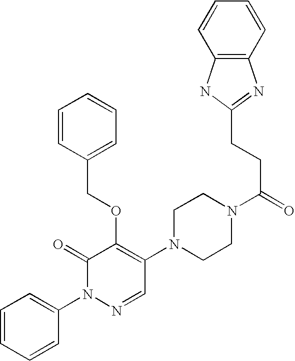 | 535 |
| 439 | 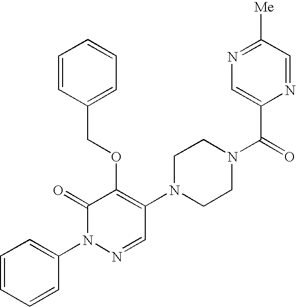 | 483 |
| 440 | 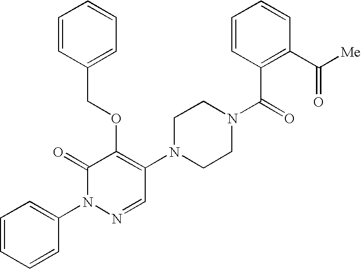 | 509 |
| 441 | 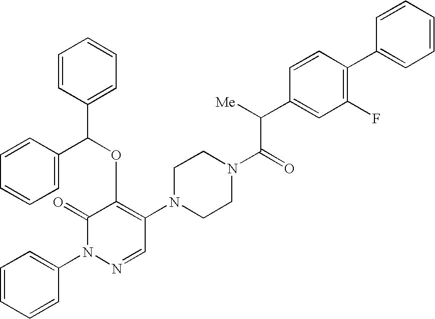 | 499 for M + 1 − 167 (Ph₂—CH₂) |
| 442 | 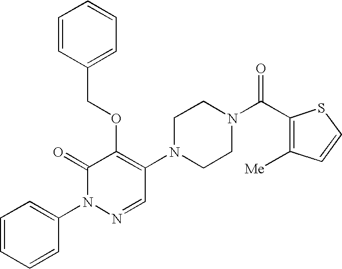 | 487 |
| 443 | 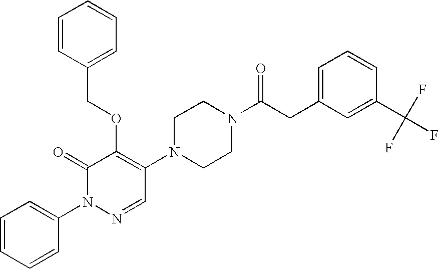 | 549 |
| 444 | 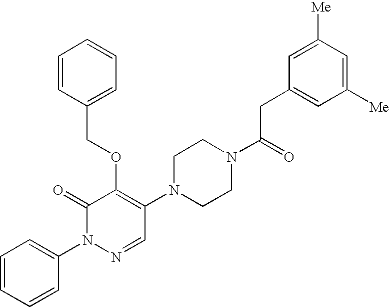 | 509 |
| 445 | 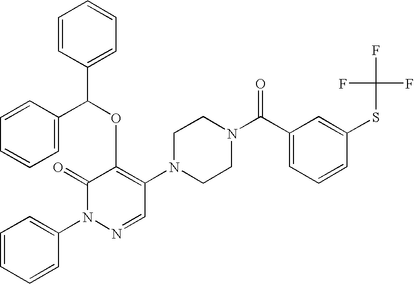 | 476 for M + 1 − 167 (Ph₂—CH₂) |
| 446 | 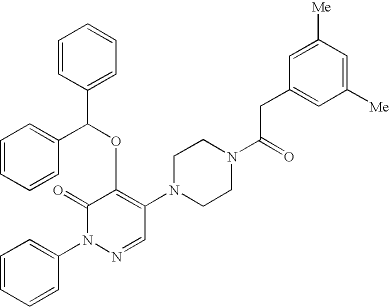 | 418 for M + 1 − 167 (Ph₂—CH₂) |
| 447 | 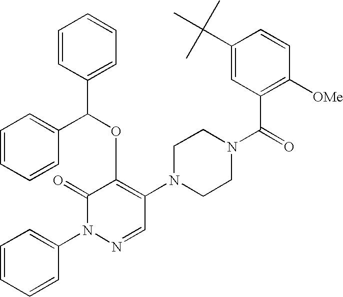 | 462 for M + 1 − 167 (Ph₂—CH₂) |
| 448 | 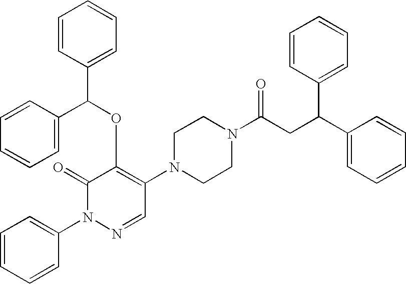 | 480 for M + 1 − 167 (Ph₂—CH₂) |
| 449 | 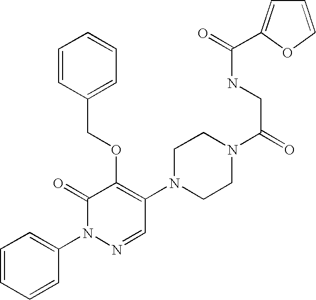 | 514 |
| 450 | 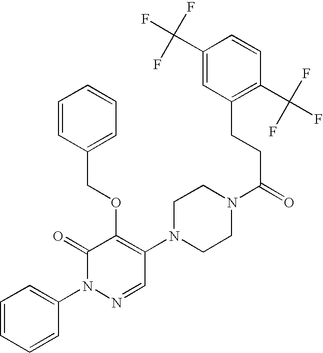 | 631 |

Compounds useful in the method of the invention were investigated for their utility as antifungal agents in the following assays.

### β(1,3) Glucan Synthase Assay:

1. Preparation of Permeabilized Saccharomyces cerevisiae Cells.

Permeabilization of yeast cells was performed according to Crotti et al. (Analytical Biochemistry, 292, 8-16, 2001) with some modifications. A 10 ml-starter culture of the S. cerevisiae strain in YPD medium (1% yeast extract, 2% bacto-peptone, 2% dextrose) with OD₆₀₀=3-4 was used to inoculate 1 liter of YPD. The culture was grown at 30° C. until OD₆₀₀=0.8. Cells were collected by centrifugation (5,300 g for 15 min at 4° C.) and resuspended in buffer (40 mM EDTA, 100 mM β-mercaptoethanol) at 1 g of cell pellet/3.5 ml buffer. The cell suspension was shaken for 30 min at 30° C., followed by centrifugation at 12,000 g for 10 min at 4° C. The cell pellet was washed with 5 ml 0.8 M sorbitol and resuspended in 6.8 ml of 2.9 mM citric acid, 11.3 mM dibasic sodium phosphate, 1 mM EDTA, 0.8 M sorbitol, with constant shaking at 30° C. for 30 min. After centrifugation at 12,000 g for 10 min at 4° C., the pellet was resuspended in 31.3 ml 50 mM Tris-HCl, pH 7.0, and incubated on ice for 5 min. The mixture was then centrifuged at 12,000 g for 10 min at 4° C., and the pellet was resuspended in 1 ml of 50 mM Tris-HCl and 33% glycerol, pH 7.5. The permeabilized cell preparation was stored at −80° C. in aliquots.

### 2. Preparation of Membrane Fraction of Yeast Cells.

The protocol was modified from Douglas et al. (Journal of Bacteriology, 176, 5686-5696, 1994). For the preparation of S. cerevisiae and C. albicans membrane fractions, 1 liter of YPD supplemented with 0.02 mg/mL adenine and 0.08 mg/mL uracil was inoculated with 10 mL starter culture of PM503 (OD₆₀₀=4) or the C. albicans strain BWP17 (OD₆₀₀=12) in the same medium and grown at 30° C. until OD₆₀₀ reached about 1. A. fumigatus (strain ND158) membranes were prepared by first preparing a spore suspension from agar slants by adding 6 mL of sterile saline, 0.1% Tween-20 solution to each slant, and resuspending by pipetting and scraping. The spore suspensions was used to inoculate two 200 mL flasks containing Sabouraud dextrose broth media. Cultures were incubated at 37 C, 250 rpm for ±8 hrs. All cells, S. cerevisiae, C. albicans or A. fumigatus were harvested by centrifugation at 5,300 g at 4° C. for 40 minutes. After washing with 100 mL of breakage buffer (0.1 M KPi, pH 7.0, 1 mM EDTA, 1 mM DTT), the cell pellet was resuspended in 50 ml ice-cold breakage buffer. The mixture was transferred to a bead-beater chamber packed in ice (BioSpec Products, Bartlesville, Okla.). To each 50 mL sample was added 50 g of acid-washed glass beads (0.45 μM, Sigma). Cells were disrupted using 12×20 second pulses with 2 min-cooling intervals. Cell debris was removed by centrifugation at 3,000 g for 20 minutes at 4° C., and the supernatant was collected and centrifuged at 100,000 g for 1 hour at 4° C. to pellet the membrane fraction. The pellet was resuspended in 5 mL of ice-cold breakage buffer containing 25% glycerol, homogenized with a Dounce tissue homogenizer and stored at −80 C in small aliquots.

### 3. Glucan Synthesis Assay and Compound Screening

The assay was performed according to Mo et al. (Journal of Biological Chemistry, 269, 31267-31274, 1994) and Taft et al. (The Journal of Antibiotics, 47, 1001-1009, 1994), in a 96-well Optiplate (PerkinElmer). To each well was added 3 μL 10× compound stock (in 100% DMSO), or 3 μL of 30 μg/mL caspofungin in 100% DMSO (as positive control), or 3 μL 100% DMSO (as negative control), followed by the addition of appropriate amount of glucan synthase sources (2 μL permeabilized PM503 cells, or 3 μL membrane preparations from either PM503, BWP17, or ND158). The reaction was initiated by adding 25 μL reaction buffer (0.6 mM UDP-Glucose, 0.6 nCi [U-¹⁴C]DUP-Glucose (327 mCi/mmol, Amersham Bioscience), 20 μM GTP-γ-S, 25 mM NaF, 7.5 mg/mL BSA, 8% glycerol in 75 mM Tris-HCl, pH 7.5). The plate was incubated on a shaker for 1.5 hour at room temperature before being quenched with 250 μL 1% TCA (Trichloroacetic Acid). The quenched reaction was mixed by pipetting, and immediately transferred to a 96-well filter plate (Glass fiber B on 0.65 μm hydrophilic durapore membrane, Millipore) pre-wetted with wash buffer (5% TCA, 60 mM NaPPi). The glucan product was retained on the filter membrane by applying vacuum to the plate using a MutiScreen Resist Vacuum Manifold (Millipore). The filter plate was further washed 4 times with 200 wash buffer. The plate was dried at 50° C. for 30 minutes. 100 μL of Microscint-0 (PerkinElmer) was added to each well, and plate was counted in a TopCount NXT plate reader (PerkinElmer).

### IC₅₀ Determinations:

Dose-response curves were plotted from inhibition data generated. IC₅₀ was determined by fitting the CPM versus the Concentration of the test compound plot with the following equation (4-parameter logistic model, ID Business Solutions XL_{fit} 4.2).

### Microbroth Susceptibility Testing Methods

Yeast susceptibility testing procedure followed the NCCLS document M27-A2 (Reference Method for Broth Dilution Antifungal Susceptibility Testing of Yeasts; Approved Standard-Second Edition (ISBN 1-56238-469-4). NCCLS, 940 West Valley Road, Suite 1400 Wayne, Pa. 19087-1898 USA, 2002) with the following modifications:

1. The final test volume was 100 μl and not 200 μl as stipulated.
2. For testing Saccharomyces cerevisiae strain PM503 YPD was used in place of RPMI 1640 broth.

Filamentous fungi susceptibility testing procedure follows the NCCLS document M38-A (Reference Method for Broth Dilution Antifungal Susceptibility Testing of Filamentous Fungi; Approved Standard (ISBN 1-56238-470-8). NCCLS, 940 West Valley Road, Suite 1400 Wayne, Pa. 19087-1898 USA, 2002) with the following modifications:

1. The final test volume was 100 μl and not 200 μl as stipulated.
2. The end point used to assess the in vitro activity of glucan synthase inhibitors may require microscopic evaluation of cell morphology in the test wells (Kurtz et al., Antimicrobial Agents and Chemotherapy, 38, 1480-1489, 1994; Arikan et al., Antimicrobial Agents and Chemotherapy, 45, 327-330, 2001). This endpoint, termed the minimum effective concentration (MEC), is characterized by changes in the fungal growth that resulted in truncated and highly branched hyphae.

The β(1,3) Glucan Synthase inhibitory activity and in vitro fungal cell activity for representative compounds of the invention are listed in the Tables that follow (ranges of IC₅₀* values in micrograms/milliliter):
* Category A: ≦0.5 μg/mL;
Category B: >0.5 μg/mL and ≦1.0 μg/mL;
Category C: >1.0 μg/mL and ≦5.0 μg/mL;
Category D: >5.0 μg/mL and ≦50 μg/mL;
Category E: >50 μg/mL (Inactive).
   Results of the glucan synthase assay for the above listed compounds used in this invention are listed in Table 1:

TABLE 1
Glucan Synthase Enzyme Inhibition for compounds used in the
present invention

| | IC₅₀ | IC₅₀ |
|---|---|---|
| | Saccharomyces | Candida |
| Compound No. | cerevisiae | albicans |
| 253 | A | C |
| 254 | B | C |
| 236 | A | B |
| 343 | A | C |
| 428 | A | C |
| 339 | A | C |
| 66 | A | C |
| 74 | A | D |
| 67 | C | D |
| 9 | C | D |
| 1 | A | C |
| 136 | A | D |
| 114 | B | D |
| 17 | A | D |
| 130 | A | D |
| 26 | A | D |
| 43 | ND | D |
| 45 | A | D |
| 186 | D | D |
| 92 | B | D |

ND: not determined

Results of the in vitro fungal cell activity assay for above-listed representative compounds used in this invention are listed in Table 2:

TABLE 2
In Vitro Fungal Cell Activity for compounds used in the present invention:

| | | | | | MEC |
|---|---|---|---|---|---|
| | MIC | MIC Candida | MIC Candida | MIC | Aspergillus |
| | Saccharomyces | albicans | albicans | Candida glabrata | fumigatus |
| Compound | cerevisiae (C51) | (C693) | (C697) | (C624) | (ND158) |
| 253 | C | D | C | C | D |
| 254 | C | D | D | C | D |
| 236 | C | C | C | B | C |
| 343 | A | D | C | C | B |
| 428 | B | D | C | D | C |
| 339 | C | D | C | C | C |
| 373 | B | D | C | C | D |
| 316 | B | D | D | D | C |
| 66 | C | D | D | B | C |
| 74 | C | D | C | B | C |
| 67 | D | D | D | C | D |
| 9 | B | D | D | B | D |
| 1 | D | D | D | C | D |
| 136 | D | D | D | D | D |
| 114 | D | D | D | D | D |
| 17 | D | D | D | D | D |
| 130 | D | D | D | C | D |

The compounds listed above can be administered to an animal orally, intravenously, by inhalation (e.g., to treat fungal infections in the lungs) or topically (e.g. to treat fungal infections of the skin or mucous membranes). Preferably the compound(s) of the invention listed above is administered orally or intravenously, more preferably orally.

For preparing pharmaceutical compositions from the compounds useful in the method of this invention, inert, pharmaceutically acceptable carriers can be either solid or liquid. Solid form preparations include powders, tablets, dispersible granules, capsules, cachets and suppositories. The powders and tablets may be comprised of from about 0.1 to about 99 percent active ingredient. Suitable solid carriers are known in the art, e.g. magnesium carbonate, magnesium stearate, talc, sugar, lactose. Tablets, powders, cachets and capsules can be used as solid dosage forms suitable for oral administration.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Liquid form preparations include solutions, suspensions and emulsions. As an example may be mentioned water or water-propylene glycol solutions for parenteral injection.

Liquid form preparations may also include solutions for intranasal administration.

Aerosol preparations suitable for inhalation may include solutions and solids in powder form, which may be in combination with a pharmaceutically acceptable carrier, such as an inert compressed gas.

Also included are solid form preparations which are intended to be converted, shortly before use, to liquid form preparations for either oral or parenteral administration. Such liquid forms include solutions, suspensions and emulsions.

The compounds useful in the method of the invention may also be deliverable transdermally. The transdermal compositions can take the form of creams, lotions, aerosols and/or emulsions and can be included in a transdermal patch of the matrix or reservoir type as are conventional in the art for this purpose.

Preferably, the pharmaceutical preparation is in unit dosage form. In such form, the preparation is subdivided into unit doses containing appropriate quantities of the active component, e.g., an effective amount to achieve the desired purpose.

The quantity of compound listed above in a unit dose of preparation may be varied or adjusted from about 0.1 mg to 1000 mg, more preferably from about 1 mg to 300 mg, according to the particular application.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small increments until the optimum effect under the circumstances is reached. For convenience, the total daily dosage may be divided and administered in portions during the day if desired.

The amount and frequency of administration of the compound listed above useful in the method of the invention will be regulated according to the judgment of the attending clinician considering such factors as age, condition and size of the patient as well as severity of the symptoms being treated. A typical recommended dosage regimen for a compound listed above is oral administration of about 10 mg to 2000 mg/day preferably 10 to 1000 mg/day, in two to four divided doses to provide relief from the fungal infection.

When the invention comprises a combination of one or more compounds listed above and one or more other antifungal agents, the active components may be co-administered simultaneously or sequentially, or a single pharmaceutical composition comprising one or more compounds listed above and one or more other antifungal agents in a pharmaceutically acceptable carrier can be administered. The components of the combination can be administered individually or together in any conventional dosage form such as capsule, tablet, powder, cachet, suspension, solution, suppository, nasal spray, etc. The dosages of the other antifungal agents can be determined from published material, and may range from 1 to 1000 mg per dose. When used in combination, the dosage levels of the individual components are preferably lower than the recommended individual dosages because of the advantageous effect of the combination.

When separate pharmaceutical compositions of compounds listed above and other antifungal agents are to be administered, they can be provided in a kit comprising in a single package, one container comprising one or more compounds of the present invention listed above in a pharmaceutically acceptable carrier, and a separate container comprising one or more other antifungal agents in a pharmaceutically acceptable carrier, with the compounds listed above and the other antifungal agents being present in amounts such that the combination is therapeutically effective. A kit is advantageous for administering a combination when, for example, the components must be administered at different time intervals or when they are in different dosage forms.

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

1. The use of at least one compound of a formula selected from the group consisting of: 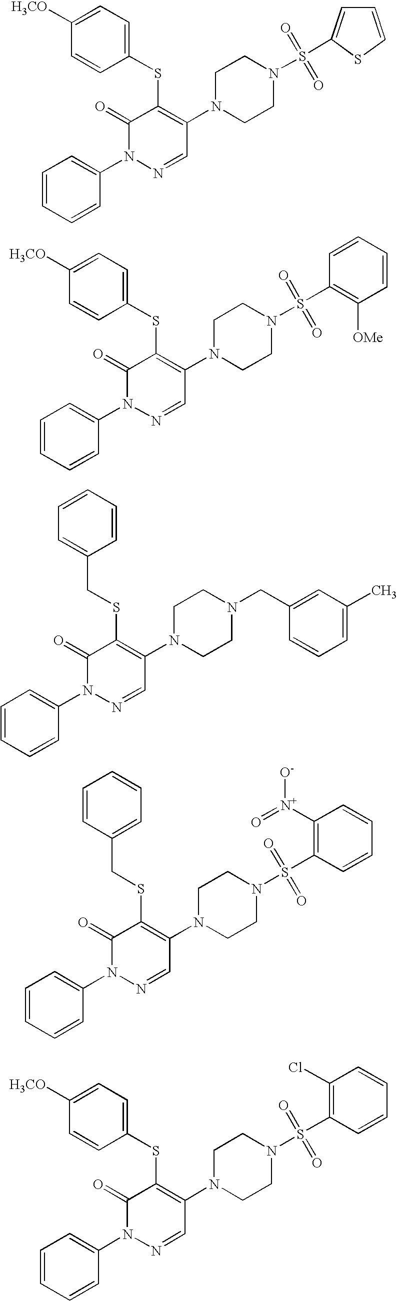 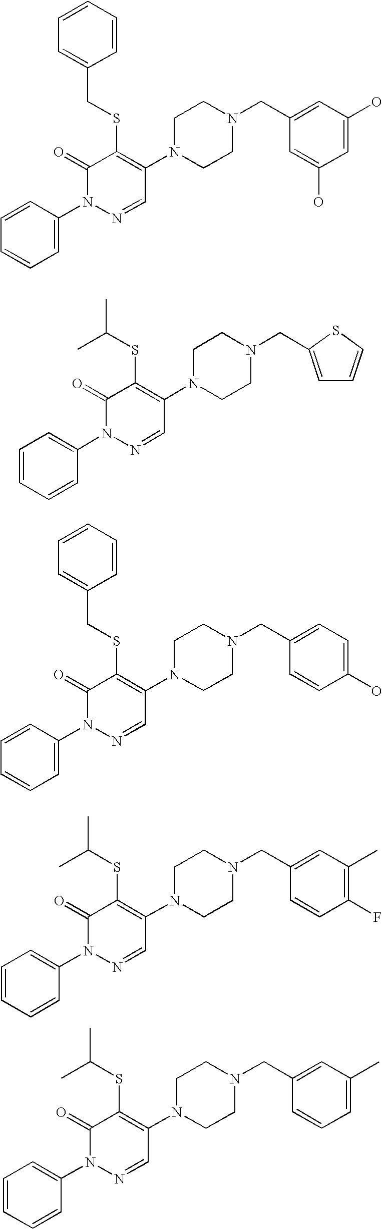 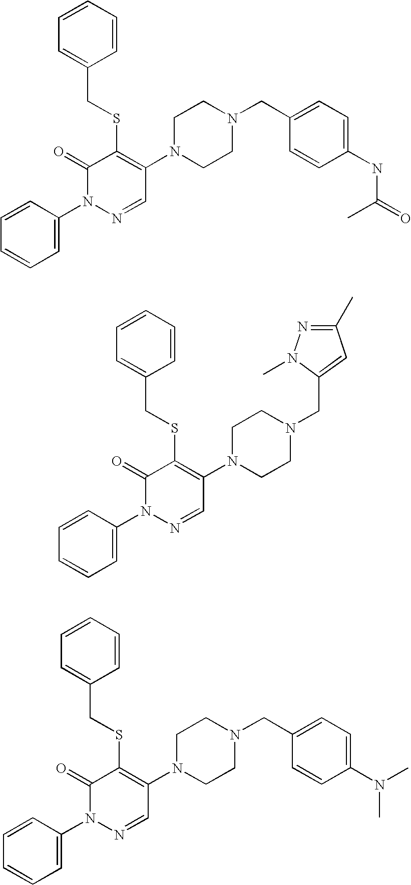 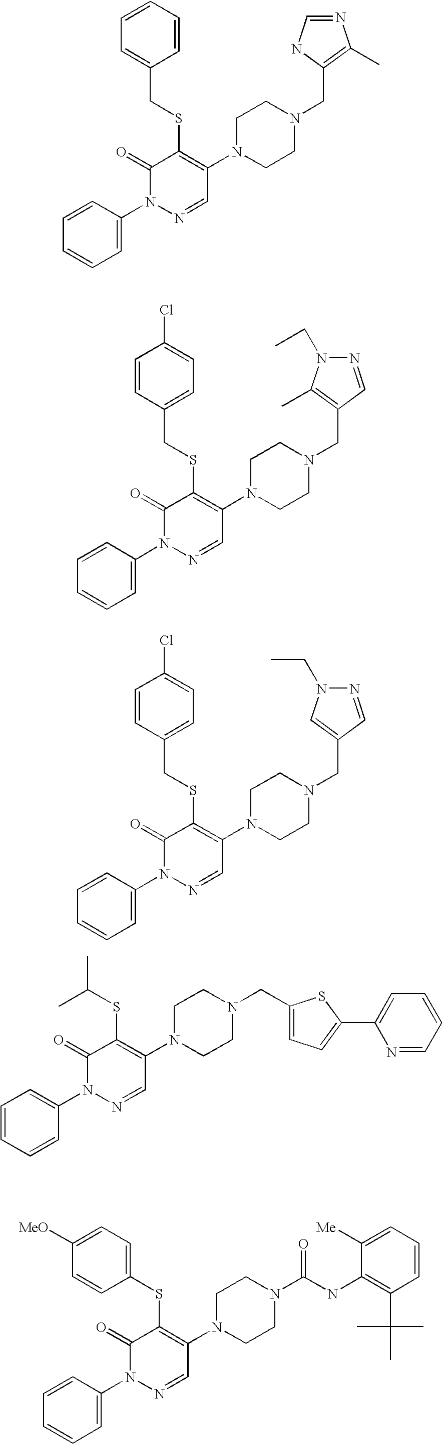 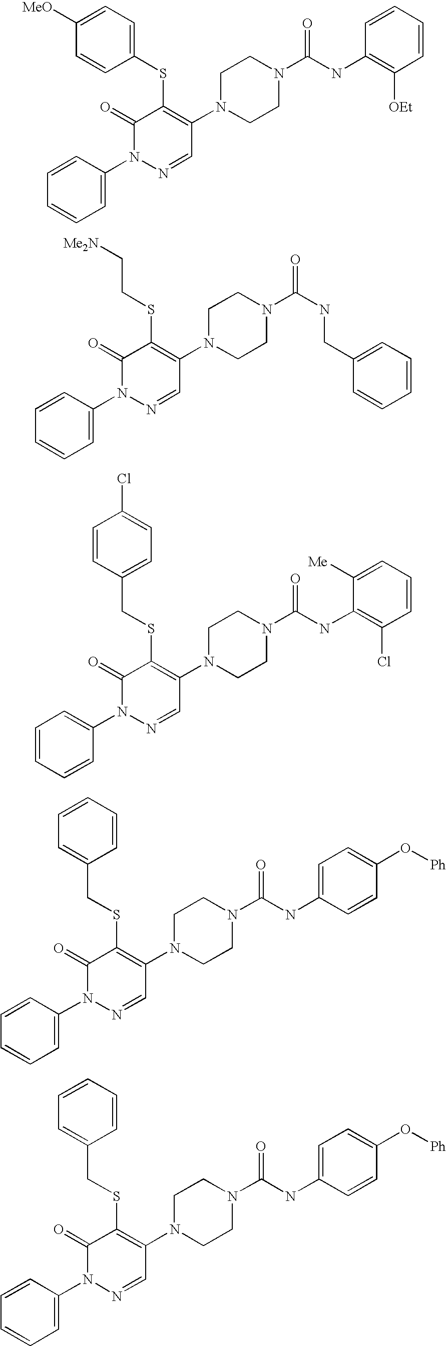 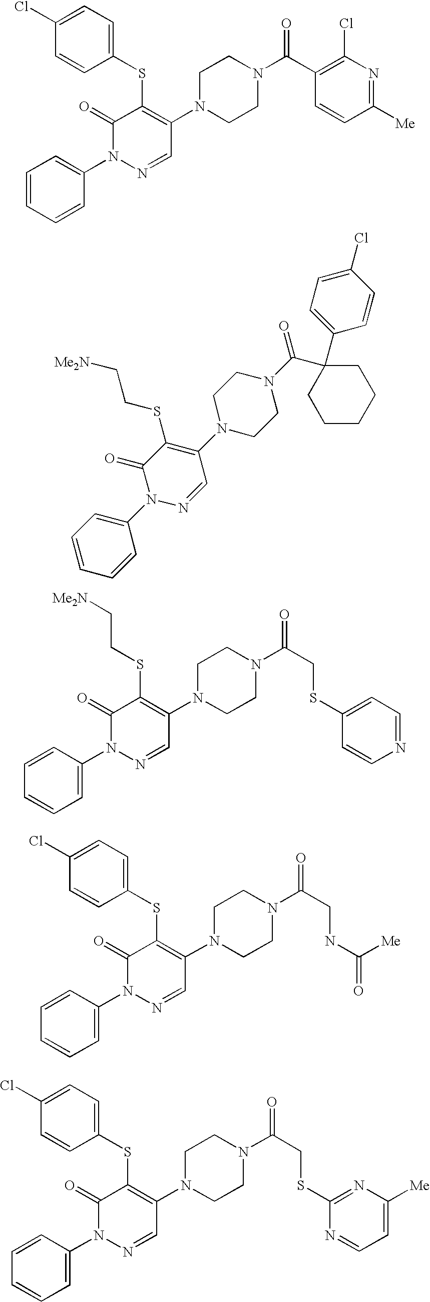 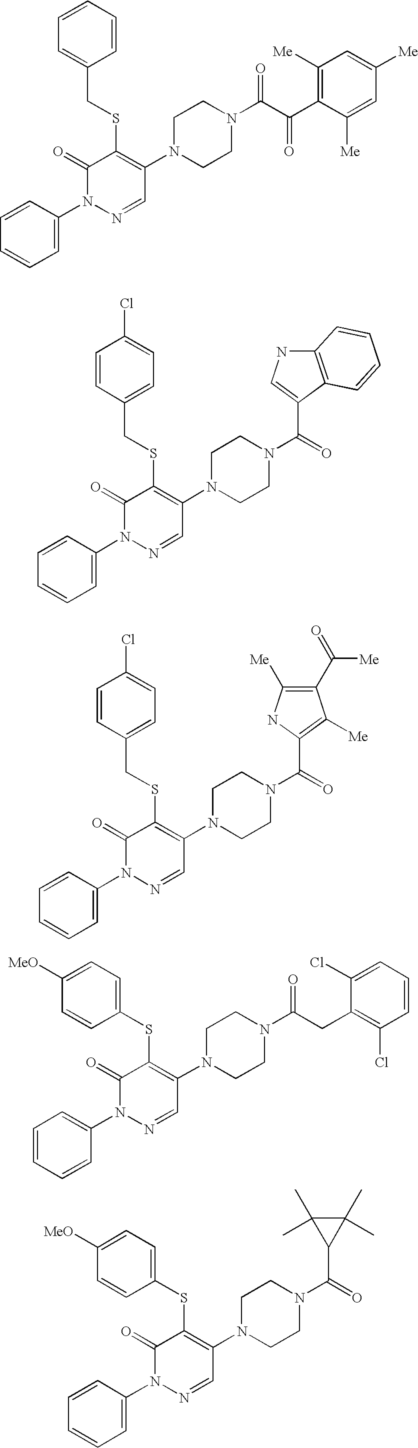 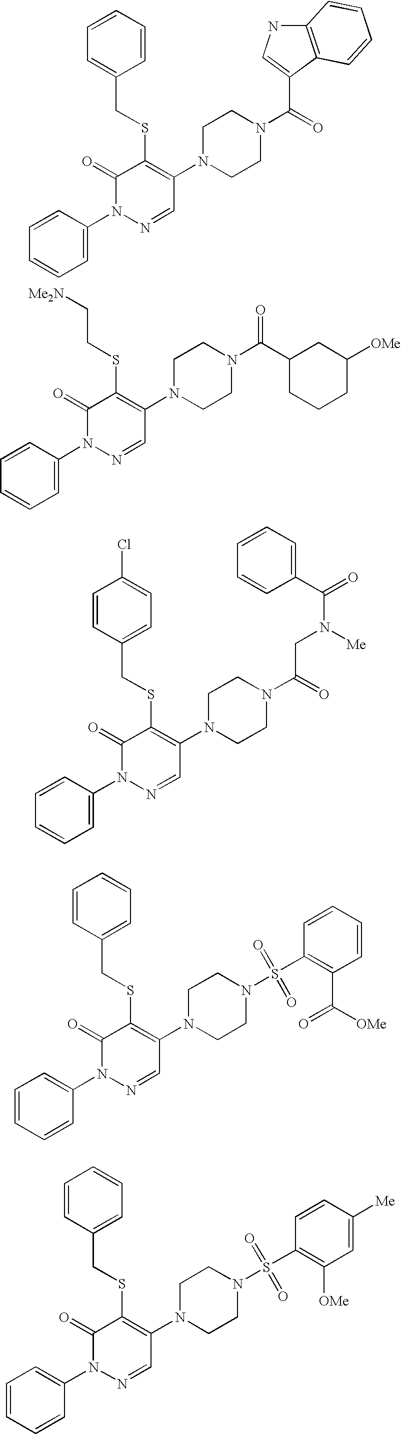 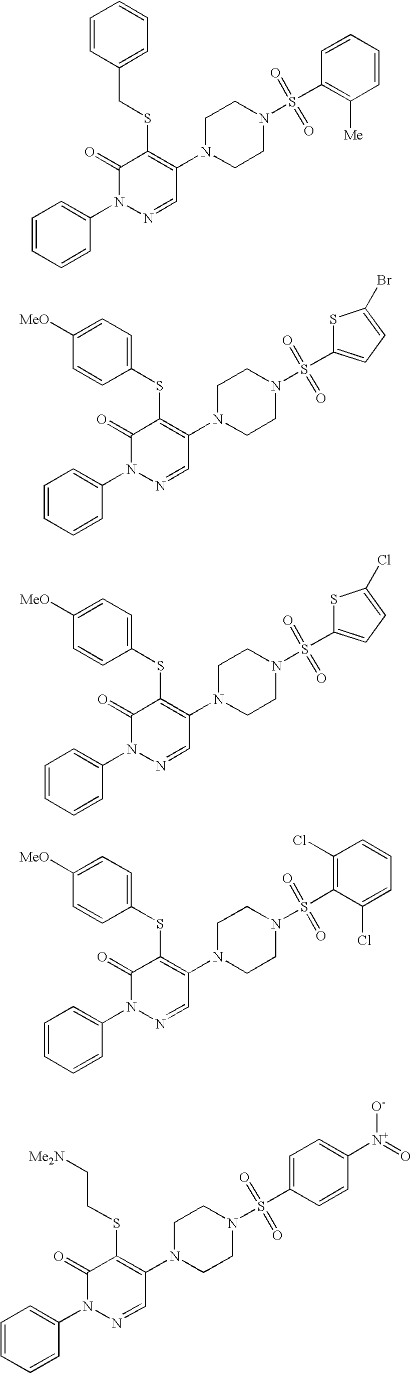 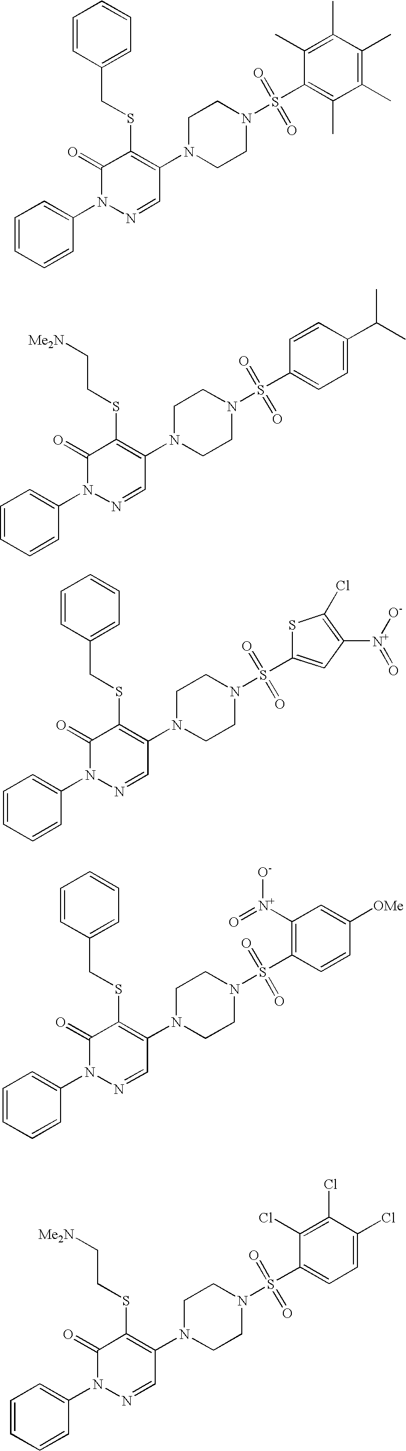 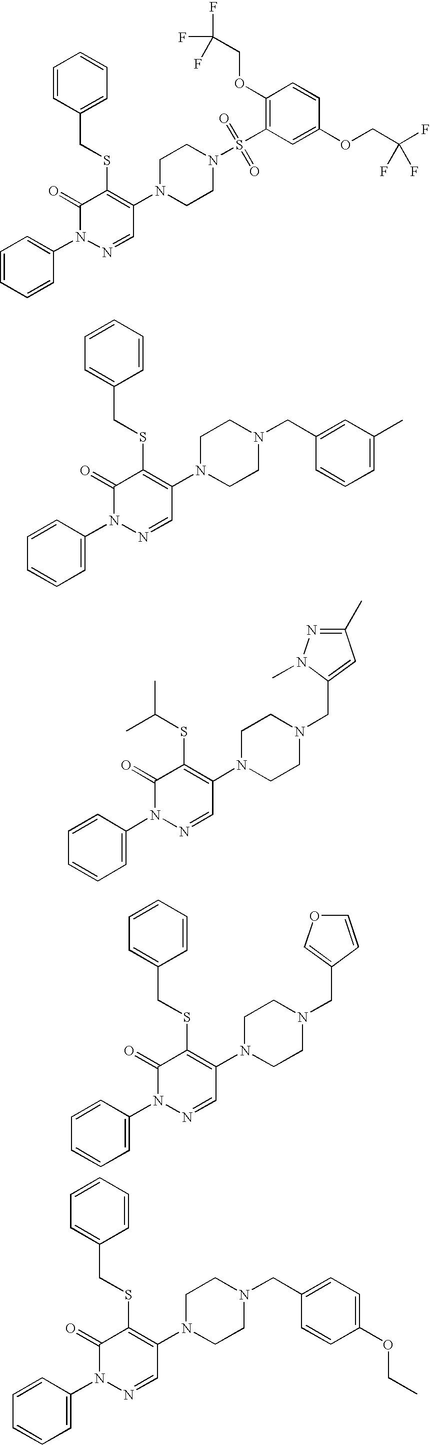 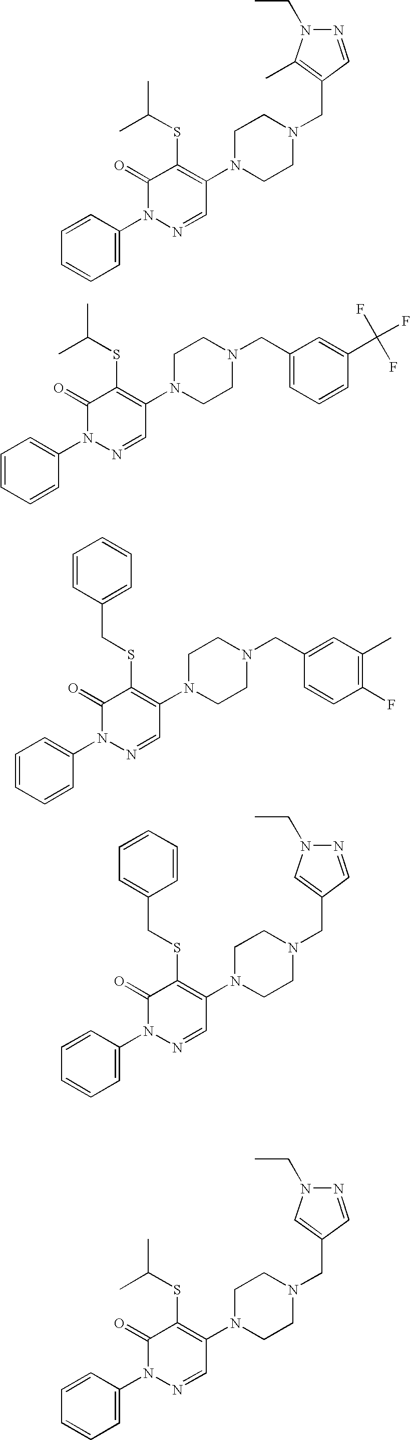 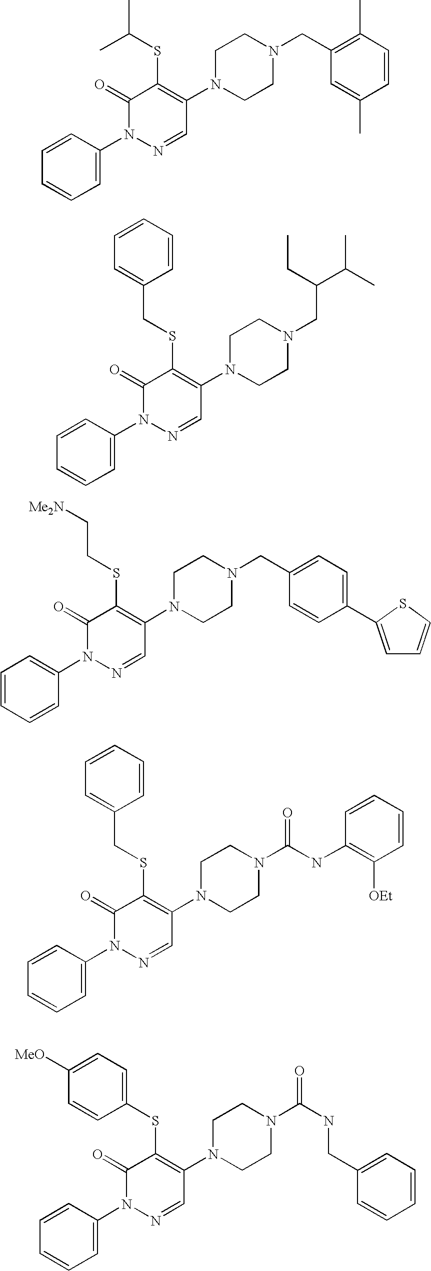 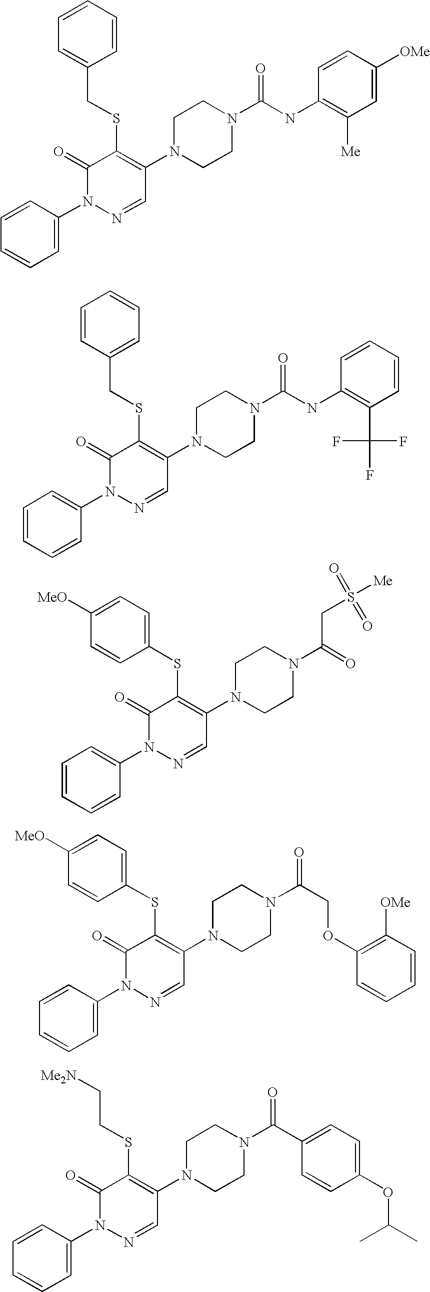 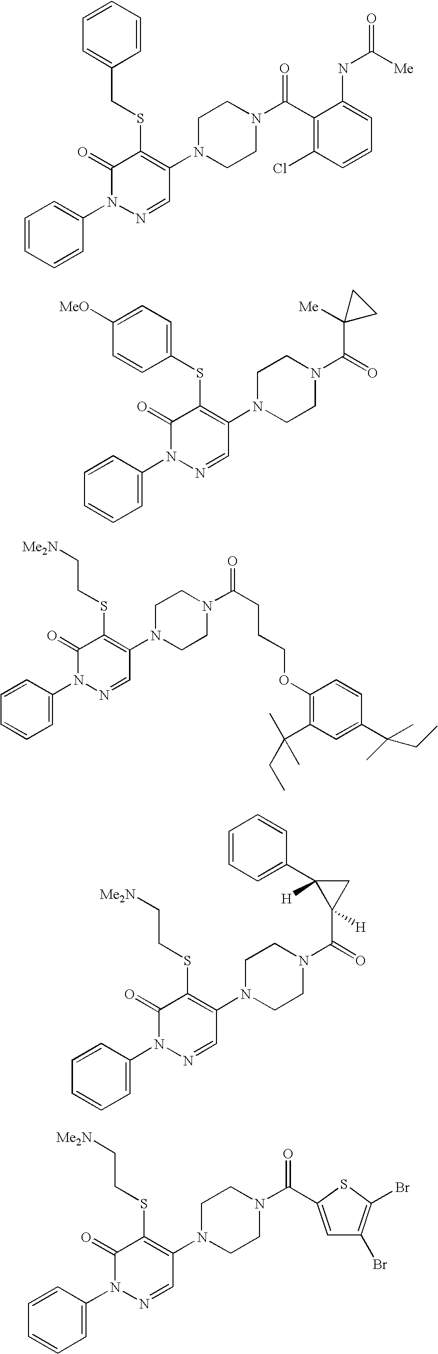 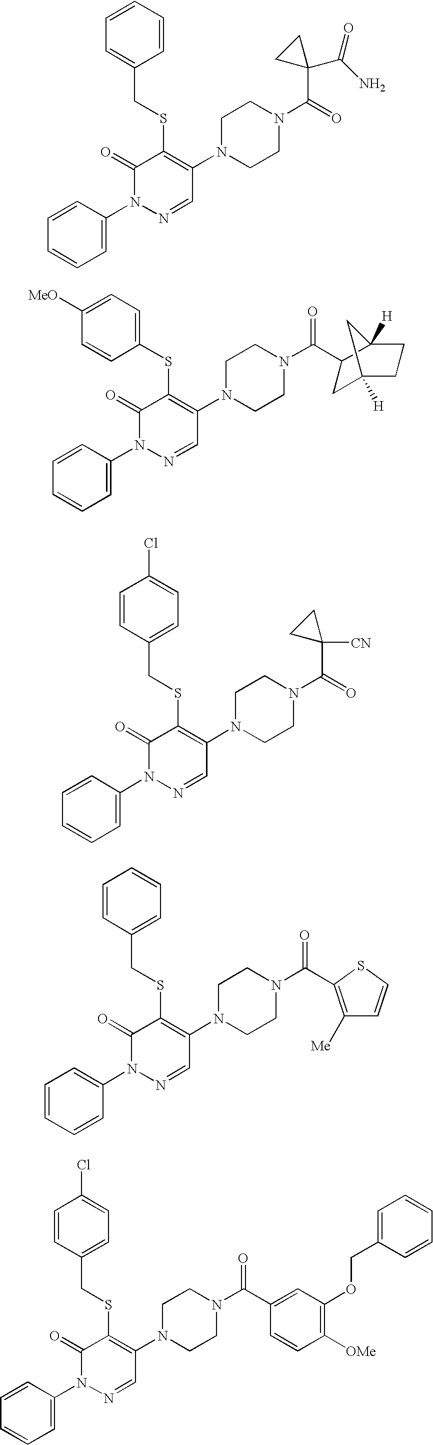 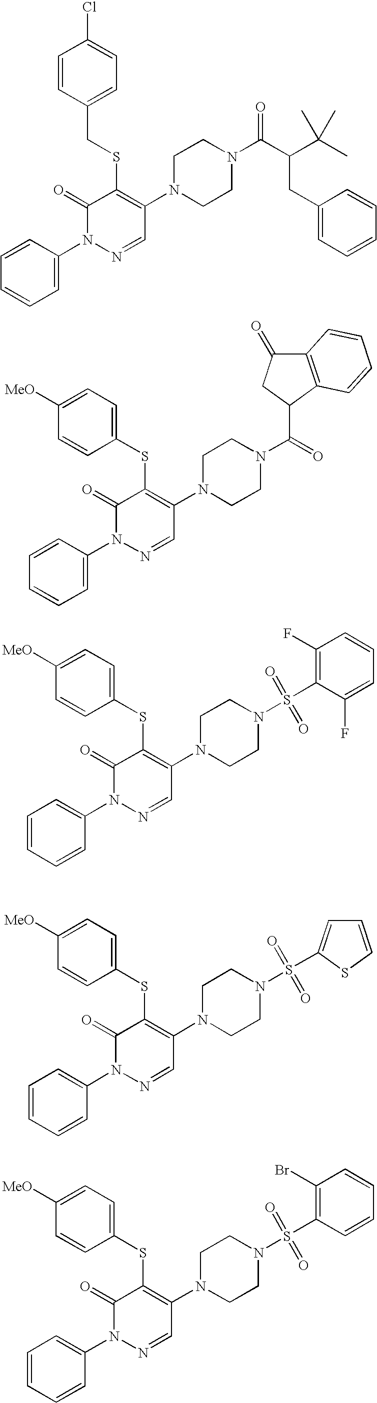 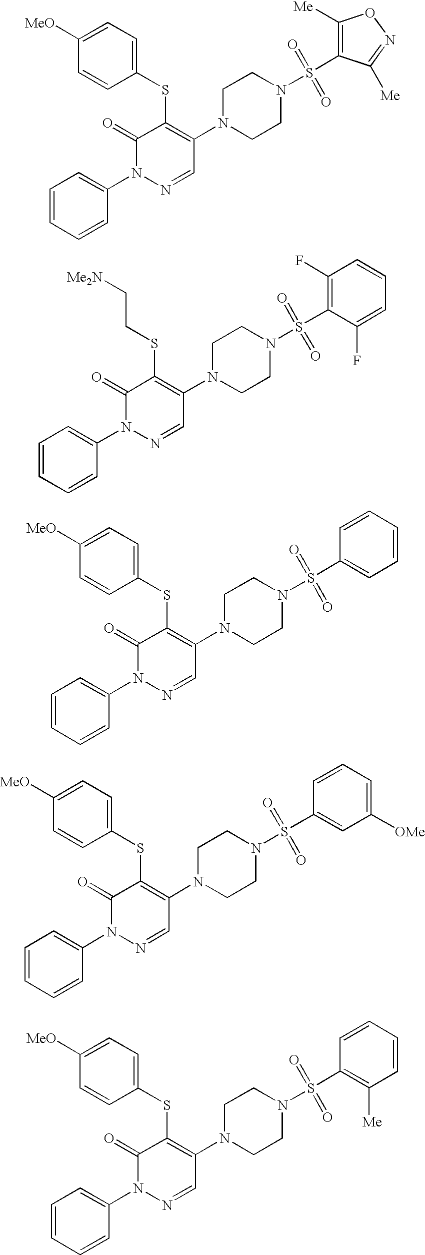 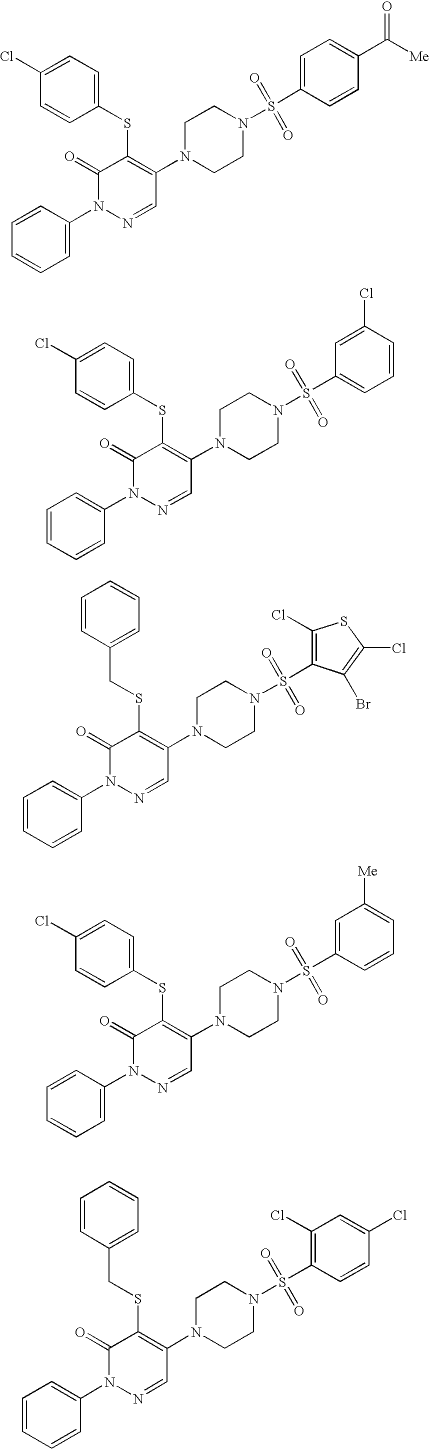 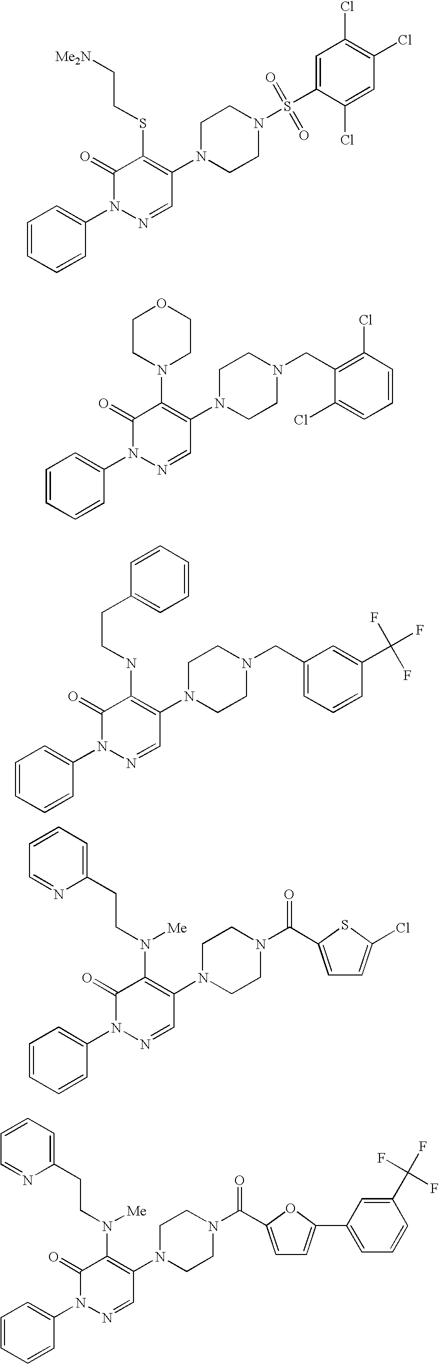 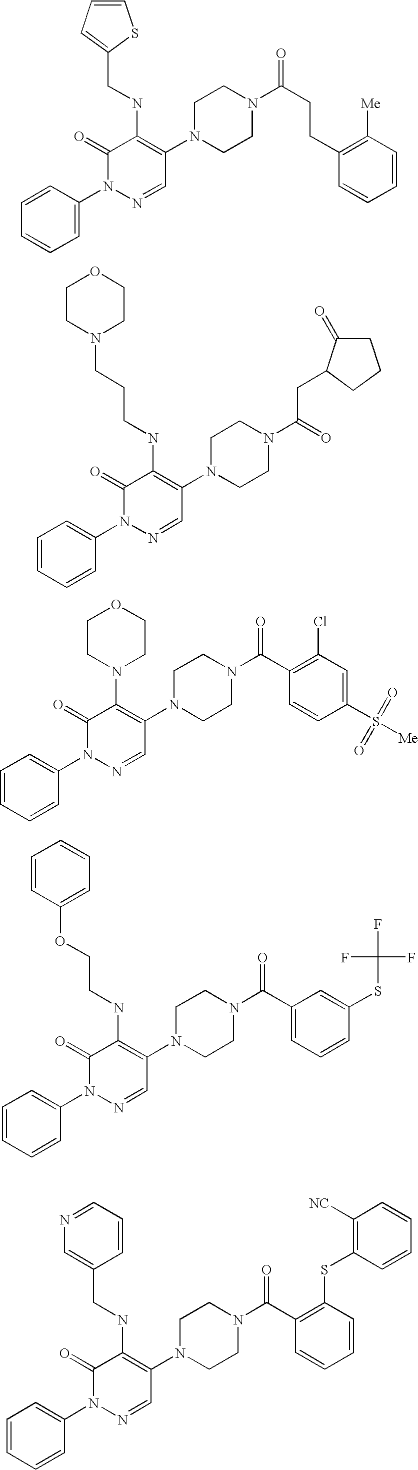 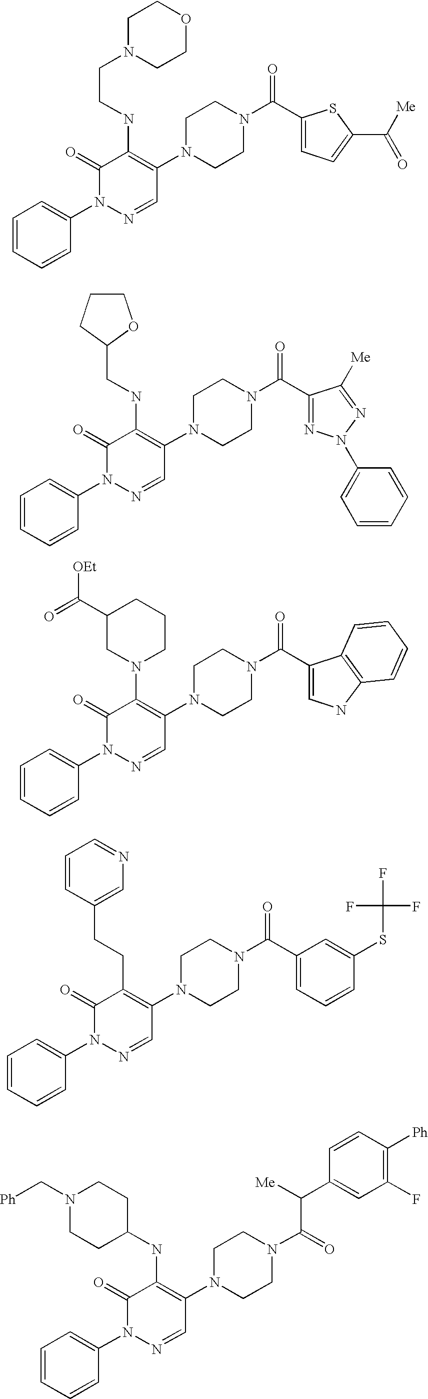 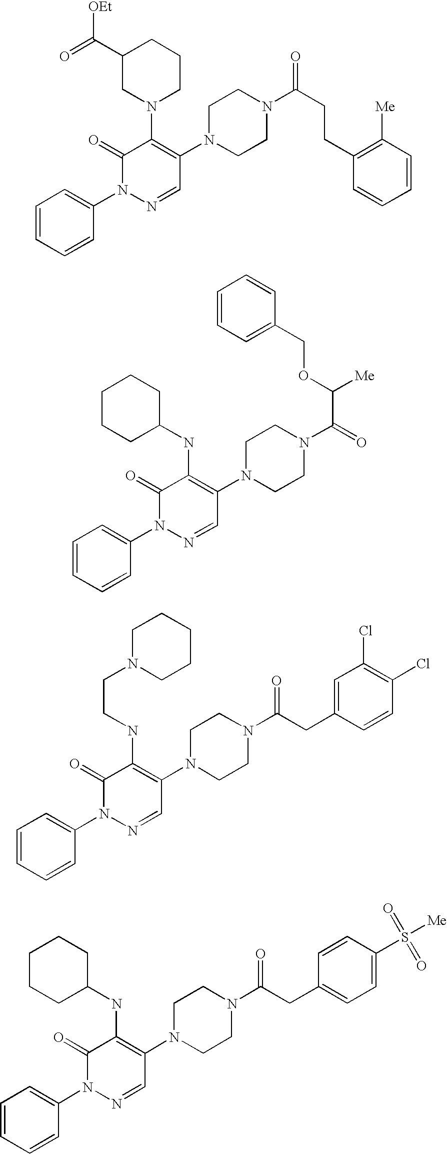 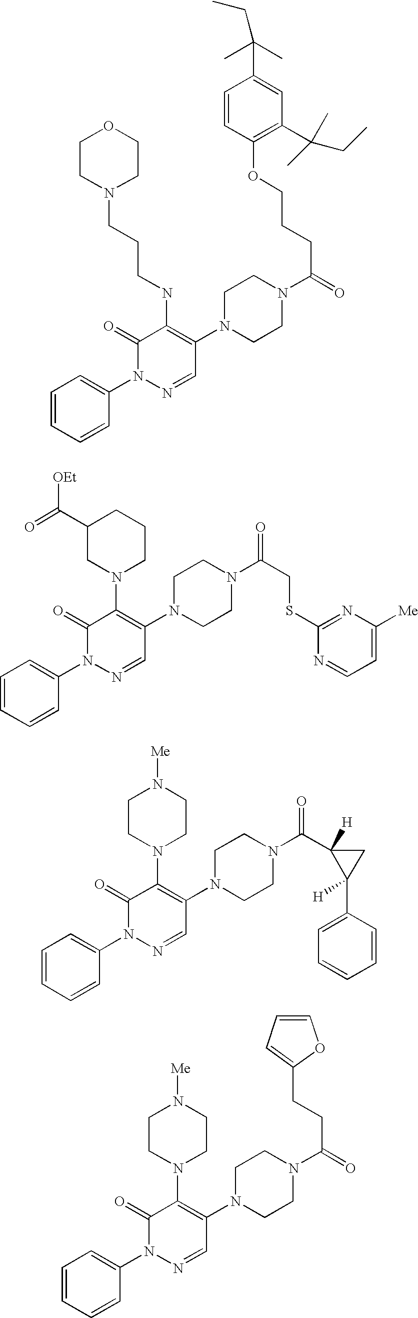 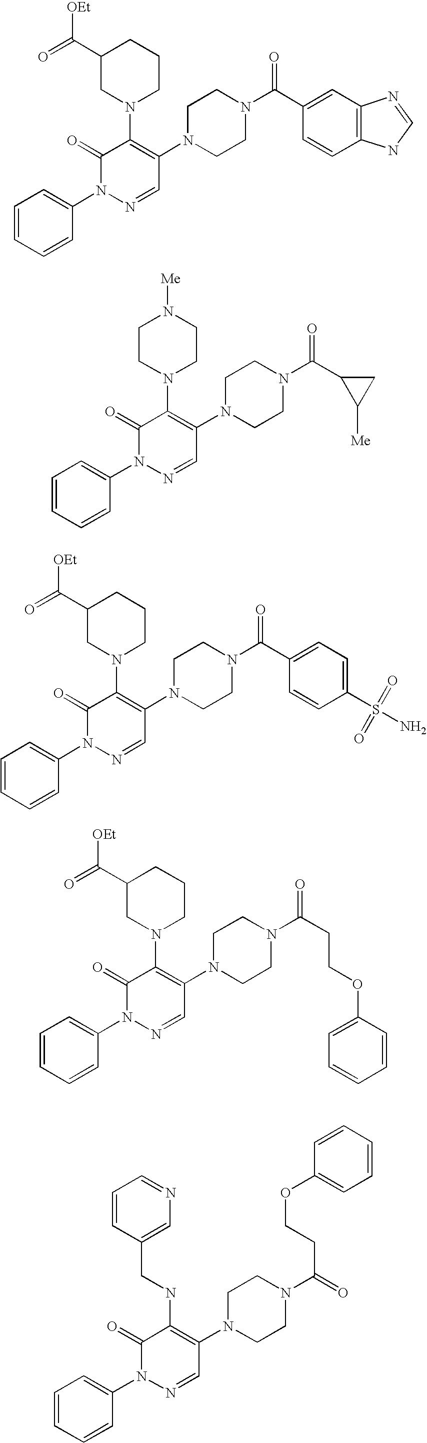 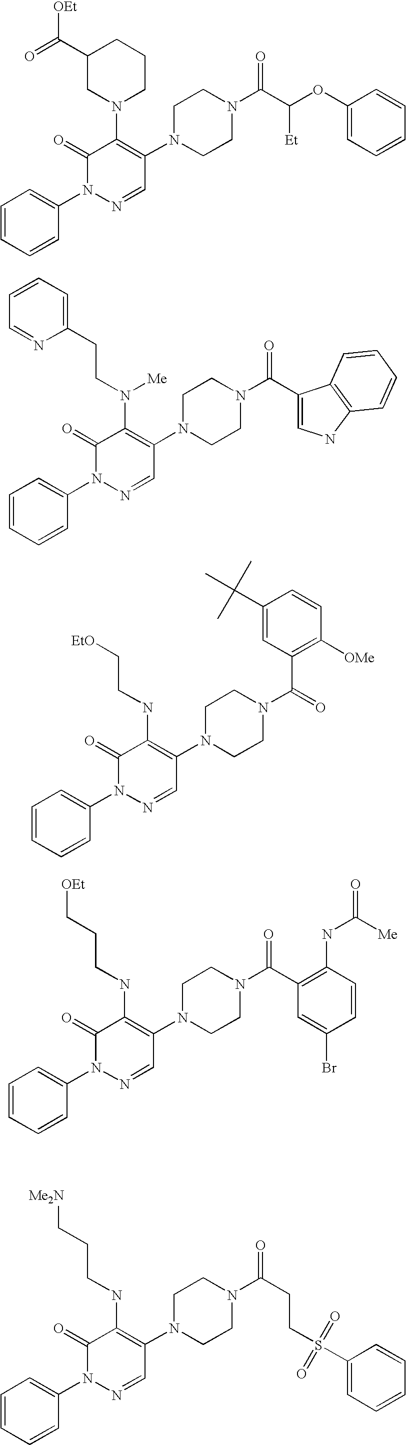 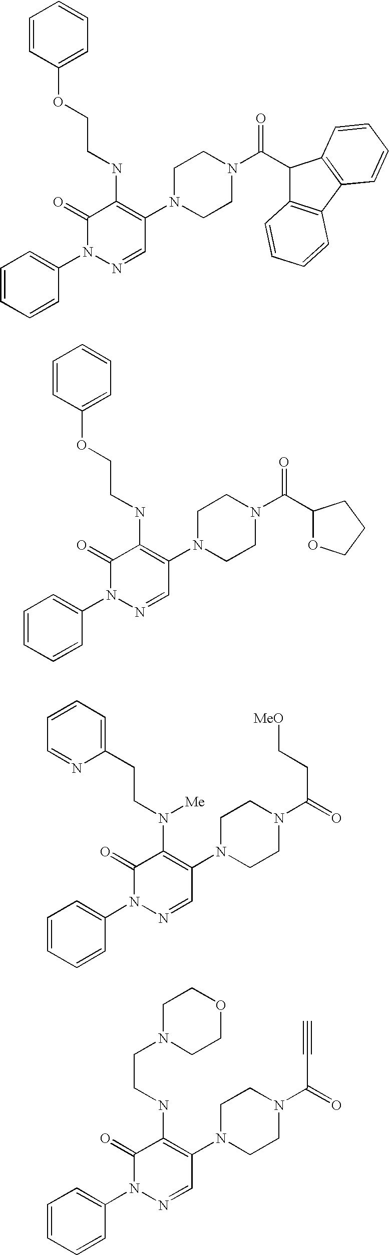 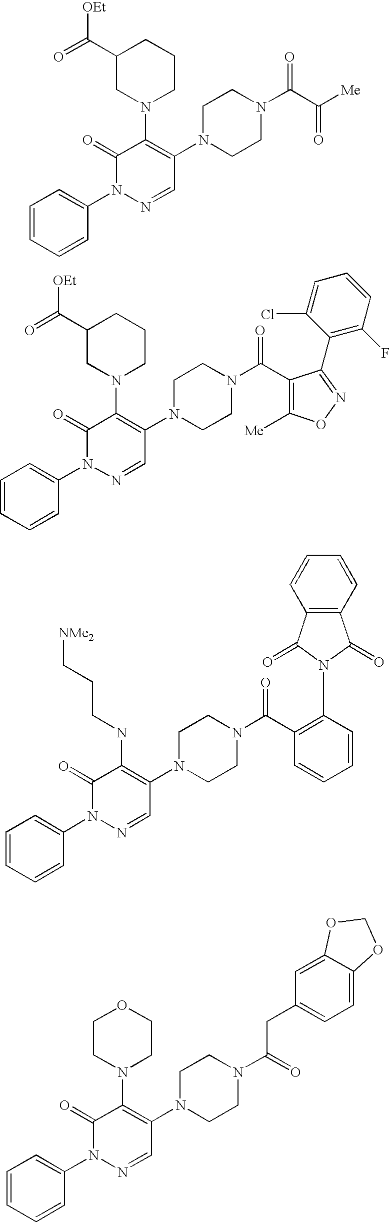 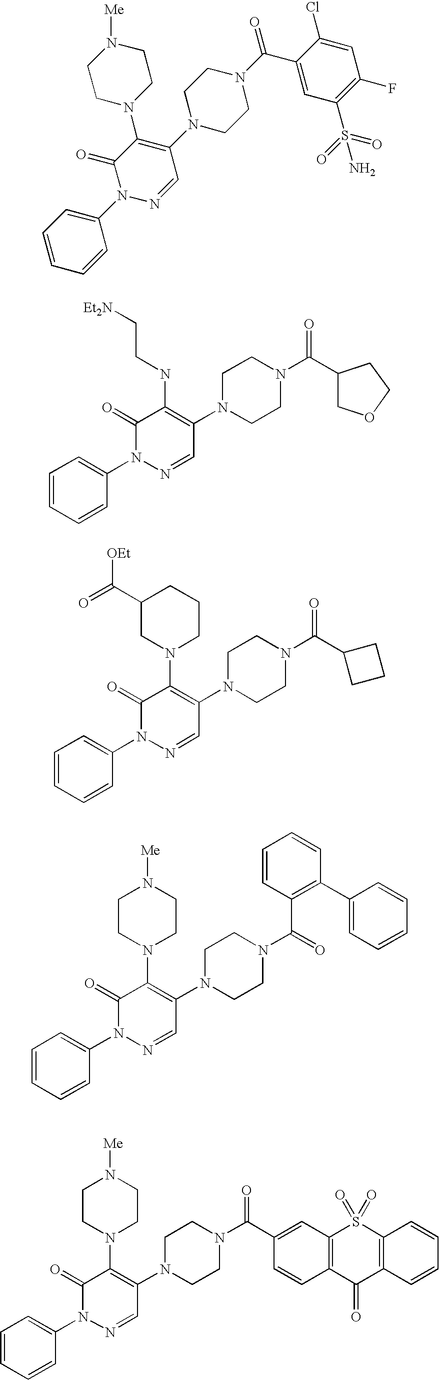 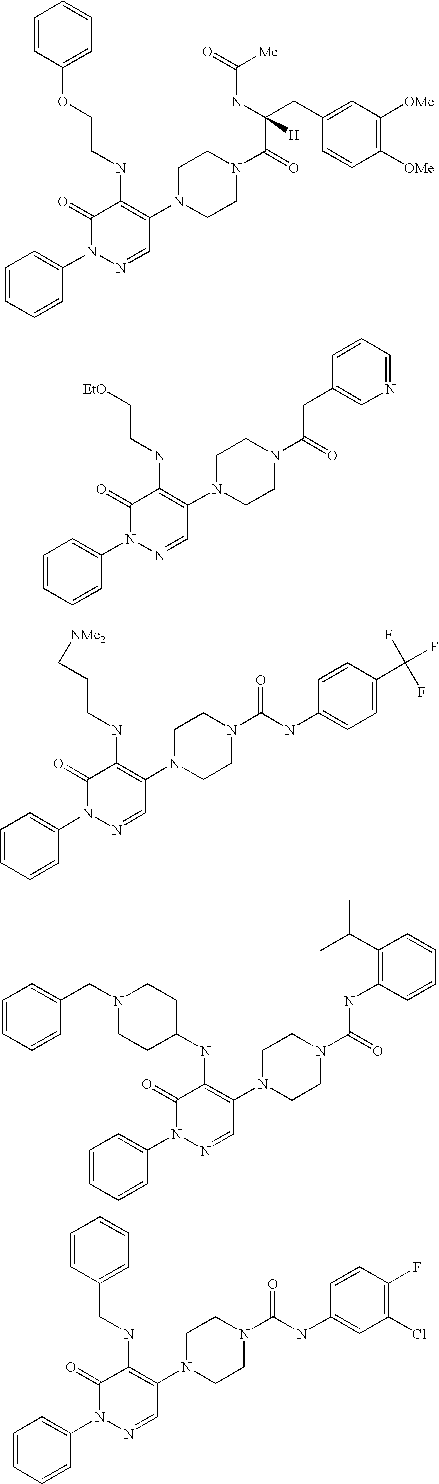 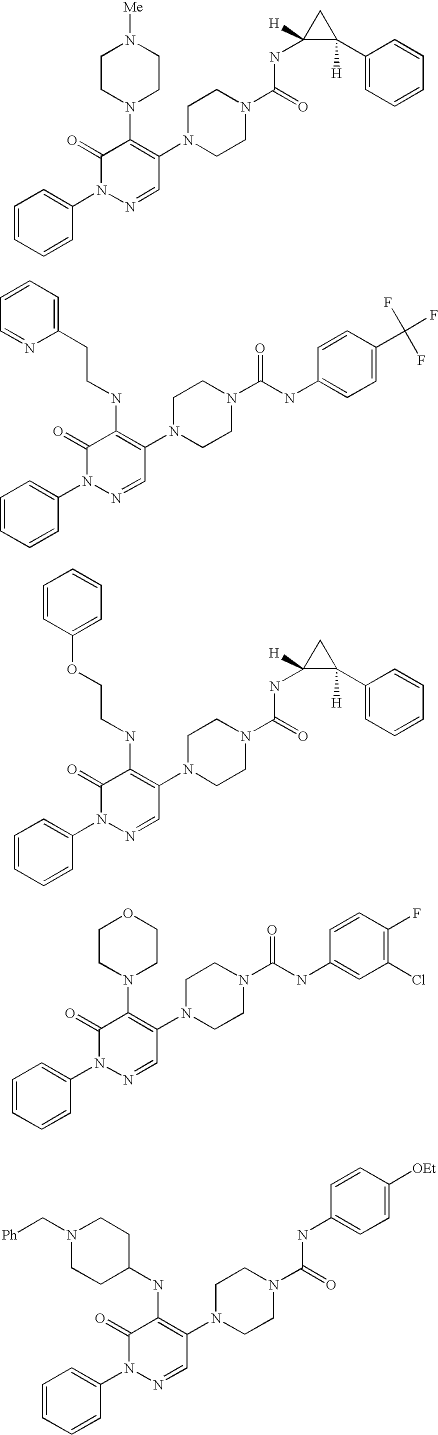 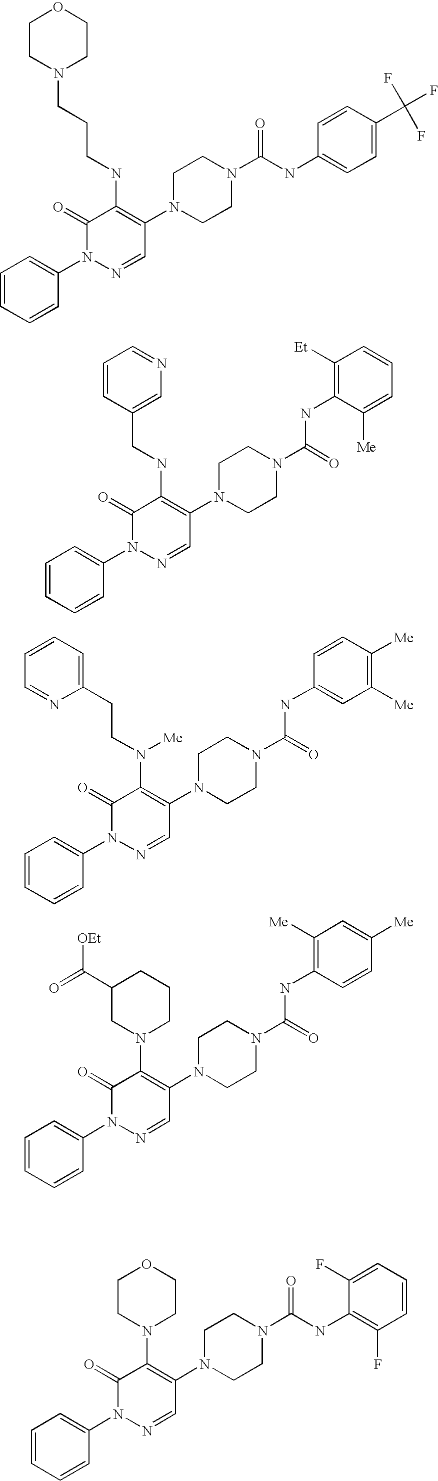 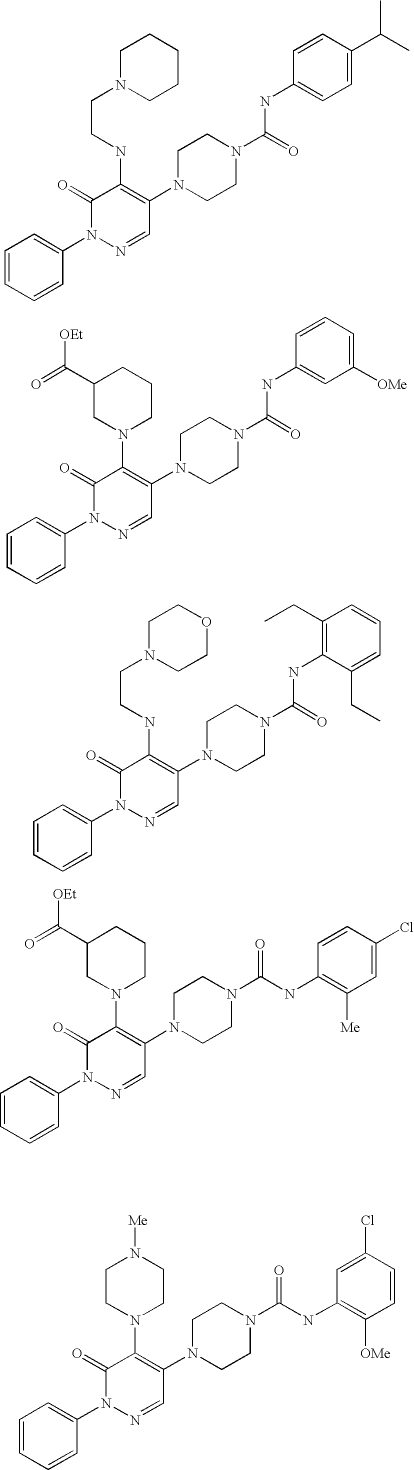 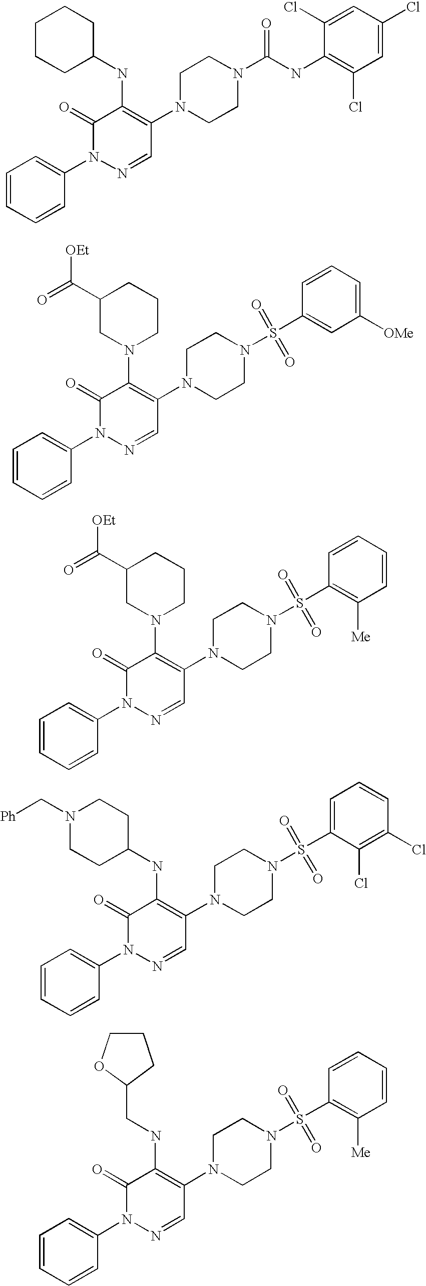 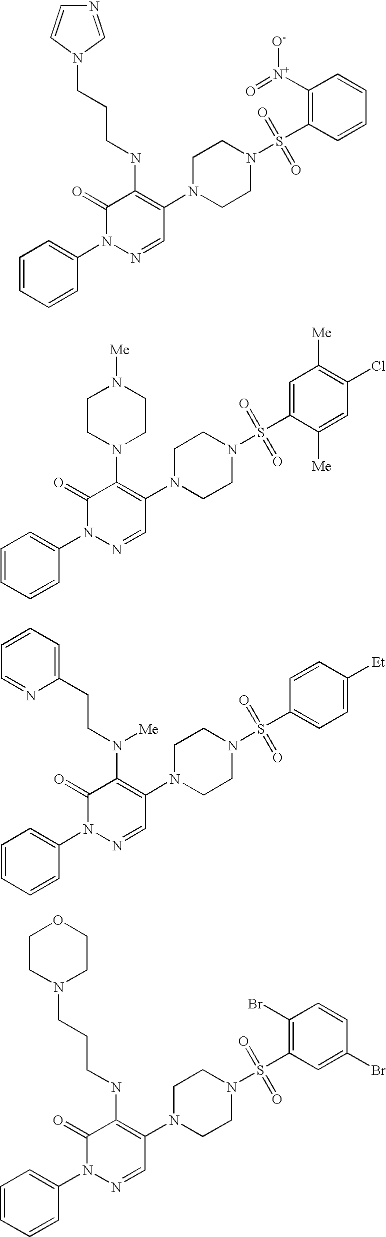 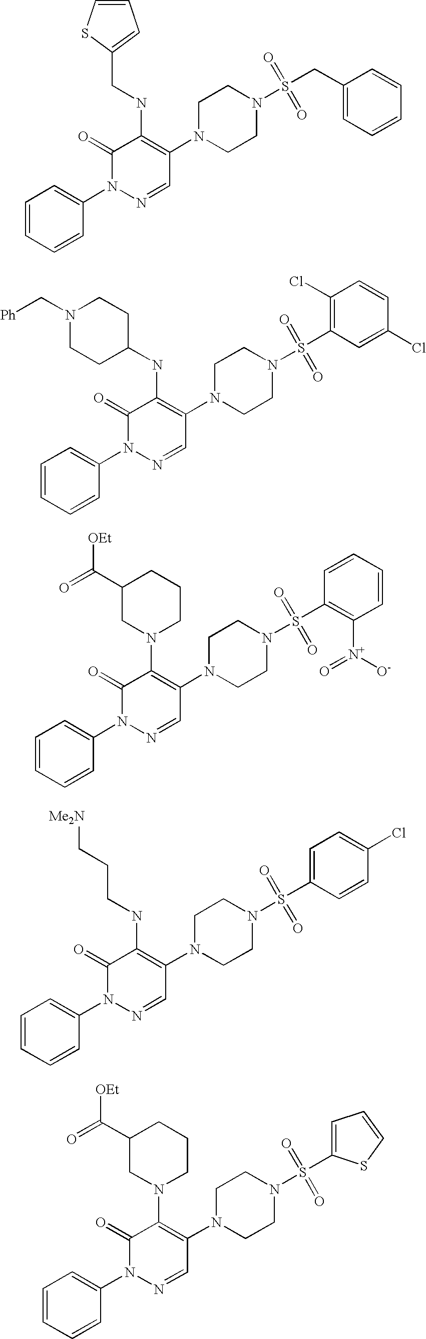 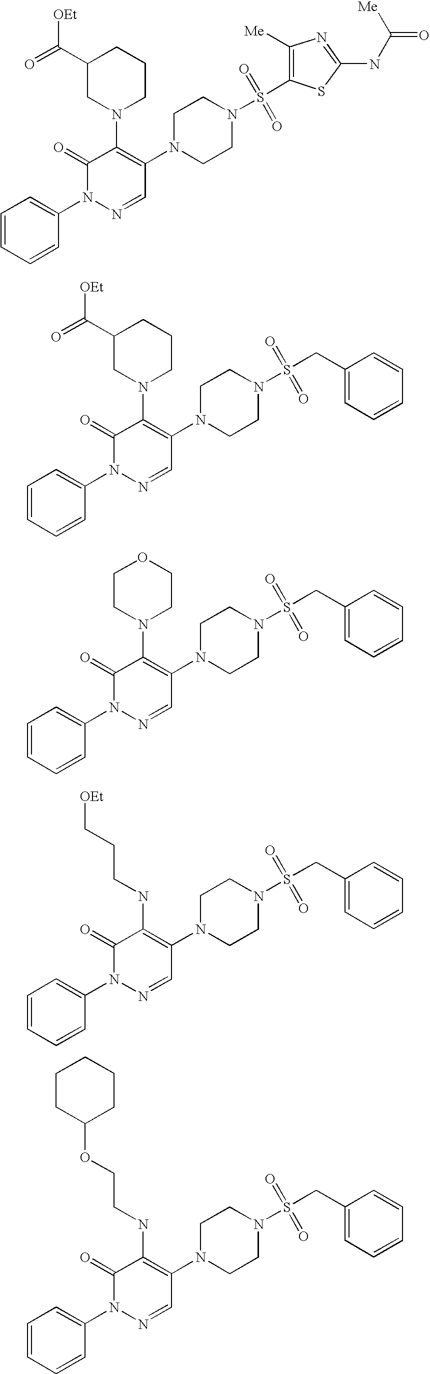 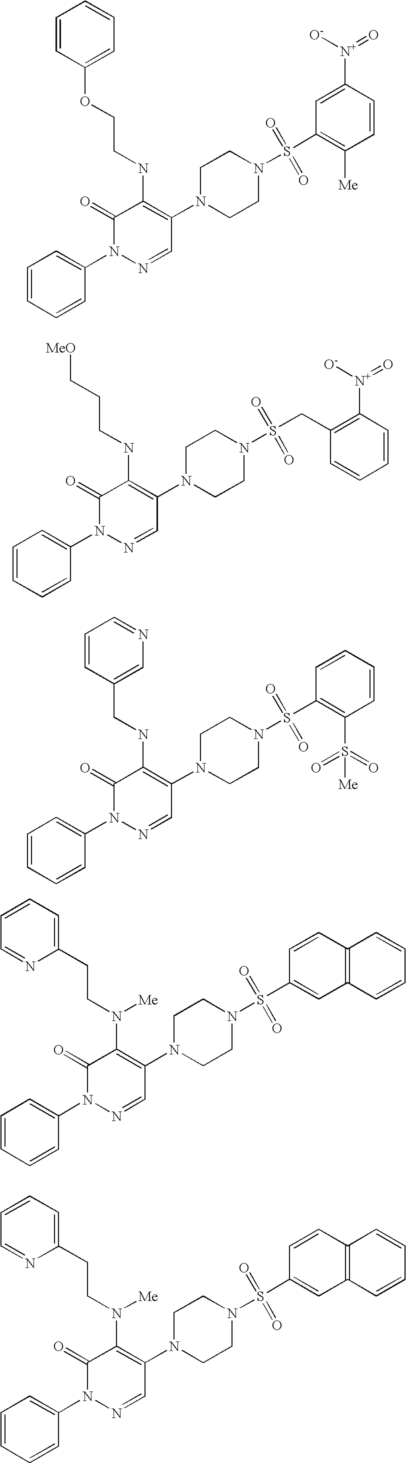 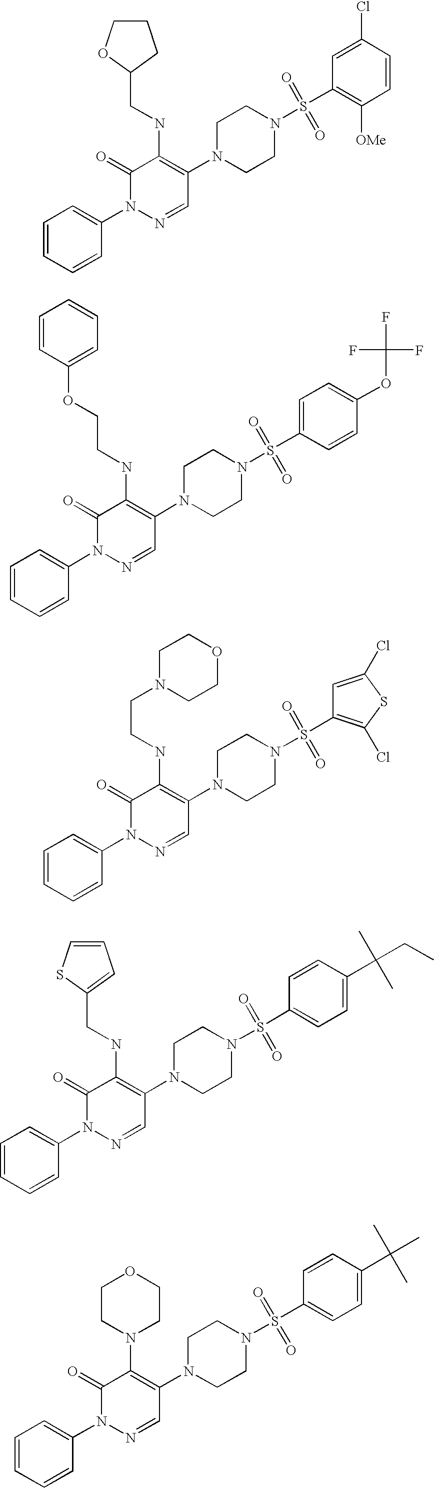 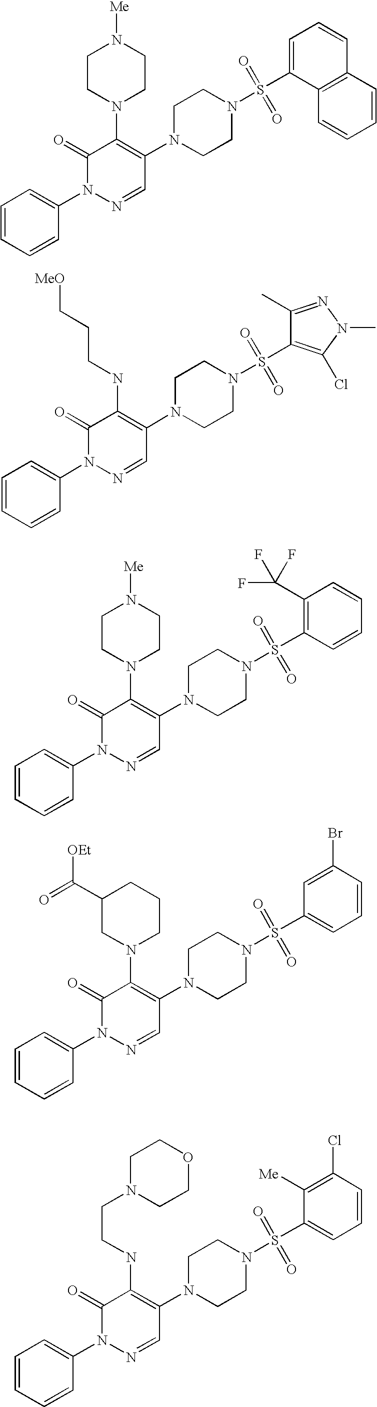 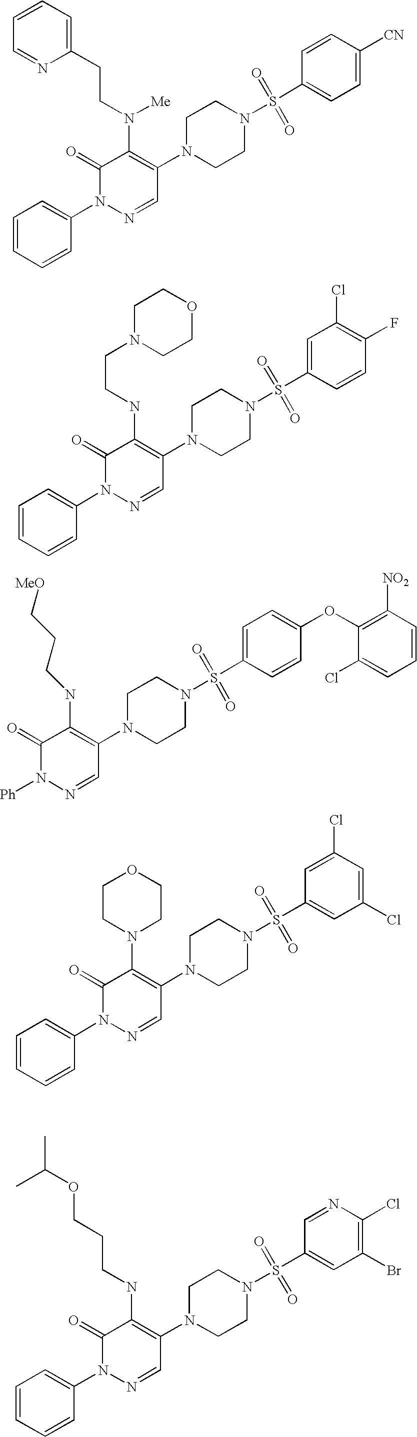 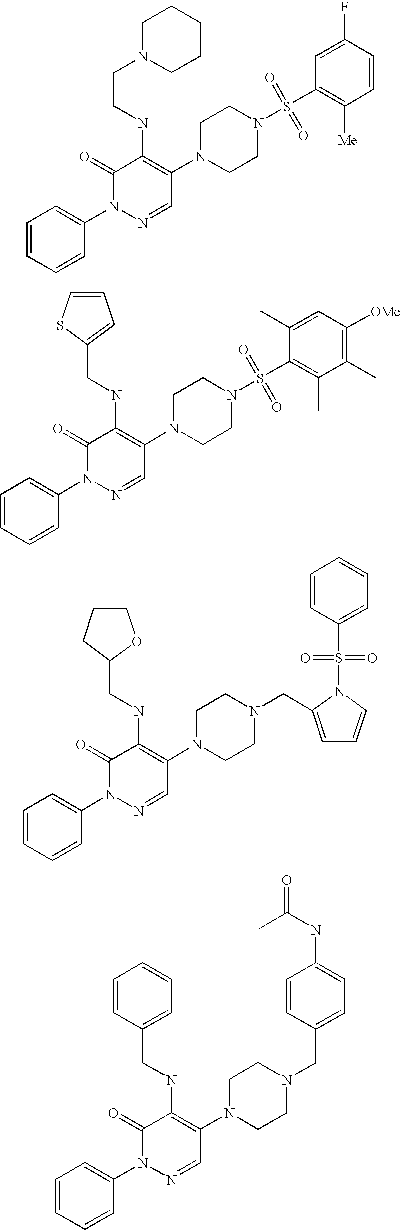 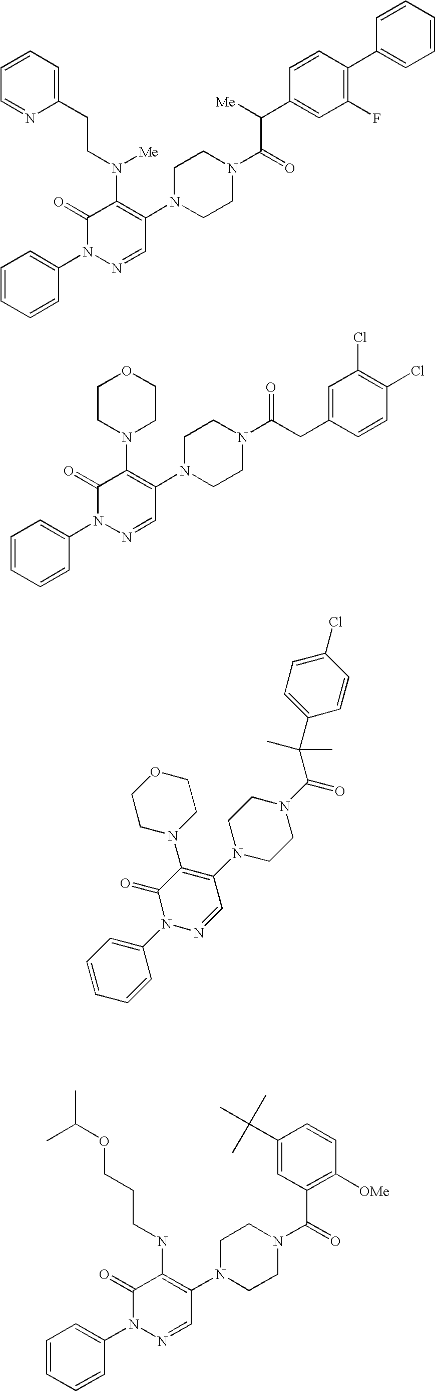 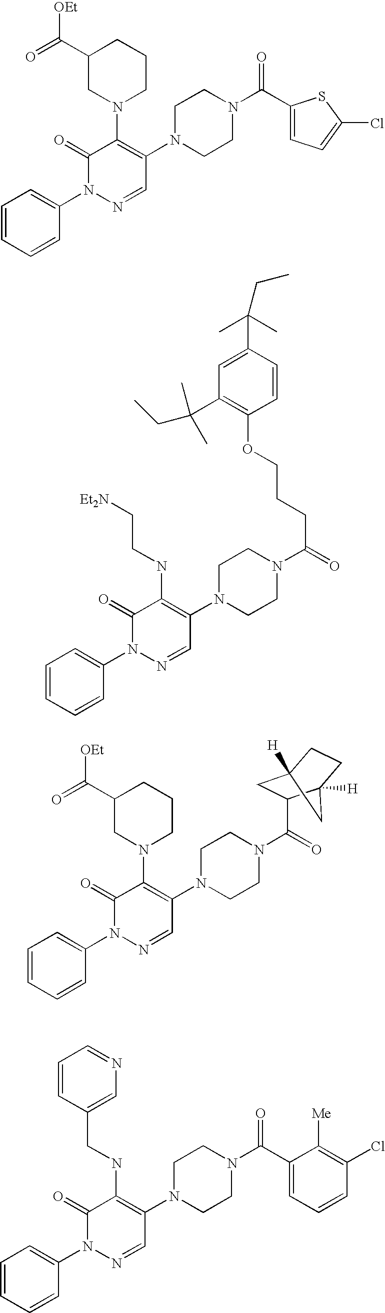 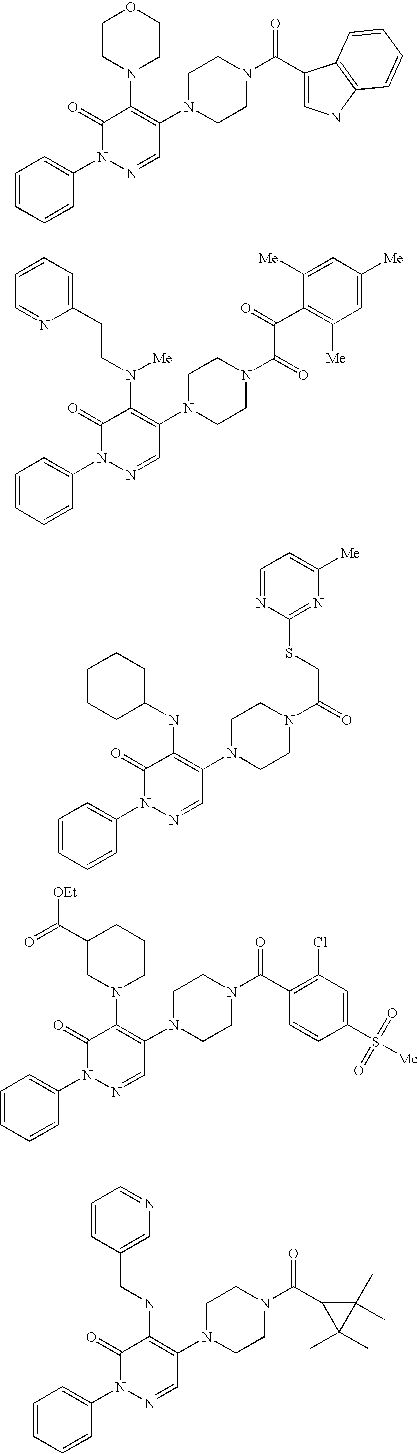 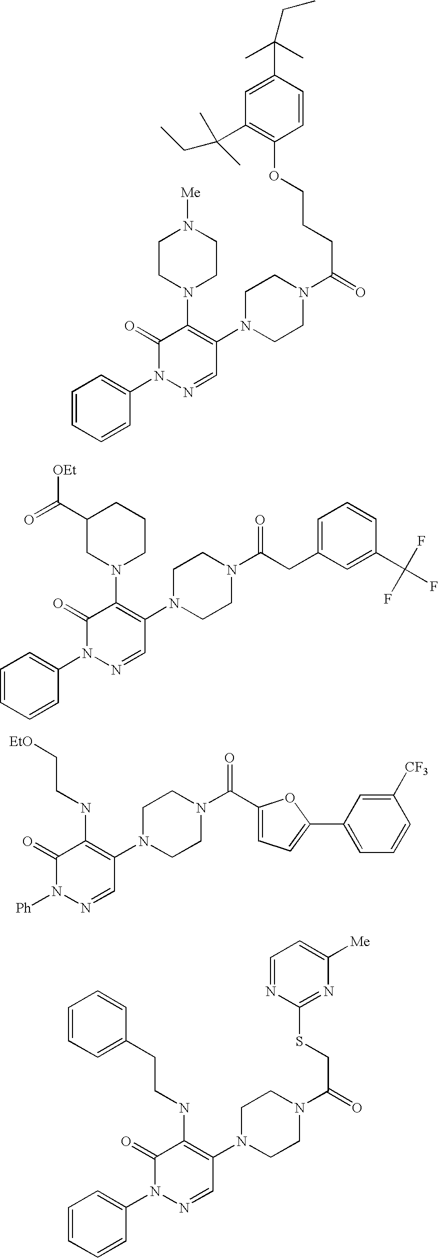 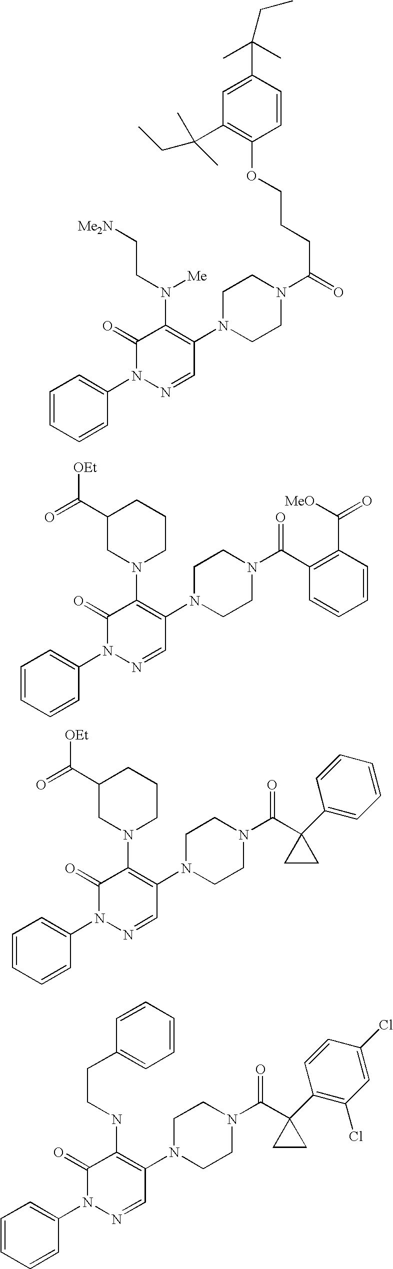 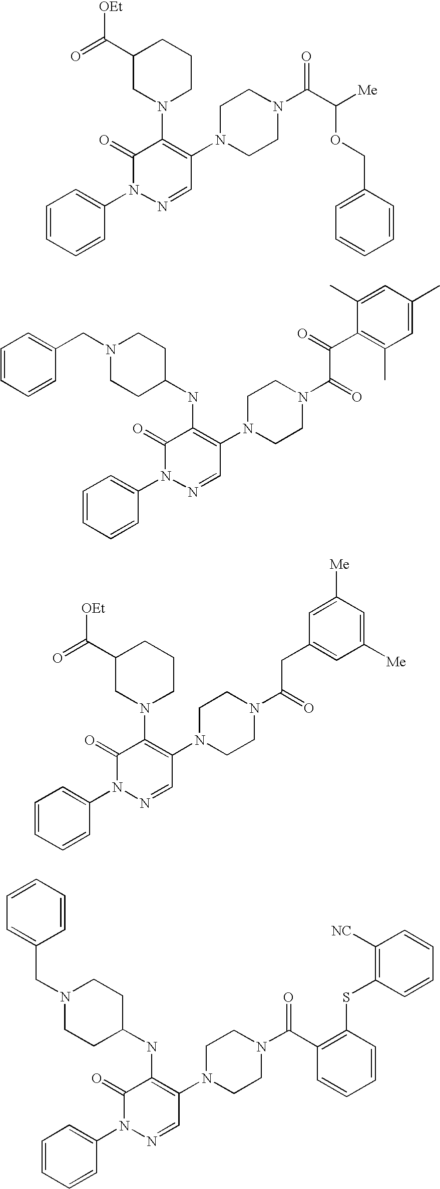 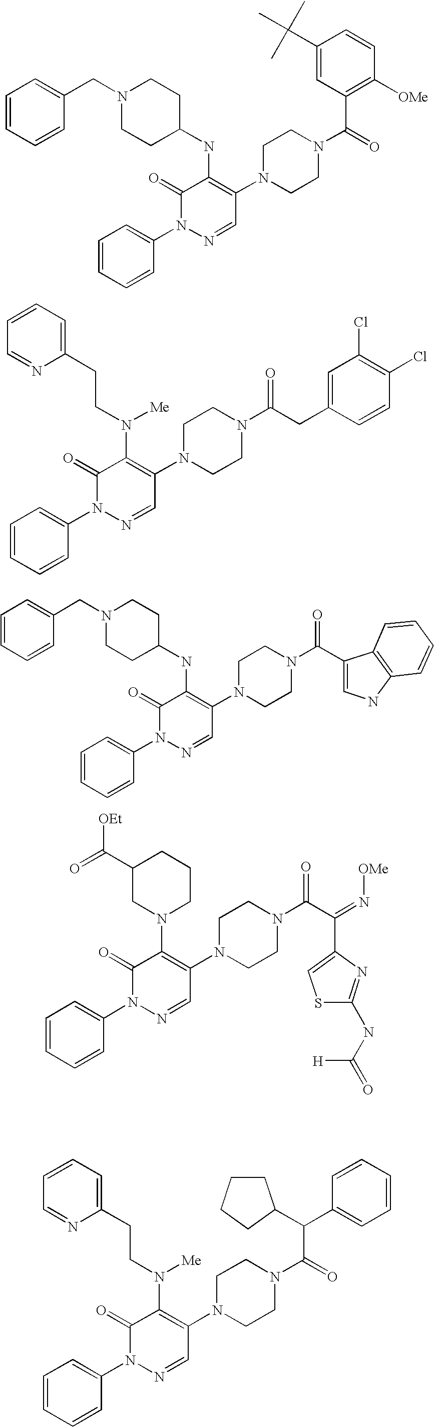 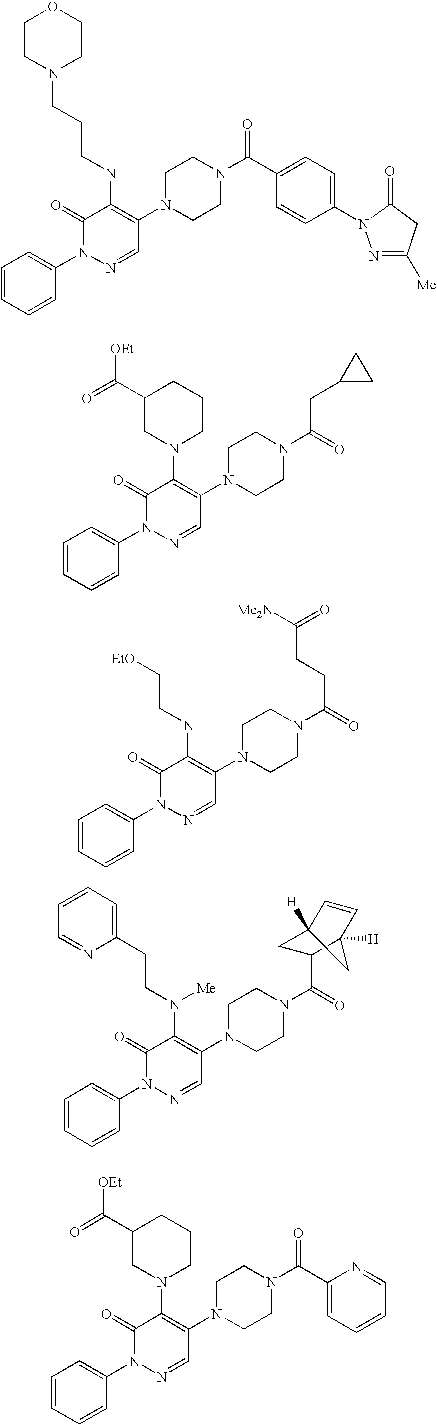 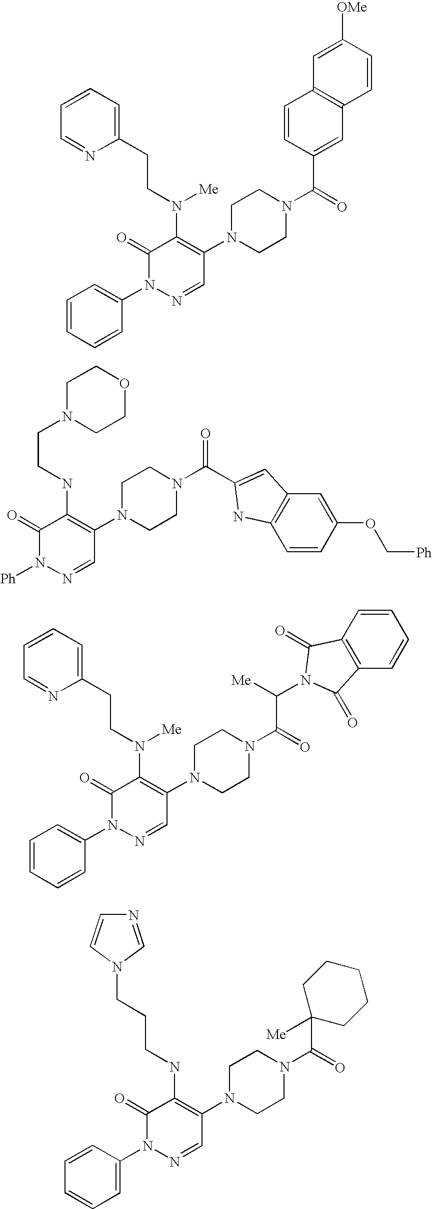 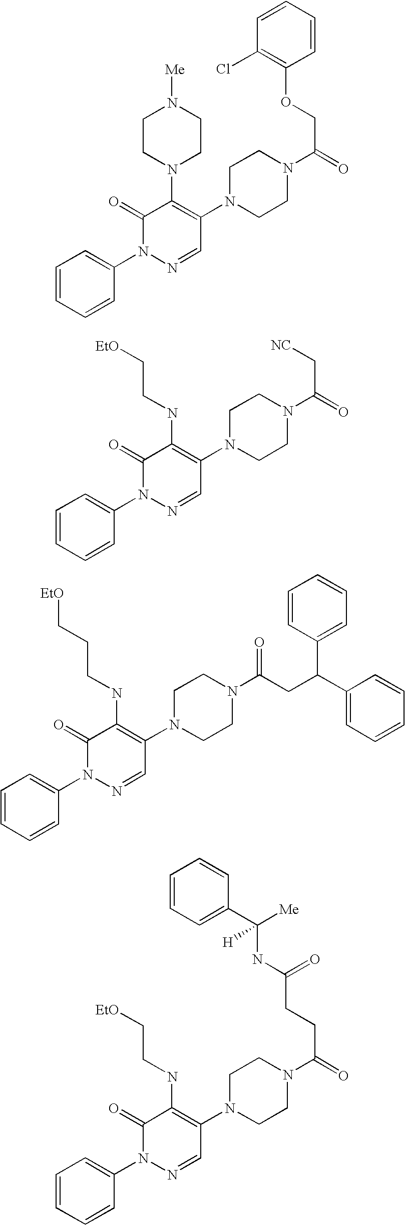 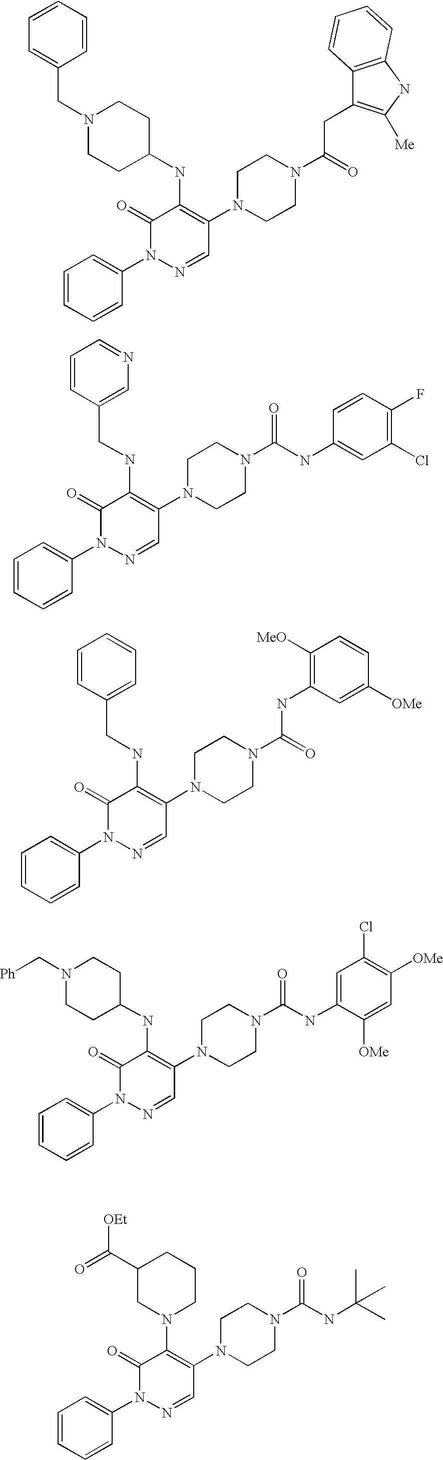 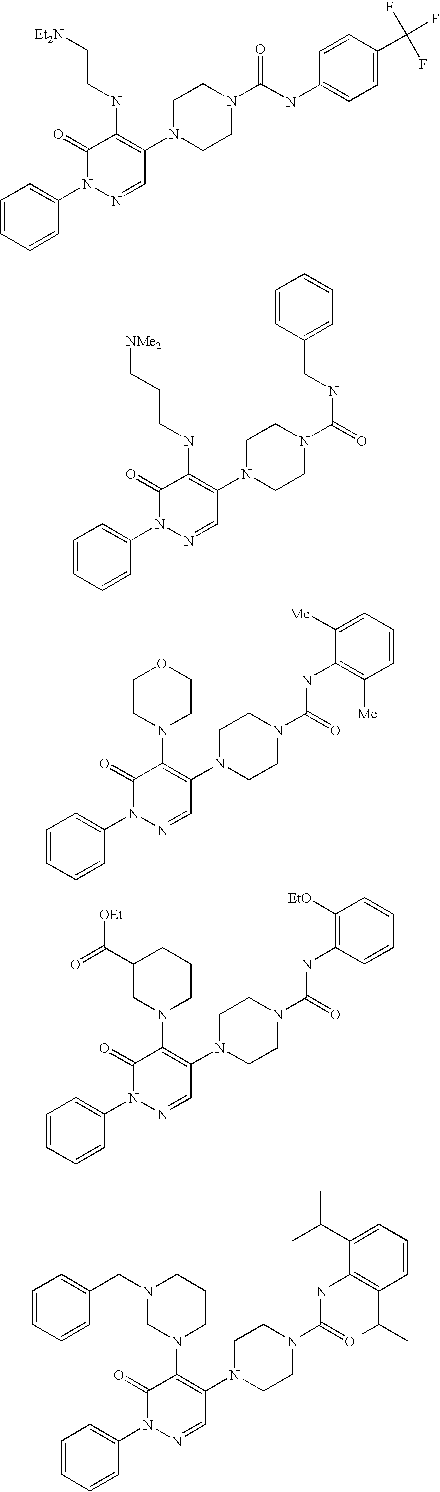 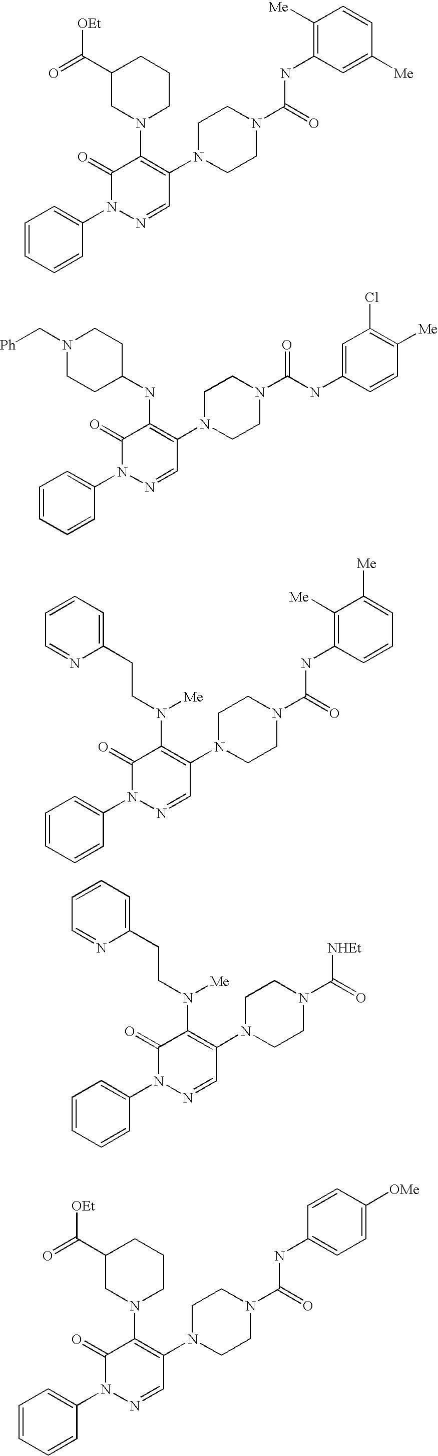 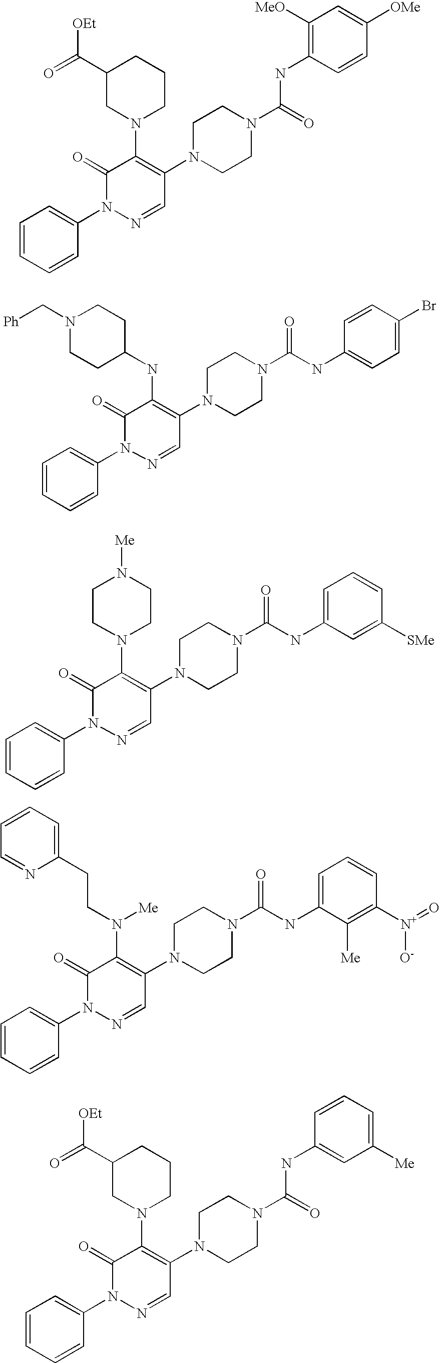 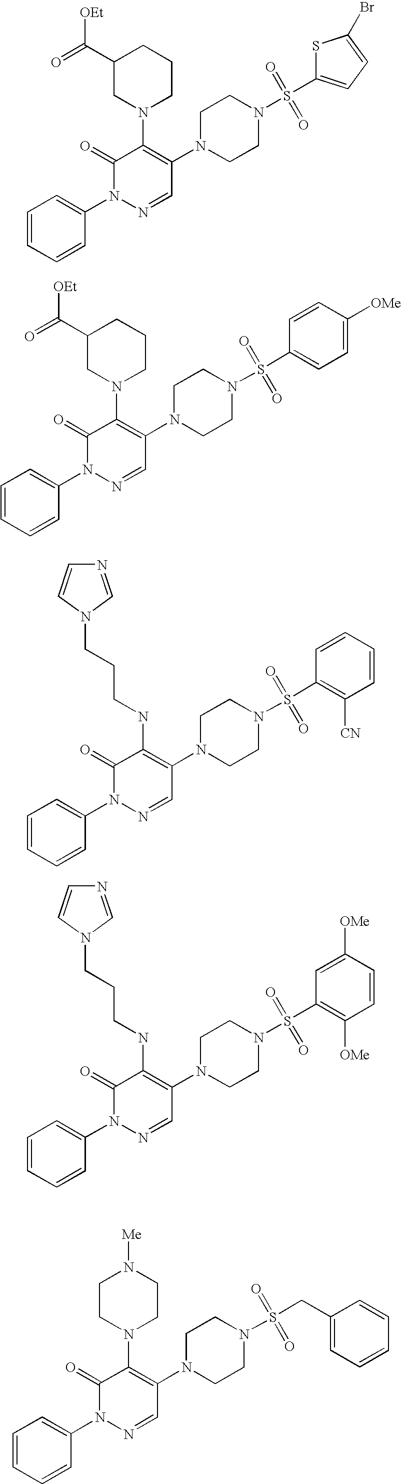 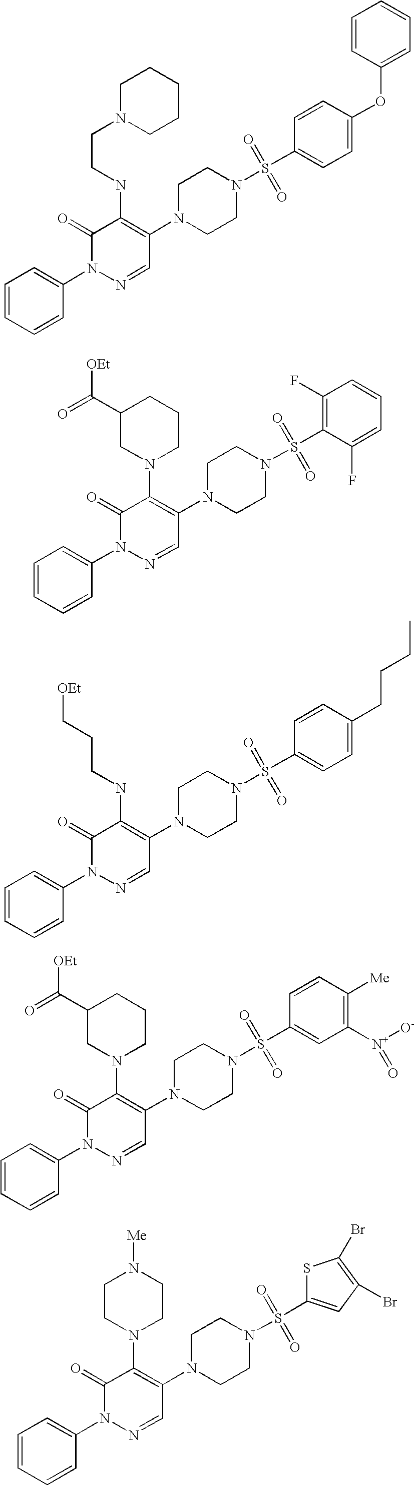 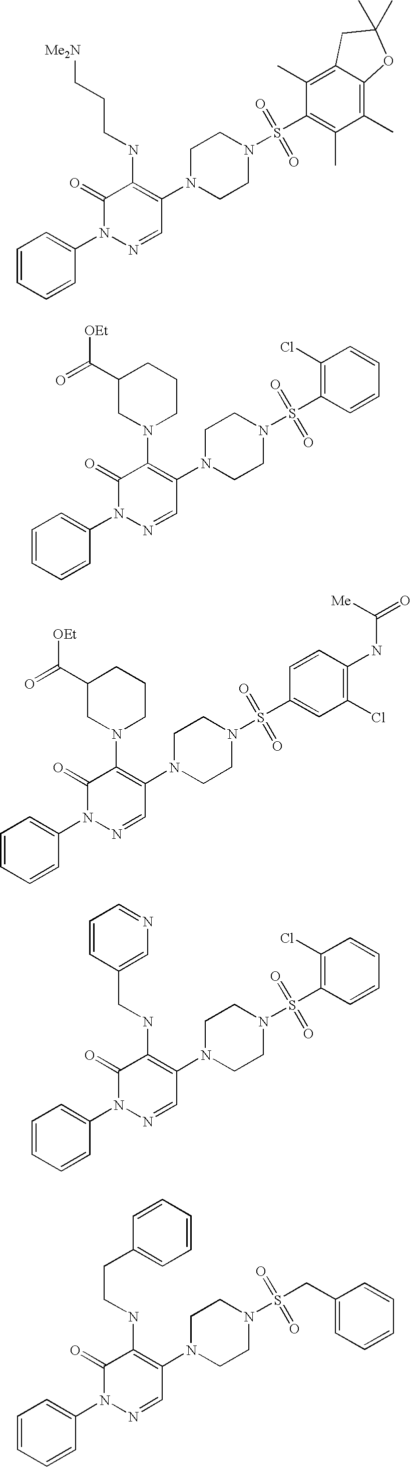 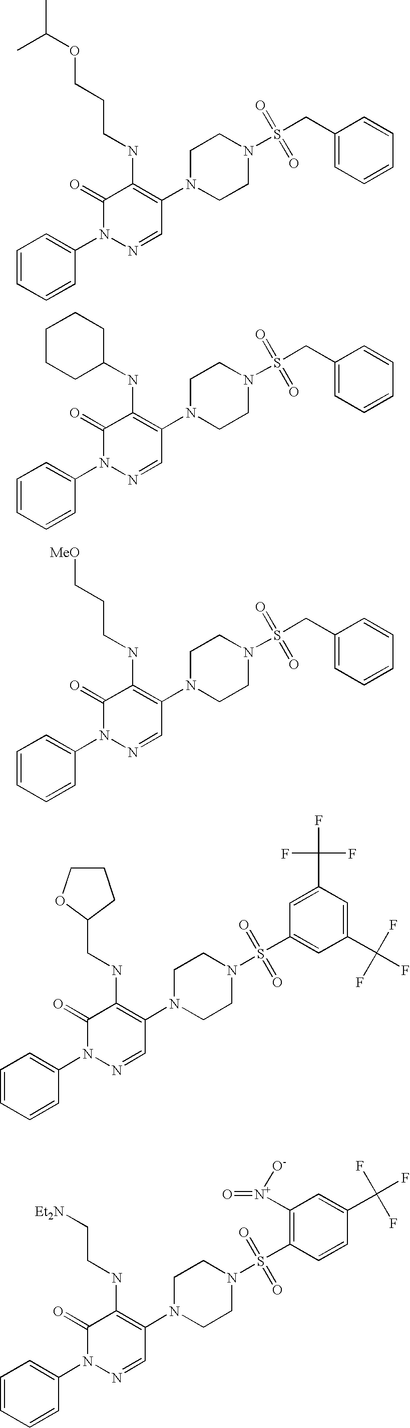 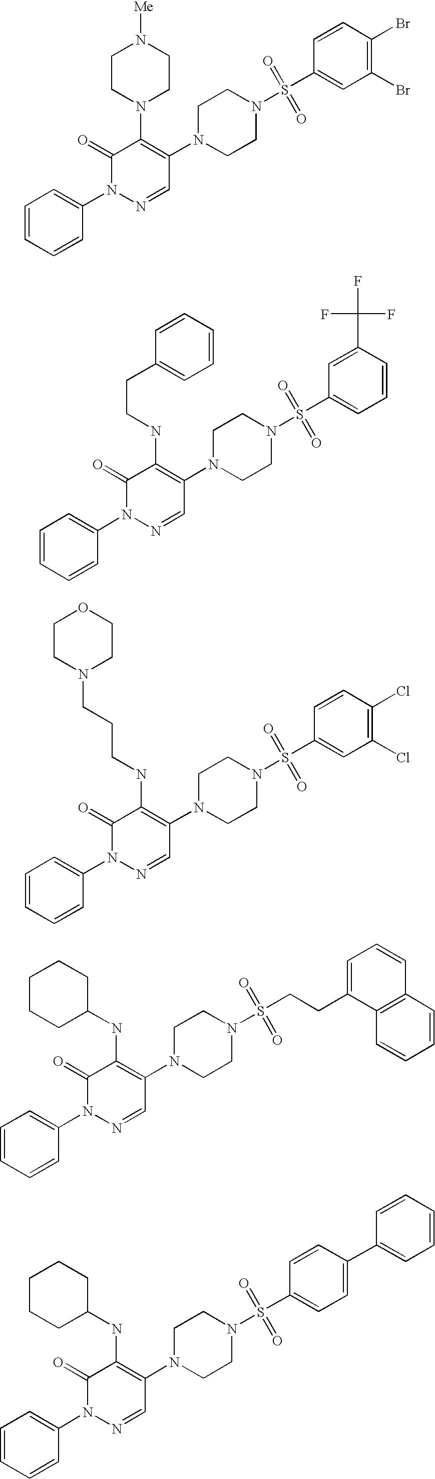 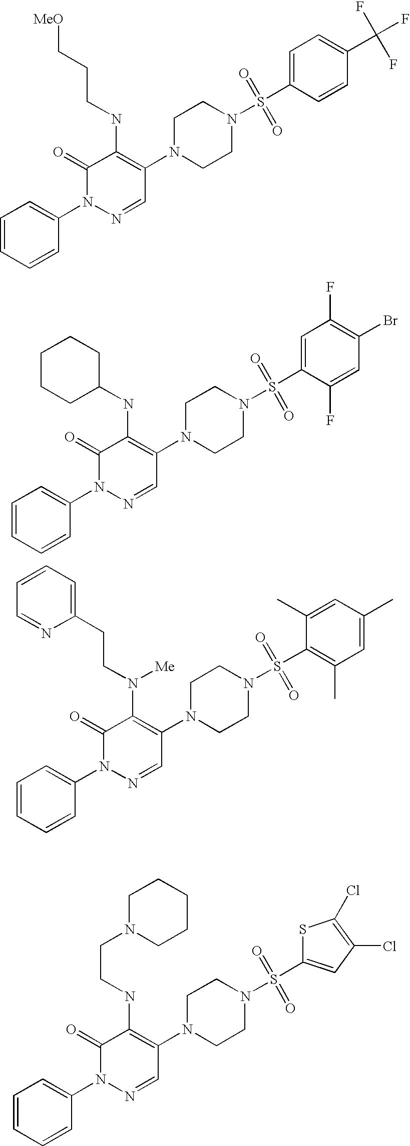 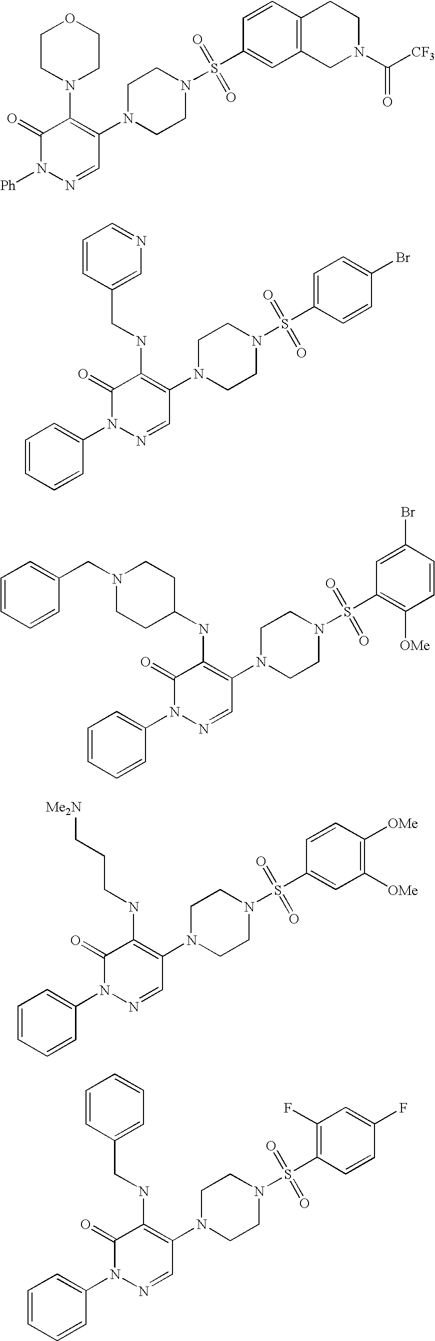 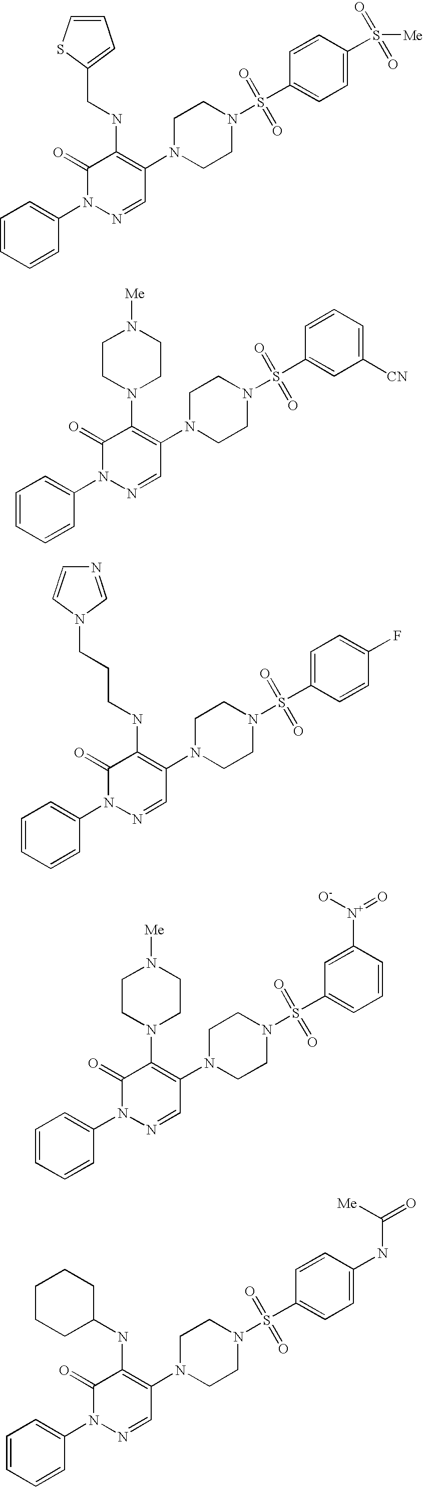 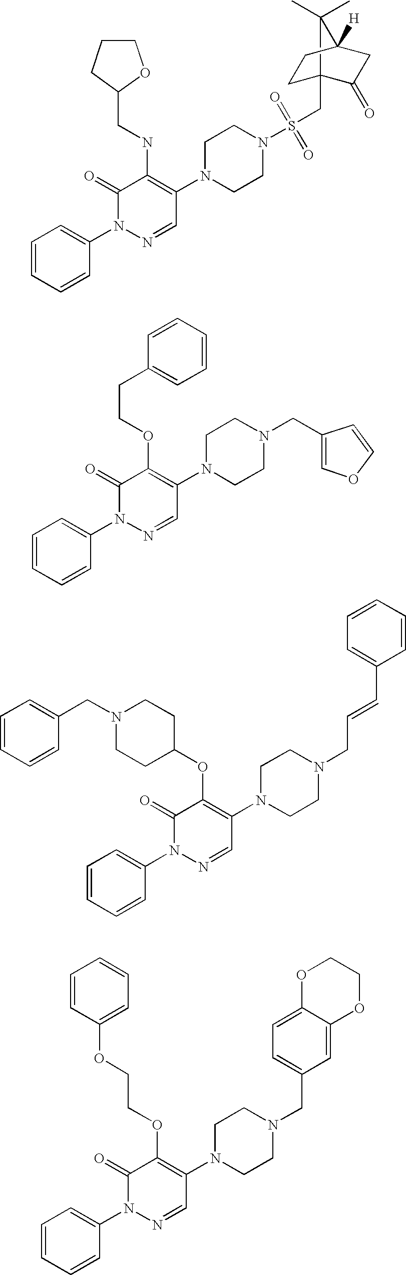 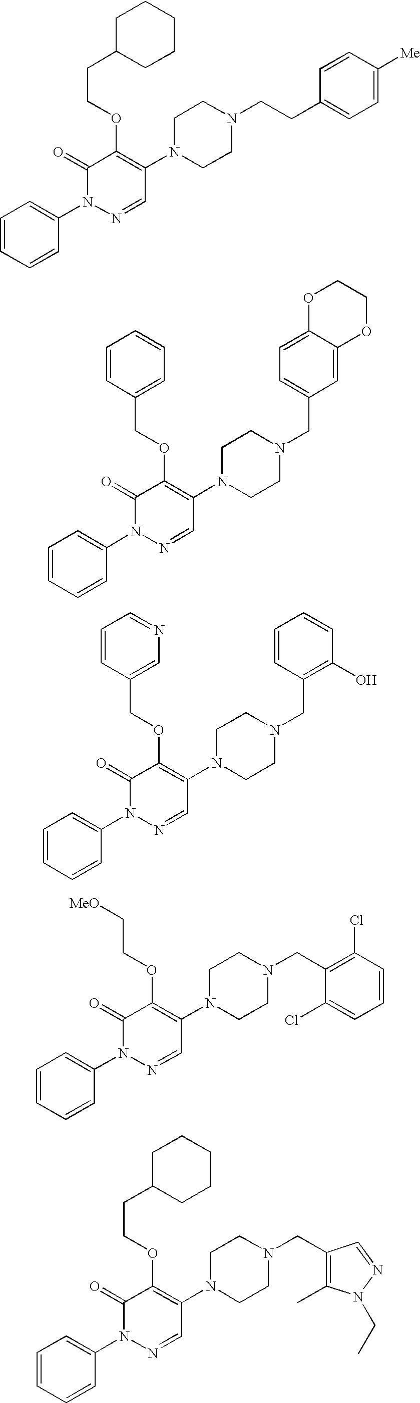 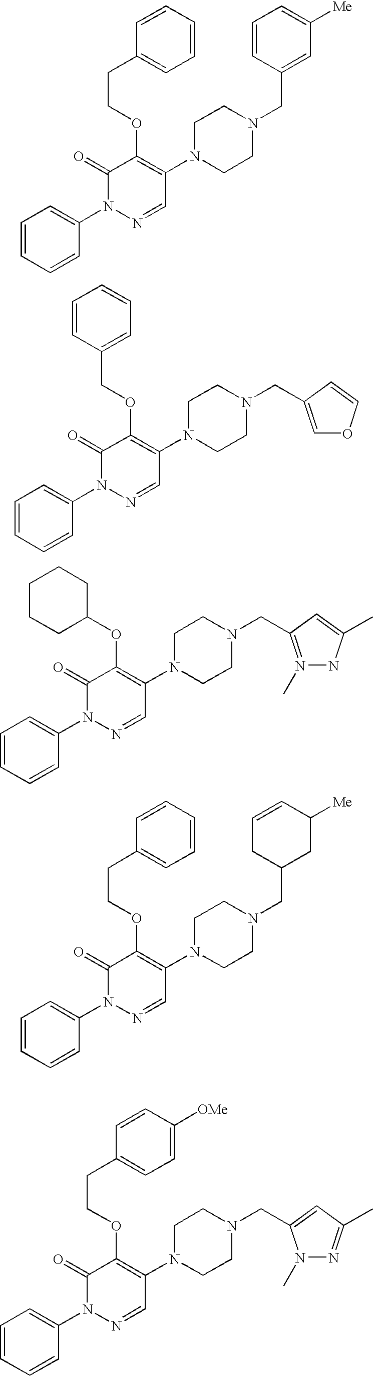 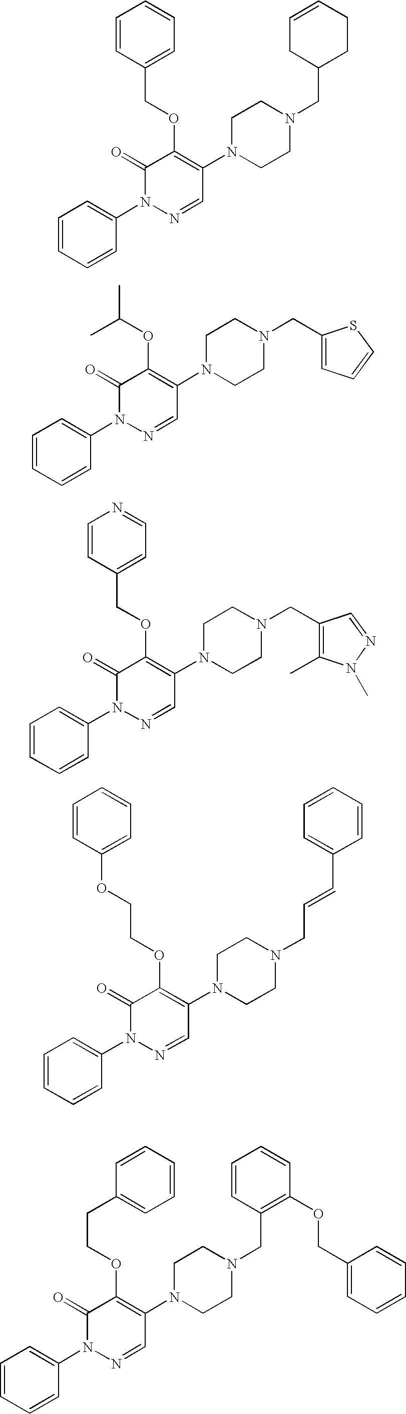 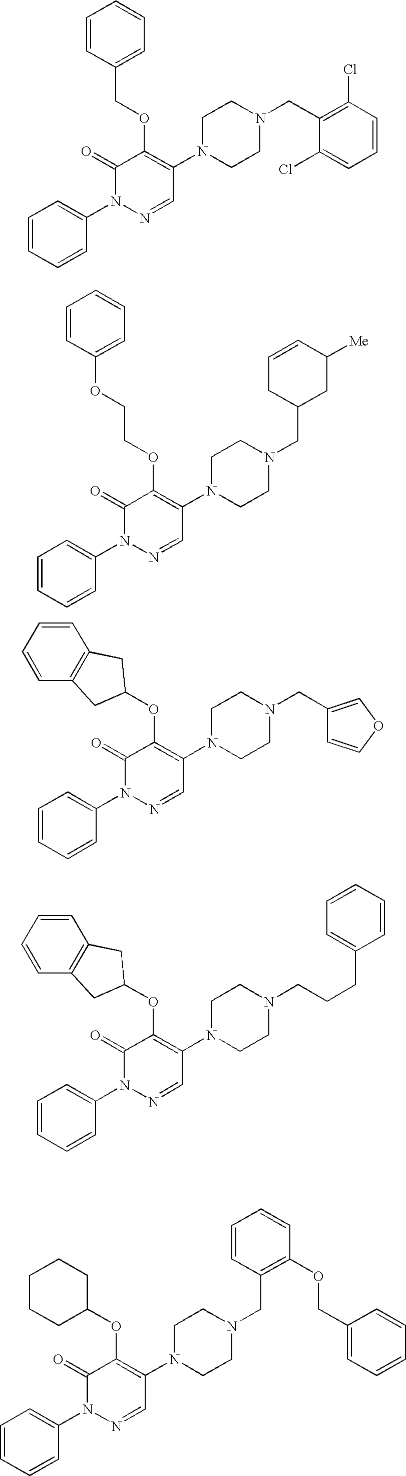 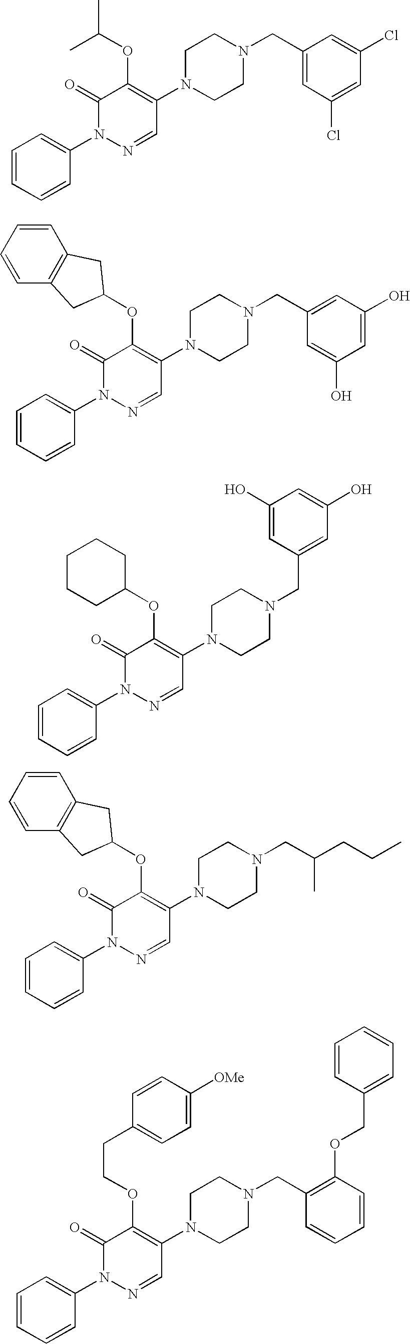 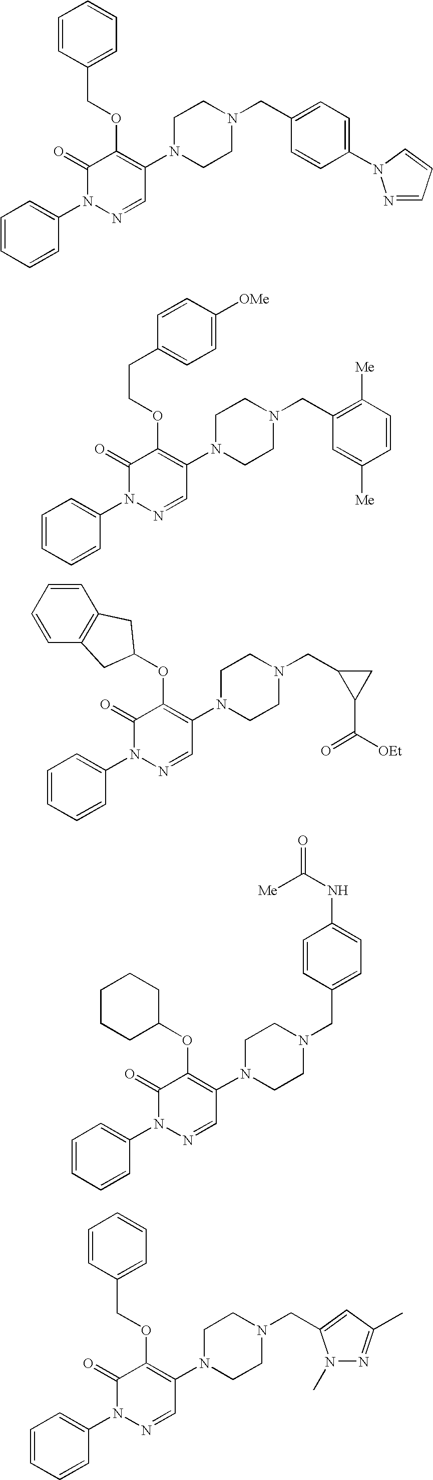 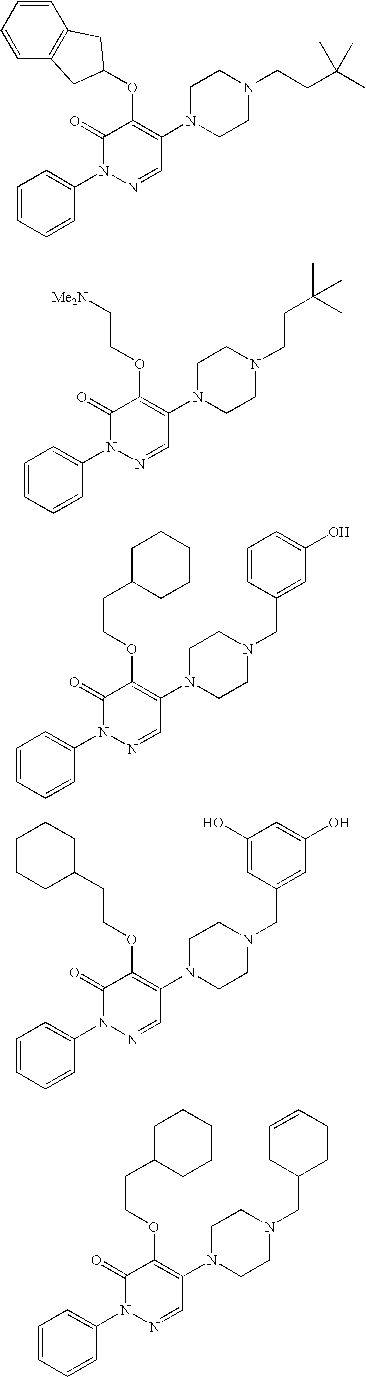 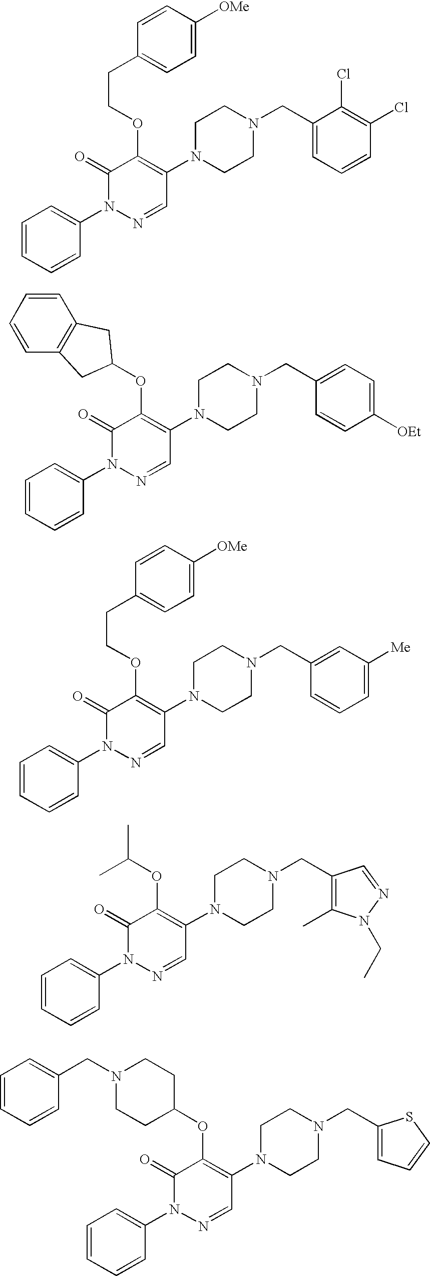 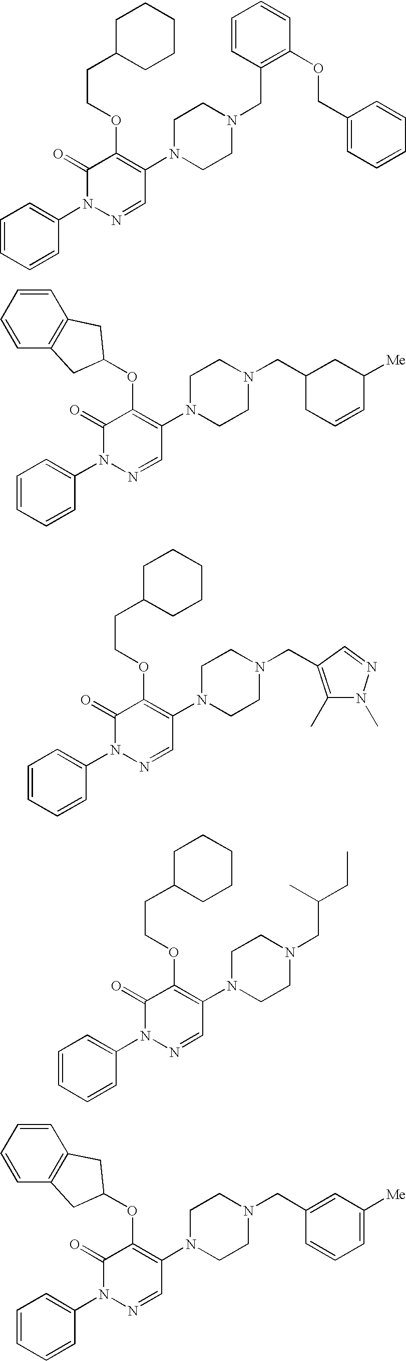 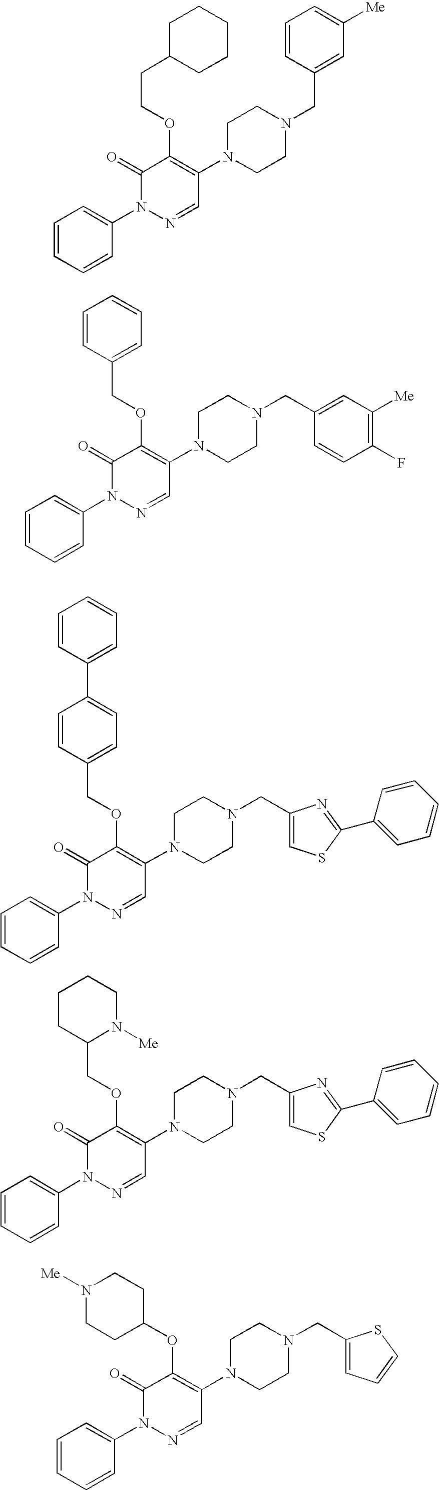 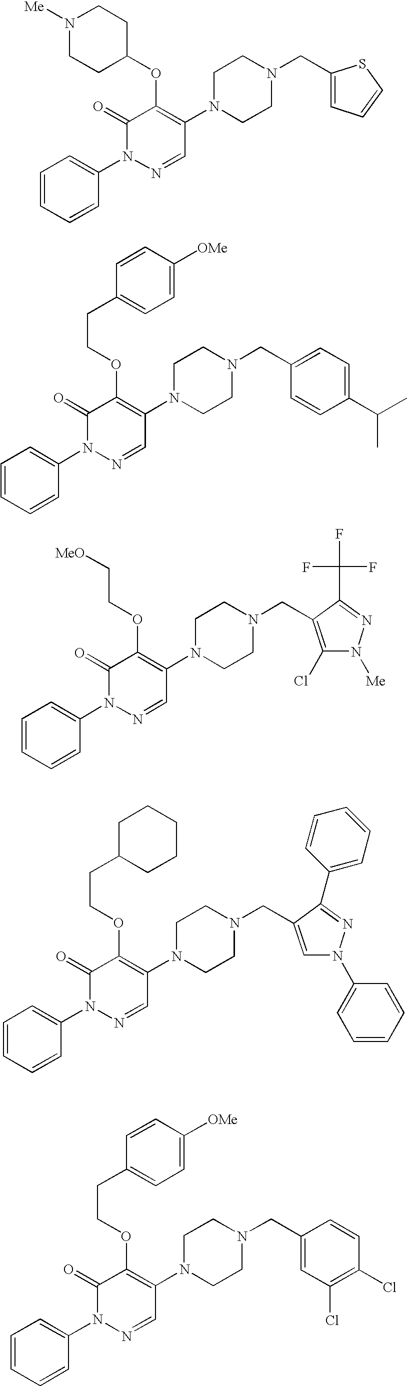 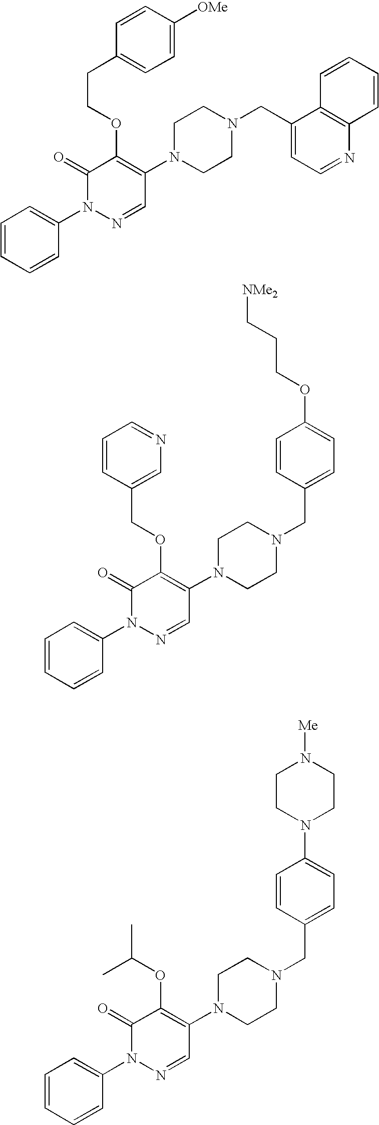 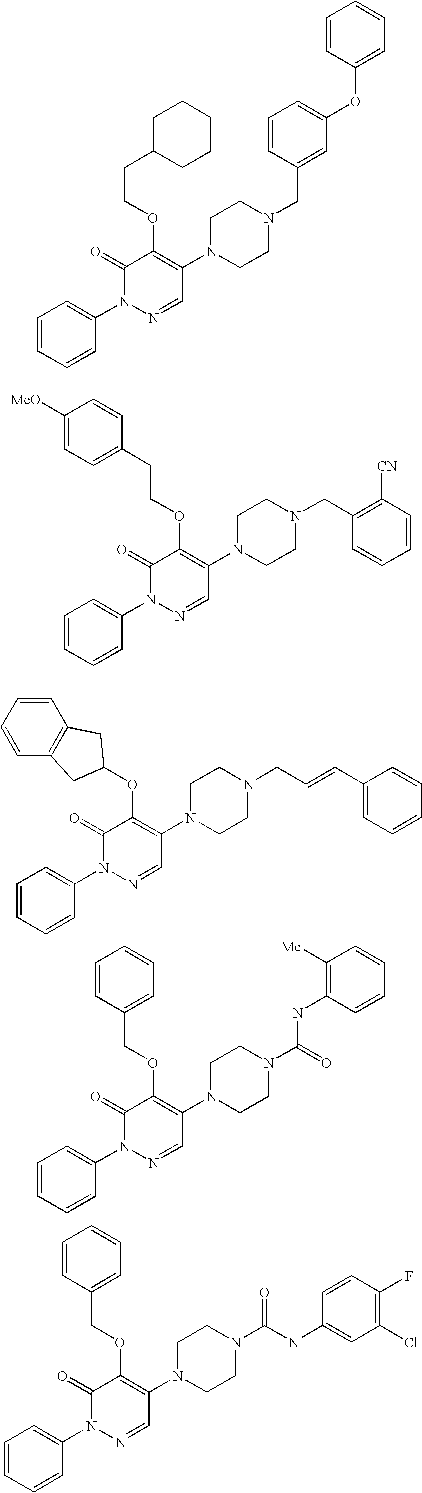 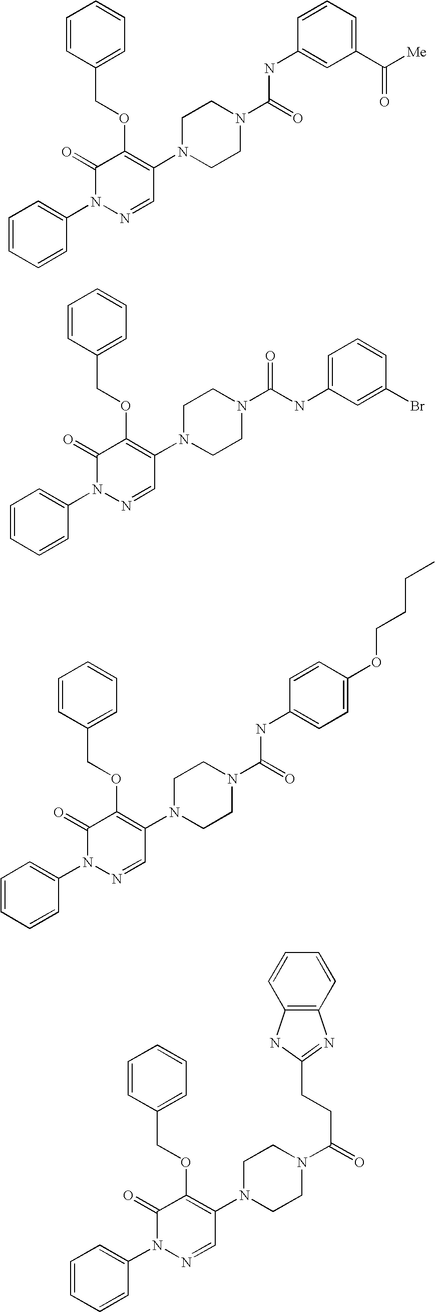 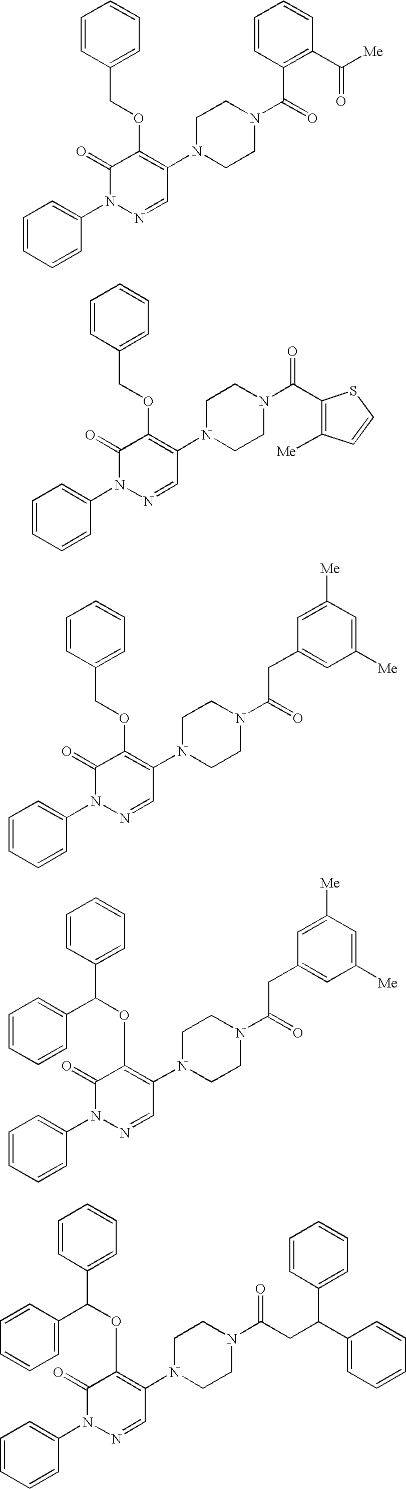 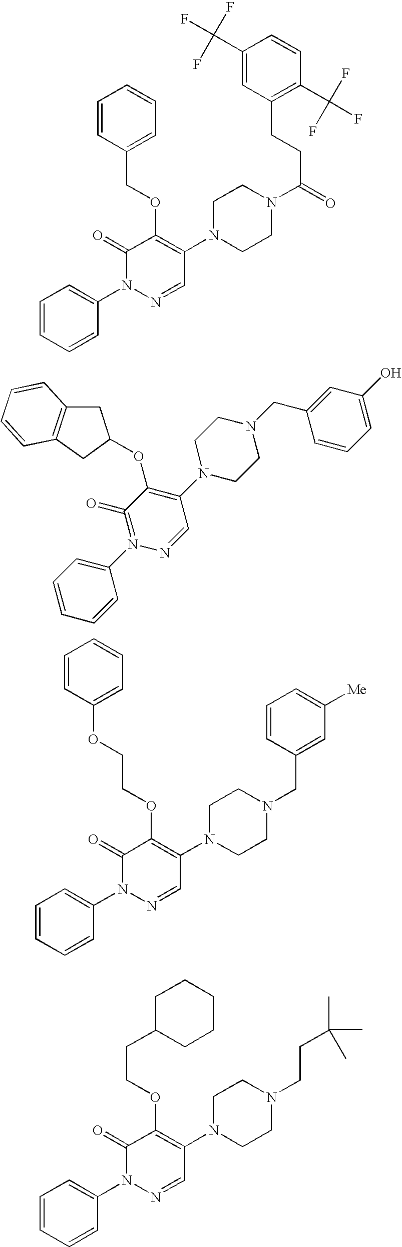 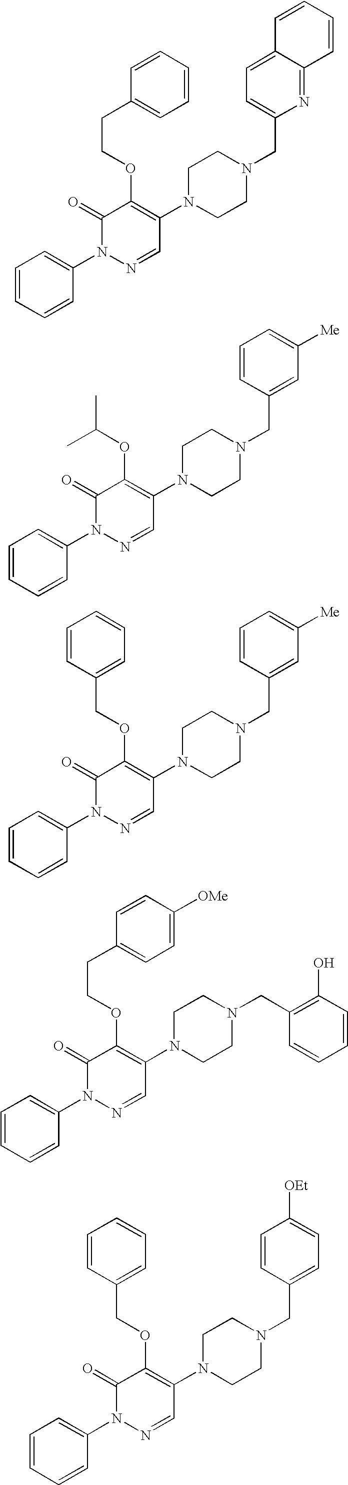 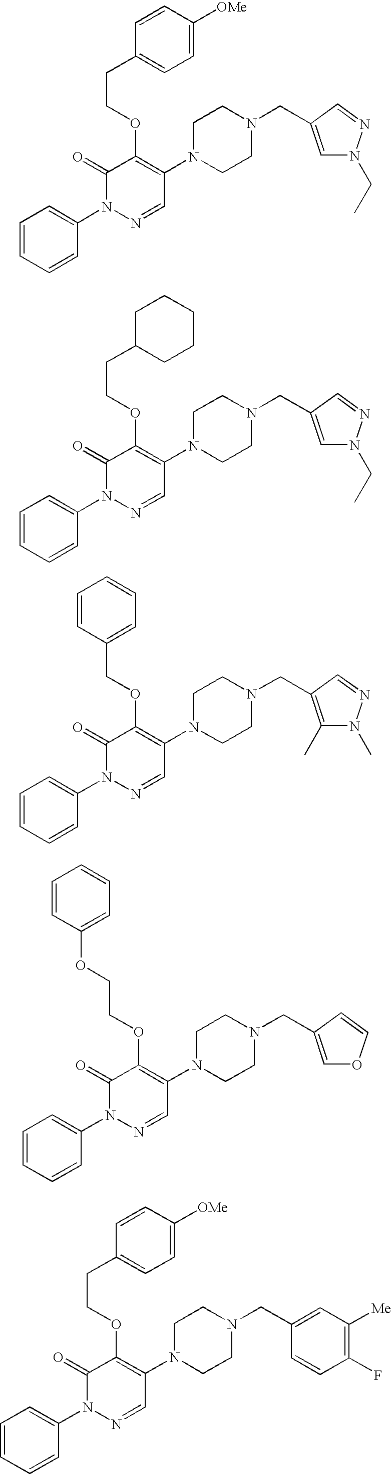 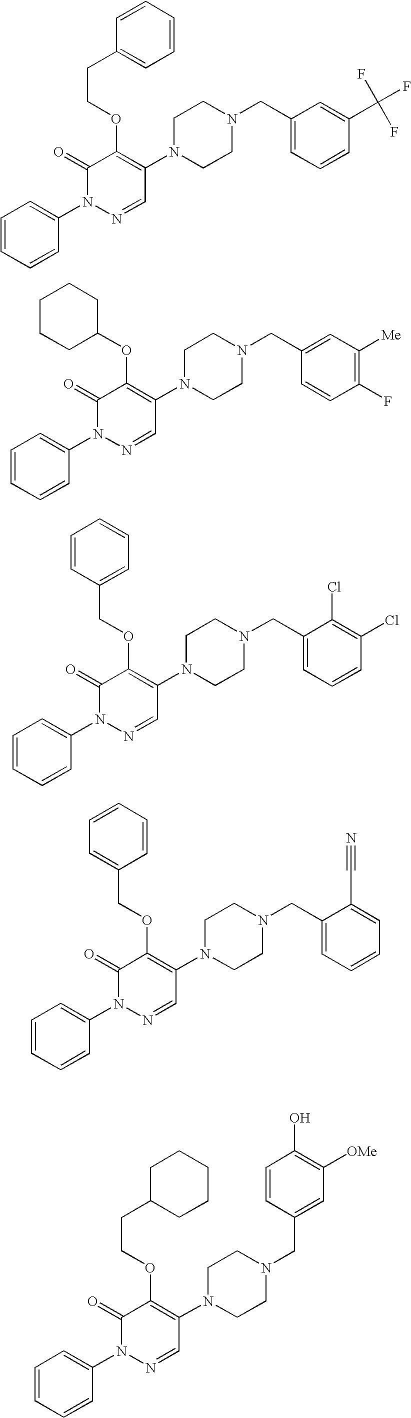 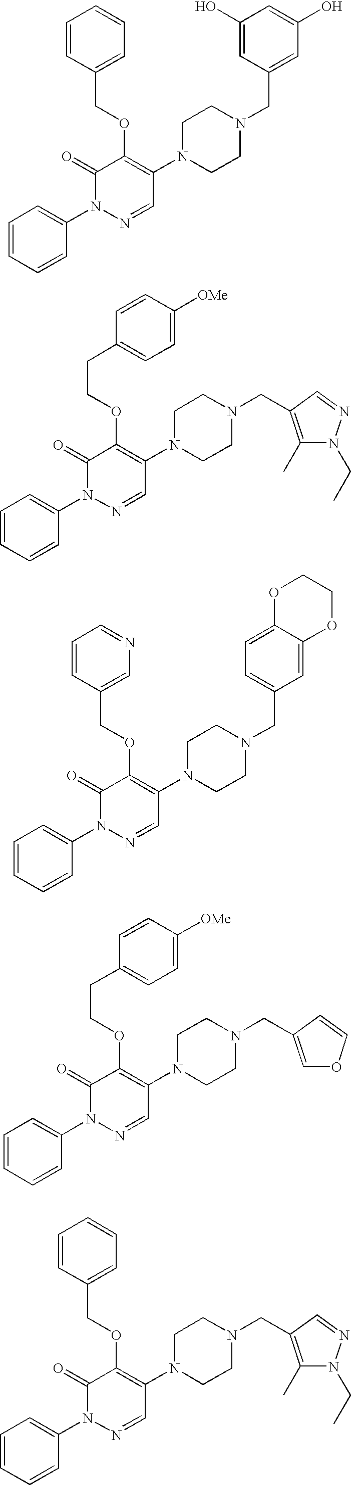 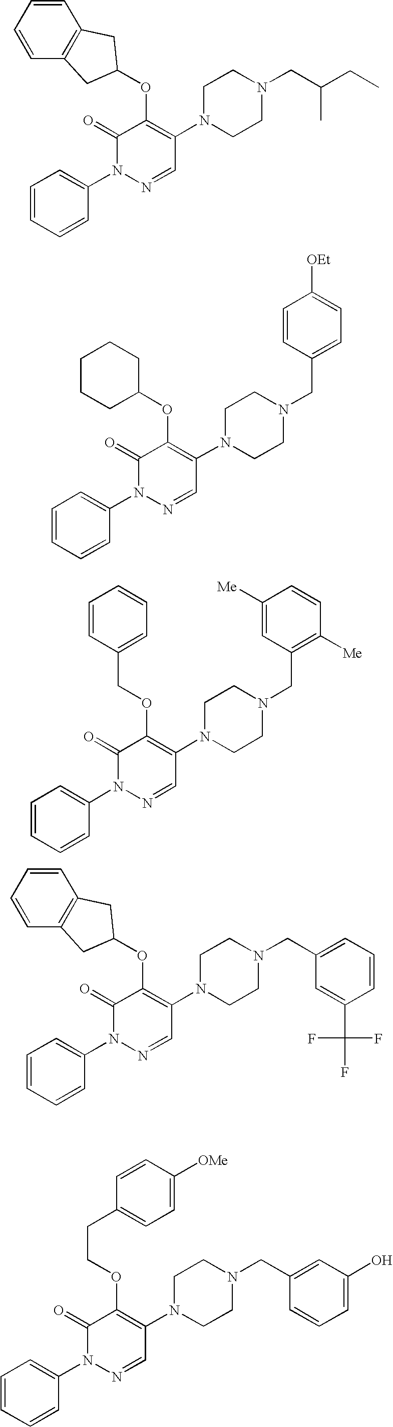 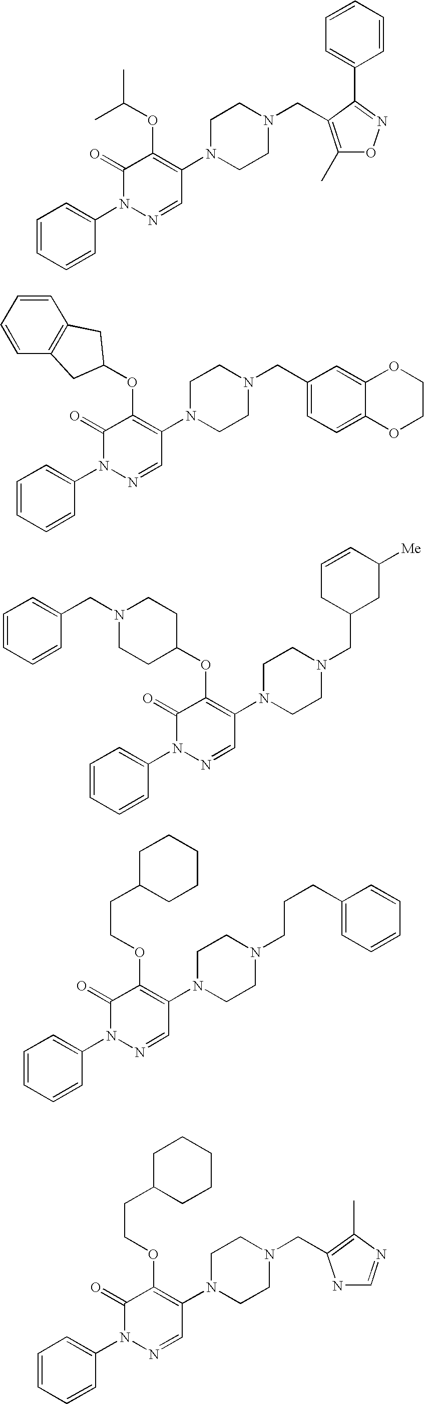 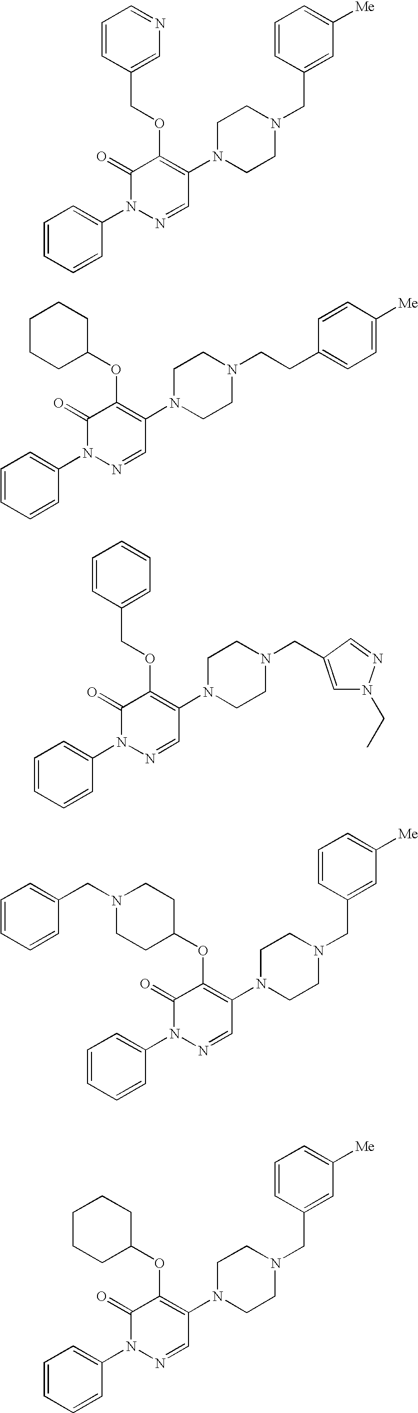 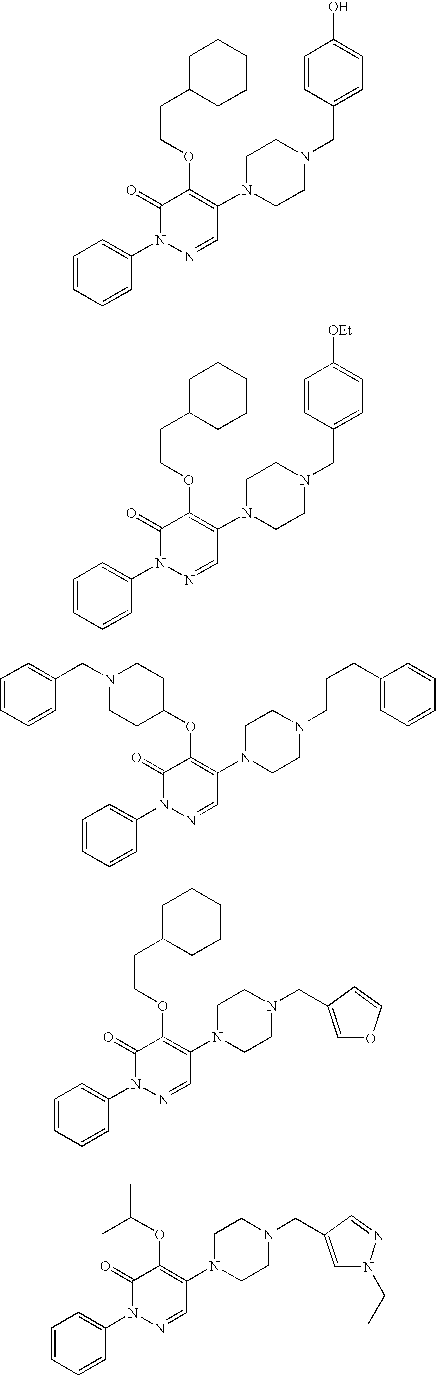 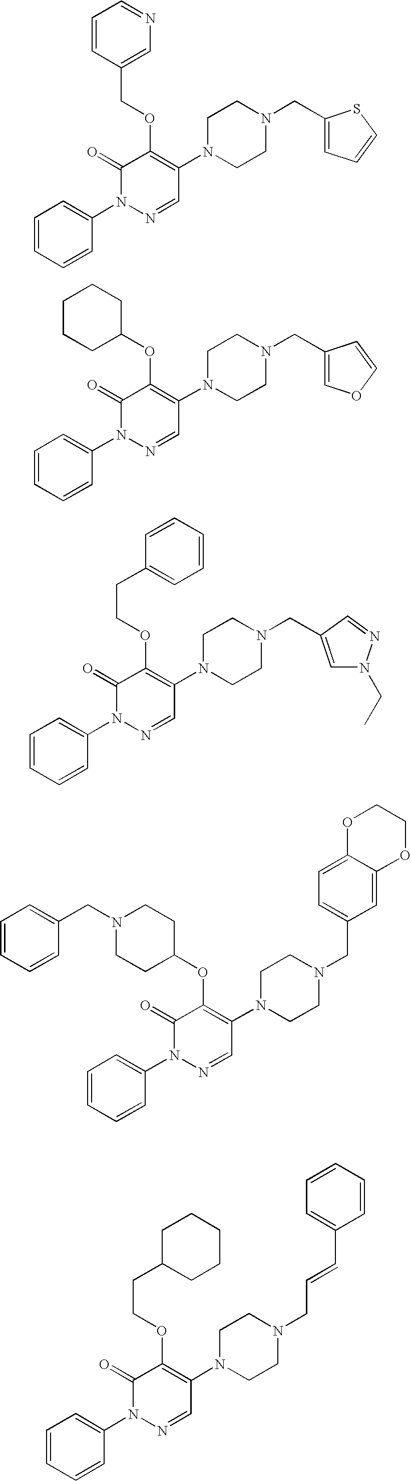 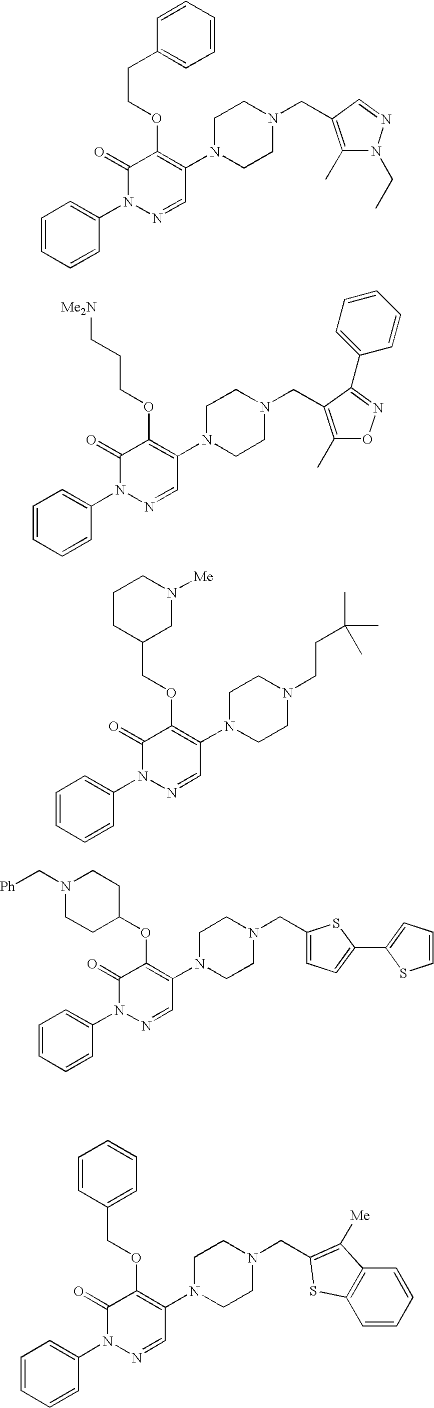 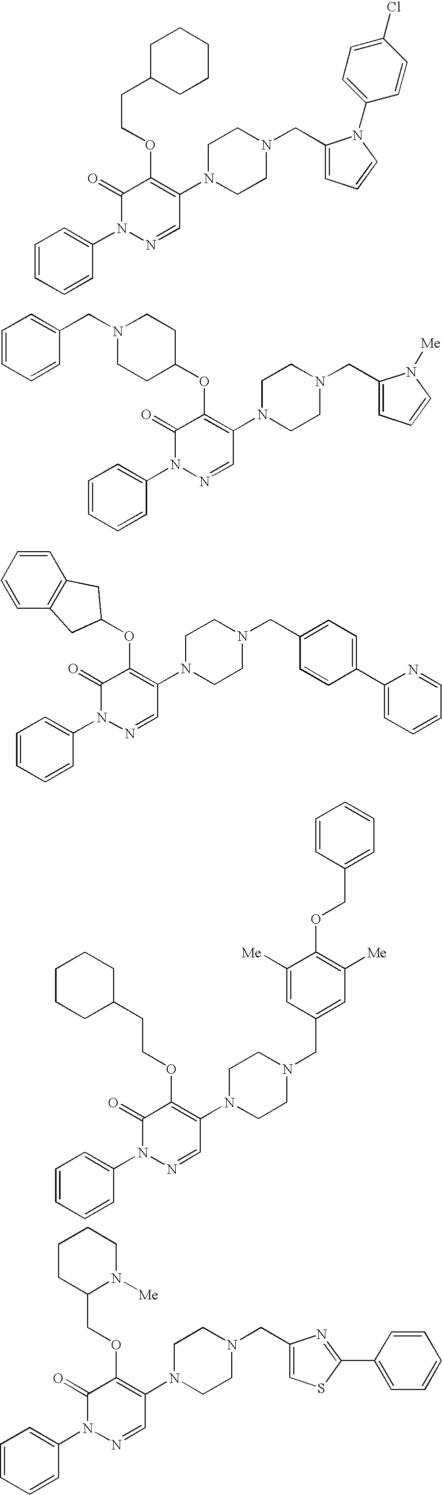 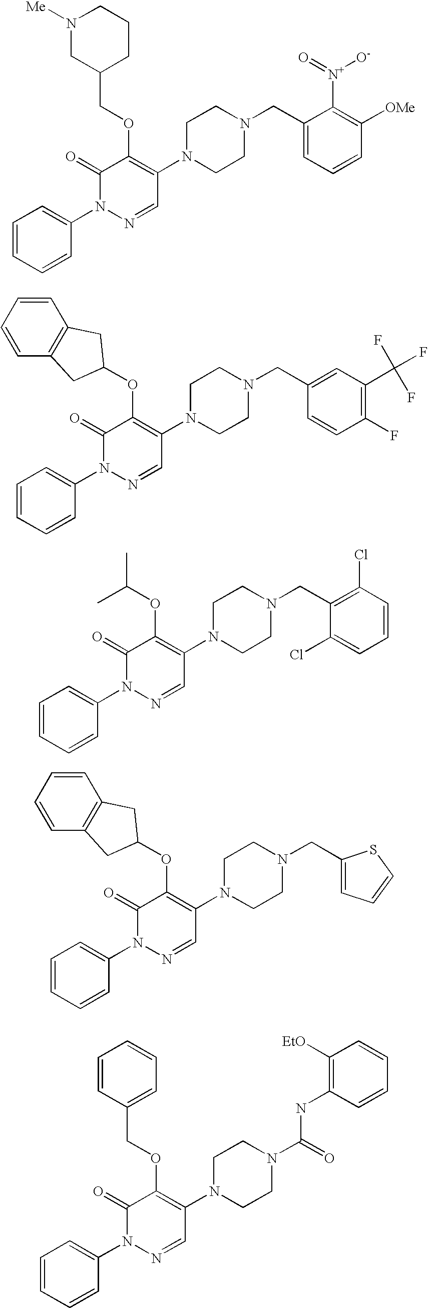 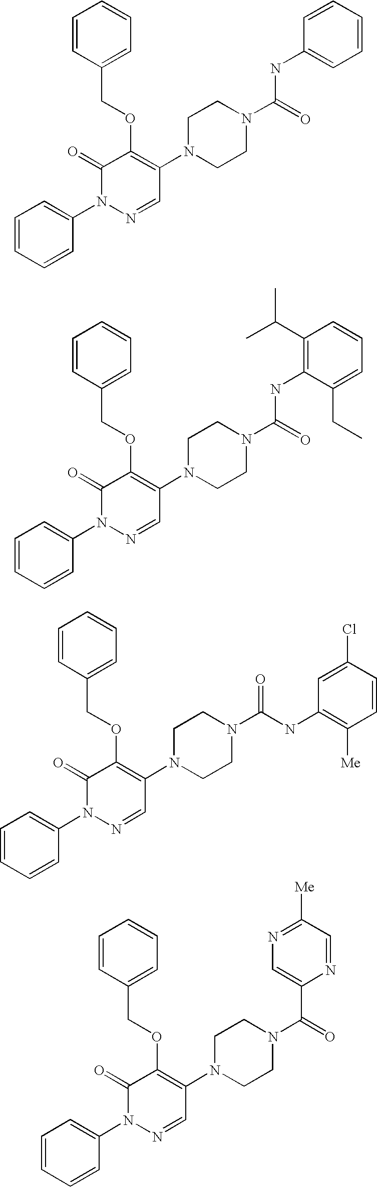 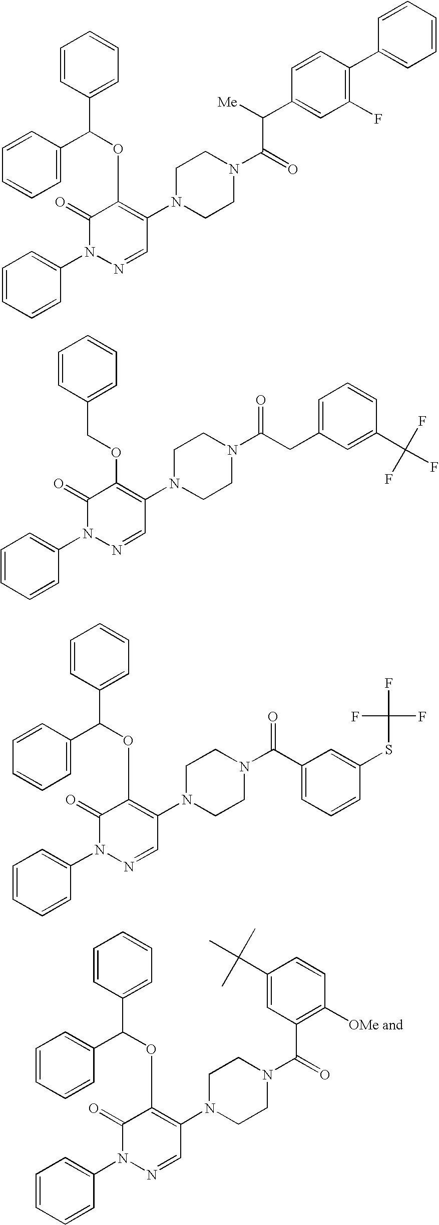 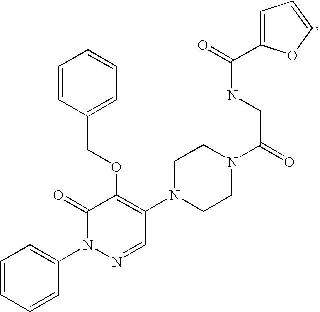
and the pharmaceutically acceptable salts and hydrates thereof, for the manufacture of a medicament for treating or preventing fungal infections in an animal or plant in need of such treatment or prevention.

2. The use of claim 1 wherein the compound is selected from the group consisting of: 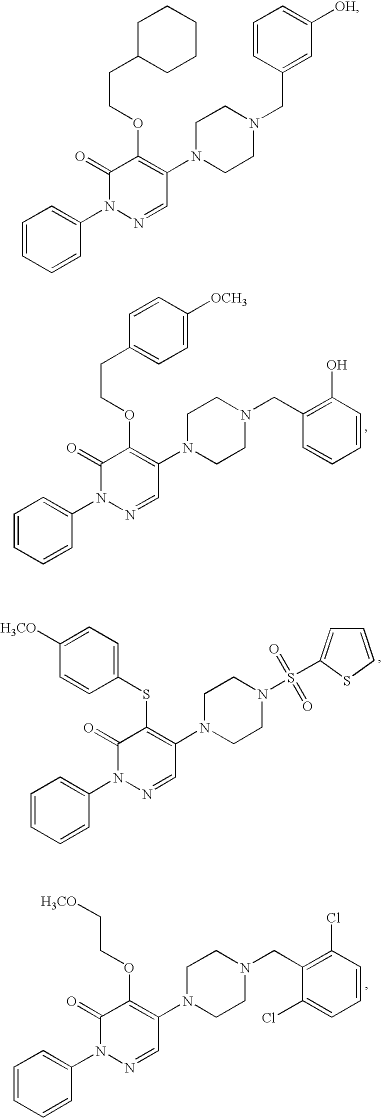 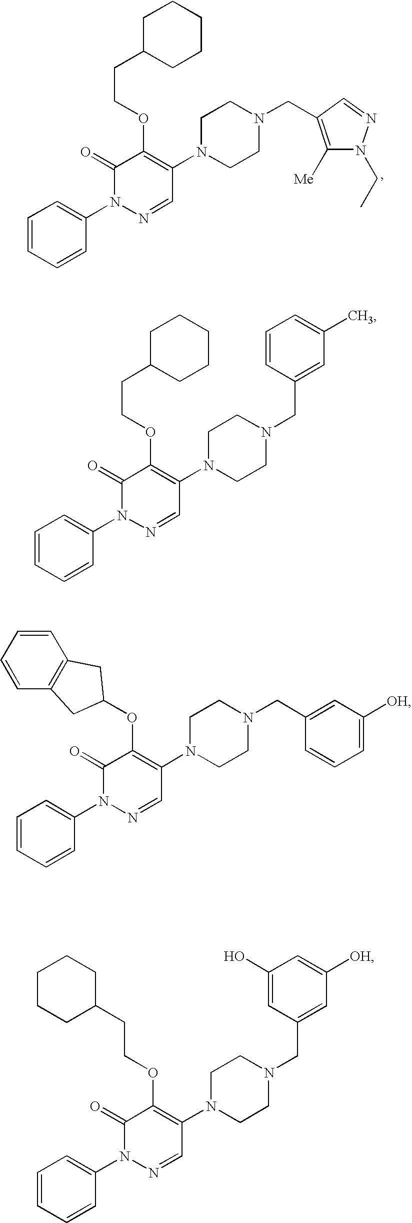 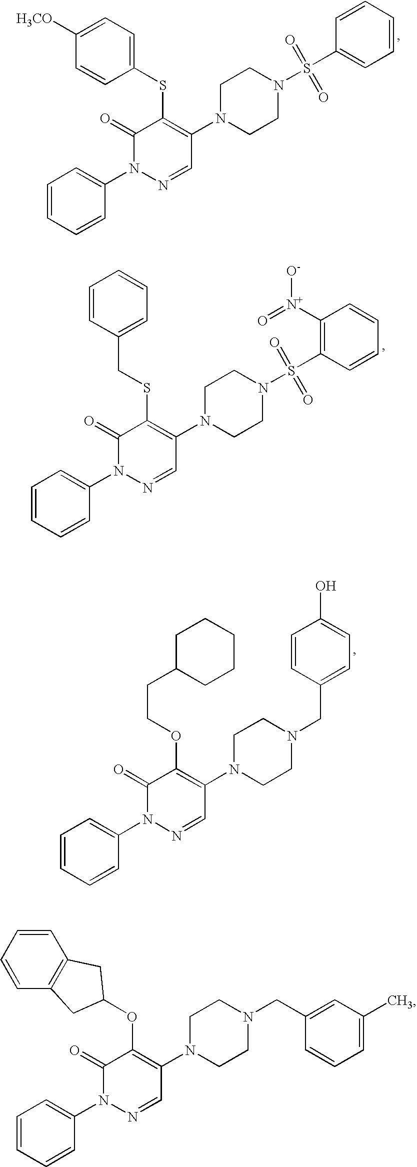 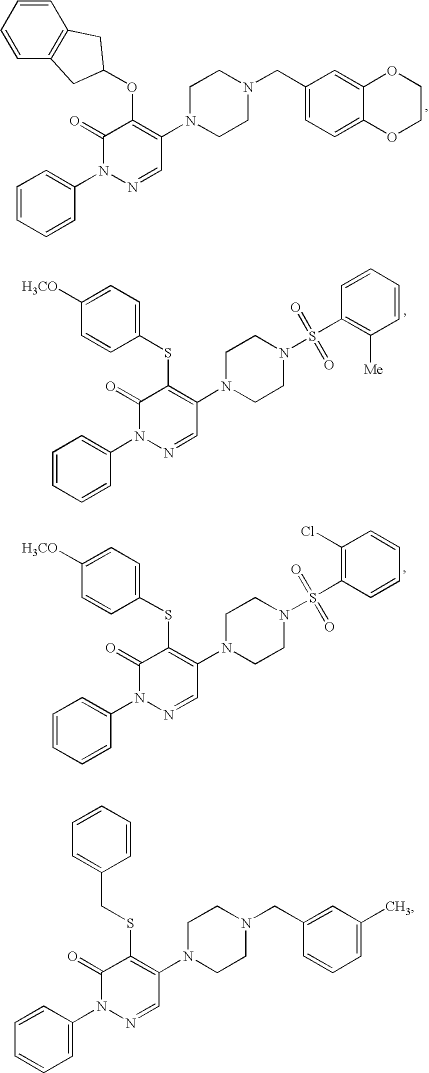 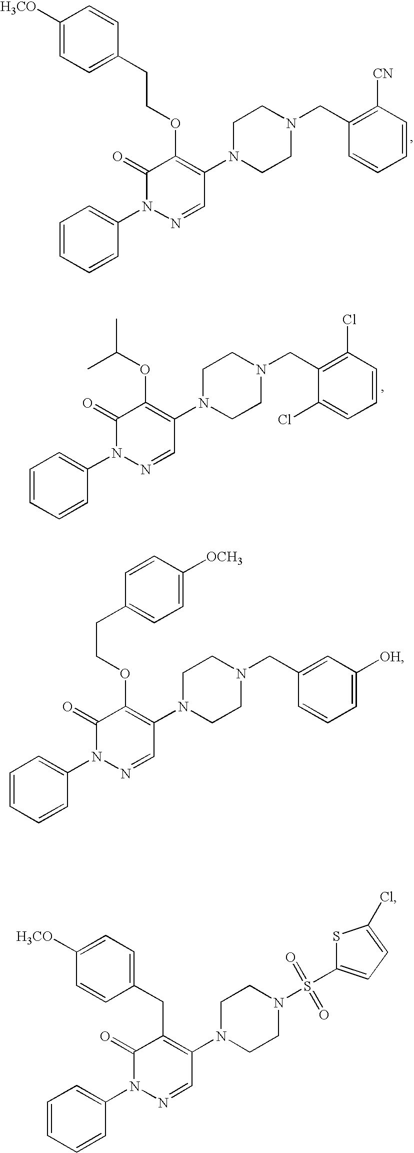 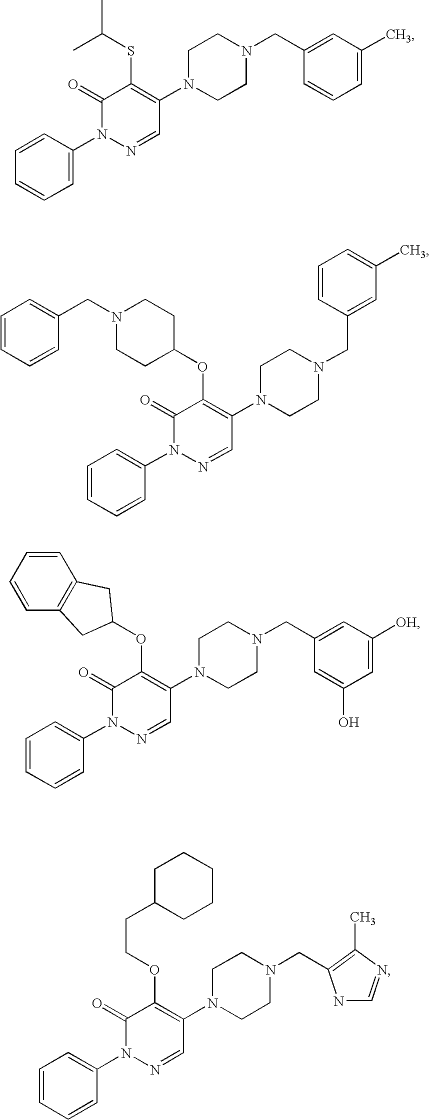 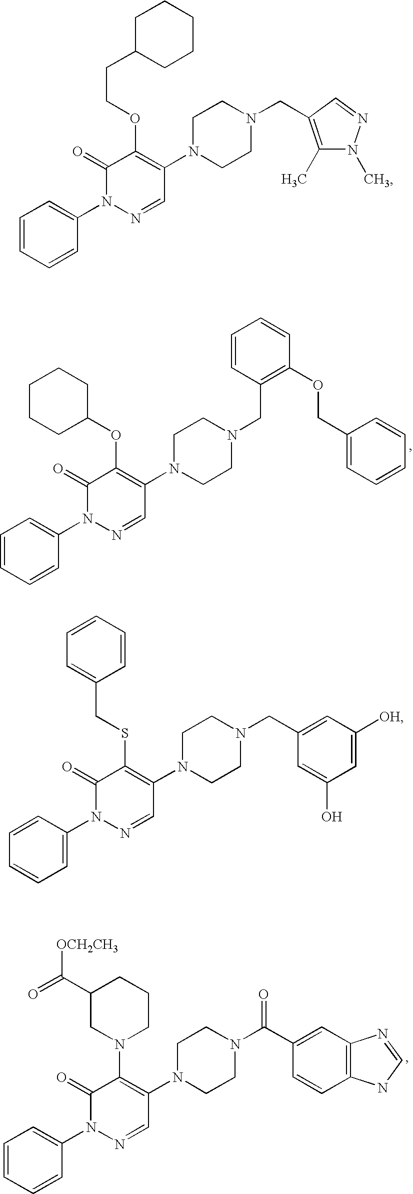 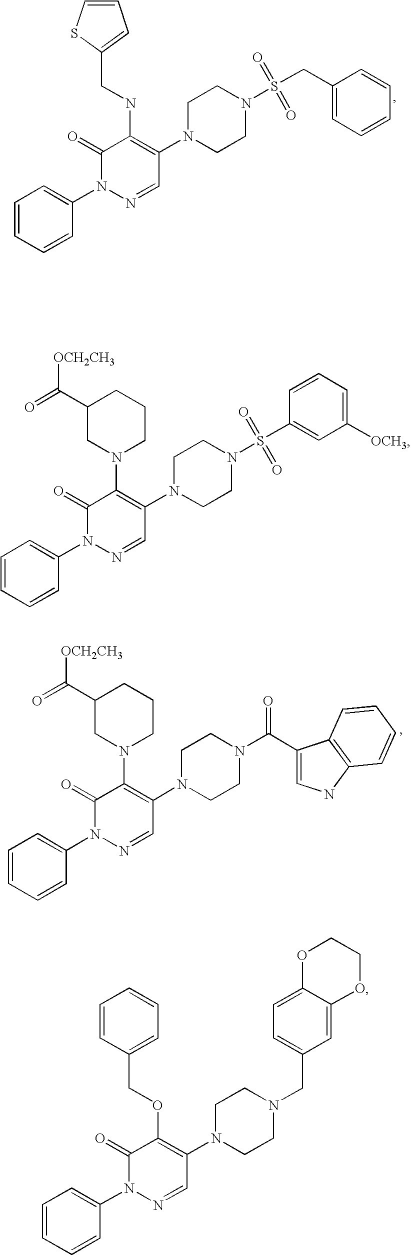 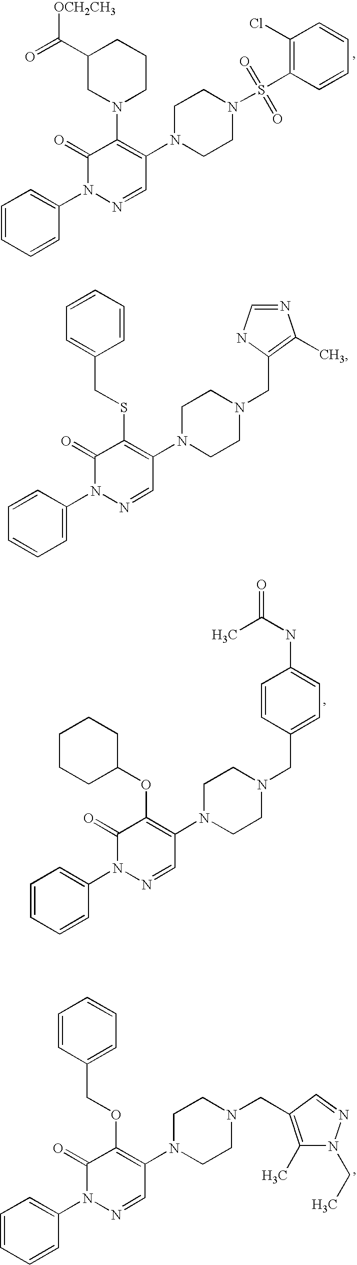 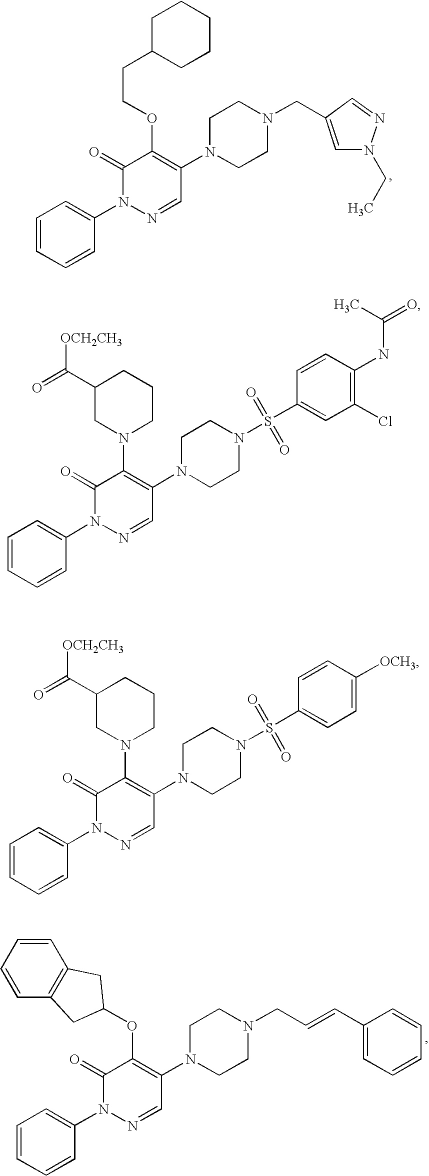 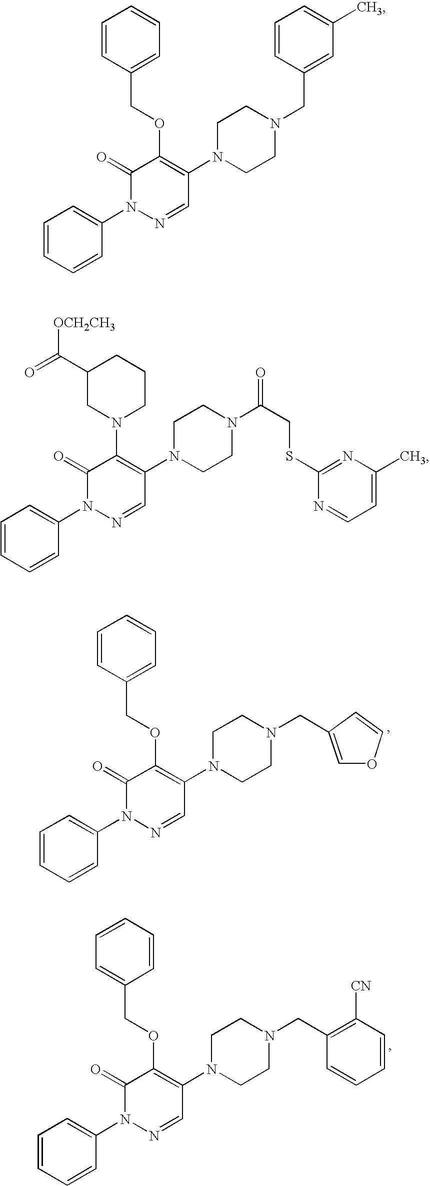 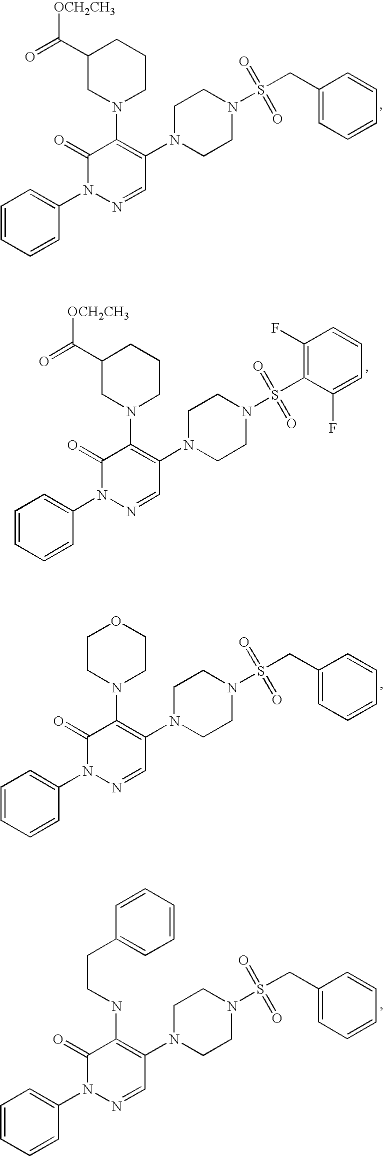 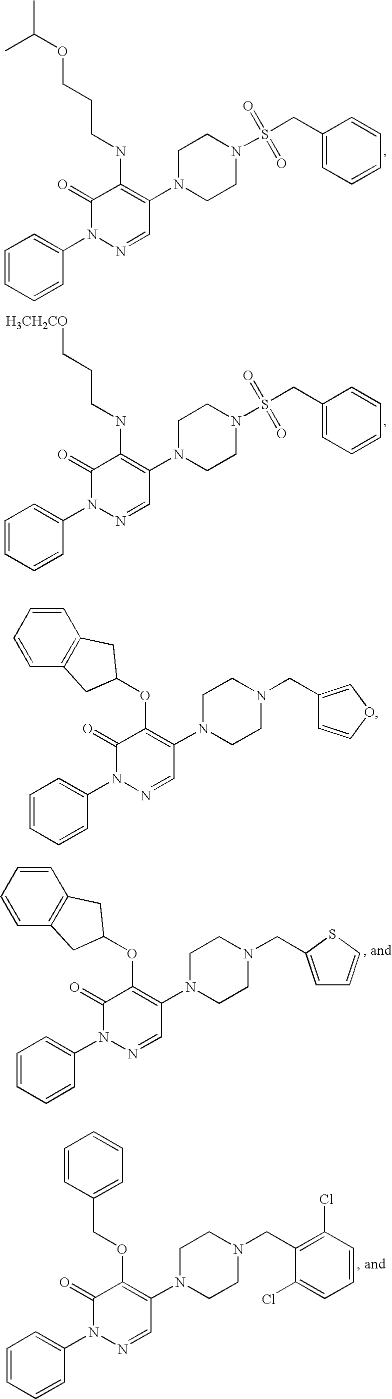
and the pharmaceutically acceptable salts and hydrates thereof.

3. The use of claim 1 wherein the compound is selected from the group consisting of: 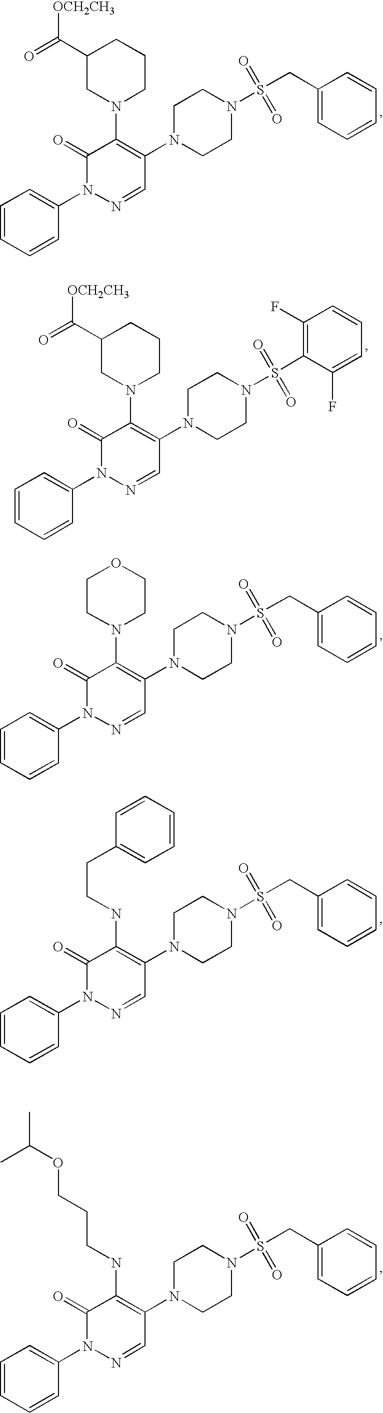 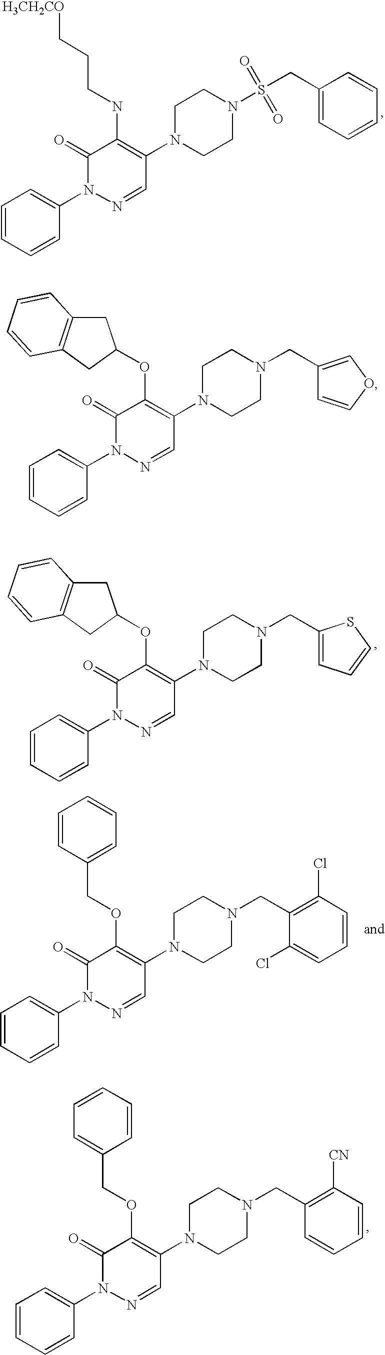
the pharmaceutically acceptable salts and hydrates thereof.

4. The use of claim 1 wherein said at least one compound is used for the manufacture of a medicament to treat or prevent infections from yeasts or moulds.

5. The use of claim 4 wherein yeasts are selected from the group comprising Candida, Cryptococcus, Pichia, Rhodotorula, Saccharomyces and Trichosporon, and moulds are selected from the group comprising Absidia, Alternaria, Apophysomyces, Aspergillus, Bjerkandera, Blastomyces, Coccidioides, Cunninghamella, Exophiala, Fusarium, Histoplasma, Microsporum, Mucor, Paecilomyces, Penicillium, Pseudallescheria, Ramichloridium, Rhizomucor, Rhizopus, Saksenaea, Scedosporium, Sporothrix, Trichophyton and Wangiella.

6. The use of claim 1 wherein the animal is a mammal, a fish, a bird, a crustacean or a reptile.

7. The use of claim 1 wherein the animal is a human.

8. The use of claim 1 wherein at least one compound as defined in claim 1 is used to treat or prevent infections from Microsporum or Malassezia.

9. The use of claim 8 wherein at least one compound as defined in claim 1 is used to treat or prevent growth of the fungal pathogens selected from the group comprising Alternaria, Aphanomyces, Ascophyta, Biploaris, Botrytis, Cercospora, Claviceps, Cochlioobolus, Colletotrichum, Erysiphe, Gibberella, Glomerella, Gomyces, Guignardia, Helminthosporium, Leptosphaeria, Magnaporthe, Microdochium, Monolinia, Mycosphaerella, Nectria, Penicillium, Plasmopara, Podosphaera, Puccinia, Pyrenophora, Pyricularia, Pythium, Phytophthora, Rhizoctonia, Sclerotina, Sclerotium, Septoria, Sphaerotheca, Thielaviopsis, Tilletia, Uncinula, Urocystis, Ustilago, Venturia and Verticillium.

10. The method of preparing a pharmaceutical composition comprising combining at least one compound as defined in claim 1 and a pharmaceutically acceptable carrier.

11. The method of claim 10 further comprising adding an additional antifungal agent.

12. The method of claim 11 wherein the additional antifungal agent is selected from the group consisting of azoles, echinocandins, polyenes, allylamines, thiocarbamates, nikkomycins, pradimicins, 5-fluorocytosines, oxaboroles, ciclopiroxolamine, griseofulvin, and morpholines.

13. The method of claim 12 wherein the additional antifungal agent is selected from the group consisting of fluconazole, miconazole, itraconazole, voriconazole, posaconazole, caspofungin, micafungin, anidulafungin, amphotericin B, liposomal formulations of amphotericin B, nystatin, terbinafine, tolnaftate, nikkomycin, pradimicin, 5-fluorocytosine, oxaborole, ciclopiroxolamine, griseofulvin, and fenpropimorph.

14. The use of claim 1 wherein said medicament further comprises one or more additional antifungal agents.

15. The use of claim 14 wherein the additional antifungal agent is selected from the group consisting of azoles, echinocandins, polyenes, allylamines, thiocarbamates, nikkomycins, pradimicins, 5-fluorocytosines, oxaboroles, ciclopiroxolamine, griseofulvin, and morpholines.

16. The use of claim 14 wherein the additional antifungal agent is selected from the group consisting of fluconazole, miconazole, itraconazole, voriconazole, posaconazole, caspofungin, micafungin, anidulafungin, amphotericin B, liposomal formulations of amphotericin B, nystatin, terbinafine, tolnaftate, nikkomycin, pradimicin, 5-fluorocytosine, oxaborole, ciclopiroxolamine, griseofulvin, and fenpropimorph.

17. The method of preparing a kit for treating or preventing fungal infections comprising in a single package, one container comprising one or more compounds as defined in claim 1 in a pharmaceutically acceptable carrier, and one or more separate containers, each comprising one or more additional antifungal agent in a pharmaceutically acceptable carrier.

18. The use of the pharmaceutical composition as defined in claim 10 for the manufacture of a medicament for treating or preventing fungal infections in an animal or plant.

19. The use of the pharmaceutical composition as defined in claim 11 for the manufacture of a medicament for treating or preventing fungal infections.

20. The use of the pharmaceutical composition as defined in claim 12 for the manufacture of a medicament for treating or preventing fungal infections.

21. The use of the pharmaceutical composition as defined in claim 13 for the manufacture of a medicament for treating or preventing fungal infections.

22. The use of claim 1, wherein said use is for treating or preventing growth of fungal pathogens on inanimate surface.

23. The use of claim 22, wherein said medicament further comprises one or more additional antifungal agents.

24. The use of at least one compound listed in claim 1 or a pharmaceutically acceptable salt, or hydrate thereof, for the manufacture of a medicament for inhibiting one or more glucan synthase.
